(19) United States
(12) Patent Application Publication
(10) Pub. No.: US 2013/0023494 A1
(43) Pub. Date: Jan. 24, 2013

(54) MODULATORS OF THE GPR119 RECEPTOR AND THE TREATMENT OF DISORDERS RELATED THERETO

(71) Applicants: Robert M. Jones, San Diego, CA (US); Sangdon Han, San Diego, CA (US); Daniel J. Buzard, San Diego, CA (US); Juerg Lehmann, San Diego, CA (US); Sanju Narayanan, Durham, NC (US); Dawei Yue, San Diego, CA (US)

(72) Inventors: Robert M. Jones, San Diego, CA (US); Sangdon Han, San Diego, CA (US); Daniel J. Buzard, San Diego, CA (US); Juerg Lehmann, San Diego, CA (US); Sanju Narayanan, Durham, NC (US); Dawei Yue, San Diego, CA (US)

(73) Assignee: ARENA PHARMACEUTICALS, INC., San Diego, CA (US)

(21) Appl. No.: 13/639,652

(22) PCT Filed: Apr. 5, 2011
(86) PCT No.: PCT/US11/31243

§ 371 Date: Oct. 5, 2012

Related U.S. Application Data
(60) Provisional application No. 61/341,866, filed on Apr. 6, 2010.
(60) Provisional application No. 61/401,863, filed on Aug. 20, 2010.

(51) Int. Cl. A61K 31/445; C07D 413/04; A61K 31/454; C07D 401/04; A61K 31/506; C07D 401/14; C07D 401/12; A61K 31/497; A61K 31/501; C07D 417/14; A61K 31/541; A61K 31/4523; A61P 19/00; A61P 25/00; A61P 3/00; A61P 3/10; A61P 3/04; A61K 31/4985; A61K 31/69; A61K 31/4745; A61K 31/522; A61K 31/53; A61K 31/519; C07D 211/46

(52) U.S. Cl. 514/64; 546/221; 514/327; 546/209; 514/326; 544/332; 514/275; 544/331; 544/408; 514/255.05; 544/238; 514/252.03; 514/252.02; 544/58.2; 514/227.8; 546/208; 514/210.18; 514/249; 514/294; 514/263.2; 514/245; 514/265.1

(57) ABSTRACT

The present invention relates to compounds of Formula (Ia) and pharmaceutically acceptable salts, solvates, and hydrates thereof, that are useful as a single agent or in combination with one or more additional pharmaceutical agents, such as, an inhibitor of DPP-IV, a biguanide, an SGLT2 inhibitor, or an alpha-glucosidase inhibitor, in the treatment of, for example, a disorder selected from: a GPR119-receptor-related disorder; a condition ameliorated by increasing a blood incretin level; a metabolic-related disorder; type 2 diabetes; obesity; and complications related thereto.

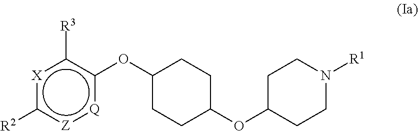

### FIELD OF THE INVENTION

The present invention relates to compounds of Formula (Ia) and pharmaceutically acceptable salts, solvates, and hydrates thereof, that are useful as a single agent or in combination with one or more additional pharmaceutical agents, such as, an inhibitor of DPP-IV, a biguanide, an SGLT2 inhibitor, or an alpha-glucosidase inhibitor, in the treatment of, for example, a disorder selected from: a GPR119-receptor-related disorder; a condition ameliorated by increasing a blood incretin level; a metabolic-related disorder; type 2 diabetes; obesity; and complications related thereto.

### BACKGROUND OF THE INVENTION

A. Diabetes Mellitus

Diabetes mellitus is a serious disease afflicting over 100 million people worldwide. In the United States, there are more than 12 million diabetics, with 600,000 new cases diagnosed each year.

Diabetes mellitus is a diagnostic term for a group of disorders characterized by abnormal glucose homeostasis resulting in elevated blood sugar. There are many types of diabetes, but the two most common are type 1 (also referred to as insulin-dependent diabetes mellitus or IDDM) and type 2 (also referred to as non-insulin-dependent diabetes mellitus or NIDDM).

The etiology of the different types of diabetes is not the same; however, everyone with diabetes has two things in common: overproduction of glucose by the liver and little or no ability to move glucose out of the blood into the cells where it becomes the body's primary fuel.

People who do not have diabetes rely on insulin, a hormone made in the pancreas, to move glucose from the blood into the cells of the body. However, people who have diabetes either don't produce insulin or can't efficiently use the insulin they produce; therefore, they can't move glucose into their cells. Glucose accumulates in the blood creating a condition called hyperglycemia, and over time, can cause serious health problems.

Diabetes is a syndrome with interrelated metabolic, vascular, and neuropathic components. The metabolic syndrome, generally characterized by hyperglycemia, comprises alterations in carbohydrate, fat and protein metabolism caused by absent or markedly reduced insulin secretion and/or ineffective insulin action. The vascular syndrome consists of abnormalities in the blood vessels leading to cardiovascular, retinal and renal complications. Abnormalities in the peripheral and autonomic nervous systems are also part of the diabetic syndrome.

About 5% to 10% of the people who have diabetes have IDDM. These individuals don't produce insulin and therefore must inject insulin to keep their blood glucose levels normal. IDDM is characterized by low or undetectable levels of endogenous insulin production caused by destruction of the insulin-producing β cells of the pancreas, the characteristic that most readily distinguishes IDDM from NIDDM. IDDM, once termed juvenile-onset diabetes, strikes young and older adults alike.

Approximately 90 to 95% of people with diabetes have type 2 (or NIDDM). NIDDM subjects produce insulin, but the cells in their bodies are insulin resistant: the cells don't respond properly to the hormone, so glucose accumulates in their blood. NIDDM is characterized by a relative disparity between endogenous insulin production and insulin requirements, leading to elevated blood glucose levels. In contrast to IDDM, there is always some endogenous insulin production in NIDDM; many NIDDM patients have normal or even elevated blood insulin levels, while other NIDDM patients have inadequate insulin production (Rotwein, R. et al. N. Engl. J. Med. 308, 65-71 (1983)). Most people diagnosed with NIDDM are age 30 or older, and half of all new cases are age 55 and older. Compared with whites and Asians, NIDDM is more common among Native Americans, African-Americans, Latinos, and Hispanics. In addition, the onset can be insidious or even clinically inapparent, making diagnosis difficult.

The primary pathogenic lesion on NIDDM has remained elusive. Many have suggested that primary insulin resistance of the peripheral tissues is the initial event. Genetic epidemiological studies have supported this view. Similarly, insulin secretion abnormalities have been argued as the primary defect in NIDDM. It is likely that both phenomena are important contributors to the disease process (Rimoin, D. L., et. al. Emery and Rimoin's Principles and Practice of Medical Genetics 3^{rd} Ed. 1:1401-1402 (1996)).

Many people with NIDDM have sedentary lifestyles and are obese: they weigh approximately 20% more than the recommended weight for their height and build. Furthermore, obesity is characterized by hyperinsulinemia and insulin resistance, a feature shared with NIDDM, hypertension and atherosclerosis.

The patient with diabetes faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions (Perry, I. J., et al., BMJ 310, 560-564 (1995)).

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina at the back of the eye and increases risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

B. Obesity

Obesity and diabetes are among the most common human health problems in industrialized societies. In industrialized countries a third of the population is at least 20% overweight. In the United States, the percentage of obese people has increased from 25% at the end of the 1970's, to 33% at the beginning the 1990's. Obesity is one of the most important risk factors for NIDDM. Definitions of obesity differ, but in general, a subject weighing at least 20% more than the recommended weight for his/her height and build is considered obese. The risk of developing NIDDM is tripled in subjects 30% overweight, and three-quarters with NIDDM are overweight.

Obesity, which is the result of an imbalance between caloric intake and energy expenditure, is highly correlated with insulin resistance and diabetes in experimental animals and human. However, the molecular mechanisms that are involved in obesity-diabetes syndromes are not clear. During early development of obesity, increased insulin secretion balances insulin resistance and protects patients from hyperglycemia (Le Stunff, et al. Diabetes 43, 696-702 (1989)). However, after several decades, β cell function deteriorates and non-insulin-dependent diabetes develops in about 20% of the obese population (Pederson, P. Diab. Metab. Rev. 5, 505-509 (1989)) and (Brancati, F. L., et al., Arch. Intern. Med. 159, 957-963 (1999)). Given its high prevalence in modern societies, obesity has thus become the leading risk factor for NIDDM (Hill, J. O., et al., Science 280, 1371-1374 (1998)). However, the factors which predispose a fraction of patients to alteration of insulin secretion in response to fat accumulation remain unknown.

Whether someone is classified as overweight or obese can be determined by a number of different methods, such as, on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see TABLE below). There are problems with this definition in that it does not take into account the proportion of body mass that is muscle in relation to fat (adipose tissue). To account for this, alternately, obesity can be defined on the basis of body fat content: greater than 25% and 30% in males and females, respectively.

CLASSIFICATION OF WEIGHT BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Obesity considerably increases the risk of developing cardiovascular diseases as well. Coronary insufficiency, atheromatous disease, and cardiac insufficiency are at the forefront of the cardiovascular complication induced by obesity. It is estimated that if the entire population had an ideal weight, the risk of coronary insufficiency would decrease by 25% and the risk of cardiac insufficiency and of cerebral vascular accidents by 35%. The incidence of coronary diseases is doubled in subjects less than 50 years of age who are 30% overweight.

C. Atherosclerosis

Atherosclerosis is a complex disease characterized by inflammation, lipid accumulation, cell death and fibrosis. Atherosclerosis is characterized by cholesterol deposition and monocyte infiltration into the subendothelial space, resulting in foam cell formation. Thrombosis subsequent to atherosclerosis leads to myocardial infarction and stroke. Atherosclerosis is the leading cause of mortality in many countries, including the United States. (See, e.g., Ruggeri, Nat Med (2002) 8:1227-1234; Arehart et al, Circ Res, Circ. Res. (2008) 102:986-993.)

D. Osteoporosis

Osteoporosis is a disabling disease characterized by the loss of bone mass and microarchitectural deterioration of skeletal structure leading to compromised bone strength, which predisposes a patient to increased risk of fragility fractures. Osteoporosis affects more than 75 million people in Europe, Japan and the United States, and causes more than 2.3 million fractures in Europe and the United States alone. In the United States, osteoporosis affects at least 25% of all post-menopausal white women, and the proportion rises to 70% in women older than 80 years. One in three women older than 50 years will have an osteoporotic fracture that causes a considerable social and financial burden on society. The disease is not limited to women; older men also can be affected. By 2050, the worldwide incidence of hip fracture in men is projected to increase by 310% and 240% in women. The combined lifetime risk for hip, forearm, and vertebral fractures presenting clinically is around 40%, equivalent to the risk for cardiovascular disease. Osteoporotic fractures therefore cause substantial mortaility, morbidity, and economic cost. With an ageing population, the number of osteoporotic fractures and their costs will at least double in the next 50 years unless effective preventive strategies are developed. (See, e.g., Atik et al., Clin Orthop Relat Res (2006) 443:19-24; Raisz, J Clin Invest (2005) 115:3318-3325; and World Health Organization Technical Report Series 921 (2003), Prevention and Management of Osteopoosis.)

E. Inflammatory Bowel Disease (IBD)

Inflammatory bowel disease (IBD) is the general name for diseases that cause inflammation in the intestines and includes, e.g. Crohn's disease, ulcerative colitis, ulcerative proctitis. U.S. medical costs of inflammatory bowel disease for 1990 have been estimated to be $1.4 to $1.8 billion. Lost productivity has been estimated to have added an additional $0.4 to $0.8 billion, making the estimated cost of inflammatory bowel disease $1.8 to $2.6 billion. (See, e.g., Pearson, Nursing Times (2004) 100:86-90; Hay et al, J Clin Gastroenterol (1992) 14:309-317; Keighley et al, Ailment Pharmacol Ther (2003) 18:66-70.)

Enteritis refers to inflammation of the intestine, especially the small intestine, a general condition that can have any of numerous different causes. Enterocolitis refers to inflammation of the small intestine and colon.

Crohn's disease (CD) is an inflammatory process that can affect any portion of the digestive tract, but is most commonly seen in the last part of the small intestine otherwise called the (terminal) ileum and cecum. Altogether this area is also known as the ileocecal region. Other cases may affect one or more of: the colon only, the small bowel only (duodenum, jejunum and/or ileum), the anus, stomach or esophagus. In contrast with ulcerative colitis, CD usually does not affect the rectum, but frequently affects the anus instead. The inflammation extends deep into the lining of the affected organ. The inflammation can cause pain and can make the intestines empty frequently, resulting in diarrhea. Crohn's disease may also be called enteritis. Granulomatous colitis is another name for Crohn's disease that affects the colon. Ileitis is CD of the ileum which is the third part of the small intestine. Crohn's colitis is CD affecting part or all of the colon.

Ulcerative colitis (UC) is an inflammatory disease of the large intestine, commonly called the colon. UC causes inflammation and ulceration of the inner lining of the colon and rectum. The inflammation of UC is usually most severe in the rectal area with severity diminishing (at a rate that varies from patient to patient) toward the cecum, where the large and small intestine join. Inflammation of the rectum is called proctitis. Inflammation of the sigmoid colon (located just above the rectum) is called sigmoiditis. Inflammation involving the entire colon is termed pancolitis. The inflammation causes the colon to empty frequently resulting in diarrhea. As the lining of the colon is destroyed ulcers form releasing mucus, pus and blood. Ulcerative proctitis is a form of UC that affects only the rectum.

F. GPR119

GPR119 is a G protein-coupled receptor (GPR119; e.g., human GPR119, GenBank® Accession No. AAP72125 and alleles thereof; e.g., mouse GPR119, GenBank® Accession No. AY288423 and alleles thereof) and is selectively expressed on pancreatic beta cells. GPR119 activation leads to elevation of a level of intracellular cAMP, consistent with GPR119 being coupled to Gs. Agonists to GPR119 stimulate glucose-dependent insulin secretion in vitro and lower an elevated blood glucose level in vivo; see, e.g., International Applications WO 04/065380 and WO 04/076413, and EP 1338651, the disclosure of each of which is herein incorporated by reference in its entirety. In the literature, GPR119 has also been referred to as RUP3 (see, International Application WO 00/31258) and as Glucose-Dependent Insulinotropic Receptor GDIR (see, Jones, et. al. Expert Opin. Ther. Patents (2009), 19 (10): 1339-1359).

GPR119 agonists also stimulate the release of Glucose-dependent Insulinotropic Poloypeptide (GIP), Glucagon-Like Peptide-1 (GLP-1), and at least one other L-cell peptide, Peptide YY (PYY) (Jones, et. al. Expert Opin. Ther. Patents (2009), 19 (10): 1339-1359); for specific references related to GPR119 agonists and the release of:

GIP, see Shah, Current Opinion in Drug Discovery & Development, (2009) 12:519-532; Jones, et al., Ann. Rep. Med. Chem., (2009) 44:149-170; WO 2007/120689; and WO 2007/120702;

GLP-1, see Shah, Current Opinion in Drug Discovery & Development, (2009) 12:519-532; Jones, et al., Ann. Rep. Med. Chem., (2009) 44:149-170; Schwartz et. al., Cell Metabolism, 2010, 11:445-447; and WO 2006/076231; and

PYY, see Schwartz et. al., Cell Metabolism, 2010, 11:445-447; and WO 2009/126245.

As mentioned above, GPR119 agonists enhance incretin release and therefore can be used in treatment of disorders related to the incretins, such as, GIP, GLP-1, and PYY. However, a number of the incretins, such as, GIP and GLP-1, are substrates for the enzyme DPP-IV. Jones and co-workers (Jones, et al., Ann. Rep. Med. Chem., (2009) 44:149-170) have demonstrated that a combined administration of a GPR119 agonist, (2-Fluoro-4-methanesulfonyl-phenyl)-{6-[4-(3-isopropyl-[1,2,4]oxadiazol-5-yl)-piperidin-1-yl]-5-nitro-pyrimidin-4-yl}-amine (see, compound B111 in WO 2004/065380), and a DPP-IV inhibitor acutely increased plasma GLP-1 levels and improved glucose tolerance to a significantly greater degree than either agent alone.

G. Glucose-Dependent Insulinotropic Poloypeptide (GIP)

Glucose-dependent insulinotropic polypeptide (GIP, also known as gastric inhibitory polypeptide) is a peptide incretin hormone of 42 amino acids that is released from duodenal endocrine K cells after meal ingestion. The amount of GIP released is largely dependent on the amount of glucose consumed. GIP has been shown to stimulate glucose-dependent insulin secretion in pancreatic beta cells. GIP mediates its actions through a specific G protein-coupled receptor, namely GIPR.

As GIP contains an alanine at position 2, it is an excellent substrate for dipeptidyl peptidase-4 (DPP-IV), an enzyme regulating the degradation of GIP. Full-length GIP(1-42) is rapidly converted to bioinactive GIP(3-42) within minutes of secretion from the gut K cell. Inhibition of DPP-IV has been shown to augment GIP bioactivity. (See, e.g., Drucker, Cell Metab (2006) 3:153-165; McIntosh et al., Regul Pept (2005) 128:159-165; Deacon, Regul Pept (2005) 128:117-124; and Ahren et al., Endocrinology (2005) 146:2055-2059). Analysis of full length bioactive GIP, for example in blood, can be carried out using N-terminal-specific assays (see, e.g., Deacon et al, J Clin Endocrinol Metab (2000) 85:3575-3581).

Recently, GIP has been shown to promote bone formation. GIP has been shown to activate osteoblastic receptors, resulting in increases in collagen type I synthesis and alkaline phosphatase activity, both associated with bone formation. GIP has been shown to inhibit osteoclast activity and differentiation in vitro. GIP administration has been shown to prevent the bone loss due to ovariectomy. GIP receptor (GIPR) knockout mice evidence a decreased bone size, lower bone mass, altered bone microarchitecture and biochemical properties, and altered parameters for bone turnover, especially in bone formation. (See, e.g., Zhong et al, Am J Physiol Endocrinol Metab (2007) 292:E543-E548; Bollag et al., Endocrinology (2000) 141:1228-1235; Bollag et al., Mol Cell Endocrinol (2001) 177:35-41; Xie et al., Bone (2005) 37:759-769; and Tsukiyama et al., Mol Endocrinol (2006) 20:1644-1651.)

The usefulness of GIP for maintaining or increasing bone density or formation has been acknowledged by the United State Trademark and Patent Office by issuance of U.S. Pat. No. 6,410,508 for the treatment of reduced bone mineralization by administration of GIP peptide. However, current GIP peptide agonists suffer from a lack of oral bioavailability, negatively impacting patient compliance. An attractive alternative approach is to develop an orally active composition for increasing an endogenous level of GIP activity.

H. Glucagon-Like Peptide-1 (GLP-1)

Glucagon-like peptide-1 (GLP-1) is an incretin hormone derived from the posttranslaltional modification of proglucagon and secreted by gut endocrine cells. GLP-1 mediates its actions through a specific G protein-coupled receptor (GPCR), namely GLP-1R. GLP-1 is best characterized as a hormone that regulates glucose homeostasis. GLP-1 has been shown to stimulate glucose-dependent insulin secretion and to increase pancreatic beta cell mass. GLP-1 has also been shown to reduce the rate of gastric emptying and to promote satiety. The efficacy of GLP-1 peptide agonists in controlling blood glucose in Type 2 diabetics has been demonstrated in several clinical studies [see, e.g., Nauck et al., Drug News Perspect (2003) 16:413-422], as has its efficacy in reducing body mass [Zander et al., Lancet (2002) 359:824-830].

GLP-1 receptor agonists are additionally useful in protecting against myocardial infarction and against cognitive and neurodegenerative disorders. GLP-1 has been shown to be cardioprotective in a rat model of myocardial infarction [Bose et al., Diabetes (2005) 54:146-151], and GLP-1R has been shown in rodent models to be involved in learning and neuroprotection [During et al., Nat. Med. (2003) 9:1173-1179; and Greig et al., Ann N Y Acad Sci (2004) 1035:290-315].

Certain disorders such as Type 2 diabetes are characterized by a deficiency in GLP-1 [see, e.g., Nauck et al., Diabetes (2004) 53 Suppl 3:S190-196].

Current GLP-1 peptide agonists suffer from a lack of oral bioavailability, negatively impacting patient compliance. Efforts to develop orally bioavailable non-peptidergic, small-molecule agonists of GLP-1R have so far been unsuccessful [Mentlein, Expert Opin Investig Drugs (2005) 14:57-64]. An attractive alternative approach is to develop an orally active composition for increasing an endogenous level of GLP-1 in the blood.

I. Peptide YY (PYY)

Peptide YY (PYY) is a 36 amino acid peptide originally isolated in 1980 from porcine intestine (Tatemoto et al, Nature (1980) 285:417-418). PYY is secreted from enteroendocrine L-cells within both the large and small intestine. It has been shown that in rat and human gut concentrations of immunoreactive PYY are low in duodenum and jenunum, high in ileum and colon, and highest in rectum (Lundberg et al, PNAS USA (1982) 79:4471-4475; Adrian et al, Gastroenterol. (1985) 89:1070-1077; Ekblad et al, Peptides (2002) 23:251-261; Ueno et al, Regul Pept (2008) 145:12-16). (PYY expression in rat been reported to also extend to alpha cells of the islets of Langerhans and to cells in the medulla oblongata (Ekblad et al, Peptides (2002) 23:251-261; PYY is released into the circulation as PYY₁₋₃₆ and PYY₃₋₃₆ (Eberlein et al, Peptides (1989) 10:797-803). PYY₃₋₃₆ is generated from PYY₁₋₃₆ by cleavage of the N-terminal Tyr and Pro residues by dipeptidyl peptidase IV. PYY₃₋₃₆ is the predominant form of PYY in human postprandial plasma (Grandt et al, Regul. Pept. (1994) 51:151-159). PYY₁₋₃₆ and PYY₃₋₃₆ have been reported to have comparable agonist activity at NPY Y2 receptor (Y2R), a G protein-coupled receptor (Parker et al, Br. J. Pharmacol. (2008) 153:420-431); however, PYY₃₋₃₆ has been reported to be a high-affinity Y2R selective agonist (Keire et al, Am. J. Physiol. Gastrointest. Liver Physiol. (2000) 279:G126-G131). PYY was subsequently reported to reduce high-fat food intake in rats after peripheral administration (Okada et al, Endocrinology Supplement (1993) 180) and to cause weight loss in mice after peripheral administration (Morley et al, Life Sciences (1987) 41:2157-2165).

Peripheral administration of PYY₃₋₃₆ has been reported to markedly reduce food intake and weight gain in rats, to decrease appetite and food intake in humans, and to decrease food intake in mice, but not in Y2R-null mice, which was said to suggest that the food intake effect requires the Y2R. In human studies, infusion of PYY₃₋₃₆ was found to significantly decrease appetite and reduce food intake by 33% over 24 hours. Infusion of PYY₃₋₃₆ to reach the normal postprandial circulatory concentrations of the peptide led to peak serum levels of PYY₃₋₃₆ within 15 minutes, followed by a rapid decline to basal levels within 30 minutes. It was reported that there was significant inhibition of food intake in the 12-hour period following the PYY₃₋₃₆ infusion, but was essentially no effect on food intake in the 12-hour to 24-hour period. In a rat study, repeated administration of PYY₃₋₃₆ intraperitoneally (injections twice daily for 7 days) reduced cumulative food intake (Batterham et al, Nature (2002) 418:650-654; Renshaw et al, Current Drug Targets (2005) 6:171-179).

Peripheral administration of PYY₃₋₃₆ has been reported to reduce food intake, body weight gain and glycemic indices in diverse rodent models of metabolic diseases of both sexes (Pittner et al, Int. J. Obes. Relat. Metab. Disord. (2004) 28:963-971). It has been reported that blockade of Y2R with the specific antagonist BIIE-246 attenuates the effect of peripherally administered endogenous and exogenous PYY₃₋₃₆ for reducing food intake (Abbott et al, Brain Res (2005) 1043:139-144). It has been reported that peripheral administration of a novel long-acting selective Y2R polyethylene glycol-conjugated peptide agonist reduces food intake and improves glucose metabolism (glucose disposal, plasma insulin and plasma glucose) in rodents (Ortiz et al, JPET (2007) 323:692-700; Lamb et al, J. Med. Chem. (2007) 50:2264-2268). It has been reported that PYY ablation in mice leads to the development of hyperinsulinemia and obesity (Boey et al, Diabetologia (2006) 49:1360-1370). It has been reported that peripheral administration of a long-acting, potent and highly selective Y2R agonist inhibits food intake and promotes fat metabolism in mice (Balasubramaniam et al, Peptides (2007) 28:235-240).

There is evidence that agents which stimulate PYY synthesis in vivo can confer protection against diet-induced and genetic obesity and can improve glucose tolerance (Boey et al, Neuropeptides (2008) 42:19-30).

It has been reported that Y2R agonists such as PYY₁₋₃₆ and PYY₃₋₃₆ can confer protection against epileptic seizures, such as against kainate seizures (El Bahh et al, Eur. J. Neurosci. (2005) 22:1417-1430; Woldbye et al, Neurobiology of Disease (2005) 20:760-772).

It has been reported that Y2R agonists such as PYY₁₋₃₆ and PYY₃₋₃₆ act as proabsorbtive (or anti-secretory) hormones, increasing upon intravenous administration the absorption of both water and sodium in various parts of the bowel (Bilchik et al, Gastroenterol. (1993) 105:1441-1448; Liu et al, J. Surg. Res. (1995) 58:6-11; Nightingale et al, Gut (1996) 39:267-272; Liu et al, Am Surg (1996) 62:232-236; Balasubramaniam et al, J. Med. Chem. (2000) 43:3420-3427). It has been reported that Y2R agonists such as PYY analogues inhibit secretion and promote absorption and growth in the intestinal epithelium (Balasubramaniam et al, J. Med. Chem. (2000) 43:3420-3427). It has been reported that PYY promotes intestinal growth in normal rats (Gomez et al, Am. J. Physiol. (1995) 268:G71-G81). It has been reported that Y2R agonists such as PYY₁₋₃₆ and PYY₃₋₃₆ inhibit bowel motility and work to prevent diarrhea (EP1902730; also see Cox, Peptides (2007) 28:345-351).

It has been reported that Y2R agonists such as PYY₁₋₃₆ and PYY₃₋₃₆ can confer protection against inflammatory bowel disease such as ulcerative colitis and Crohn's disease (WO 03/105763). It has been reported that PYY-deficient mice exhibit an osteopenic phenotype, i.e. that PYY can increase bone mass and/or can confer protection against loss of bone mass (e.g., decreases loss of bone mass) (Wortley et al, Gastroenterol. (2007) 133:1534-1543). It has been reported that PYY₃₋₃₆ can confer protection in rodent models of pancreatitis (Vona-Davis et al, Peptides (2007) 28:334-338).

It has been reported that angiogenesis is impaired in Y2R-deficient mice (Lee et al, Peptides (2003) 24:99-106), i.e. that agonists of Y2R such as PYY₁₋₃₆ and PYY₃₋₃₆ promote angiogenesis. It has been reported that would healing is impaired in Y2R-deficient mice (Ekstrand et al, PNAS USA (2003) 100:6033-6038), i.e. that agonists of Y2R such as PYY₁₋₃₆ and PYY₃₋₃₆ promote wound healing. It has been reported that ischemic angiogenesis is impaired in Y2R-deficient mice (Lee et al, J. Clin. Invest. (2003) 111:1853-1862), i.e. that agonists of Y2R such as PYY₁₋₃₆ and PYY₃₋₃₆ promotes revascularization and restoration of function of ischemic tissue. It has been reported that agonists of Y2R such as PYY₁₋₃₆ and PYY₃₋₃₆ mediate increases in collateral-dependent blood flow in a rat model of peripheral arterial disease (Cruze et al, Peptides (2007) 28:269-280).

It has been reported that PYY and Y2R agonists such as PYY₃₋₃₆ can suppress tumor growth in the cases of, e.g., pancreatic cancer such as pancreatic ductal adenocarcinoma, breast cancer such as breast infiltrative ductal adenocarcinoma, colon cancer such as colon adenocarcinoma and Barrett's adenocarcinoma (Liu et al, Surgery (1995) 118:229-236; Liu et al, J. Surg. Res. (1995) 58:707-712; Grise et al, J. Surg. Res. (1999) 82:151-155; Tseng et al, Peptides (2002) 23:389-395; McFadden et al, Am. J. Surg. (2004) 188:516-519).

It has been reported that stimulation of Y2R such as by PYY₃₋₃₆ leads to an increase in plasma adiponectin (Ortiz et al, JPET (2007) 323:692-700). Adiponectin is an adipokine with potent anti-inflammatory properties (Ouchi et al, Clin Chim Acta (2007) 380:24-30; Tilg et al, Nat. Rev. Immunol. (2006) 6:772-783). Adiponectin exerts anti-atherogenic effects by targeting vascular endothelial cells and macrophages and insulin-sensitizing effects, predominantly in muscle and liver (Kubota et al, J. Biol. Chem. (2002) 277:25863-25866; Maeda et al, Nat. Med. (2002) 8:731-737). Low adiponectin levels have been reported to be associated with atherogenic lipoproteins in dyslipidemia (elevated triglycerides, small dense LDL cholesterol, low HDL cholesterol) (Marso et al, Diabetes Care (2008) February 5 Epub ahead of print). Adiponectin has been implicated in high density lipoprotein (HDL) assembly (Oku et al, FEBS Letters (2007) 581:5029-5033). Adiponectin has been found to ameliorate the abnormalities of metabolic syndrome, including insulin resistance, hyperglycemia, and dyslipidemia, in a mouse model of obesity-linked metabolic syndrome associated with decreased adiponectin levels (Hara et al, Diabetes Care (2006) 29:1357-1362). Adiponectin has been reported to stimulate angiogenesis in response to tissue ischemia (Shibata et al, J. Biol. Chem. (2004) 279:28670-28674). Adiponectin has been reported to prevent cerebral ischemic injury through endothelial nitric oxide synthase-dependent mechanisms (Nishimura et al, Circulation (2008) 117:216-223). Adiponectin has been reported to confer protection against myocardial ischemia-reperfusion injury (Shibata et al, Nat Med (2005) 11:1096-1103; Tao et al, Circulation (2007) 115:1408-1416). Adiponectin has been reported to confer protection against myocardial ischaemia-reperfusion injury via AMP-activated protein kinase, Akt, and nitric oxide (Gonon et al, Cardiovasc Res. (2008) 78:116-122). Adiponectin has been reported to confer protection against the development of systolic dysfunction following myocardial infarction, through its abilities to suppress cardiac hypertrophy and interstitial fibrosis, and protect against myocyte and capillary loss (Shibata et al, J. Mol. Cell Cardiol. (2007) 42:1065-1074). Adiponectin has been reported to confer protection against inflammatory lung disease; adiponectin-deficient mice exhibit an emphysema-like phenotype (Summer et al, Am J. Physiol. Lung Cell Mol. Physiol (Mar. 7, 2008)). Adiponectin has been reported to confer protection against allergic airway inflammation and airway hyperresponsiveness such as may be associated with asthma (Shore et al, J. Allergy Clin. Immunol (2006) 118:389-395). Adiponectin has been suggested to confer protection against pulmonary arterial hypertension by virtue of its insulin-sensitizing effects (Hansmann et al, Circulation (2007) 115:1275-1284). Adiponectin has been reported to ameliorate obesity-related hypertension, with said amelioration of hypertension being associated in part with upregulated prostacyclin expression (Ohashi et al, Hypertension (2006) 47:1108-1116). Adiponectin has been reported to decrease tumor necrosis factor (TNF)-α-induced expression of the adhesion molecules VCAM-1, E-selectin and ICAM-1 in human aortic endothelial cells (HAECs) (Ouchi et al, Circulation (1999) 100:2473-2476) and to inhibit production of TNF-α in macrophages (Yokota et al, Blood (2000) 96:1723-1732). Adiponectin has been reported to confer protection against restenosis after vascular intervention (Matsuda et al, J Biol Chem (2002) 277:37487-37491). The central role of TNF-α in inflammation has been demonstrated by the ability of agents that block the action of TNF-α to treat a range of inflammatory conditions. TNF-α-mediated inflammatory conditions encompass rheumatoid arthritis, inflammatory bowel disease such as Crohn's disease, ankylosing spondylitis, psoriasis, ischemic brain injury, cardiac allograft rejection, asthma, and the like (Bradley, J Pathol (2008) 214:149-160). See, e.g., Yamamoto et al, Clinical Science (2002) 103:137-142; Behre, Scand J Clin Lab Invest (2007) 67:449-458; Guerre-Millo, Diabetes & Metabolism (2008) 34:12-18; Parker et al, Br. J. Pharmacol. (2008) 153:420-431.

### SUMMARY OF THE INVENTION

One aspect of the present invention is directed to compounds, as described herein, and pharmaceutically acceptable salts, solvates, and hydrates thereof, which bind to and modulate the activity of a GPCR, referred to herein as GPR119, and uses thereof.

One aspect of the present invention encompasses, inter alia, certain cyclohexane derivatives selected from compounds of Formula (Ia) and pharmaceutically acceptable salts, solvates, and hydrates thereof:

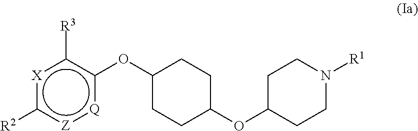

wherein:

Q is N or CR⁴;

Z is N or CR⁵;

X is N, N(O), or CR⁶;

R¹ is selected from the group consisting of H, S(O)₂R⁷, C(O)R⁷, CH₂R⁸, C(O)OR⁹, and C(O)SR⁹; or R¹ is selected from the group consisting of heteroaryl and phenyl, each optionally substituted with one or more substituents selected independently from the group consisting of C₂-C₆ alkenyl, C₁-C₆ alkoxy, C₁-C₆ alkyl, halogen, C₁-C₆ haloalkoxy, and C₁-C₆ haloalkyl;

R² is selected from the group consisting of H, C₁-C₆ alkyl, cyano, C₃-C₆ cycloalkyl, halogen, C₁-C₆ haloalkyl, heteroaryl, heterocyclyl, S(O)ₙR¹¹, and C(O)NR¹²R¹³; wherein said C₁-C₆ alkyl is optionally substituted with one or more substituents selected independently from the group consisting of amino, C₁-C₆ alkylsulfonyl, cyano, and C(O)NR¹²R¹³; said C₃-C₆ cycloalkyl is optionally substituted with C(O)NR¹²R¹³; said heteroaryl is optionally substituted with C₁-C₆ alkyl; and said heterocyclyl is optionally substituted with one or more substituents selected independently from the group consisting of amino, C₁-C₆ alkylsulfonyl, hydroxyl, and halogen;

R³, R⁴, R⁵, and R⁶ are each independently selected from the group consisting of H, C₁-C₆ alkyl, C₁-C₆ alkylsulfonyl, and halogen;

R⁷ is selected from the group consisting of C₁-C₆ alkyl, C₃-C₆ cycloalkyl, and C₁-C₆ haloalkyl; wherein said C₃-C₆ cycloalkyl is optionally substituted with one or more C₁-C₆ alkyl;

R⁸ is selected from the group consisting of C₃-C₆ cycloalkyl, C₁-C₆ haloalkyl, heteroaryl, phenyl, and C(O)OR⁹; wherein said C₃-C₆ cycloalkyl and said heteroaryl are each optionally substituted with one or more substituents selected independently from the group consisting of C₁-C₆ haloalkyl and C₁-C₆ alkyl;

R⁹ is selected from the group consisting of C₁-C₆ alkyl, C₃-C₆ cycloalkyl, C₁-C₆ haloalkyl, heterocyclyl, and phenyl; said C₁-C₆ alkyl and said C₃-C₆ cycloalkyl are each optionally substituted with one or more substituents selected independently from the group consisting of C₁-C₆ alkyl, halogen, hydroxyl, C₁-C₆ alkoxy, and R¹⁰, wherein said C₁-C₆ alkoxy is optionally substituted with phenyl;

R¹⁰ is heterocyclyl optionally substituted with C₁-C₆ alkyl;

R¹¹ is selected from the group consisting of C₁-C₆ alkyl, C₃-C₆ cycloalkyl, C₁-C₆ haloalkyl, and heterocyclyl; wherein said C₁-C₆ alkyl and heterocyclyl are each optionally substituted with one or more substituents selected independently from the group consisting of halogen, hydroxyl, and NR¹²R¹³; and said heterocyclyl is optionally substituted with one or more substituents selected independently from the group consisting of amino, C₁-C₆ alkyl, and hydroxyl;

R¹² and R¹³ are each independently selected from the group consisting of H, C₁-C₆ alkyl, and C₃-C₆ cycloalkyl; wherein said C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, hydroxyl, and C₁-C₆ alkoxy; and

n is 0, 1, or 2.

One aspect of the present invention pertains to compositions comprising a compound of the present invention.

One aspect of the present invention pertains to compositions comprising a compound of the present invention and a pharmaceutically acceptable carrier.

One aspect of the present invention pertains to methods for preparing a composition comprising the step of admixing a compound of the present invention and a pharmaceutically acceptable carrier.

One aspect of the present invention pertains to pharmaceutical products selected from: a pharmaceutical composition, a formulation, a dosage form, a combined preparation, a twin pack, and a kit; comprising a compound of the present invention.

One aspect of the present invention pertains to compositions comprising a compound of the present invention and a second pharmaceutical agent.

One aspect of the present invention pertains to methods for preparing a composition comprising the step of admixing a compound of the present invention and a second pharmaceutical agent.

One aspect of the present invention pertains to compositions comprising a compound of the present invention, a second pharmaceutical agent, and a pharmaceutically acceptable carrier.

One aspect of the present invention pertains to methods for preparing a composition comprising the step of admixing a compound of the present invention, a second pharmaceutical agent, and a pharmaceutically acceptable carrier.

One aspect of the present invention pertains to compositions obtained by the methods of the present invention as described herein.

One aspect of the present invention pertains to a pharmaceutical product selected from: a pharmaceutical composition, a formulation, a dosage form, a combined preparation, a twin pack, and a kit; comprising a compound of the present invention and a second pharmaceutical agent.

One aspect of the present invention pertains to methods for modulating the activity of a GPR119 receptor, comprising administering to an individual in need thereof, a therapeutically effective amount of: a compound of the present invention; a composition of the present invention; or a pharmaceutical product of the present invention.

One aspect of the present invention pertains to the use of a compound of the present invention; a composition of the present invention; or a pharmaceutical product of the present invention; in the manufacture of a medicament for modulating the activity of a GPR119 receptor in an individual.

One aspect of the present invention pertains to a compound of the present invention; a composition of the present invention; or a pharmaceutical product of the present invention; for use in a method of treating the human or animal by therapy.

One aspect of the present invention pertains to a compound of the present invention; a composition of the present invention; or a pharmaceutical product of the present invention; for use in a method of modulating the activity of a GPR119 receptor in an individual.

One aspect of the present invention pertains to a pharmaceutical product selected from: a pharmaceutical composition, a formulation, a dosage form, a combined preparation, a twin pack, and a kit; comprising a compound of the present invention; for use in a method of treating the human or animal by therapy.

One aspect of the present invention pertains to a pharmaceutical product selected from: a pharmaceutical composition, a formulation, a dosage form, a combined preparation, a twin pack, and a kit; comprising a compound of the present invention; for modulating the activity of a GPR119 receptor in an individual.

One aspect of the present invention pertains to compounds, methods, compositions, uses of compounds, and pharmaceutical products, as described herein, for agonizing the GPR119 receptor.

One aspect of the present invention pertains to compounds, methods, compositions, uses of compounds, and pharmaceutical products, as described herein, increasing the secretion of an incretin.

One aspect of the present invention pertains to compounds, methods, compositions, uses of compounds, and pharmaceutical products, as described herein, increasing a blood incretin level.

One aspect of the present invention pertains to compounds, methods, compositions, uses of compounds, and pharmaceutical products, as described herein, treating a disorder, wherein the disorder is selected from: a GPR119-receptor-related disorder; a condition ameliorated by increasing a blood incretin level; a condition characterized by low bone mass; a neurological disorder; a metabolic-related disorder; and obesity.

One aspect of the present invention pertains to compounds, methods, compositions, uses of compounds, and pharmaceutical products, as described herein, in combination with a second pharmaceutical agent.

One aspect of the present invention pertains to compounds, methods, compositions, uses of compounds, and pharmaceutical products, as described herein, in combination with a second pharmaceutical agent, wherein the second pharmaceutical agent is selected from: an inhibitor of DPP-IV, a biguanide, an alpha-glucosidase inhibitor, an insulin analogue, a sulfonylurea, a SGLT2 inhibitor, a meglitinide, a thiazolidinedione, and an anti-diabetic peptide analogue.

These and other aspects of the invention disclosed herein will be set forth in greater detail as the patent disclosure proceeds.

### BRIEF DESCRIPTION OF THE DRAWINGS

FIG. 1 shows the in vivo effects of Compound 28 on glucose homeostasis in male diabetic ZDF rats (oral glucose tolerance test (oGTT)).

FIG. 2 shows the in vivo effects of Compound 28 on percent glycemic inhibition in male diabetic ZDF rats.

FIG. 3 shows the in vivo effects of Compound 62 on glucose homeostasis in male diabetic ZDF rats (oral glucose tolerance test (oGTT)).

FIG. 4 shows the in vivo effects of Compound 62 on percent glycemic inhibition in male diabetic ZDF rats.

FIG. 5 shows the in vivo effects of Compound 28 on glucose homeostasis in male 129SVE mice (oral glucose tolerance test (oGTT)).

FIG. 6 shows the in vivo effects of Compound 62 on percent glycemic inhibition in male 129SVE mice.

FIG. 7 shows the in vivo effects of representative compounds of the present invention on incretin hormone GIP release.

FIG. 8 shows a general synthetic scheme for the preparation of compounds of Formula (Ia) utilizing cyclohexane-1,4-diol as the starting material. It is understood that 1s,4s (i.e., cis), 1r,4r (i.e., trans), or a mixture of 1s,4s and 1r,4r (i.e., cis and trans) cyclohexane-1,4-diol can be used in the preparation of compounds of Formula (Ia).

FIG. 9 shows a general synthetic method for the preparation of the useful intermediate tert-butyl 4-(4-hydroxycyclohexyl oxy)piperidine-1-carboxylate as substantially pure 1s,4s (i.e., cis), and substantially stereochemically pure 1r,4r (i.e., trans).

FIG. 10 shows a general synthetic method for the preparation of useful intermediates (1s,4s)-4-(1-methylpiperidin-4-yloxy)cyclohexanol (i.e., cis) and (1r,4r)-4-(1-methylpiperidin-4-yloxy)cyclohexanol (i.e., trans), see Example 1.114.

FIG. 11 shows a general synthetic scheme for the preparation of intermediates that are useful in the synthesis of compounds of Formula (Ia).

FIG. 12 shows a general synthetic scheme for the preparation of certain compounds of Formula (Ia), wherein R¹ is an optionally substituted oxadiazolyl group.

FIG. 13 shows a general synthetic scheme for the preparation of certain compounds of Formula (Ia), wherein R¹ is —S(O)₂R⁷, —C(O)R⁷, or —CH₂R⁸.

FIG. 14 shows a general synthetic scheme for the preparation of certain compounds of Formula (Ia), wherein R¹ is C(O)OR⁹, an optionally substituted heteroaryl, or an optionally substituted phenyl.

FIG. 15 shows a general synthetic scheme for the preparation of 1r,4r (i.e., trans) compounds of Formula (Ia), 1s,4s (i.e., cis) compounds of Formula (Ia) can be prepared in an analogous manner with the exception that Method A would be used with (1s,4s)-4-(1-methylpiperidin-4-yloxy)cyclohexanol to retain the cis stereochemistry while Method B would be used with (1r,4r)-4-(1-methylpiperidin-4-yloxy)cyclohexanol to invent the stereocenter thus providing the cis stereochemistry.

FIG. 16 shows the in vivo effects of Compound 83 on glucose homeostasis in male diabetic ZDF rats (oral glucose tolerance test (oGTT)).

FIG. 17 shows the in vivo effects of Compound 83 on percent glycemic inhibition in male diabetic ZDF rats.

FIG. 18 shows a powder X-ray diffraction (PXRD) pattern for Compound 28.

FIG. 19 shows two powder X-ray diffraction (PXRD) patterns for Compound 83, one sample prepared from a slurry in ethanol and a second sample that was ground prior to PXRD analysis.

FIG. 20 shows a powder X-ray diffraction (PXRD) pattern for Compound 85.

FIG. 21 shows a powder X-ray diffraction (PXRD) pattern for Compound 109.

FIG. 22 shows a powder X-ray diffraction (PXRD) pattern for Compound 122.

FIG. 23 shows a thermogravimetric analysis (TGA) thermogram and a differential scanning calorimetry (DSC) thermogram for Compound 28.

FIG. 24 shows a TGA thermogram and a DSC thermogram for Compound 83.

FIG. 25 shows a TGA thermogram and a DSC thermogram for Compound 85.

FIG. 26 shows a TGA thermogram and a DSC thermogram for Compound 109.

FIG. 27 shows a TGA thermogram and a DSC thermogram for Compound 122.

### DETAILED DESCRIPTION OF THE INVENTION

### Definitions

For clarity and consistency, the following definitions will be used throughout this patent document.

The term “agonist” as used herein refers to a moiety that interacts with and activates a G-protein-coupled receptor, for instance a GPR119-receptor, and can thereby initiate a physiological or pharmacological response characteristic of that receptor. For example, an agonist may activate an intracellular response upon binding to a receptor, or enhance GTP binding to a membrane.

The term “antagonist” as used herein refers to a moiety that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

The term “hydrate” as used herein refers to a compound of the invention or a salt thereof, that further includes a stoichiometric or non-stoichiometric amount of water bound by non-covalent intermolecular forces.

The term “solvate” as used herein refers to a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “in need of treatment” and the term “in need thereof” when referring to treatment are used interchangeably and refer to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” refers to any animal, including mammals, preferably mice, rats, other rodents, rabbits, dogs, cats, swine, cattle, sheep, horses, or primates, and most preferably humans.

The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist, or decreases GTP binding to a membrane. Preferably, the baseline intracellular response is inhibited in the presence of the inverse agonist by at least 30%, more preferably by at least 50% and most preferably by at least 75%, as compared with the baseline response in the absence of the inverse agonist.

The term “modulate or modulating” refers to an increase or decrease in the amount, quality, response or effect of a particular activity, function or molecule.

The term “pharmaceutical composition” refers to a composition comprising at least one active ingredient; including but not limited to, salts, solvates, and hydrates of compounds of the present invention, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “therapeutically effective amount” refers to the amount of active compound or pharmaceutical agent that elicits the biological or medicinal response in a tissue, system, animal, individual or human that is being sought by a researcher, veterinarian, medical doctor or other clinician or caregiver or by an individual, which includes one or more of the following:

(1) Preventing the disease, for example, preventing a disease, condition or disorder in an individual that may be predisposed to the disease, condition or disorder but does not yet experience or display the pathology or symptomatology of the disease;

(2) Inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) Ameliorating the disease, for example, ameliorating a disease, condition or disorder in an individual that is experiencing or displaying the pathology or symptomatology of the disease, condition or disorder (i.e., reversing the pathology and/or symptomatology).

### Chemical Group, Moiety or Radical

The term “amino” refers to the group —NH₂.

The term “C₁-C₆ alkoxy” refers to a radical comprising a C₁-C₆ alkyl group attached directly to an oxygen atom, wherein C₁-C₆ alkyl has the same definition as found herein. Some embodiments contain 1 to 5 carbons. Some embodiments contain 1 to 4 carbons. Some embodiments contain 1 to 3 carbons. Some embodiments contain one or two carbons. Examples of an alkoxy group include, but are not limited to methoxy, ethoxy, n-propoxy, isopropoxy, n-butoxy, t-butoxy, isobutoxy, s-butoxy, and the like.

The term “C₁-C₆ alkyl” refers to a straight or branched carbon radical containing 1 to 6 carbons. Some embodiments contain 1 to 5 carbons. Some embodiments contain 1 to 4 carbons. Some embodiments contain 1 to 3 carbons. Some embodiments contain one or two carbons. Examples of an alkyl group include, but are not limited to, methyl, ethyl, n-propyl, isopropyl, n-butyl, s-butyl, isobutyl, t-butyl, pentyl, isopentyl, t-pentyl, neopentyl, 1-methylbutyl [i.e., —CH(CH₃)CH₂CH₂CH₃], 2-methylbutyl [i.e., —CH₂CH(CH₃)CH₂CH₃], n-hexyl, and the like.

The term “C₁-C₆ alkylsulfonyl” refers to a radical comprising a C₁-C₆ alkyl group attached to the sulfur of a sulfonyl group, wherein the C₁-C₆ alkyl radical has the same definition as described herein. Examples include, but are not limited to, methylsulfonyl, ethylsulfonyl, n-propylsulfonyl, isopropylsulfonyl, n-butylsulfonyl, s-butylsulfonyl, isobutylsulfonyl, t-butylsulfonyl, and the like.

The term “C₃-C₆ cycloalkyl” refers to a saturated ring radical containing 3 to 6 carbons. Some embodiments contain 3 to 4 carbons. Some embodiments contain 3 to 5 carbons. Some embodiments contain 4 to 6 carbons. Some embodiments contain 5 to 6 carbons. Examples include cyclopropyl, cyclobutyl, cyclopentyl, cyclohexyl, cycloheptyl, and the like.

The term “cyano” refers to the group —CN.

The term “C₁-C₆ haloalkoxy” refers to a radical comprising a C₁-C₆ haloalkyl group directly attached to an oxygen atom, wherein C₁-C₆ haloalkyl has the same definition as found herein. Examples include, but are not limited to, difluoromethoxy, trifluoromethoxy, 2,2,2-trifluoroethoxy, pentafluoroethoxy, 2-fluoropropan-2-yloxy, 1,1-difluoropropyloxy, 1,3-difluoropropan-2-yloxy, (S)-1-fluoropropan-2-yloxy, (R)-1-fluoropropan-2-yloxy, 1,1,1-trifluoropropan-2-yloxy, 1,1,1,3,3,3-hexafluoropropan-2-yloxy, and the like.

The term “C₁-C₆ haloalkyl” refers to a radical comprising a C₁-C₆ alkyl group substituted with one or more halogens, wherein C₁-C₆ alkyl has the same definition as found herein. The C₁-C₆ haloalkyl may be fully substituted in which case it can be represented by the formula C_{q}L_{2q+1}, wherein L is a halogen and “q” is 1, 2, 3, 4, 5 or 6. When more than one halogen is present then they may be the same or different and selected from: fluorine, chlorine, bromine, and iodine. In some embodiments, haloalkyl contains 1 to 5 carbons. In some embodiments, haloalkyl contains 1 to 4 carbons. In some embodiments, haloalkyl contains 1 to 3 carbons. In some embodiments, haloalkyl contains one or two carbons. Examples of haloalkyl groups include, but are not limited to, fluoromethyl, difluoromethyl, trifluoromethyl, 2,2,2-trifluoroethyl, pentafluoroethyl, 2-fluoropropan-2-yl, 1,1-difluoropropyl, 1,3-difluoropropan-2-yl, (S)-1-fluoropropan-2-yl, (R)-1-fluoropropan-2-yl, 1,1,1-trifluoropropan-2-yl, 1,1,1,3,3,3-hexafluoropropan-2-yl, and the like.

The term “halogen” refers to a fluoro, chloro, bromo or iodo group.

The term “heteroaryl” refers to a ring system containing 5 to 10 ring atoms, that may contain a single ring or two fused rings, and wherein at least one ring is aromatic and at least one ring atom of the aromatic ring is a heteroatom selected from, for example: O, S and N, wherein N is optionally substituted with H, C₁-C₄ acyl, C₁-C₄ alkyl, or O (i.e., forming an N-oxide) and S is optionally substituted with one or two oxygens. In some embodiments, the aromatic ring contains one heteroatom. In some embodiments, the aromatic ring contains two heteroatoms. In some embodiments, the aromatic ring contains three heteroatoms. Examples include furanyl, thienyl, pyrrolyl, imidazolyl, oxazolyl, thiazolyl, isoxazolyl, pyrazolyl, isothiazolyl, oxadiazolyl, triazolyl, tetrazolyl, thiadiazolyl, pyridinyl, pyrazinyl, pyrimidinyl, pyridazinyl, triazinyl, quinolinyl, isoquinolinyl, cinnolinyl, phthalazinyl, quinazolinyl, quinoxalinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran, 2,3-dihydrobenzofuranyl, 4H-benzo[1,3]dioxinyl, 3,4-dihydro-1H-isoquinolinyl, 1,4,6,7-tetrahydro-imidazo[4,5-c]pyridinyl, 7,8-dihydro-5H-[1,6]naphthyridinyl, 5,6-dihydro-8H-[1,2,4]triazolo[4,3-a]pyrazinyl, benzo[1,3]dioxolyl, pyrazolo[1,5-a]pyrimidinyl, 1,2,3,4-tetrahydroquinolinyl, and the like. Some embodiments are directed to 5-membered heteroaryl rings. Examples of a 5-membered heteroaryl ring include furanyl, thienyl, pyrrolyl, imidazolyl, oxazolyl, thiazolyl, isoxazolyl, pyrazolyl, isothiazolyl, oxadiazolyl, triazolyl, tetrazolyl, thiadiazolyl, and the like. Some embodiments are directed to 6-membered heteroaryl rings. Examples of a 6-membered heteroaryl ring include pyridinyl, pyrazinyl, pyrimidinyl, pyridazinyl, triazinyl, and the like. Some embodiments are directed to 8 to 10-membered heteroaryl rings. Examples of a 8 to 10-membered heteroaryl ring include quinolinyl, isoquinolinyl, cinnolinyl, phthalazinyl, quinazolinyl, quinoxalinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran, 2,3-dihydrobenzofuranyl, 4H-benzo[1,3]dioxinyl, 3,4-dihydro-1H-isoquinolinyl, 1,4,6,7-tetrahydro-imidazo[4,5-c]pyridinyl, 7,8-dihydro-5H-[1,6]naphthyridinyl, 5,6-dihydro-8H-[1,2,4]triazolo[4,3-a]pyrazinyl, benzo[1,3]dioxolyl, pyrazolo[1,5-a]pyrimidinyl, 1,2,3,4-tetrahydroquinolinyl, and the like.

The term “heterocyclyl” refers to a non-aromatic ring radical containing 3 to 7 ring atoms, wherein one, two or three ring atoms are heteroatoms is selected independently from, for example: O, S, and N, wherein N is optionally substituted with H, C₁-C₄ acyl or C₁-C₄ alkyl; and S is optionally substituted with one or two oxygens. Examples of a heterocyclyl group include, but are not limited to, aziridinyl, azetidinyl, piperidinyl, morpholinyl, piperazinyl, pyrrolidinyl, [1,3]-dioxolanyl, thiomorpholinyl, [1,4]oxazepanyl, 1,1-dioxothiomorpholinyl, azepanyl, tetrahydrofuranyl, tetrahydropyranyl, tetrahydrothiopyranyl, 1-oxo-hexahydro-1λ⁴-thiopyranyl, 1,1-dioxo-hexahydro-1λ⁶-thiopyranyl, and the like.

The term “hydroxyl” refers to the group —OH.

The term “phenyl” refers to the group —C₆H₅.

### COMPOUNDS OF THE INVENTION

One aspect of the present invention provides, inter alia, compounds of Formula (Ia) and pharmaceutically acceptable salts, solvates, and hydrates thereof:

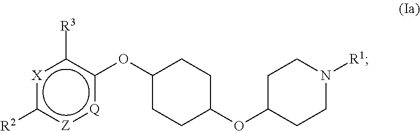

wherein R¹, R², R³, Q, Z, X, and variables related thereto (i.e., R⁴, R⁵, R⁶, R⁷, R⁸, R⁹, R¹⁰, R¹¹, R¹², and R¹³), have the same definitions as described herein, supra and infra.

One aspect of the present invention encompasses, inter alia, certain cyclohexane derivatives selected from compounds of Formula (Ia) and pharmaceutically acceptable salts, solvates, and hydrates thereof, wherein:

Q is N or CR⁴;

Z is N or CR⁵;

X is N or CR⁶;

R¹ is selected from the group consisting of H, S(O)₂R⁷, C(O)R⁷, CH₂R⁸, and C(O)OR⁹; or R¹ is selected from the group consisting of heteroaryl and phenyl, each optionally substituted with one or more substituents selected independently from the group consisting of C₂-C₆ alkenyl, C₁-C₆ alkoxy, C₁-C₆ alkyl, halogen, C₁-C₆ haloalkoxy, and C₁-C₆ haloalkyl;

R² is selected from the group consisting of H, C₁-C₆ alkyl, cyano, C₃-C₆ cycloalkyl, halogen, C₁-C₆ haloalkyl, heteroaryl, heterocyclyl, S(O)₂R¹¹, and C(O)NR¹²R¹³; wherein said C₁-C₆ alkyl is optionally substituted with one or more substituents selected independently from the group consisting of amino, C₁-C₆ alkylsulfonyl, cyano, and C(O)NR¹²R¹³; said C₃-C₆ cycloalkyl is optionally substituted with C(O)NR¹²R¹³; said heteroaryl is optionally substituted with C₁-C₆ alkyl; and said heterocyclyl is optionally substituted with one or more substituents selected independently from the group consisting of amino, C₁-C₆ alkylsulfonyl, hydroxyl, and halogen;

R³, R⁴, R⁵, and R⁶ are each independently selected from the group consisting of H, C₁-C₆ alkyl, C₁-C₆ alkylsulfonyl, and halogen;

R⁷ is selected from the group consisting of C₁-C₆ alkyl, C₃-C₆ cycloalkyl, and C₁-C₆ haloalkyl; wherein said C₃-C₆ cycloalkyl is optionally substituted with one C₁-C₆ alkyl substituent;

R⁸ is selected from the group consisting of heteroaryl, phenyl, and C(O)OR⁹; wherein said heteroaryl is optionally substituted with C₁-C₆ alkyl;

R⁹ is selected from the group consisting of C₁-C₆ alkyl, C₃-C₆ cycloalkyl, C₁-C₆ haloalkyl, heterocyclyl, and phenyl; wherein said C₁-C₆ alkyl is optionally substituted with one or more substituents selected independently from the group consisting of hydroxyl, C₁-C₆ alkoxy, and R¹⁰, wherein said C₁-C₆ alkoxy is optionally substituted with phenyl; and said C₃-C₆ cycloalkyl is optionally substituted with one or more substituents selected independently from the group consisting of C₁-C₆ alkyl and halogen;

R¹⁰ is heterocyclyl optionally substituted with C₁-C₆ alkyl;

R¹¹ is selected from the group consisting of C₁-C₆ alkyl, C₃-C₆ cycloalkyl, C₁-C₆ haloalkyl, and heterocyclyl; wherein said C₁-C₆ alkyl is optionally substituted with one or more substituents selected independently from the group consisting of hydroxyl and NR¹²R¹³; and said heterocyclyl is optionally substituted with one or more substituents selected independently from the group consisting of amino, C₁-C₆ alkyl, and hydroxyl; and

R¹² and R¹³ are each independently selected from the group consisting of H, C₁-C₆ alkyl, and C₃-C₆ cycloalkyl; wherein said C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, hydroxyl, and C₁-C₆ alkoxy.

One aspect of the present invention encompasses, inter alia, certain cyclohexane derivatives selected from compounds of Formula (Ia) and pharmaceutically acceptable salts, solvates, and hydrates thereof, wherein:

Q is N or CR⁴;

Z is N or CR⁵;

X is N or CR⁶;

R¹ is selected from the group consisting of H, S(O)₂R⁷, C(O)R⁷, CH₂R⁸, and C(O)OR⁹; or R¹ is heteroaryl or phenyl, each optionally substituted with one or more substituents selected independently from the group consisting of C₂-C₆ alkenyl, C₁-C₆ alkoxy, C₁-C₆ alkyl, halogen, C₁-C₆ haloalkoxy, and C₁-C₆ haloalkyl;

R² is selected from the group consisting of H, C₁-C₆ alkyl, cyano, C₃-C₆ cycloalkyl, halogen, C₁-C₆ haloalkyl, heteroaryl, heterocyclyl, S(O)₂R¹¹, and C(O)NR¹²R¹³; wherein said C₁-C₆ alkyl is optionally substituted with one or more substituents selected independently from the group consisting of amino, C₁-C₆ alkylsulfonyl, cyano, and C(O)NR¹²R¹³; said C₃-C₆ cycloalkyl is optionally substituted with C(O)NR¹²R¹³; said heteroaryl is optionally substituted with C₁-C₆ alkyl; and said heterocyclyl is optionally substituted with one or more substituents selected independently from the group consisting of amino, C₁-C₆ alkylsulfonyl, hydroxyl, and halogen;

R³, R⁴, R⁵, and R⁶ are each independently selected from the group consisting of H, C₁₋₆ alkyl, C₁₋₆ alkylsulfonyl, and halogen;

R⁷ is selected from the group consisting of C₁-C₆ alkyl, C₃-C₆ cycloalkyl, and C₁-C₆ haloalkyl; wherein said C₃-C₆ cycloalkyl is optionally substituted with one C₁-C₆ alkyl substituent;

R⁸ is selected from the group consisting of heteroaryl, phenyl, and C(O)OR⁹; wherein said heteroaryl is optionally substituted with C₁-C₆ alkyl;

R⁹ is selected from the group consisting of C₁-C₆ alkyl, C₃-C₆ cycloalkyl, C₁-C₆ haloalkyl, and heterocyclyl; wherein said C₁-C₆ alkyl is optionally substituted with one or more substituents selected independently from the group consisting of hydroxyl and R¹⁰; and said C₃-C₆ cycloalkyl is optionally substituted with one C₁-C₆ alkyl substituent;

R¹⁰ is heterocyclyl optionally substituted with C₁-C₆ alkyl;

R¹¹ is selected from the group consisting of C₁-C₆ alkyl, C₃-C₆ cycloalkyl, C₁-C₆ haloalkyl, and heterocyclyl; wherein said C₁-C₆ alkyl is optionally substituted with one or more substituents selected independently from the group consisting of hydroxyl and NR¹²R¹³, and said heterocyclyl is optionally substituted with one or more substituents selected independently from the group consisting of amino, C₁-C₆ alkyl, and hydroxyl; and

R¹² and R¹³ are each independently selected from the group consisting of H, C₁-C₆ alkyl, and C₃-C₆ cycloalkyl; wherein said C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, and hydroxyl.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables (e.g., R¹, R², R³, R⁴, R⁵, R⁶, R⁷, R⁸, R⁹, R¹⁰, R¹¹, R¹², R¹³, Q, Z, and X) contained within the generic chemical formulae described herein, for example, (Ia), (Ic), (Ie), (If), (Ig), (Ii), (Ik), (Im), (Io), (Iq), (Is), (Iu), (Iw), (Ix), (Iy), (IIa), (IIc), and (IIe), are specifically embraced by the present invention just as if each and every combination was individually and explicitly recited, to the extent that such combinations embrace compounds that result in stable compounds (i.e., compounds that can be isolated, characterized and tested for biological activity). In addition, all subcombinations of the chemical groups listed in the embodiments describing such variables, as well as all subcombinations of uses and medical indications described herein, are also specifically embraced by the present invention just as if each and every subcombination of chemical groups and subcombination of uses and medical indications was individually and explicitly recited herein. In addition, some embodiments include every combination of one or more pharmaceutical agents, such as an inhibitor of DPP-IV, a biguanide, an alpha-glucosidase inhibitor, and the like, either specifically disclosed herein or specifically disclosed in any reference recited herein just as if each and every combination was individually and explicitly recited. Still further, some embodiments of the present invention include every combination of one or more embodiments pertaining to the chemical groups represented by the variables and generic chemical formulae as described herein or every combination of one or more compounds of Formula (Ia) together/in combination with every combination of one or more pharmaceutical agents, such as an inhibitor of DPP-IV, a biguanide, an alpha-glucosidase inhibitor, and the like, either specifically disclosed herein or specifically disclosed in any reference recited herein just as if each and every combination was individually and explicitly recited.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by one or two substituents, a phenyl group can be substituted by 1, 2, 3, 4, or 5 substituents, a naphthyl group can be substituted by 1, 2, 3, 4, 5, 6, or 7 substituents, and the like. Likewise, “substituted with one or more substituents” refers to the substitution of a group with one substituent up to the total number of substituents physically allowed by the group. Further, when a group is substituted with more than one group they can be identical or they can be different.

Compounds of the invention can also include tautomeric forms, such as keto-enol tautomers and the like. Tautomeric forms can be in equilibrium or sterically locked into one form by appropriate substitution. It is understood that the various tautomeric forms are within the scope of the compounds of the present invention.

It is understood and appreciated that compounds of Formula (Ia) and formulae related thereto may have one or more chiral centers and therefore can exist as enantiomers and/or diastereoisomers. The invention is understood to extend to and embrace all such enantiomers, diastereoisomers and mixtures thereof, including but not limited to racemates. It is understood that compounds of Formula (Ia) and formulae used throughout this disclosure represent all individual enantiomers and mixtures thereof, unless stated or shown otherwise.

It is understood and appreciated that compounds of Formula (Ia) and formulae related thereto exist as meso isomers. Such meso isomers may be referred to as cis and trans. The cis meso isomers of compounds of Formula (Ia) are named herein using the designation (1s,4s) and the trans meso isomers of compounds of Formula (Ia) are named herein using the designation (1r,4r) as shown below:

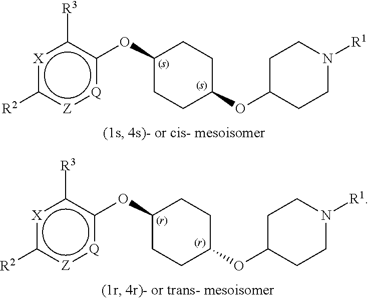

One aspect of the present invention encompasses certain cyclohexane derivatives selected from compounds of Formula (Ic) and pharmaceutically acceptable salts, solvates, and hydrates thereof:

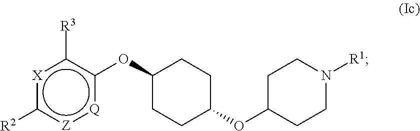

wherein each variable in Formula (Ic) has the same meaning as described herein, supra and infra.

One aspect of the present invention encompasses certain cyclohexane derivatives selected from compounds of Formula (Ie) and pharmaceutically acceptable salts, solvates, and hydrates thereof:

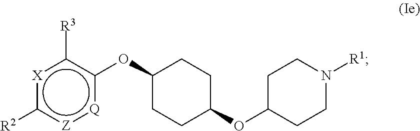

wherein each variable in Formula (Ie) has the same meaning as described herein, supra and infra.

It is further understood and appreciated that certain compounds of Formula (Ia) can bear a 2-amino-3-(3,3-difluoroazetidin-1-yl)-3-oxopropyl substituent on the phenyl ring (i.e., Q is CR⁴, Z is CR⁵, and X is CR⁶). When the absolute stereochemistry of the 2-amino-3-(3,3-difluoroazetidin-1-yl)-3-oxopropyl group is (S) then certain trans compounds of Formula (Ia) are named herein using the prefix (1S,4r) as shown below:

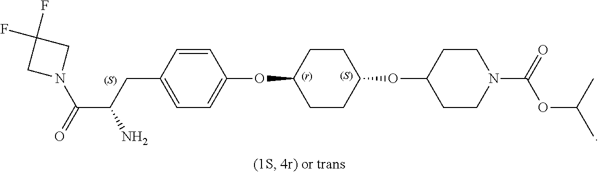

Alternatively, when the absolute stereochemistry of the 2-amino-3-(3,3-difluoroazetidin-1-yl)-3-oxopropyl group is (R), then certain trans compounds of Formula (Ia) are named herein using the prefix (1R,4r) as shown below:

### The Group Q:

In some embodiments, Q is N or CR⁴.

In some embodiments, Q is N.

In some embodiments, Q is CR⁴.

### The Group Z:

In some embodiments, Z is N or CR⁵.

In some embodiments, Z is N.

In some embodiments, Z is CR⁵.

### The Group X:

In some embodiments, X is N, N(O), or CR⁶.

In some embodiments, X is N or CR⁶.

In some embodiments, X is N.

In some embodiments, X is N(O). It is understood that the group N(O) refers to an N-oxide group.

One aspect of the present invention encompasses certain cyclohexane derivatives selected from compounds of Formula (If) and pharmaceutically acceptable salts, solvates, and hydrates thereof:

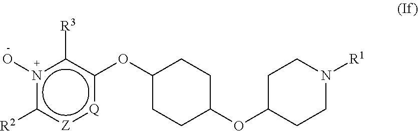

wherein each variable in Formula (If) has the same meaning as described herein, supra and infra.

In some embodiments, X is CR⁶.

### Certain Combinations of the Q, Z, and X Groups

In some embodiments, Q is N or CR⁴; Z is N or CR⁵; and X is N or CR⁶.

In some embodiments, Q is CR⁴, Z is CR⁵, and X is CR⁶.

One aspect of the present invention encompasses certain cyclohexane derivatives selected from compounds of Formula (Ig) and pharmaceutically acceptable salts, solvates, and hydrates thereof:

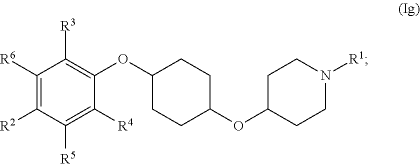

wherein each variable in Formula (Ig) has the same meaning as described herein, supra and infra.

One aspect of the present invention encompasses certain cyclohexane derivatives selected from compounds of Formula (Ii) and pharmaceutically acceptable salts, solvates, and hydrates thereof:

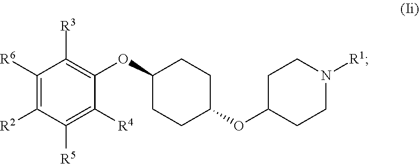

wherein each variable in Formula (Ii) has the same meaning as described herein, supra and infra.

One aspect of the present invention encompasses certain cyclohexane derivatives selected from compounds of Formula (Ik) and pharmaceutically acceptable salts, solvates, and hydrates thereof:

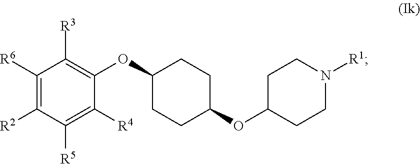

wherein each variable in Formula (Ik) has the same meaning as described herein, supra and infra.

In some embodiments, Q is N, Z is CR⁵, and X is CR⁶.

One aspect of the present invention encompasses certain cyclohexane derivatives selected from compounds of Formula (Im) and pharmaceutically acceptable salts, solvates, and hydrates thereof:

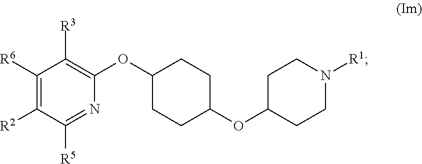

wherein each variable in Formula (Im) has the same meaning as described herein, supra and infra.

One aspect of the present invention encompasses certain cyclohexane derivatives selected from compounds of Formula (Io) and pharmaceutically acceptable salts, solvates, and hydrates thereof:

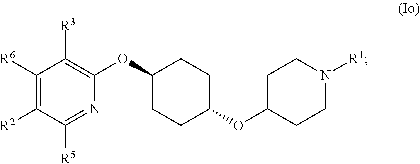

wherein each variable in Formula (Io) has the same meaning as described herein, supra and infra.

One aspect of the present invention encompasses certain cyclohexane derivatives selected from compounds of Formula (Iq) and pharmaceutically acceptable salts, solvates, and hydrates thereof:

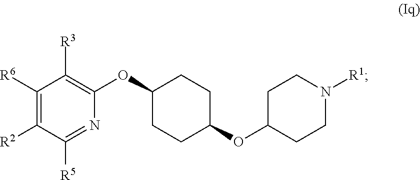

wherein each variable in Formula (Iq) has the same meaning as described herein, supra and infra.

In some embodiments, Q is N, Z is CR⁵, and X is N.

One aspect of the present invention encompasses certain cyclohexane derivatives selected from compounds of Formula (Is) and pharmaceutically acceptable salts, solvates, and hydrates thereof:

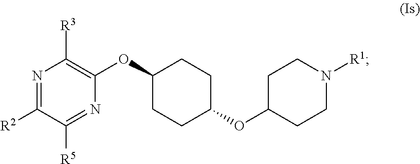

wherein each variable in Formula (Is) has the same meaning as described herein, supra and infra.

In some embodiments, Q is N, Z is N, and X is CR⁶.

One aspect of the present invention encompasses certain cyclohexane derivatives selected from compounds of Formula (Iu) and pharmaceutically acceptable salts, solvates, and hydrates thereof:

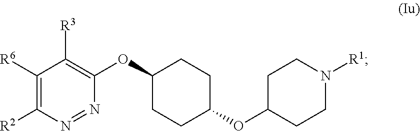

wherein each variable in Formula (Iu) has the same meaning as described herein, supra and infra.

In some embodiments, Q is CR⁴, Z is CR⁵, and X is N.

One aspect of the present invention encompasses certain cyclohexane derivatives selected from compounds of Formula (Iw) and pharmaceutically acceptable salts, solvates, and hydrates thereof:

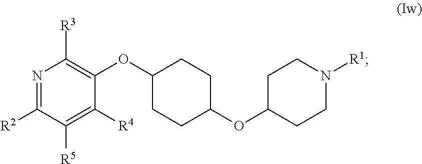

wherein each variable in Formula (Iw) has the same meaning as described herein, supra and infra.

One aspect of the present invention encompasses certain cyclohexane derivatives selected from compounds of Formula (Ix) and pharmaceutically acceptable salts, solvates, and hydrates thereof:

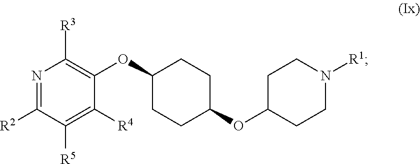

wherein each variable in Formula (Ix) has the same meaning as described herein, supra and infra.

One aspect of the present invention encompasses certain cyclohexane derivatives selected from compounds of Formula (Iy) and pharmaceutically acceptable salts, solvates, and hydrates thereof:

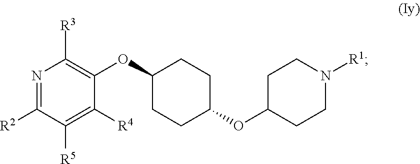

wherein each variable in Formula (Iy) has the same meaning as described herein, supra and infra.

In some embodiments, Q is N, Z is CR⁵, and X is N or CR⁶.

In some embodiments, Q is N or CR⁴, Z is CR⁵, and X is N.

One aspect of the present invention encompasses certain cyclohexane derivatives selected from compounds of Formula (IIa) and pharmaceutically acceptable salts, solvates, and hydrates thereof:

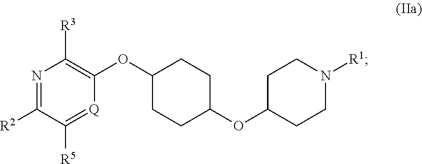

wherein each variable in Formula (IIa) has the same meaning as described herein, supra and infra.

One aspect of the present invention encompasses certain cyclohexane derivatives selected from compounds of Formula (IIc) and pharmaceutically acceptable salts, solvates, and hydrates thereof:

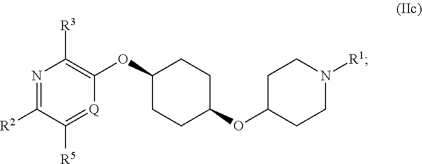

wherein each variable in Formula (IIc) has the same meaning as described herein, supra and infra.

One aspect of the present invention encompasses certain cyclohexane derivatives selected from compounds of Formula (IIe) and pharmaceutically acceptable salts, solvates, and hydrates thereof:

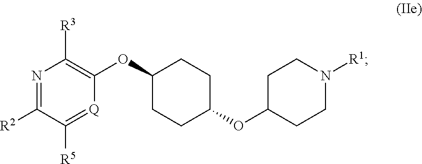

wherein each variable in Formula (IIe) has the same meaning as described herein, supra and infra.

One aspect of the present invention encompasses certain cyclohexane derivatives wherein at least one Q, Z, and X is other than N.

### The R¹ Group and Related R⁷, R⁸, R⁹, and R¹⁰ Groups

In some embodiments, R¹ is selected from the group consisting of H, S(O)₂R⁷, C(O)R⁷, CH₂R⁸, and C(O)OR⁹; or R¹ is selected from the group consisting of heteroaryl and phenyl, each optionally substituted with one or more substituents selected independently from the group consisting of C₂-C₆ alkenyl, C₁-C₆ alkoxy, C₁-C₆ alkyl, halogen, C₁-C₆ haloalkoxy, and C₁-C₆ haloalkyl;

R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;

R⁸ is selected from the group consisting of heteroaryl, phenyl, and C(O)OR⁹; wherein the heteroaryl is optionally substituted with C₁-C₆ alkyl;

R⁹ is selected from the group consisting of C₁-C₆ alkyl, C₃-C₆ cycloalkyl, C₁-C₆ haloalkyl, heterocyclyl, and phenyl; wherein the C₁-C₆ alkyl is optionally substituted with one or more substituents selected independently from the group consisting of hydroxyl, C₁-C₆ alkoxy, and R¹⁰, wherein the C₁-C₆ alkoxy is optionally substituted with phenyl; and the C₃-C₆ cycloalkyl is optionally substituted with one or more substituents selected independently from the group consisting of C₁-C₆ alkyl and halogen; and

R¹⁰ is heterocyclyl optionally substituted with C₁-C₆ alkyl.

In some embodiments, R¹ is selected from the group consisting of H, S(O)₂R⁷, C(O)R⁷, CH₂R⁸, C(O)OR⁹, and C(O)SR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of prop-1-en-2-yl, ethoxy, methoxy, tert-butyl, ethyl, isopropyl, methyl, chloro, fluoro, trifluoromethoxy, 2-fluoropropan-2-yl, and trifluoromethyl;

R⁷ is selected from the group consisting of 2,2-dimethylpropyl, isopropyl, 2-methylcyclopropyl, 1,1-difluoropropyl, and cyclopropyl;

R⁸ is selected from the group consisting of 1,2,4-oxadiazolyl, cyclopropyl, 1,1,2,2-tetrafluoroethyl, cyclobutyl, trifluoromethyl, and C(O)OR⁹; wherein said 1,2,4-oxadiazole, cyclopropyl, and cyclobutyl are each optionally substituted with one group selected from the group consisting of isopropyl and trifluoromethyl; and

R⁹ is selected from the group consisting of isobutyl, isopropyl, sec-butyl, tert-butyl, cyclopentyl, (3-methyloxetan-3-yl)methyl, 1-methylcyclopropyl, 2-methylcyclopropyl, 1,3-difluoropropan-2-yl, 1-fluoropropan-2-yl, 1,1,1,3,3,3-hexafluoropropan-2-yl, 1,1,1-trifluoropropan-2-yl, tetrahydrofuran-3-yl, 1-hydroxypropan-2-yl, phenyl, 2,2,3,3-tetrafluorocyclobutyl, 1-(benzyloxy)propan-2-yl, 1,1,1-trifluoro-2-methylpropan-2-yl, and cyclopropyl.

In some embodiments, R¹ is selected from the group consisting of H, S(O)₂R⁷, C(O)R⁷, CH₂R⁸, and C(O)OR⁹; or R¹ is selected from the group consisting of heteroaryl and phenyl, each optionally substituted with one or more substituents selected independently from the group consisting of C₂-C₆ alkenyl, C₁-C₆ alkoxy, C₁-C₆ alkyl, halogen, C₁-C₆ haloalkoxy, and C₁-C₆ haloalkyl;

R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;

R⁸ is selected from the group consisting of heteroaryl, phenyl, and C(O)OR⁹; wherein the heteroaryl is optionally substituted with C₁-C₆ alkyl;

R⁹ is selected from the group consisting of C₁-C₆ alkyl, C₃-C₆ cycloalkyl, C₁-C₆ haloalkyl, and heterocyclyl; wherein the C₁-C₆ alkyl is optionally substituted with one or more substituents selected independently from the group consisting of hydroxyl and R¹⁰; and the C₃-C₆ cycloalkyl is optionally substituted with one C₁-C₆ alkyl substituent; and

R¹⁰ is heterocyclyl optionally substituted with C₁-C₆ alkyl.

In some embodiments, R¹ is selected from the group consisting of H, S(O)₂R⁷, C(O)R⁷, CH₂R⁸, and C(O)OR⁹; or R¹ is selected from the group consisting of a five-membered heteroaryl, phenyl, and a six-membered heteroaryl, each optionally substituted with one or two substituents selected independently from the group consisting of C₂-C₆ alkenyl, C₁-C₄ alkoxy, C₁-C₆ alkyl, halogen, C₁-C₄ haloalkoxy, and C₁-C₆ haloalkyl;

R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;

R⁸ is selected from the group consisting of a five-membered heteroaryl and C(O)OR⁹; wherein the five-membered heteroaryl is optionally substituted with one C₁-C₆ alkyl substituent;

R⁹ is selected from the group consisting of C₁-C₆ alkyl, C₃-C₆ cycloalkyl, C₁-C₆ haloalkyl, heterocyclyl, and phenyl; wherein the C₁-C₆ alkyl is optionally substituted with one or more substituents selected independently from the group consisting of hydroxyl, C₁-C₆ alkoxy, and R¹⁰, wherein the C₁-C₆ alkoxy is optionally substituted with phenyl; and the C₃-C₆ cycloalkyl is optionally substituted with one or more substituents selected independently from the group consisting of C₁-C₆ alkyl and halogen; and

R¹⁰ is heterocyclyl optionally substituted with one C₁-C₆ alkyl substituent.

In some embodiments, R¹ is selected from the group consisting of H, S(O)₂R⁷, C(O)R⁷, CH₂R⁸, and C(O)OR⁹; or R¹ is selected from the group consisting of a five-membered heteroaryl, phenyl, and a six-membered heteroaryl, each optionally substituted with one or two substituents selected independently from the group consisting of C₂-C₆ alkenyl, C₁-C₄ alkoxy, C₁-C₆ alkyl, halogen, C₁-C₄ haloalkoxy, and C₁-C₆ haloalkyl;

R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;

R⁸ is selected from the group consisting of a five-membered heteroaryl and C(O)OR⁹; wherein the five-membered heteroaryl is optionally substituted with one C₁-C₆ alkyl substituent;

R⁹ is selected from the group consisting of C₁-C₆ alkyl, C₃-C₆ cycloalkyl, C₁-C₆ haloalkyl, and heterocyclyl; wherein the C₁-C₆ alkyl is optionally substituted with one or more substituents selected independently from the group consisting of hydroxyl and R¹⁰; and the C₃-C₆ cycloalkyl is optionally substituted with one C₁-C₆ alkyl substituent; and

R¹⁰ is heterocyclyl optionally substituted with one C₁-C₆ alkyl substituent.

In some embodiments, R¹ is selected from the group consisting of H, S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of C₂-C₆ alkenyl, C₁-C₄ alkoxy, C₁-C₆ alkyl, halogen, C₁-C₄ haloalkoxy, and C₁-C₆ haloalkyl;

R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;

R⁸ is selected from the group consisting of a five-membered heteroaryl and C(O)OR⁹; wherein the five-membered heteroaryl is optionally substituted with one C₁-C₆ alkyl substituent;

R⁹ is selected from the group consisting of C₁-C₆ alkyl, C₃-C₆ cycloalkyl, C₁-C₆ haloalkyl, heterocyclyl, and phenyl; wherein the C₁-C₆ alkyl is optionally substituted with one or more substituents selected independently from the group consisting of hydroxyl, C₁-C₆ alkoxy, and R¹⁰, wherein the C₁-C₆ alkoxy is optionally substituted with phenyl; and the C₃-C₆ cycloalkyl is optionally substituted with one or more substituents selected independently from the group consisting of C₁-C₆ alkyl and halogen; and

R¹⁰ is heterocyclyl optionally substituted with one C₁-C₆ alkyl substituent.

In some embodiments, R¹ is selected from the group consisting of H, S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of C₂-C₆ alkenyl, C₁-C₄ alkoxy, C₁-C₆ alkyl, halogen, C₁-C₄ haloalkoxy, and C₁-C₆ haloalkyl;

R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;

R⁸ is selected from the group consisting of a five-membered heteroaryl and C(O)OR⁹; wherein the five-membered heteroaryl is optionally substituted with one C₁-C₆ alkyl substituent;

R⁹ is selected from the group consisting of C₁-C₆ alkyl, C₃-C₆ cycloalkyl, C₁-C₆ haloalkyl, and heterocyclyl; wherein the C₁-C₆ alkyl is optionally substituted with one or more substituents selected independently from the group consisting of hydroxyl and R¹⁰; and the C₃-C₆ cycloalkyl is optionally substituted with one C₁-C₆ alkyl substituent; and

R¹⁰ is heterocyclyl optionally substituted with one C₁-C₆ alkyl substituent.

In some embodiments, R¹ is selected from the group consisting of H, S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of prop-1-en-2-yl, ethoxy, methoxy, tert-butyl, ethyl, isopropyl, methyl, chloro, fluoro, trifluoromethoxy, 2-fluoropropan-2-yl, and trifluoromethyl;

R⁷ is selected from the group consisting of 2,2-dimethylpropyl, isopropyl, 2-methylcyclopropyl, 1,1-difluoropropyl, and cyclopropyl;

R⁸ is selected from the group consisting of 1,2,4-oxadiazolyl and C(O)OR⁹; wherein the 1,2,4-oxadiazole is optionally substituted with one isopropyl group; and

R⁹ is selected from the group consisting of isobutyl, isopropyl, sec-butyl, tert-butyl, cyclopentyl, (3-methyloxetan-3-yl)methyl, 1-methylcyclopropyl, 2-methylcyclopropyl, 1,3-difluoropropan-2-yl, 1-fluoropropan-2-yl, 1,1,1,3,3,3-hexafluoropropan-2-yl, 1,1,1-trifluoropropan-2-yl, tetrahydrofuran-3-yl, 1-hydroxypropan-2-yl, phenyl, 2,2,3,3-tetrafluorocyclobutyl, and 1-(benzyloxy)propan-2-yl.

In some embodiments, R¹ is selected from the group consisting of H, S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of prop-1-en-2-yl, ethoxy, methoxy, tert-butyl, ethyl, isopropyl, methyl, chloro, fluoro, trifluoromethoxy, 2-fluoropropan-2-yl, and trifluoromethyl;

R⁷ is selected from the group consisting of 2,2-dimethylpropyl, isopropyl, 2-methylcyclopropyl, 1,1-difluoropropyl, and cyclopropyl;

R⁸ is selected from the group consisting of 1,2,4-oxadiazolyl and C(O)OR⁹; wherein the 1,2,4-oxadiazole is optionally substituted with one isopropyl group; and

R⁹ is selected from the group consisting of isobutyl, isopropyl, sec-butyl, tert-butyl, cyclopentyl, (3-methyloxetan-3-yl)methyl, 1-methylcyclopropyl, 2-methylcyclopropyl, 1,3-difluoropropan-2-yl, 1-fluoropropan-2-yl, 1,1,1,3,3,3-hexafluoropropan-2-yl, 1,1,1-trifluoropropan-2-yl, tetrahydrofuran-3-yl, and 1-hydroxypropan-2-yl.

In some embodiments, R¹ is selected from the group consisting of H, cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (tetrahydrofuran-3-yloxy)carbonyl, (1,1,1-trifluoropropan-2-yloxy)carbonyl, (1,3-difluoropropan-2-yloxy)carbonyl, (1-fluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, 5-methoxy-pyrimidin-2-yl, (1-hydroxypropan-2-yloxy)carbonyl, phenoxycarbonyl, 5-(2-fluoropropan-2-yl)-1,2,4-oxadiazol-3-yl, (2,2,3,3-tetrafluorocyclobutoxy)carbonyl, (1-(benzyloxy)propan-2-yloxy)carbonyl, isopropylthiocarbonyl, 5-methylpyridin-2-yl, 5-ethylpyridin-2-yl, (1,1,1-trifluoro-2-methylpropan-2-yloxy)carbonyl, cyclopropylthiocarbonyl, (1-(trifluoromethyl)cyclopropyl)methyl, 2,2,3,3-tetrafluoropropyl, (1-(trifluoromethyl)cyclobutyl)methyl, and 2,2,2-trifluoroethyl.

In some embodiments, R¹ is selected from the group consisting of cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (tetrahydrofuran-3-yloxy)carbonyl, (1,1,1-trifluoropropan-2-yloxy)carbonyl, (1,3-difluoropropan-2-yloxy)carbonyl, (1-fluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, 5-methoxy-pyrimidin-2-yl, (1-hydroxypropan-2-yloxy)carbonyl, phenoxycarbonyl, 5-(2-fluoropropan-2-yl)-1,2,4-oxadiazol-3-yl, (2,2,3,3-tetrafluorocyclobutoxy)carbonyl, (1-(benzyloxy)propan-2-yloxy)carbonyl, isopropylthiocarbonyl, 5-methylpyridin-2-yl, 5-ethylpyridin-2-yl, (1,1,1-trifluoro-2-methylpropan-2-yloxy)carbonyl, cyclopropylthiocarbonyl, (1-(trifluoromethyl)cyclopropyl)methyl, 2,2,3,3-tetrafluoropropyl, (1-(trifluoromethyl)cyclobutyl)methyl, and 2,2,2-trifluoroethyl.

In some embodiments, R¹ is selected from the group consisting of H, cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (tetrahydrofuran-3-yloxy)carbonyl, (1,1,1-trifluoropropan-2-yloxy)carbonyl, (1,3-difluoropropan-2-yloxy)carbonyl, (1-fluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, 5-methoxy-pyrimidin-2-yl, (1-hydroxypropan-2-yloxy)carbonyl, phenoxycarbonyl, 5-(2-fluoropropan-2-yl)-1,2,4-oxadiazol-3-yl, (2,2,3,3-tetrafluorocyclobutoxy)carbonyl, and (1-(benzyloxy)propan-2-yloxy)carbonyl.

In some embodiments, R¹ is selected from the group consisting of H, cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (tetrahydrofuran-3-yloxy)carbonyl, (1,1,1-trifluoropropan-2-yloxy)carbonyl, (1,3-difluoropropan-2-yloxy)carbonyl, (1-fluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, 5-methoxy-pyrimidin-2-yl, and (1-hydroxypropan-2-yloxy)carbonyl.

The compound according to any one of claims 1 to 10, wherein: R¹ is selected from the group consisting of S(O)₂R⁷ and C(O)R⁷; and

R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent.

In some embodiments, R¹ is selected from the group consisting of S(O)₂R⁷ and C(O)R⁷; and

R⁷ is selected from the group consisting of 2,2-dimethylpropyl, isopropyl, 2-methylcyclopropyl, 1,1-difluoropropyl, and cyclopropyl.

In some embodiments, R¹ is selected from the group consisting of cyclopropylsulfonyl, isopropylsulfonyl, 3-isobutyryl, 3,3-dimethylbutanoyl, 2-methylcyclopropanecarbonyl, and 2,2-difluorobutanoyl.

In some embodiments, R¹ is CH₂R⁸;

R⁸ is selected from the group consisting of a five-membered heteroaryl and C(O)OR⁹; wherein the five-membered heteroaryl is optionally substituted with one C₁-C₆ alkyl substituent; and

R⁹ is C₁-C₆ alkyl.

In some embodiments, R¹ is CH₂R⁸;

R⁸ is selected from the group consisting of 1,2,4-oxadiazolyl and C(O)OR⁹; wherein the 1,2,4-oxadiazole is optionally substituted with one isopropyl group; and

R⁹ is tert-butyl.

In some embodiments, R¹ is selected from the group consisting of (3-isopropyl-1,2,4-oxadiazol-5-yl)methyl and 2-tert-butoxy-2-oxoethyl.

In some embodiments, R¹ is C(O)OR⁹;

R⁹ is selected from the group consisting of C₁-C₆ alkyl, C₃-C₆ cycloalkyl, C₁-C₆ haloalkyl, heterocyclyl, and phenyl; wherein the C₁-C₆ alkyl is optionally substituted with one or more substituents selected independently from the group consisting of hydroxyl, C₁-C₆ alkoxy, and R¹⁰, wherein C₁-C₆ alkoxy is optionally substituted with phenyl; and the C₃-C₆ cycloalkyl is optionally substituted with one or more substituents selected independently from the group consisting of C₁-C₆ alkyl and halogen; and

R¹⁰ is heterocyclyl optionally substituted with one C₁-C₆ alkyl substituent.

In some embodiments, R¹ is C(O)OR⁹;

R⁹ is selected from the group consisting of C₁-C₆ alkyl, C₃-C₆ cycloalkyl, C₁-C₆ haloalkyl, and heterocyclyl; wherein the C₁-C₆ alkyl is optionally substituted with one or more substituents selected independently from the group consisting of hydroxyl and R¹⁰; and the C₃-C₆ cycloalkyl is optionally substituted with one C₁-C₆ alkyl substituent; and

R¹⁰ is heterocyclyl optionally substituted with one C₁-C₆ alkyl substituent.

In some embodiments, R¹ is C(O)OR⁹; and

R⁹ is selected from the group consisting of isobutyl, isopropyl, sec-butyl, tert-butyl, cyclopentyl, (3-methyloxetan-3-yl)methyl, 1-methylcyclopropyl, 1,3-difluoropropan-2-yl, 1-fluoropropan-2-yl, 1,1,1,3,3,3-hexafluoropropan-2-yl, 1,1,1-trifluoropropan-2-yl, tetrahydrofuran-3-yl, 1-hydroxypropan-2-yl, phenyl, 2,2,3,3-tetrafluorocyclobutyl, and 1-(benzyloxy)propan-2-yl.

In some embodiments, R¹ is C(O)OR⁹; and

R⁹ is selected from the group consisting of isobutyl, isopropyl, sec-butyl, tert-butyl, cyclopentyl, (3-methyloxetan-3-yl)methyl, 1-methylcyclopropyl, 1,3-difluoropropan-2-yl, 1-fluoropropan-2-yl, 1,1,1,3,3,3-hexafluoropropan-2-yl, 1,1,1-trifluoropropan-2-yl, tetrahydrofuran-3-yl, and 1-hydroxypropan-2-yl.

In some embodiments, R¹ is selected from the group consisting of tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (tetrahydrofuran-3-yloxy)carbonyl, (1,1,1-trifluoropropan-2-yloxy)carbonyl, (1,3-difluoropropan-2-yloxy)carbonyl, (1-fluoropropan-2-yloxy)carbonyl, (1-hydroxypropan-2-yloxy)carbonyl, phenoxycarbonyl, (2,2,3,3-tetrafluorocyclobutoxy)carbonyl, and (1-(benzyloxy)propan-2-yloxy)carbonyl.

In some embodiments, R¹ is selected from the group consisting of tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (tetrahydrofuran-3-yloxy)carbonyl, (1,1,1-trifluoropropan-2-yloxy)carbonyl, (1,3-difluoropropan-2-yloxy)carbonyl, (1-fluoropropan-2-yloxy)carbonyl, and (1-hydroxypropan-2-yloxy)carbonyl.

In some embodiments, R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of C₂-C₆ alkenyl, C₁-C₄ alkoxy, C₁-C₆ alkyl, halogen, C₁-C₄ haloalkoxy, and C₁-C₆ haloalkyl.

In some embodiments, R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of prop-1-en-2-yl, ethoxy, methoxy, tert-butyl, ethyl, isopropyl, methyl, chloro, fluoro, trifluoromethoxy, 2-fluoropropan-2-yl, and trifluoromethyl.

In some embodiments, R¹ is selected from the group consisting of 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, 5-methoxy-pyrimidin-2-yl, and 5-(2-fluoropropan-2-yl)-1,2,4-oxadiazol-3-yl.

In some embodiments, R¹ is selected from the group consisting of 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, and 5-methoxy-pyrimidin-2-yl.

In some embodiments, R¹ is selected from the group consisting of 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, and 5-(2-fluoropropan-2-yl)-1,2,4-oxadiazol-3-yl.

In some embodiments, R¹ is selected from the group consisting of 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, and 3-(prop-1-en-2-yl)-1,2,4-oxadiazol-5-yl.

In some embodiments, R¹ is selected from the group consisting of p-tolyl, 4-(trifluoromethyl)phenyl, 4-(trifluoromethoxy)phenyl, 4-methoxyphenyl, 3-(trifluoromethyl)phenyl, 4-fluorophenyl, and 4-chloro-2-fluorophenyl.

In some embodiments, R¹ is selected from the group consisting of 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, and 5-methoxy-pyrimidin-2-yl.

In some embodiments, R¹ is H.

In some embodiments, R¹ is a group other than H.

In some embodiments, R¹ is cyclopropylsulfonyl.

In some embodiments, R¹ is isopropylsulfonyl.

In some embodiments, R¹ is 3-isobutyryl.

In some embodiments, R¹ is 3,3-dimethylbutanoyl.

In some embodiments, R¹ is 2-methylcyclopropanecarbonyl.

In some embodiments, R¹ is 2,2-difluorobutanoyl.

In some embodiments, R¹ is (3-isopropyl-1,2,4-oxadiazol-5-yl)methyl.

In some embodiments, R¹ is 2-tert-butoxy-2-oxoethyl (i.e., 2-tert-butoxy-2-oxoethyl).

In some embodiments, R¹ is tert-butoxycarbonyl.

In some embodiments, R¹ is isopropoxycarbonyl.

In some embodiments, R¹ is isobutoxycarbonyl.

In some embodiments, R¹ is cyclopentyloxycarbonyl.

In some embodiments, R¹ is (1,1,1,3,3,3-hexafluoropropan-2-yloxy)carbonyl.

In some embodiments, R¹ is ((3-methyloxetan-3-yl)methoxy)carbonyl.

In some embodiments, R¹ is (1-methylcyclopropoxy)carbonyl.

In some embodiments, R¹ is sec-butoxycarbonyl.

In some embodiments, R¹ is (tetrahydrofuran-3-yloxy)carbonyl.

In some embodiments, R¹ is (1,1,1-trifluoropropan-2-yloxy)carbonyl.

In some embodiments, R¹ is (1,3-difluoropropan-2-yloxy)carbonyl.

In some embodiments, R¹ is (1-fluoropropan-2-yloxy)carbonyl.

In some embodiments, R¹ is 3-isopropyl-1,2,4-oxadiazol-5-yl.

In some embodiments, R¹ is 5-isopropyl-1,2,4-oxadiazol-3-yl.

In some embodiments, R¹ is 3-(2-fluoropropan-2-yl)-1,2,4-oxadiazol-5-yl.

In some embodiments, R¹ is 3-tert-butyl-1,2,4-oxadiazol-5-yl.

In some embodiments, R¹ is 3-(prop-1-en-2-yl)-1,2,4-oxadiazol-5-yl.

In some embodiments, R¹ is p-tolyl.

In some embodiments, R¹ is 4-(trifluoromethyl)phenyl.

In some embodiments, R¹ is 4-(trifluoromethoxy)phenyl.

In some embodiments, R¹ is 4-methoxyphenyl.

In some embodiments, R¹ is 3-(trifluoromethyl)phenyl.

In some embodiments, R¹ is 4-fluorophenyl.

In some embodiments, R¹ is 4-chloro-2-fluorophenyl.

In some embodiments, R¹ is 5-ethyl-pyrimidin-2-yl.

In some embodiments, R¹ is 5-chloro-pyrimidin-2-yl.

In some embodiments, R¹ is 5-methyl-pyrimidin-2-yl.

In some embodiments, R¹ is 5-(trifluoromethyl)pyridin-2-yl.

In some embodiments, R¹ is 3-methyl-pyridazin-6-yl.

In some embodiments, R¹ is 2-methyl-pyrazin-5-yl.

In some embodiments, R¹ is 5-chloro-pyridin-2-yl.

In some embodiments, R¹ is 3-ethoxy-pyridazin-6-yl.

In some embodiments, R¹ is 5-fluoro-pyridin-2-yl.

In some embodiments, R¹ is 5-methoxy-pyrimidin-2-yl.

In some embodiments, R¹ is (1-hydroxypropan-2-yloxy)carbonyl.

In some embodiments, R¹ is phenoxycarbonyl.

In some embodiments, R¹ is 5-(2-fluoropropan-2-yl)-1,2,4-oxadiazol-3-yl.

In some embodiments, R¹ is (2,2,3,3-tetrafluorocyclobutoxy)carbonyl.

In some embodiments, R¹ is (1-(benzyloxy)propan-2-yloxy)carbonyl.

In some embodiments, R¹ is isopropylthiocarbonyl.

In some embodiments, R¹ is 5-methylpyridin-2-yl.

In some embodiments, R¹ is 5-ethylpyridin-2-yl.

In some embodiments, R¹ is (1,1,1-trifluoro-2-methylpropan-2-yloxy)carbonyl.

In some embodiments, R¹ is cyclopropylthiocarbonyl.

In some embodiments, R¹ is (1-(trifluoromethyl)cyclopropyl)methyl.

In some embodiments, R¹ is 2,2,3,3-tetrafluoropropyl.

In some embodiments, R¹ is (1-(trifluoromethyl)cyclobutyl)methyl.

In some embodiments, R¹ is 2,2,2-trifluoroethyl.

### The R², R³, R⁴, R⁵, and R⁶ Groups; and Related R¹¹, R¹², and R¹³ Groups

In some embodiments, R² is selected from the group consisting of H, C₁-C₆ alkyl, cyano, C₃-C₆ cycloalkyl, halogen, C₁-C₆ haloalkyl, heteroaryl, heterocyclyl, S(O)₂R¹¹, and C(O)NR¹²R¹³; wherein the C₁-C₆ alkyl is optionally substituted with one or more substituents selected independently from the group consisting of amino, C₁-C₆ alkylsulfonyl, cyano, and C(O)NR¹²R¹³; the C₃-C₆ cycloalkyl is optionally substituted with C(O)NR¹²R¹³; the heteroaryl is optionally substituted with C₁-C₆ alkyl; and the heterocyclyl is optionally substituted with one or more substituents selected independently from the group consisting of amino, C₁-C₆ alkylsulfonyl, hydroxyl, and halogen;

R³, R⁴, R⁵, and R⁶ are each independently selected from the group consisting of H, C₁-C₆ alkyl, C₁-C₆ alkylsulfonyl, and halogen;

R¹¹ is selected from the group consisting of C₁-C₆ alkyl, C₃-C₆ cycloalkyl, C₁-C₆ haloalkyl, and heterocyclyl; wherein the C₁-C₆ alkyl is optionally substituted with one or more substituents selected independently from the group consisting of hydroxyl and NR¹²R¹³; and the heterocyclyl is optionally substituted with one or more substituents selected independently from the group consisting of amino, C₁-C₆ alkyl, and hydroxyl; and

R¹² and R¹³ are each independently selected from the group consisting of H, C₁-C₆ alkyl, and C₃-C₆ cycloalkyl; wherein the C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, hydroxyl, and C₁-C₆ alkoxy.

In some embodiments, R² is selected from the group consisting of H, C₁-C₆ alkyl, cyano, C₃-C₆ cycloalkyl, halogen, C₁-C₆ haloalkyl, heteroaryl, heterocyclyl, S(O)₂R¹¹, and C(O)NR¹²R¹³; wherein the C₁-C₆ alkyl is optionally substituted with one or more substituents selected independently from the group consisting of amino, C₁-C₆ alkylsulfonyl, cyano, and C(O)NR¹²R¹³; the C₃-C₆ cycloalkyl is optionally substituted with C(O)NR¹²R¹³; the heteroaryl is optionally substituted with C₁-C₆ alkyl; and the heterocyclyl is optionally substituted with one or more substituents selected independently from the group consisting of amino, C₁-C₆ alkylsulfonyl, hydroxyl, and halogen;

R³, R⁴, R⁵, and R⁶ are each independently selected from the group consisting of H, C₁-C₆ alkyl, C₁-C₆ alkylsulfonyl, and halogen;

R¹¹ is selected from the group consisting of C₁-C₆ alkyl, C₃-C₆ cycloalkyl, C₁-C₆ haloalkyl, and heterocyclyl; wherein the C₁-C₆ alkyl is optionally substituted with one or more substituents selected independently from the group consisting of hydroxyl and NR¹²R¹³; and the heterocyclyl is optionally substituted with one or more substituents selected independently from the group consisting of amino, C₁-C₆ alkyl, and hydroxyl; and

R¹² and R¹³ are each independently selected from the group consisting of H, C₁-C₆ alkyl, and C₃-C₆ cycloalkyl; wherein the C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, and hydroxyl.

In some embodiments, R² is selected from the group consisting of H, C₁-C₆ alkyl, cyano, halogen, heteroaryl, heterocyclyl, S(O)ₙR¹¹, and C(O)NR¹²R¹³; wherein said C₁-C₆ alkyl is optionally substituted with one or more substituents selected independently from the group consisting of amino, cyano, and C(O)NR¹²R¹³;

R¹¹ is selected from the group consisting of C₁-C₆ alkyl, C₃-C₆ cycloalkyl, and heterocyclyl; wherein said heterocyclyl is optionally substituted with one or two halogens; and

R¹² and R¹³ are each independently C₁-C₆ alkyl; or R¹² and R¹³ together with the nitrogen to which they are both bonded form a heterocyclyl optionally substituted with one or more substituents selected independently from the group consisting of halogen and C₁-C₆ alkoxy.

In some embodiments, R² is selected from the group consisting of H, C₁-C₆ alkyl, cyano, halogen, heteroaryl, heterocyclyl, S(O)₂R¹¹, and C(O)NR¹²R¹³; wherein the C₁-C₆ alkyl is optionally substituted with one or more substituents selected independently from the group consisting of amino, cyano, and C(O)NR¹²R¹³;

R¹¹ is selected from the group consisting of C₁-C₆ alkyl and C₃-C₆ cycloalkyl; and

R¹² and R¹³ are each independently C₁-C₆ alkyl; or R¹² and R¹³ together with the nitrogen to which they are both bonded form a heterocyclyl optionally substituted with one or more substituents selected independently from the group consisting of halogen and C₁-C₆ alkoxy.

In some embodiments, R² is selected from the group consisting of H, C₁-C₆ alkyl, cyano, halogen, heteroaryl, heterocyclyl, S(O)₂R¹¹, and C(O)NR¹²R¹³; wherein the C₁-C₆ alkyl is optionally substituted with cyano;

R¹¹ is C₁-C₆ alkyl; and

R¹² and R¹³ are each independently C₁-C₆ alkyl.

In some embodiments, R² is selected from the group consisting of H, methyl, isopropylsulfonyl, methylsulfonyl, cyano, dimethylcarbamoyl, bromo, fluoro, pyridazin-4-yl, 1H-1,2,4-triazol-1-yl, 1,1-dioxo-thiomorpholin-4-yl, morpholin-4-yl, 2-cyanoethyl, cyclopropylsulfonyl, 2-amino-3-(3,3-difluoroazetidin-1-yl)-3-oxopropyl, ethylsulfonyl, pyrimidin-5-yl, 3-methoxyazetidine-1-carbonyl, and 3,3-difluoroazetidin-1-ylsulfonyl.

In some embodiments, R² is selected from the group consisting of H, methyl, isopropylsulfonyl, methylsulfonyl, cyano, dimethylcarbamoyl, bromo, fluoro, pyridazin-4-yl, 1H-1,2,4-triazol-1-yl, 1,1-dioxo-thiomorpholin-4-yl, morpholin-4-yl, 2-cyanoethyl, cyclopropylsulfonyl, 2-amino-3-(3,3-difluoroazetidin-1-yl)-3-oxopropyl, ethylsulfonyl, pyrimidin-5-yl, and 3-methoxyazetidine-1-carbonyl.

In some embodiments, R² is selected from the group consisting of H, methyl, isopropylsulfonyl, methylsulfonyl, cyano, dimethylcarbamoyl, bromo, fluoro, pyridazin-4-yl, 1H-1,2,4-triazol-1-yl, 1,1-dioxo-thiomorpholin-4-yl, morpholin-4-yl, and 2-cyanoethyl.

In some embodiments, R³, R⁴, R⁵, and R⁶ are each independently selected from the group consisting of H, methyl, methylsulfonyl, and fluoro.

In some embodiments,

R² is selected from the group consisting of H, C₁-C₆ alkyl, cyano, halogen, heteroaryl, heterocyclyl, S(O)₂R¹¹, and C(O)NR¹²R¹³; wherein the C₁-C₆ alkyl is optionally substituted with one or more substituents selected independently from the group consisting of amino, cyano, and C(O)NR¹²R¹³;

R³ is selected from the group consisting of H and C₁-C₄ alkylsulfonyl;

R⁴ is selected from the group consisting of H and halogen;

R⁵ is selected from the group consisting of H, halogen and C₁-C₆ alkyl;

R⁶ is selected from the group consisting of H and halogen;

R¹¹ is selected from the group consisting of C₁-C₆ alkyl and C₃-C₆ cycloalkyl; and

R¹² and R¹³ are each independently C₁-C₆ alkyl; or R¹² and R¹³ together with the nitrogen to which they are both bonded form a heterocyclyl optionally substituted with one or more substituents selected independently from the group consisting of halogen and C₁-C₆ alkoxy.

In some embodiments,

R² is selected from the group consisting of H, C₁-C₆ alkyl, cyano, halogen, heteroaryl, heterocyclyl, S(O)₂R¹¹, and C(O)NR¹²R¹³; wherein the C₁-C₆ alkyl is optionally substituted with cyano;

R³ is selected from the group consisting of H and C₁-C₄ alkylsulfonyl;

R⁴ is selected from the group consisting of H and halogen;

R⁵ is selected from the group consisting of H, halogen, and C₁-C₆ alkyl;

R⁶ is selected from the group consisting of H and halogen;

R¹¹ is C₁-C₆ alkyl; and

R¹² and R¹³ are each independently C₁-C₆ alkyl.

In some embodiments,

R² is selected from the group consisting of H, methyl, isopropylsulfonyl, methylsulfonyl, cyano, dimethylcarbamoyl, bromo, fluoro, pyridazin-4-yl, 1H-1,2,4-triazol-1-yl, 1,1-dioxo-thiomorpholin-4-yl, morpholin-4-yl, 2-cyanoethyl, cyclopropylsulfonyl, 2-amino-3-(3,3-difluoroazetidin-1-yl)-3-oxopropyl, ethylsulfonyl, pyrimidin-5-yl, and 3-methoxyazetidine-1-carbonyl;

R³ is selected from the group consisting of H and methylsulfonyl;

R⁴ is selected from the group consisting of H and fluoro;

R⁵ is selected from the group consisting of H, fluoro, and methyl; and

R⁶ is selected from the group consisting of H and fluoro.

In some embodiments,

R² is selected from the group consisting of H, methyl, isopropylsulfonyl, methylsulfonyl, cyano, dimethylcarbamoyl, bromo, fluoro, pyridazin-4-yl, 1H-1,2,4-triazol-1-yl, 1,1-dioxo-thiomorpholin-4-yl, morpholin-4-yl, and 2-cyanoethyl;

R³ is selected from the group consisting of H and methylsulfonyl;

R⁴ is selected from the group consisting of H and fluoro;

R⁵ is selected from the group consisting of H, fluoro, and methyl; and

R⁶ is selected from the group consisting of H and fluoro.

In some embodiments, R² is H.

In some embodiments, R² is C₁-C₄ alkylsulfonyl.

In some embodiments, R² is cyano.

In some embodiments, R² is C₂-C₆ dialkylcarboxamide

In some embodiments, R² is halogen.

In some embodiments, R² is heteroaryl.

In some embodiments, R² is a five-membered heteroaryl.

In some embodiments, R² is a six-membered heteroaryl.

In some embodiments, R² is a heterocyclyl.

In some embodiments, R² is C₁-C₆ alkyl optionally substituted with one cyano group.

In some embodiments, R² is isopropylsulfonyl.

In some embodiments, R² is methylsulfonyl.

In some embodiments, R² is cyano.

In some embodiments, R² is dimethylcarbamoyl.

In some embodiments, R² is bromo.

In some embodiments, R² is 2-cyanoethyl.

In some embodiments, R² is 1H-1,2,4-triazol-1-yl.

In some embodiments, R² is pyridazin-4-yl.

In some embodiments, R² is 1,1-dioxo-thiomorpholin-4-yl.

In some embodiments, R² is morpholin-4-yl.

In some embodiments, R² is cyclopropylsulfonyl.

In some embodiments, R² is 2-amino-3-(3,3-difluoroazetidin-1-yl)-3-oxopropyl.

In some embodiments, R² is ethylsulfonyl, pyrimidin-5-yl.

In some embodiments, R² is 3-methoxyazetidine-1-carbonyl.

In some embodiments, R² is 3,3-difluoroazetidin-1-ylsulfonyl.

In some embodiments, R³ is H.

In some embodiments, R³ is C₁-C₄ alkylsulfonyl.

In some embodiments, R³ is methylsulfonyl.

In some embodiments, R⁴ is H.

In some embodiments, R⁴ is halogen.

In some embodiments, R⁴ is fluoro.

In some embodiments, R⁵ is H.

In some embodiments, R⁵ is halogen.

In some embodiments, R⁵ is C₁-C₆ alkyl.

In some embodiments, R⁵ is fluoro.

In some embodiments, R⁵ is methyl.

In some embodiments, R⁶ is H.

In some embodiments, R⁶ is halogen.

In some embodiments, R⁶ is fluoro.

### Certain Combinations

### Certain Embodiments

### R⁹, R¹², and R¹³

In some embodiments, R⁹ is selected from the group consisting of C₁-C₆ alkyl, C₃-C₆ cycloalkyl, C₁-C₆ haloalkyl, heterocyclyl, and phenyl; wherein the C₁-C₆ alkyl is optionally substituted with one or more substituents selected independently from the group consisting of hydroxyl, C₁-C₆ alkoxy, and R¹⁰, wherein the C₁-C₆ alkoxy is optionally substituted with phenyl; and the C₃-C₆ cycloalkyl is optionally substituted with one or more substituents selected independently from the group consisting of C₁-C₆ alkyl and halogen; and

R¹² and R¹³ are each independently selected from the group consisting of H, C₁-C₆ alkyl, and C₃-C₆ cycloalkyl; wherein the C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, hydroxyl, and C₁-C₆ alkoxy.

In some embodiments, R⁹ is selected from the group consisting of C₁-C₆ alkyl, C₃-C₆ cycloalkyl, C₁-C₆ haloalkyl, and heterocyclyl; wherein the C₁-C₆ alkyl is optionally substituted with one or more substituents selected independently from the group consisting of hydroxyl and R¹⁰; and the C₃-C₆ cycloalkyl is optionally substituted with one C₁-C₆ alkyl substituent; and

R¹² and R¹³ are each independently selected from the group consisting of H, C₁-C₆ alkyl, and C₃-C₆ cycloalkyl; wherein the C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, and hydroxyl.

### Certain Embodiments Wherein Q is CR⁴, Z is CR⁵, and X is CR⁶

One aspect of the present invention encompasses certain cyclohexyl derivatives selected from compounds of Formula (Ig) and pharmaceutically acceptable salts, solvates, and hydrates thereof:

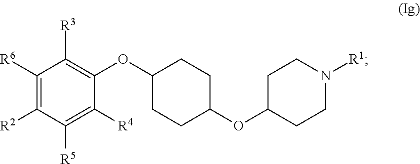

wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of C₁-C₄ alkoxy, C₁-C₆ alkyl, halogen, C₁-C₄ haloalkoxy, and C₁-C₆ haloalkyl;
R² is selected from the group consisting of H, C₁-C₆ alkyl, cyano, heteroaryl, and S(O)₂R¹¹; wherein the C₁-C₆ alkyl is optionally substituted with one or more substituents selected independently from the group consisting of amino, cyano, and C(O)NR¹²R¹³;
R³, R⁴, R⁵, and R⁶ are each independently selected from the group consisting of H, C₁-C₆ alkylsulfonyl, and halogen;
R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;
R⁸ is selected from the group consisting of a five-membered heteroaryl and C(O)OR⁹; wherein the five-membered heteroaryl is optionally substituted with one C₁-C₆ alkyl substituent;
R⁹ is selected from the group consisting of C₁-C₆ alkyl, C₃-C₆ cycloalkyl, and C₁-C₆ haloalkyl; wherein the C₁-C₆ alkyl is optionally substituted with one R¹⁰ substituent; and the C₃-C₆ cycloalkyl is optionally substituted with one C₁-C₆ alkyl substituent;
R¹⁰ is heterocyclyl optionally substituted with one C₁-C₆ alkyl substituent;
R¹¹ is C₁-C₆ alkyl; and
R¹² and R¹³ together with the nitrogen to which they are both bonded form a heterocyclyl optionally substituted with one or two halogens.

One aspect of the present invention encompasses certain cyclohexyl derivatives selected from compounds of Formula (Ig) and pharmaceutically acceptable salts, solvates, and hydrates thereof, wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of C₁-C₄ alkoxy, C₁-C₆ alkyl, halogen, C₁-C₄ haloalkoxy, and C₁-C₆ haloalkyl;
R² is selected from the group consisting of H, C₁-C₆ alkyl, cyano, heteroaryl, and S(O)₂R¹¹; wherein the C₁-C₆ alkyl is optionally substituted with cyano;
R³, R⁴, R⁵, and R⁶ are each independently selected from the group consisting of H, C₁-C₆ alkylsulfonyl, and halogen;
R⁷ is selected from the group consisting of C₁-C₆ alkyl, or C₃-C₆ cycloalkyl, and C₁-C₆ haloalkyl; wherein the C₃-C₆ cycloalkyl is optionally substituted with one C₁-C₆ alkyl substituent;
R⁸ is selected from the group consisting of a five-membered heteroaryl and C(O)OR⁹; wherein the five-membered heteroaryl is optionally substituted with one C₁-C₆ alkyl substituent;
R⁹ is selected from the group consisting of C₁-C₆ alkyl, C₃-C₆ cycloalkyl, and C₁-C₆ haloalkyl; wherein the C₁-C₆ alkyl is optionally substituted with one R¹⁰ substituent; and the C₃-C₆ cycloalkyl is optionally substituted with one C₁-C₆ alkyl substituent;
R¹⁰ is heterocyclyl optionally substituted with one C₁-C₆ alkyl substituent; and
R¹¹ is C₁-C₆ alkyl.

One aspect of the present invention encompasses certain cyclohexyl derivatives selected from compounds of Formula (Ig) and pharmaceutically acceptable salts, solvates, and hydrates thereof, wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of ethoxy, methoxy, tert-butyl, ethyl, isopropyl, methyl, chloro, fluoro, trifluoromethoxy, 2-fluoropropan-2-yl, and trifluoromethyl;
R² is selected from the group consisting of H, methylsulfonyl, cyano, 1H-1,2,4-triazol-1-yl, 2-cyanoethyl, and 2-amino-3-(3,3-difluoroazetidin-1-yl)-3-oxopropyl;
R³, R⁴, R⁵, and R⁶ are each independently selected from the group consisting of H, methylsulfonyl, and fluoro;
R⁷ is selected from the group consisting of 2,2-dimethylpropyl, isopropyl, 2-methylcyclopropyl, 1,1-difluoropropyl, and cyclopropyl;
R⁸ is selected from the group consisting of 1,2,4-oxadiazolyl and C(O)OR⁹; wherein the 1,2,4-oxadiazole is optionally substituted with one isopropyl group; and
R⁹ is selected from the group consisting of isobutyl, isopropyl, sec-butyl, tert-butyl, cyclopentyl, (3-methyloxetan-3-yl)methyl, 1-methylcyclopropyl, 1,1,1,3,3,3-hexafluoropropan-2-yl, and 1,1,1-trifluoropropan-2-yl.

One aspect of the present invention encompasses certain cyclohexyl derivatives selected from compounds of Formula (Ig) and pharmaceutically acceptable salts, solvates, and hydrates thereof, wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of ethoxy, methoxy, tert-butyl, ethyl, isopropyl, methyl, chloro, fluoro, trifluoromethoxy, 2-fluoropropan-2-yl, and trifluoromethyl;
R² is selected from the group consisting of H, methylsulfonyl, cyano, 1H-1,2,4-triazol-1-yl, and 2-cyanoethyl;
R³, R⁴, R⁵, and R⁶ are each independently selected from the group consisting of H, methylsulfonyl, and fluoro;
R⁷ is selected from the group consisting of 2,2-dimethylpropyl, isopropyl, 2-methylcyclopropyl, 1,1-difluoropropyl, and cyclopropyl;
R⁸ is selected from the group consisting of 1,2,4-oxadiazolyl and C(O)OR⁹; wherein the 1,2,4-oxadiazole is optionally substituted with one isopropyl group; and
R⁹ is selected from the group consisting of isobutyl, isopropyl, sec-butyl, tert-butyl, cyclopentyl, (3-methyloxetan-3-yl)methyl, 1-methylcyclopropyl, 1,1,1,3,3,3-hexafluoropropan-2-yl, and 1,1,1-trifluoropropan-2-yl.

One aspect of the present invention encompasses certain cyclohexyl derivatives selected from compounds of Formula (Ig) and pharmaceutically acceptable salts, solvates, and hydrates thereof, wherein:
R¹ is selected from the group consisting of cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (1,1,1-trifluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, and 5-methoxy-pyrimidin-2-yl;
R² is selected from the group consisting of H, methylsulfonyl, cyano, 1H-1,2,4-triazol-1-yl, 2-cyanoethyl, and 2-amino-3-(3,3-difluoroazetidin-1-yl)-3-oxopropyl;
R³ is selected from the group consisting of H and methylsulfonyl;
R⁴ is selected from the group consisting of H and fluoro;
R⁵ is selected from the group consisting of H, and fluoro; and
R⁶ is selected from the group consisting of H and fluoro.

One aspect of the present invention encompasses certain cyclohexyl derivatives selected from compounds of Formula (Ig) and pharmaceutically acceptable salts, solvates, and hydrates thereof, wherein:
R¹ is selected from the group consisting of cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (1,1,1-trifluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, and 5-methoxy-pyrimidin-2-yl;
R² is selected from the group consisting of H, methylsulfonyl, cyano, 1H-1,2,4-triazol-1-yl, and 2-cyanoethyl;
R³ is selected from the group consisting of H and methylsulfonyl;
R⁴ is selected from the group consisting of H and fluoro;
R⁵ is selected from the group consisting of H, and fluoro; and
R⁶ is selected from the group consisting of H and fluoro.

One aspect of the present invention encompasses certain cyclohexyl derivatives selected from compounds of Formula (Ii) and pharmaceutically acceptable salts, solvates, and hydrates thereof:

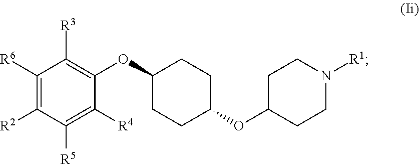

wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of C₁-C₄ alkoxy, C₁-C₆ alkyl, halogen, C₁-C₄ haloalkoxy, and C₁-C₆ haloalkyl;
R² is selected from the group consisting of H, C₁-C₆ alkyl, cyano, heteroaryl, and S(O)₂R¹¹; wherein the C₁-C₆ alkyl is optionally substituted with one or more substituents selected independently from the group consisting of amino, cyano, and C(O)NR¹²R¹³;
R³, R⁴, R⁵, and R⁶ are each independently selected from the group consisting of H, C₁-C₆ alkylsulfonyl, and halogen;
R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;
R⁸ is selected from the group consisting of a five-membered heteroaryl and C(O)OR⁹; wherein the five-membered heteroaryl is optionally substituted with one C₁-C₆ alkyl substituent;
R⁹ is selected from the group consisting of C₁-C₆ alkyl, C₃-C₆ cycloalkyl, and C₁-C₆ haloalkyl; wherein the C₁-C₆ alkyl is optionally substituted with one R¹⁰ substituent; and the C₃-C₆ cycloalkyl is optionally substituted with one C₁-C₆ alkyl substituent;
R¹⁰ is heterocyclyl optionally substituted with one C₁-C₆ alkyl substituent;
R¹¹ is C₁-C₆ alkyl; and
R¹² and R¹³ together with the nitrogen to which they are both bonded form a heterocyclyl optionally substituted with one or two halogens.

One aspect of the present invention encompasses certain cyclohexyl derivatives selected from compounds of Formula (Ii) and pharmaceutically acceptable salts, solvates, and hydrates thereof, wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of C₁-C₄ alkoxy, C₁-C₆ alkyl, halogen, C₁-C₄ haloalkoxy, and C₁-C₆ haloalkyl;
R² is selected from the group consisting of H, C₁-C₆ alkyl, cyano, heteroaryl, and S(O)₂R¹¹; wherein the C₁-C₆ alkyl is optionally substituted with cyano;
R³, R⁴, R⁵, and R⁶ are each independently selected from the group consisting of H, C₁-C₆ alkylsulfonyl, and halogen;
R⁷ is selected from the group consisting of C₁-C₆ alkyl, C₃-C₆ cycloalkyl, and C₁-C₆ haloalkyl; wherein the C₃-C₆ cycloalkyl is optionally substituted with one C₁-C₆ alkyl substituent;
R⁸ is selected from the group consisting of a five-membered heteroaryl and C(O)OR⁹; wherein the five-membered heteroaryl is optionally substituted with one C₁-C₆ alkyl substituent;
R⁹ is selected from the group consisting of C₁-C₆ alkyl, C₃-C₆ cycloalkyl, and C₁-C₆ haloalkyl; wherein the C₁-C₆ alkyl is optionally substituted with one R¹⁰ substituent; and the C₃-C₆ cycloalkyl is optionally substituted with one C₁-C₆ alkyl substituent;
R¹⁰ is heterocyclyl optionally substituted with one C₁-C₆ alkyl substituent; and
R¹¹ is C₁-C₆ alkyl.

One aspect of the present invention encompasses certain cyclohexyl derivatives selected from compounds of Formula (Ii) and pharmaceutically acceptable salts, solvates, and hydrates thereof, wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of ethoxy, methoxy, tert-butyl, ethyl, isopropyl, methyl, chloro, fluoro, trifluoromethoxy, 2-fluoropropan-2-yl, and trifluoromethyl;
R² is selected from the group consisting of H, methylsulfonyl, cyano, 1H-1,2,4-triazol-1-yl, 2-cyanoethyl, and 2-amino-3-(3,3-difluoroazetidin-1-yl)-3-oxopropyl;
R³, R⁴, R⁵, and R⁶ are each independently selected from the group consisting of H, methylsulfonyl, and fluoro;
R⁷ is selected from the group consisting of 2,2-dimethylpropyl, isopropyl, 2-methylcyclopropyl, 1,1-difluoropropyl, and cyclopropyl;
R⁸ is selected from the group consisting of 1,2,4-oxadiazolyl and C(O)OR⁹; wherein the 1,2,4-oxadiazole is optionally substituted with one isopropyl group; and
R⁹ is selected from the group consisting of isobutyl, isopropyl, sec-butyl, tert-butyl, cyclopentyl, (3-methyloxetan-3-yl)methyl, 1-methylcyclopropyl, 1,1,1,3,3,3-hexafluoropropan-2-yl, and 1,1,1-trifluoropropan-2-yl.

One aspect of the present invention encompasses certain cyclohexyl derivatives selected from compounds of Formula (Ii) and pharmaceutically acceptable salts, solvates, and hydrates thereof, wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of ethoxy, methoxy, tert-butyl, ethyl, isopropyl, methyl, chloro, fluoro, trifluoromethoxy, 2-fluoropropan-2-yl, and trifluoromethyl;
R² is selected from the group consisting of H, methylsulfonyl, cyano, 1H-1,2,4-triazol-1-yl, and 2-cyanoethyl;
R³, R⁴, R⁵, and R⁶ are each independently selected from the group consisting of H, methylsulfonyl, and fluoro;
R⁷ is selected from the group consisting of 2,2-dimethylpropyl, isopropyl, 2-methylcyclopropyl, 1,1-difluoropropyl, and cyclopropyl;
R⁸ is selected from the group consisting of 1,2,4-oxadiazolyl and C(O)OR⁹; wherein the 1,2,4-oxadiazole is optionally substituted with one isopropyl group; and
R⁹ is selected from the group consisting of isobutyl, isopropyl, sec-butyl, tert-butyl, cyclopentyl, (3-methyloxetan-3-yl)methyl, 1-methylcyclopropyl, 1,1,1,3,3,3-hexafluoropropan-2-yl, and 1,1,1-trifluoropropan-2-yl.

One aspect of the present invention encompasses certain cyclohexyl derivatives selected from compounds of Formula (Ii) and pharmaceutically acceptable salts, solvates, and hydrates thereof, wherein:
R¹ is selected from the group consisting of cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (1,1,1-trifluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, and 5-methoxy-pyrimidin-2-yl;
R² is selected from the group consisting of H, methylsulfonyl, cyano, 1H-1,2,4-triazol-1-yl, 2-cyanoethyl, and 2-amino-3-(3,3-difluoroazetidin-1-yl)-3-oxopropyl;
R³ is selected from the group consisting of H and methylsulfonyl;
R⁴ is selected from the group consisting of H and fluoro;
R⁵ is selected from the group consisting of H and fluoro; and
R⁶ is selected from the group consisting of H and fluoro.

One aspect of the present invention encompasses certain cyclohexyl derivatives selected from compounds of Formula (Ii) and pharmaceutically acceptable salts, solvates, and hydrates thereof, wherein:
R¹ is selected from the group consisting of cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (1,1,1-trifluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, and 5-methoxy-pyrimidin-2-yl;
R² is selected from the group consisting of H, methylsulfonyl, cyano, 1H-1,2,4-triazol-1-yl, and 2-cyanoethyl;
R³ is selected from the group consisting of H and methylsulfonyl;
R⁴ is selected from the group consisting of H and fluoro;
R⁵ is selected from the group consisting of H and fluoro; and
R⁶ is selected from the group consisting of H and fluoro.

One aspect of the present invention encompasses certain cyclohexyl derivatives selected from compounds of Formula (Ik) and pharmaceutically acceptable salts, solvates, and hydrates thereof:

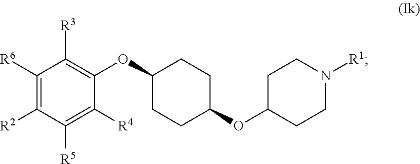

wherein:
R¹ is C(O)OR⁹; or R¹ is selected from the group consisting of 1,2,4-oxadiazolyl and pyrimidinyl, each optionally substituted with one C₁-C₆ alkyl substituent;
R² is selected from the group consisting of H and S(O)₂R¹¹;
R³, R⁴, R⁵, and R⁶ are each independently selected from the group consisting of H and halogen;
R⁹ is C₁-C₆ alkyl; and
R¹¹ is C₁-C₆ alkyl.

One aspect of the present invention encompasses certain cyclohexyl derivatives selected from compounds of Formula (Ik) and pharmaceutically acceptable salts, solvates, and hydrates thereof, wherein:
R¹ is C(O)OR⁹; or R¹ is selected from the group consisting of 1,2,4-oxadiazolyl and pyrimidinyl, each optionally substituted with one or more substituents selected independently from the group consisting of ethyl and isopropyl;
R² is methylsulfonyl;
R³, R⁴, R⁵, and R⁶ are each independently selected from the group consisting of H, and fluoro; and
R⁹ is tert-butyl.

One aspect of the present invention encompasses certain cyclohexyl derivatives selected from compounds of Formula (Ik) and pharmaceutically acceptable salts, solvates, and hydrates thereof, wherein:
R¹ is selected from the group consisting of tert-butoxycarbonyl, 3-isopropyl-1,2,4-oxadiazol-5-yl, and 5-ethyl-pyrimidin-2-yl;
R² is methylsulfonyl;
R³ is H;
R⁴ is selected from the group consisting of H and fluoro;
R⁵ is H; and
R⁶ is H.

### Certain Embodiments Wherein One or Two of Q, Z, and X are N

One aspect of the present invention encompasses certain cyclohexyl derivatives selected from compounds of Formula (Ia) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶; or
Q is CR⁴; Z is CR⁵; and X is N;
R¹ is selected from the group consisting of C(O)R⁷, CH₂R⁸, and C(O)OR⁹; or R¹ is selected from the group consisting of 1,2,4-oxadiazolyl and pyrimidinyl, each optionally substituted with 1 substituent selected from the group consisting of C₂-C₆ alkenyl, C₁-C₆ alkyl, and C₁-C₆ haloalkyl;
R² is selected from the group consisting of H, cyano, halogen, heteroaryl, heterocyclyl, S(O)₂R¹¹, and C(O)NR¹²R¹³;
R³, R⁴, R⁵, and R⁶ are each independently selected from the group consisting of H and C₁-C₆ alkyl;
R⁷ is C₁-C₆ haloalkyl;
R⁸ is a five-membered heteroaryl optionally substituted with one C₁-C₆ alkyl substituent;
R⁹ is selected from the group consisting of C₁-C₆ alkyl, C₃-C₆ cycloalkyl, C₁-C₆ haloalkyl, heterocyclyl, and phenyl; wherein the C₁-C₆ alkyl is optionally substituted with one or more substituents selected independently from the group consisting of hydroxyl, C₁-C₆ alkoxy, and R¹⁰, wherein C₁-C₆ alkoxy is optionally substituted with phenyl; and the C₃-C₆ cycloalkyl is optionally substituted with one or more substituents selected independently from the group consisting of C₁-C₆ alkyl and halogen;
R¹¹ is selected from the group consisting of C₁-C₆ alkyl and C₃-C₆ cycloalkyl; and
R¹² and R¹³ are each independently selected from the group consisting of C₁-C₆ alkyl; or R¹² and R¹³ together with the nitrogen to which they are both bonded form a heterocyclyl optionally substituted with C₁-C₆ alkoxy.

One aspect of the present invention encompasses certain cyclohexyl derivatives selected from compounds of Formula (Ia) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶; or
Q is CR⁴; Z is CR⁵; and X is N;
R¹ is selected from the group consisting of C(O)R⁷, CH₂R⁸, and C(O)OR⁹; or R¹ is selected from the group consisting of 1,2,4-oxadiazolyl and pyrimidinyl, each optionally substituted with 1 substituent selected from the group consisting of prop-1-en-2-yl, ethyl, 2-fluoropropan-2-yl, and isopropyl;
R² is selected from the group consisting of H, isopropylsulfonyl, methylsulfonyl, cyano, dimethylcarbamoyl, bromo, pyridazin-4-yl, 1H-1,2,4-triazol-1-yl, 1,1-dioxo-thiomorpholin-4-yl, morpholin-4-yl, cyclopropylsulfonyl, ethylsulfonyl, pyrimidin-5-yl, and 3-methoxyazetidine-1-carbonyl;
R³, R⁴, R⁵, and R⁶ are each independently selected from the group consisting of H and methyl;
R⁷ is 1,1-difluoropropyl;
R⁸ is 1,2,4-oxadiazolyl optionally substituted with one isopropyl group; and
R⁹ is selected from the group consisting of isopropyl, tert-butyl, 1-methylcyclopropyl, 1,3-difluoropropan-2-yl, 1-fluoropropan-2-yl, tetrahydrofuran-3-yl, 1-hydroxypropan-2-yl, phenyl, 2,2,3,3-tetrafluorocyclobutyl, 1,1,1,3,3,3-hexafluoropropan-2-yl, 1,1,1-trifluoropropan-2-yl, 1-(benzyloxy)propan-2-yl, and 1-hydroxypropan-2-yl.

One aspect of the present invention encompasses certain cyclohexyl derivatives selected from compounds of Formula (Ia) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶; or
Q is CR⁴; Z is CR⁵; and X is N;
R¹ is selected from the group consisting of 2,2-difluorobutanoyl, (3-isopropyl-1,2,4-oxadiazol-5-yl)methyl, tert-butoxycarbonyl, isopropoxycarbonyl, (1-methylcyclopropoxy)carbonyl, (tetrahydrofuran-3-yloxy)carbonyl, (1,3-difluoropropan-2-yloxy)carbonyl, (1-fluoropropan-2-yloxy)carbonyl, 3-(2-fluoropropan-2-yl)-1,2,4-oxadiazol-5-yl, 3-(prop-1-en-2-yl)-1,2,4-oxadiazol-5-yl, 5-ethyl-pyrimidin-2-yl, (1-hydroxypropan-2-yloxy)carbonyl, phenoxycarbonyl, 5-isopropyl-1,2,4-oxadiazol-3-yl, 3-isopropyl-1,2,4-oxadiazol-5-yl, 5-(2-fluoropropan-2-yl)-1,2,4-oxadiazol-3-yl, (2,2,3,3-tetrafluorocyclobutoxy)carbonyl, (1,1,1,3,3,3-hexafluoropropan-2-yloxy)carbonyl, (1,1,1-trifluoropropan-2-yloxy)carbonyl, and (1-(benzyloxy)propan-2-yloxy)carbonyl;
R² is selected from the group consisting of H, isopropylsulfonyl, methylsulfonyl, cyano, dimethylcarbamoyl, bromo, pyridazin-4-yl, 1H-1,2,4-triazol-1-yl, 1,1-dioxo-thiomorpholin-4-yl, morpholin-4-yl, cyclopropylsulfonyl, ethylsulfonyl, pyrimidin-5-yl, and 3-methoxyazetidine-1-carbonyl;
R³ is selected from the group consisting of H, and methyl;
R⁴ is H;
R⁵ is selected from the group consisting of H and methyl; and
R⁶ is H.

One aspect of the present invention encompasses certain cyclohexyl derivatives selected from compounds of Formula (Ia) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶;
R¹ is selected from the group consisting of C(O)R⁷, CH₂R⁸, and C(O)OR⁹; or R¹ is selected from the group consisting of 1,2,4-oxadiazolyl and pyrimidinyl, each optionally substituted with 1 substituent selected from the group consisting of C₂-C₆ alkenyl, C₁-C₆ alkyl, and C₁-C₆ haloalkyl;
R² is selected from the group consisting of cyano, halogen, heteroaryl, heterocyclyl, S(O)₂R¹¹, and C(O)NR¹²R¹³;
R³, R⁵, and R⁶ are each independently selected from the group consisting of H and C₁-C₆ alkyl;
R⁷ is C₁-C₆ haloalkyl;
R⁸ is a five-membered heteroaryl optionally substituted with one C₁-C₆ alkyl substituent;
R⁹ is selected from the group consisting of C₁-C₆ alkyl, C₃-C₆ cycloalkyl, C₁-C₆ haloalkyl, and heterocyclyl; wherein the C₁-C₆ alkyl is optionally substituted with hydroxyl; and the C₃-C₆ cycloalkyl is optionally substituted with one C₁-C₆ alkyl substituent;
R¹¹ is C₁-C₆ alkyl; and
R¹² and R¹³ are each independently C₁-C₆ alkyl.

One aspect of the present invention encompasses certain cyclohexyl derivatives selected from compounds of Formula (Ia) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶;
R¹ is selected from the group consisting of C(O)R⁷, CH₂R⁸, and C(O)OR⁹; or R¹ is selected from the group consisting of 1,2,4-oxadiazolyl and pyrimidinyl, each optionally substituted with 1 substituent selected from the group consisting of prop-1-en-2-yl, ethyl, and 2-fluoropropan-2-yl;
R² is selected from the group consisting of isopropylsulfonyl, methylsulfonyl, cyano, dimethylcarbamoyl, bromo, pyridazin-4-yl, 1H-1,2,4-triazol-1-yl, 1,1-dioxo-thiomorpholin-4-yl, and morpholin-4-yl;
R³, R⁵, and R⁶ are each independently selected from the group consisting of H and methyl;
R⁷ is 1,1-difluoropropyl;
R⁸ is 1,2,4-oxadiazolyl optionally substituted with one isopropyl group; and
R⁹ is selected from the group consisting of isopropyl, tert-butyl, 1-methylcyclopropyl, 1,3-difluoropropan-2-yl, 1-fluoropropan-2-yl, tetrahydrofuran-3-yl, and 1-hydroxypropan-2-yl.

One aspect of the present invention encompasses certain cyclohexyl derivatives selected from compounds of Formula (Ia) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶;
R¹ is selected from the group consisting of 2,2-difluorobutanoyl, (3-isopropyl-1,2,4-oxadiazol-5-yl)methyl, tert-butoxycarbonyl, isopropoxycarbonyl, (1-methylcyclopropoxy)carbonyl, (tetrahydrofuran-3-yloxy)carbonyl, (1,3-difluoropropan-2-yloxy)carbonyl, (1-fluoropropan-2-yloxy)carbonyl, 3-(2-fluoropropan-2-yl)-1,2,4-oxadiazol-5-yl, 3-(prop-1-en-2-yl)-1,2,4-oxadiazol-5-yl, 5-ethyl-pyrimidin-2-yl, and (1-hydroxypropan-2-yloxy)carbonyl;
R² is isopropylsulfonyl, methylsulfonyl, cyano, dimethylcarbamoyl, bromo, pyridazin-4-yl, 1H-1,2,4-triazol-1-yl, 1,1-dioxo-thiomorpholin-4-yl, and morpholin-4-yl;
R³ is H;
R⁵ is selected from the group consisting of H, and methyl; and
R⁶ is H.

In some embodiments, one Q, Z, and X is N, and compounds are selected from compounds of Formula (Im) and pharmaceutically acceptable salts, solvates, and hydrates thereof:

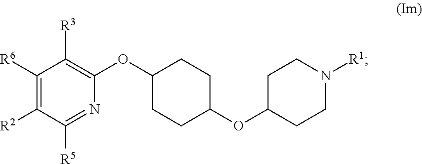

wherein each variable in Formula (Im) has the same meaning as described herein, supra and infra.

In some embodiments, one Q, Z, and X is N, the stereochemistry is (1r,4r) (i.e., trans). In some embodiments, compounds are selected from compounds of Formula (Io) and pharmaceutically acceptable salts, solvates, and hydrates thereof:

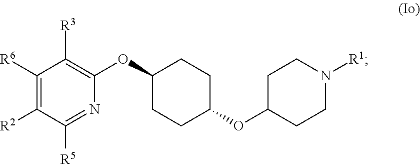

wherein each variable in Formula (Io) has the same meaning as described herein, supra and infra.

In some embodiments, one Q, Z, and X is N, the stereochemistry is (1s,4s) (i.e., cis). In some embodiments, compounds are selected from compounds of Formula (Iq) and pharmaceutically acceptable salts, solvates, and hydrates thereof:

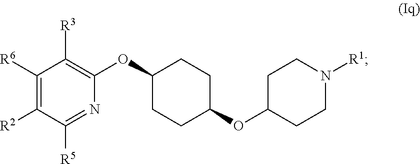

wherein each variable in Formula (Iq) has the same meaning as described herein, supra and infra.

In some embodiments, two Q, Z, and X are N, and the stereochemistry is (1r,4r) (i.e., trans). In some embodiments, compounds are selected from compounds of Formula (Is) and pharmaceutically acceptable salts, solvates, and hydrates thereof:

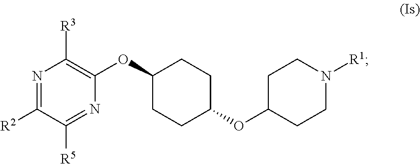

wherein each variable in Formula (Is) has the same meaning as described herein, supra and infra.

In some embodiments, two Q, Z, and X are N, the stereochemistry is (1r,4r) (i.e., trans). In some embodiments, compounds are selected from compounds of Formula (Iu) and pharmaceutically acceptable salts, solvates, and hydrates thereof:

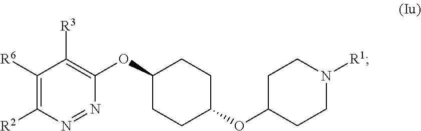

wherein each variable in Formula (Iu) has the same meaning as described herein, supra and infra.

One aspect of the present invention encompasses certain cyclohexyl derivatives selected from compounds of Formula (Ic) and pharmaceutically acceptable salts, solvates, and hydrates thereof:

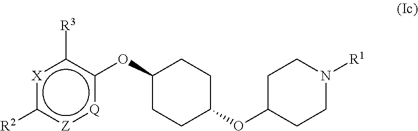

wherein:
Q is N; Z is CR⁵; and X is CR⁶; or
Q is N; Z is CR⁵; and X is N; or
Q is N; Z is N; and X is CR⁶; or
Q is CR⁴; Z is CR⁵; and X is N;
R¹ is selected from the group consisting of C(O)R⁷, CH₂R⁸, and C(O)OR⁹; or R¹ is selected from the group consisting of 1,2,4-oxadiazolyl and pyrimidinyl, each optionally substituted with 1 substituent selected from the group consisting of C₂-C₆ alkenyl, C₁-C₆ alkyl, and C₁-C₆ haloalkyl;
R² is selected from the group consisting of H, cyano, halogen, heteroaryl, heterocyclyl, S(O)₂R¹¹, and C(O)NR¹²R¹³;
R³, R⁴, R⁵, and R⁶ are each independently selected from the group consisting of H and C₁-C₆ alkyl;
R⁷ is C₁-C₆ haloalkyl;
R⁸ is a five-membered heteroaryl optionally substituted with one C₁-C₆ alkyl substituent;
R⁹ is selected from the group consisting of C₁-C₆ alkyl, C₃-C₆ cycloalkyl, C₁-C₆ haloalkyl, heterocyclyl, and phenyl; wherein the C₁-C₆ alkyl is optionally substituted with one or more substituents selected independently from the group consisting of hydroxyl, C₁-C₆ alkoxy, and R¹⁰, wherein C₁-C₆ alkoxy is optionally substituted with phenyl; and the C₃-C₆ cycloalkyl is optionally substituted with one or more substituents selected independently from the group consisting of C₁-C₆ alkyl and halogen;
R¹¹ is selected from the group consisting of C₁-C₆ alkyl and C₃-C₆ cycloalkyl; and
R¹² and R¹³ are each independently C₁-C₆ alkyl; or R¹² and R¹³ together with the nitrogen to which they are both bonded form a heterocyclyl optionally substituted with C₁-C₆ alkoxy.

One aspect of the present invention encompasses certain cyclohexyl derivatives selected from compounds of Formula (Ic) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶; or
Q is CR⁴; Z is CR⁵; and X is N;
R¹ is selected from the group consisting of C(O)R⁷, CH₂R⁸, and C(O)OR⁹; or R¹ is selected from the group consisting of 1,2,4-oxadiazolyl and pyrimidinyl, each optionally substituted with 1 substituent selected from the group consisting of prop-1-en-2-yl, ethyl, 2-fluoropropan-2-yl, and isopropyl;
R² is selected from the group consisting of H, isopropylsulfonyl, methylsulfonyl, cyano, dimethylcarbamoyl, bromo, pyridazin-4-yl, 1H-1,2,4-triazol-1-yl, 1,1-dioxo-thiomorpholin-4-yl, morpholin-4-yl, cyclopropylsulfonyl, ethylsulfonyl, pyrimidin-5-yl, and 3-methoxyazetidine-1-carbonyl;
R³, R⁴, R⁵, and R⁶ are each independently selected from the group consisting of H and methyl;
R⁷ is 1,1-difluoropropyl;
R⁸ is 1,2,4-oxadiazolyl optionally substituted with one isopropyl group; and
R⁹ is selected from the group consisting of isopropyl, tert-butyl, 1-methylcyclopropyl, 1,3-difluoropropan-2-yl, 1-fluoropropan-2-yl, tetrahydrofuran-3-yl, 1-hydroxypropan-2-yl, phenyl, 2,2,3,3-tetrafluorocyclobutyl, 1,1,1,3,3,3-hexafluoropropan-2-yl, 1,1,1-trifluoropropan-2-yl, and 1-hydroxypropan-2-yl.

One aspect of the present invention encompasses certain cyclohexyl derivatives selected from compounds of Formula (Ic) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶; or
Q is CR⁴; Z is CR⁵; and X is N;
R¹ is selected from the group consisting of 2,2-difluorobutanoyl, (3-isopropyl-1,2,4-oxadiazol-5-yl)methyl, tert-butoxycarbonyl, isopropoxycarbonyl, (1-methylcyclopropoxy)carbonyl, (tetrahydrofuran-3-yloxy)carbonyl, (1,3-difluoropropan-2-yloxy)carbonyl, (1-fluoropropan-2-yloxy)carbonyl, 3-(2-fluoropropan-2-yl)-1,2,4-oxadiazol-5-yl, 3-(prop-1-en-2-yl)-1,2,4-oxadiazol-5-yl, 5-ethyl-pyrimidin-2-yl, (1-hydroxypropan-2-yloxy)carbonyl, phenoxycarbonyl, 5-isopropyl-1,2,4-oxadiazol-3-yl, 3-isopropyl-1,2,4-oxadiazol-5-yl, 5-(2-fluoropropan-2-yl)-1,2,4-oxadiazol-3-yl, (2,2,3,3-tetrafluorocyclobutoxy)carbonyl, (1,1,1,3,3,3-hexafluoropropan-2-yloxy)carbonyl, (1,1,1-trifluoropropan-2-yloxy)carbonyl, and (1-(benzyloxy)propan-2-yloxy)carbonyl;
R² is selected from the group consisting of H, isopropylsulfonyl, methylsulfonyl, cyano, dimethylcarbamoyl, bromo, pyridazin-4-yl, 1H-1,2,4-triazol-1-yl, 1,1-dioxo-thiomorpholin-4-yl, morpholin-4-yl, cyclopropylsulfonyl, ethylsulfonyl, pyrimidin-5-yl, and 3-methoxyazetidine-1-carbonyl;
R³ is selected from the group consisting of H and methyl;
R⁴ is H;
R⁵ is selected from the group consisting of H and methyl; and
R⁶ is H.

One aspect of the present invention encompasses certain cyclohexyl derivatives selected from compounds of Formula (Ic) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶;
R¹ is selected from the group consisting of C(O)R⁷, CH₂R⁸, and C(O)OR⁹; or R¹ is selected from the group consisting of 1,2,4-oxadiazolyl and pyrimidinyl, each optionally substituted with 1 substituent selected from the group consisting of C₂-C₆ alkenyl, C₁-C₆ alkyl, and C₁-C₆ haloalkyl;
R² is selected from the group consisting of cyano, halogen, heteroaryl, heterocyclyl, S(O)₂R¹¹, and C(O)NR¹²R¹³;
R³, R⁴, R⁵, and R⁶ are each independently selected from the group consisting of H and C₁-C₆ alkyl;
R⁷ is C₁-C₆ haloalkyl;
R⁸ is a five-membered heteroaryl optionally substituted with one C₁-C₆ alkyl substituent;
R⁹ is selected from the group consisting of C₁-C₆ alkyl, C₃-C₆ cycloalkyl, C₁-C₆ haloalkyl, and heterocyclyl; wherein the C₁-C₆ alkyl is optionally substituted with hydroxyl; and the C₃-C₆ cycloalkyl is optionally substituted with one C₁-C₆ alkyl substituent;
R¹¹ is C₁-C₆ alkyl; and
R¹² and R¹³ are each independently C₁-C₆ alkyl.

One aspect of the present invention encompasses certain cyclohexyl derivatives selected from compounds of Formula (Ic) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶;
R¹ is selected from the group consisting of C(O)R⁷, CH₂R⁸, and C(O)OR⁹; or R¹ is selected from the group consisting of 1,2,4-oxadiazolyl and pyrimidinyl, each optionally substituted with 1 substituent selected from the group consisting of prop-1-en-2-yl, ethyl, and 2-fluoropropan-2-yl;
R² is selected from the group consisting of isopropylsulfonyl, methylsulfonyl, cyano, dimethylcarbamoyl, bromo, pyridazin-4-yl, 1H-1,2,4-triazol-1-yl, 1,1-dioxo-thiomorpholin-4-yl, and morpholin-4-yl;
R³, R⁴, R⁵, and R⁶ are each independently selected from the group consisting of H and methyl;
R⁷ is 1,1-difluoropropyl;
R⁸ is 1,2,4-oxadiazolyl optionally substituted with one isopropyl group; and
R⁹ is selected from the group consisting of isopropyl, tert-butyl, 1-methylcyclopropyl, 1,3-difluoropropan-2-yl, 1-fluoropropan-2-yl, tetrahydrofuran-3-yl, and 1-hydroxypropan-2-yl.

One aspect of the present invention encompasses certain cyclohexyl derivatives selected from compounds of Formula (Ic) and pharmaceutically acceptable salts, solvates, and hydrates thereof, wherein:
Q is N; Z is CR⁵; and X is CR⁶; or
Q is N; Z is CR⁵; and X is N; or
Q is N; Z is N; and X is CR⁶;
R¹ is selected from the group consisting of 2,2-difluorobutanoyl, (3-isopropyl-1,2,4-oxadiazol-5-yl)methyl, tert-butoxycarbonyl, isopropoxycarbonyl, (1-methylcyclopropoxy)carbonyl, (tetrahydrofuran-3-yloxy)carbonyl, (1,3-difluoropropan-2-yloxy)carbonyl, (1-fluoropropan-2-yloxy)carbonyl, 3-(2-fluoropropan-2-yl)-1,2,4-oxadiazol-5-yl, 3-(prop-1-en-2-yl)-1,2,4-oxadiazol-5-yl, 5-ethyl-pyrimidin-2-yl, and (1-hydroxypropan-2-yloxy)carbonyl;
R² is selected from the group consisting of isopropylsulfonyl, methylsulfonyl, cyano, dimethylcarbamoyl, bromo, pyridazin-4-yl, 1H-1,2,4-triazol-1-yl, 1,1-dioxo-thiomorpholin-4-yl, and morpholin-4-yl;
R³ is H;
R⁵ is selected from the group consisting of H and methyl; and
R⁶ is H.

One aspect of the present invention encompasses certain cyclohexyl derivatives selected from compounds of Formula (IIe) and pharmaceutically acceptable salts, solvates, and hydrates thereof:

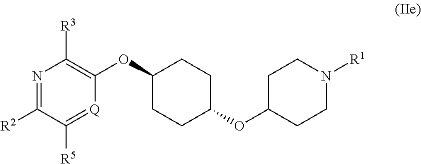

wherein:
Q is N or CR⁴;
R¹ is selected from the group consisting of C(O)R⁷ and C(O)OR⁹; or R¹ is 1,2,4-oxadiazolyl optionally substituted with 1 substituent selected from the group consisting of C₂-C₆ alkenyl, C₁-C₆ alkyl, and C₁-C₆ haloalkyl;
R² is selected from the group consisting of H, cyano, heteroaryl, heterocyclyl, S(O)₂R¹¹, and C(O)NR¹²R¹³;
R³, R⁴, and R⁵ are each independently selected from the group consisting of H and C₁-C₆ alkyl;
R⁷ is C₁-C₆ haloalkyl;
R⁹ is selected from the group consisting of C₁-C₆ alkyl, C₃-C₆ cycloalkyl, C₁-C₆ haloalkyl, heterocyclyl, and phenyl; wherein the C₁-C₆ alkyl is optionally substituted with one or more substituents selected independently from the group consisting of hydroxyl and C₁-C₆ alkoxy, wherein C₁-C₆ alkoxy is optionally substituted with phenyl;

and the C₃-C₆ cycloalkyl is optionally substituted with one or more substituents selected independently from the group consisting of C₁-C₆ alkyl and halogen;
R¹¹ is selected from the group consisting of C₁-C₆ alkyl and C₃-C₆ cycloalkyl; and
R¹² and R¹³ are each independently C₁-C₆ alkyl; or R¹² and R¹³ together with the nitrogen to which they are both bonded form a heterocyclyl optionally substituted with C₁-C₆ alkoxy.

One aspect of the present invention encompasses certain cyclohexyl derivatives selected from compounds of Formula (IIe) and pharmaceutically acceptable salts, solvates, and hydrates thereof, wherein:
Q is N or CR⁴;
R¹ is selected from the group consisting of C(O)R⁷ and C(O)OR⁹; or R¹ is 1,2,4-oxadiazolyl optionally substituted with 1 substituent selected from the group consisting of prop-1-en-2-yl, 2-fluoropropan-2-yl, and isopropyl;
R² is selected from the group consisting of H, isopropylsulfonyl, methylsulfonyl, cyano, pyridazin-4-yl, 1H-1,2,4-triazol-1-yl, 1,1-dioxo-thiomorpholin-4-yl, morpholin-4-yl, cyclopropylsulfonyl, bromo, dimethylcarbamoyl, ethylsulfonyl, pyrimidin-5-yl, and 3-methoxyazetidine-1-carbonyl;
R³, R⁴, and R⁵ are each independently selected from the group consisting of H and methyl;
R⁷ is 1,1-difluoropropyl; and
R⁹ is selected from the group consisting of isopropyl, tert-butyl, 1-methylcyclopropyl, 1,3-difluoropropan-2-yl, 1-fluoropropan-2-yl, tetrahydrofuran-3-yl, 1-hydroxypropan-2-yl, phenyl, 2,2,3,3-tetrafluorocyclobutyl, 1,1,1,3,3,3-hexafluoropropan-2-yl, 1,1,1-trifluoropropan-2-yl, 1-(benzyloxy)propan-2-yl, and 1-hydroxypropan-2-yl.

One aspect of the present invention encompasses certain cyclohexyl derivatives selected from compounds of Formula (IIe) and pharmaceutically acceptable salts, solvates, and hydrates thereof, wherein:
Q is N or CR⁴;
R¹ is selected from the group consisting of 2,2-difluorobutanoyl, tert-butoxycarbonyl, isopropoxycarbonyl, (1-methylcyclopropoxy)carbonyl, (tetrahydrofuran-3-yloxy)carbonyl, (1,3-difluoropropan-2-yloxy)carbonyl, (1-fluoropropan-2-yloxy)carbonyl, 3-(2-fluoropropan-2-yl)-1,2,4-oxadiazol-5-yl, 5-(2-fluoropropan-2-yl)-1,2,4-oxadiazol-3-yl, 3-(prop-1-en-2-yl)-1,2,4-oxadiazol-5-yl, (1-hydroxypropan-2-yloxy)carbonyl, phenoxycarbonyl, 5-isopropyl-1,2,4-oxadiazol-3-yl, 3-isopropyl-1,2,4-oxadiazol-5-yl, (2,2,3,3-tetrafluorocyclobutoxy)carbonyl, (1,1,1,3,3,3-hexafluoropropan-2-yloxy)carbonyl, (1,1,1-trifluoropropan-2-yloxy)carbonyl, and (1-(benzyloxy)propan-2-yloxy)carbonyl;
R² is selected from the group consisting of H, isopropylsulfonyl, methylsulfonyl, cyano, pyridazin-4-yl, 1H-1,2,4-triazol-1-yl, 1,1-dioxo-thiomorpholin-4-yl, morpholin-4-yl, cyclopropylsulfonyl, bromo, dimethylcarbamoyl, ethylsulfonyl, pyrimidin-5-yl, and 3-methoxyazetidine-1-carbonyl; and
R³, R⁴, and R⁵ are each independently selected from the group consisting of H and methyl.

One aspect of the present invention encompasses certain cyclohexyl derivatives selected from compounds of Formula (Is) and pharmaceutically acceptable salts, solvates, and hydrates thereof:

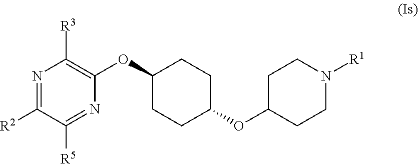

wherein:
R¹ is selected from the group consisting of C(O)R⁷ and C(O)OR⁹; or R¹ is 1,2,4-oxadiazolyl optionally substituted with 1 substituent selected from the group consisting of C₂-C₆ alkenyl and C₁-C₆ haloalkyl;
R² is selected from the group consisting of cyano, heteroaryl, heterocyclyl, and S(O)₂R¹¹;
R³ and R⁵ are each H;
R⁷ is C₁-C₆ haloalkyl;
R⁹ is selected from the group consisting of C₁-C₆ alkyl, C₃-C₆ cycloalkyl, C₁-C₆ haloalkyl, and heterocyclyl; wherein the C₁-C₆ alkyl is optionally substituted with hydroxyl; and the C₃-C₆ cycloalkyl is optionally substituted with one C₁-C₆ alkyl substituent; and
R¹¹ is C₁-C₆ alkyl.

One aspect of the present invention encompasses certain cyclohexyl derivatives selected from compounds of Formula (Is) and pharmaceutically acceptable salts, solvates, and hydrates thereof, wherein:
R¹ is selected from the group consisting of C(O)R⁷ and C(O)OR⁹; or R¹ is 1,2,4-oxadiazolyl optionally substituted with 1 substituent selected from the group consisting of prop-1-en-2-yl and 2-fluoropropan-2-yl;
R² is selected from the group consisting of isopropylsulfonyl, methylsulfonyl, cyano, pyridazin-4-yl, 1H-1,2,4-triazol-1-yl, 1,1-dioxo-thiomorpholin-4-yl, and morpholin-4-yl;
R³ and R⁵ are each H;
R⁷ is 1,1-difluoropropyl; and
R⁹ is selected from the group consisting of isopropyl, tert-butyl, 1-methylcyclopropyl, 1,3-difluoropropan-2-yl, 1-fluoropropan-2-yl, tetrahydrofuran-3-yl, and 1-hydroxypropan-2-yl.

One aspect of the present invention encompasses certain cyclohexyl derivatives selected from compounds of Formula (Is) and pharmaceutically acceptable salts, solvates, and hydrates thereof, wherein:
R¹ is selected from the group consisting of 2,2-difluorobutanoyl, tert-butoxycarbonyl, isopropoxycarbonyl, (1-methylcyclopropoxy)carbonyl, (tetrahydrofuran-3-yloxy)carbonyl, (1,3-difluoropropan-2-yloxy)carbonyl, (1-fluoropropan-2-yloxy)carbonyl, 3-(2-fluoropropan-2-yl)-1,2,4-oxadiazol-5-yl, 5-(2-fluoropropan-2-yl)-1,2,4-oxadiazol-3-yl, 3-(prop-1-en-2-yl)-1,2,4-oxadiazol-5-yl, and (1-hydroxypropan-2-yloxy)carbonyl;
R² is selected from the group consisting of isopropylsulfonyl, methylsulfonyl, cyano, pyridazin-4-yl, 1H-1,2,4-triazol-1-yl, 1,1-dioxo-thiomorpholin-4-yl, and morpholin-4-yl; and
R³ and R⁵ are each H.

Some embodiments of the present invention include every combination of one or more compound selected from the following group:

tert-butyl 4-(4-(4-(methylsulfonyl)phenoxy)cyclohexyloxy)piperidine-1-carboxylate; 3-isopropyl-5-(4-(4-(4-(methylsulfonyl)phenoxy)cyclohexyloxy)piperidin-1-yl)-1,2,4-oxadiazole; tert-butyl 4-(4-(2-fluoro-4-(methylsulfonyl)phenoxy)cyclohexyloxy)piperidine-1-carboxylate; tert-butyl 4-(4-(5-(methylsulfonyl)pyridin-2-yloxy)cyclohexyloxy)piperidine-1-carboxylate; 5-ethyl-2-(4-(4-(5-(methylsulfonyl)pyridin-2-yloxy)cyclohexyloxy)piperidin-1-yl)pyrimidine; 5-ethyl-2-(4-(4-(2-fluoro-4-(methylsulfonyl)phenoxy)cyclohexyloxy)piperidin-1-yl)pyrimidine; tert-butyl 4-(4-(5-(methylsulfonyl)pyridin-2-yloxy)cyclohexyloxy)piperidine-1-carboxylate; isopropyl 4-(4-(5-(methylsulfonyl)pyridin-2-yloxy)cyclohexyloxy)piperidine-1-carboxylate; isopropyl 4-(4-(2-fluoro-4-(methylsulfonyl)phenoxy)cyclohexyloxy)piperidine-1-carboxylate; 5-((4-(4-(2-fluoro-4-(methylsulfonyl)phenoxy)cyclohexyloxy)piperidin-1-yl)methyl)-3-isopropyl-1,2,4-oxadiazole; 3-isopropyl-5-((4-(4-(5-(methylsulfonyl)pyridin-2-yloxy)cyclohexyloxy)piperidin-1-yl)methyl)-1,2,4-oxadiazole; 5-(4-(4-(2-fluoro-4-(methylsulfonyl)phenoxy)cyclohexyloxy)piperidin-1-yl)-3-(2-fluoropropan-2-yl)-1,2,4-oxadiazole; tert-butyl 4-(4-(5-(methylsulfonyl)pyrazin-2-yloxy)cyclohexyloxy)piperidine-1-carboxylate; 3-(2-fluoropropan-2-yl)-5-(4-(4-(5-(methylsulfonyl)pyridin-2-yloxy)cyclohexyloxy)piperidin-1-yl)-1,2,4-oxadiazole; tert-butyl 4-(4-(5-(dimethylcarbamoyl)-6-methylpyridin-2-yloxy)cyclohexyloxy)piperidine-1-carboxylate; tert-butyl 4-(4-(6-bromopyridazin-3-yloxy)cyclohexyloxy)piperidine-1-carboxylate; tert-butyl 4-(4-(3-fluoro-4-(methylsulfonyl)phenoxy)cyclohexyloxy)piperidine-1-carboxylate; tert-butyl 4-(4-(5-fluoro-2-(methylsulfonyl)phenoxy)cyclohexyloxy)piperidine-1-carboxylate; tert-butyl 4-(4-(4-cyanophenoxy)cyclohexyloxy)piperidine-1-carboxylate; tert-butyl 4-(4-(6-(methylsulfonyl)pyridazin-3-yloxy)cyclohexyloxy)piperidine-1-carboxylate; 3-tert-butyl-5-(4-(4-(2-fluoro-4-(methylsulfonyl)phenoxy)cyclohexyloxy)piperidin-1-yl)-1,2,4-oxadiazole; 3-tert-butyl-5-(4-(4-(4-(methylsulfonyl)phenoxy)cyclohexyloxy)piperidin-1-yl)-1,2,4-oxadiazole; 3-(2-fluoropropan-2-yl)-5-(4-(4-(4-(methylsulfonyl)phenoxy)cyclohexyloxy)piperidin-1-yl)-1,2,4-oxadiazole; isopropyl 4-(4-(5-(methylsulfonyl)pyrazin-2-yloxy)cyclohexyloxy)piperidine-1-carboxylate; tert-butyl 4-(4-(5-(1H-1,2,4-triazol-1-yl)pyrazin-2-yloxy)cyclohexyloxy)piperidine-1-carboxylate; 3-(2-fluoropropan-2-yl)-5-(4-(4-(5-(methylsulfonyl)pyrazin-2-yloxy)cyclohexyloxy)piperidin-1-yl)-1,2,4-oxadiazole; tert-butyl 4-(4-(4-(2-cyanoethyl)phenoxy)cyclohexyloxy)piperidine-1-carboxylate; 3,3-dimethyl-1-(4-(4-(4-(methylsulfonyl)phenoxy)cyclohexyloxy)piperidin-1-yl)butan-1-one; isobutyl 4-(4-(4-(methylsulfonyl)phenoxy)cyclohexyloxy)piperidine-1-carboxylate; 5-chloro-2-(4-(4-(4-(methylsulfonyl)phenoxy)cyclohexyloxy)piperidin-1-yl)pyrimidine; tert-butyl 2-(4-(4-(4-(methylsulfonyl)phenoxy)cyclohexyloxy)piperidin-1-yl)acetate; cyclopentyl 4-(4-(4-(methylsulfonyl)phenoxy)cyclohexyloxy)piperidine-1-carboxylate; 5-isopropyl-3-(4-(4-(4-(methylsulfonyl)phenoxy)cyclohexyloxy)piperidin-1-yl)-1,2,4-oxadiazole; 5-methyl-2-(4-(4-(4-(methylsulfonyl)phenoxy)cyclohexyloxy)piperidin-1-yl)pyrimidine; 2-(4-(4-(4-(methylsulfonyl)phenoxy)cyclohexyloxy)piperidin-1-yl)-5-(trifluoromethyl)pyridine; 2-methyl-1-(4-(4-(4-(methylsulfonyl)phenoxy)cyclohexyloxy)piperidin-1-yl)propan-1-one; 3-methyl-6-(4-(4-(4-(methylsulfonyl)phenoxy)cyclohexyloxy)piperidin-1-yl)pyridazine; 1,1,1,3,3,3-hexafluoropropan-2-yl 4-(4-(4-(methylsulfonyl)phenoxy)cyclohexyloxy)piperidine-1-carboxylate; (3-methyloxetan-3-yl)methyl-4-(4-(4-(methylsulfonyl)phenoxy)cyclohexyloxy)piperidine-1-carboxylate; 2-methyl-5-(4-(4-(4-(methylsulfonyl)phenoxy)cyclohexyloxy)piperidin-1-yl)pyrazine; (2-methylcyclopropyl)(4-(4-(4-(methylsulfonyl)phenoxy)cyclohexyloxy)piperidin-1-yl)methanone; 1-methylcyclopropyl-4-(4-(4-(methylsulfonyl)phenoxy)cyclohexyloxy)piperidine-1-carboxylate; tert-butyl 4-(4-(4-(1H-1,2,4-triazol-1-yl)phenoxy)cyclohexyloxy)piperidine-1-carboxylate; 2,2-difluoro-1-(4-(4-(4-(methylsulfonyl)phenoxy)cyclohexyloxy)piperidin-1-yl)butan-1-one; 5-chloro-2-(4-(4-(4-(methylsulfonyl)phenoxy)cyclohexyloxy)piperidin-1-yl)pyridine; 1-(cyclopropylsulfonyl)-4-(4-(4-(methylsulfonyl)phenoxy)cyclohexyloxy)piperidine; 3-ethoxy-6-(4-(4-(4-(methylsulfonyl)phenoxy)cyclohexyloxy)piperidin-1-yl)pyridazine; 5-fluoro-2-(4-(4-(4-(methylsulfonyl)phenoxy)cyclohexyloxy)piperidin-1-yl)pyridine; sec-butyl 4-(4-(4-(methylsulfonyl)phenoxy)cyclohexyloxy)piperidine-1-carboxylate; sec-butyl 4-(4-(4-(methylsulfonyl)phenoxy)cyclohexyloxy)piperidine-1-carboxylate; 1-(isopropylsulfonyl)-4-(4-(4-(methylsulfonyl)phenoxy)cyclohexyloxy)piperidine; tetrahydrofuran-3-yl4-(4-(5-(methylsulfonyl)pyrazin-2-yloxy)cyclohexyloxy)piperidine-1-carboxylate; 4-(4-(4-(methylsulfonyl)phenoxy)cyclohexyloxy)-1-p-tolylpiperidine; 4-(4-(4-(methylsulfonyl)phenoxy)cyclohexyloxy)-1-(4-(trifluoromethyl)phenyl)piperidine; 4-(4-(4-(methylsulfonyl)phenoxy)cyclohexyloxy)-1-(4-(trifluoromethoxy)phenyl)piperidine; 1-(4-methoxyphenyl)-4-(4-(4-(methylsulfonyl)phenoxy)cyclohexyloxy)piperidine; 4-(4-(4-(methylsulfonyl)phenoxy)cyclohexyloxy)-1-(3-(trifluoromethyl)phenyl)piperidine; 5-(2-fluoropropan-2-yl)-3-(4-(4-(5-(methylsulfonyl)pyrazin-2-yloxy)cyclohexyloxy)piperidin-1-yl)-1,2,4-oxadiazole; 1,1,1-trifluoropropan-2-yl 4-(4-(4-(methylsulfonyl)phenoxy)cyclohexyloxy)piperidine-1-carboxylate; 1-methylcyclopropyl 4-(4-(5-(methylsulfonyl)pyrazin-2-yloxy)cyclohexyloxy)piperidine-1-carboxylate; isopropyl 4-(4-(5-(1H-1,2,4-triazol-1-yl)pyrazin-2-yloxy)cyclohexyloxy)piperidine-1-carboxylate; 1-(4-fluorophenyl)-4-(4-(4-(methylsulfonyl)phenoxy)cyclohexyloxy)piperidine; 1-(4-chloro-2-fluorophenyl)-4-(4-(4-(methylsulfonyl)phenoxy)cyclohexyloxy)piperidine; tert-butyl 4-(4-(5-cyanopyrazin-2-yloxy)cyclohexyloxy)piperidine-1-carboxylate; 5-(4-(4-(5-(methylsulfonyl)pyrazin-2-yloxy)cyclohexyloxy)piperidin-1-yl)-3-(prop-1-en-2-yl)-1,2,4-oxadiazole; 5-methoxy-2-(4-(4-(4-(methylsulfonyl)phenoxy)cyclohexyloxy)piperidin-1-yl)pyrimidine; 1,3-difluoropropan-2-yl 4-(4-(5-(methylsulfonyl)pyrazin-2-yloxy)cyclohexyloxy)piperidine-1-carboxylate; tert-butyl 4-(4-(5-(isopropylsulfonyl)pyrazin-2-yloxy)cyclohexyloxy)piperidine-1-carboxylate; 2,2-difluoro-1-(4-(4-(5-(methylsulfonyl)pyrazin-2-yloxy)cyclohexyloxy)piperidin-1-yl)butan-1-one; 1-fluoropropan-2-yl 4-(4-(5-(methylsulfonyl)pyrazin-2-yloxy)cyclohexyloxy)piperidine-1-carboxylate; 1-hydroxypropan-2-yl 4-(4-(5-(methylsulfonyl)pyrazin-2-yloxy)cyclohexyloxy)piperidine-1-carboxylate; isopropyl 4-(4-(5-(pyridazin-4-yl)pyrazin-2-yloxy)cyclohexyloxy)piperidine-1-carboxylate; isopropyl 4-((4-(5-(1,1-dioxo-thiomorpholin-4-yl)pyrazin-2-yloxy)cyclohexyloxy)piperidine-1-carboxylate; isopropyl 4-((4-(5-morpholinopyrazin-2-yloxy)cyclohexyloxy)piperidine-1-carboxylate; and 1,1,1-trifluoropropan-2-yl 4-(4-(5-(methylsulfonyl)pyrazin-2-yloxy)cyclohexyloxy)piperidine-1-carboxylate.

Some embodiments of the present invention include every combination of one or more compound selected from the following group:

isopropyl 4-(4-(2-methylpyridin-3-yloxy)cyclohexyloxy)piperidine-1-carboxylate; isopropyl 4-(4-((6-(cyclopropylsulfonyl)-2-methylpyridin-3-yloxy)cyclohexyloxy)piperidine-1-carboxylate; phenyl 4-(4-(5-(methylsulfonyl)pyrazin-2-yloxy)cyclohexyloxy)piperidine-1-carboxylate; 5-isopropyl-3-(4-(4-(5-(methylsulfonyl)pyrazin-2-yloxy)cyclohexyloxy)piperidin-1-yl)-1,2,4-oxadiazole; 3-isopropyl-5-(4-(4-(5-(methylsulfonyl)pyrazin-2-yloxy)cyclohexyloxy)piperidin-1-yl)-1,2,4-oxadiazole; 3-(4-(4-(5-(1H-1,2,4-triazol-1-yl)pyrazin-2-yloxy)cyclohexyloxy)piperidin-1-yl)-5-(2-fluoropropan-2-yl)-1,2,4-oxadiazole; 2,2,3,3-tetrafluorocyclobutyl 4-(4-(5-(methylsulfonyl)pyrazin-2-yloxy)cyclohexyloxy)piperidine-1-carboxylate; 1,1,1,3,3,3-hexafluoropropan-2-yl 4-(4-(5-(methylsulfonyl)pyrazin-2-yloxy)cyclohexyloxy)piperidine-1-carboxylate; isopropyl 4-(4-(6-bromopyridin-3-yloxy)cyclohexyloxy)piperidine-1-carboxylate; isopropyl 4-(4-(6-(methylsulfonyl)pyridin-3-yloxy)cyclohexyloxy)piperidine-1-carboxylate; isopropyl 4-(4-(4-((S)-2-amino-3-(3,3-difluoroazetidin-1-yl)-3-oxopropyl)phenoxy)cyclohexyloxy)piperidine-1-carboxylate; isopropyl 4-(4-(6-(1H-1,2,4-triazol-1-yl)pyridin-3-yloxy)cyclohexyloxy)piperidine-1-carboxylate; 5-(4-(4-(5-(1H-1,2,4-triazol-1-yl)pyrazin-2-yloxy)cyclohexyloxy)piperidin-1-yl)-3-isopropyl-1,2,4-oxadiazole; isopropyl 4-(4-(4-((R)-2-amino-3-(3,3-difluoroazetidin-1-yl)-3-oxopropyl)phenoxy)cyclohexyloxy)piperidine-1-carboxylate; 5-(4-(1-(3-isopropyl-1,2,4-oxadiazol-5-yl)piperidin-4-yloxy)cyclohexyloxy)-N,N-dimethylpyrazine-2-carboxamide; (R)-1,1,1-trifluoropropan-2-yl 4-(4-(5-(ethylsulfonyl)pyrazin-2-yloxy)cyclohexyloxy)piperidine-1-carboxylate; isopropyl 4-(4-(6-bromo-2-methylpyridin-3-yloxy)cyclohexyloxy)piperidine-1-carboxylate; isopropyl 4-(4-(2-methyl-6-(methylsulfonyl)pyridin-3-yloxy)cyclohexyloxy)piperidine-1-carboxylate; isopropyl 4-(4-(2-methyl-6-(pyrimidin-5-yl)pyridin-3-yloxy)cyclohexyloxy)piperidine-1-carboxylate; (5-(4-(1-(3-isopropyl-1,2,4-oxadiazol-5-yl)piperidin-4-yloxy)cyclohexyloxy)pyrazin-2-yl)(3-methoxyazetidin-1-yl)methanone; isopropyl 4-(4-(6-(ethylsulfonyl)-2-methylpyridin-3-yloxy)cyclohexyloxy)piperidine-1-carboxylate; 3-isopropyl-5-(4-(4-(2-methyl-6-(methylsulfonyl)pyridin-3-yloxy)cyclohexyloxy)piperidin-1-yl)-1,2,4-oxadiazole; isopropyl 4-(4-(6-cyano-2-methylpyridin-3-yloxy)cyclohexyloxy)piperidine-1-carboxylate; (S)-1-(benzyloxy)propan-2-yl 4-(4-(2-methyl-6-(methylsulfonyl)pyridin-3-yloxy)cyclohexyloxy)piperidine-1-carboxylate; (S)-1-hydroxypropan-2-yl 4-(4-(2-methyl-6-(methylsulfonyl)pyridin-3-yloxy)cyclohexyloxy)piperidine-1-carboxylate; (R)-1,1,1-trifluoropropan-2-yl 4-(4-(2-methyl-6-(methylsulfonyl)pyridin-3-yloxy)cyclohexyloxy)piperidine-1-carboxylate; and (S)-1,1,1-trifluoropropan-2-yl 4-(4-(2-methyl-6-(methylsulfonyl)pyridin-3-yloxy)cyclohexyloxy)piperidine-1-carboxylate.

Some embodiments of the present invention include every combination of one or more compound selected from the following group:

isopropyl 4-(4-(3-methyl-5-(methylsulfonyl)pyrazin-2-yloxy)cyclohexyloxy)piperidine-1-carboxylate; (R)-1,1,1-trifluoropropan-2-yl 4-(4-(3-methyl-5-(methylsulfonyl)pyrazin-2-yloxy)cyclohexyloxy)piperidine-1-carboxylate; isopropyl 4-(4-(4-(3,3-difluoroazetidin-1-ylsulfonyl)phenoxy)cyclohexyloxy)piperidine-1-carboxylate; 5-ethyl-2-(4-(4-(2-methyl-6-(methylsulfonyl)pyridin-3-yloxy)cyclohexyloxy)piperidin-1-yl)pyrimidine; 5-methyl-2-(4-(4-(2-methyl-6-(methylsulfonyl)pyridin-3-yloxy)cyclohexyloxy)piperidin-1-yl)pyrimidine; S-isopropyl 4-(4-(2-methyl-6-(methylsulfonyl)pyridin-3-yloxy)cyclohexyloxy)piperidine-1-carbothioate; 2-methyl-3-(4-(1-(5-methylpyridin-2-yl)piperidin-4-yloxy)cyclohexyloxy)-6-(methylsulfonyl)pyridine; 3-(4-(1-(5-ethylpyridin-2-yl)piperidin-4-yloxy)cyclohexyloxy)-2-methyl-6-(methylsulfonyl)pyridine; 2-methyl-6-(methylsulfonyl)-3-(4-(1-(((R)-1,1,1-trifluoropropan-2-yloxy)carbonyl)piperidin-4-yloxy)cyclohexyloxy)pyridine 1-oxide; isopropyl 4-(4-(4-methyl-6-(methylsulfonyl)pyridin-3-yloxy)cyclohexyloxy)piperidine-1-carboxylate; isopropyl 4-(4-(5-methyl-6-(methylsulfonyl)pyridin-3-yloxy)cyclohexyloxy)piperidine-1-carboxylate; 1,1,1-trifluoro-2-methylpropan-2-yl 4-(4-(2-methyl-6-(methylsulfonyl)pyridin-3-yloxy)cyclohexyloxy)piperidine-1-carboxylate; 1-methylcyclopropyl 4-(4-(2-methyl-6-(methylsulfonyl)pyridin-3-yloxy)cyclohexyloxy)piperidine-1-carboxylate; S-cyclopropyl 4-(4-(2-methyl-6-(methylsulfonyl)pyridin-3-yloxy)cyclohexyloxy)piperidine-1-carbothioate; 2-(methylsulfonyl)-5-(4-(1-((1-(trifluoromethyl)cyclopropyl)methyl)piperidin-4-yloxy)cyclohexyloxy)pyrazine; 2-(methylsulfonyl)-5-(4-(1-(2,2,3,3-tetrafluoropropyl)piperidin-4-yloxy)cyclohexyloxy)pyrazine; 2-(methylsulfonyl)-5-(4-(1-((1-(trifluoromethyl)cyclobutyl)methyl)piperidin-4-yloxy)cyclohexyloxy)pyrazine; and 2-(methylsulfonyl)-5-(4-(1-(2,2,2-trifluoroethyl)piperidin-4-yloxy)cyclohexyloxy)pyrazine.

Some embodiments of the present invention include every combination of one or more compounds selected from the following group shown in Table A.

TABLE A

| Cmpd | |
|---|---|
| No. | Chemical Structure and Name |
| 1 | 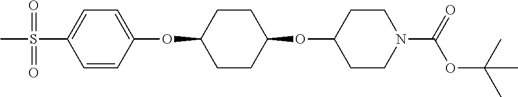 [SMILES: CC(C)(C)OC(=O)N1CCC(O[C@H]2CC[C@@H](Oc3ccc(S(C)(=O)=O)cc3)CC2)CC1] tert-butyl 4-((1s,4s)-4-(4- (methylsulfonyl)phenoxy)cyclohexyloxy)piperidine-1-carboxylate |
| 2 | 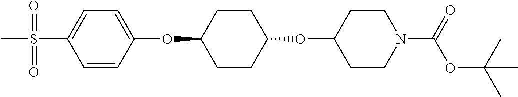 [SMILES: CC(C)(C)OC(=O)N1CCC(O[C@H]2CC[C@H](Oc3ccc(S(C)(=O)=O)cc3)CC2)CC1] tert-butyl 4-((1r,4r)-4-(4- (methylsulfonyl)phenoxy)cyclohexyloxy)piperidine-1-carboxylate |
| 3 | 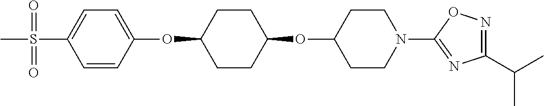 [SMILES: CC(C)c1noc(N2CCC(O[C@H]3CC[C@@H](Oc4ccc(S(C)(=O)=O)cc4)CC3)CC2)n1] 3-isopropyl-5-(4-((1s,4s)-4-(4- (methylsulfonyl)phenoxy)cyclohexyloxy)piperidin-1-yl)-1,2,4-oxadiazole |
| 4 | 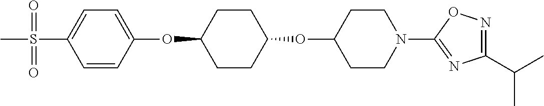 [SMILES: CC(C)c1noc(N2CCC(O[C@H]3CC[C@H](Oc4ccc(S(C)(=O)=O)cc4)CC3)CC2)n1] 3-isopropyl-5-(4-((1r,4r)-4-(4- (methylsulfonyl)phenoxy)cyclohexyloxy)piperidin-1-yl)-1,2,4-oxadiazole |
| 5 | 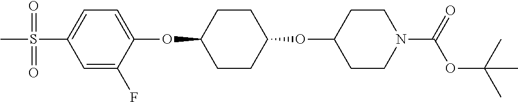 [SMILES: CC(C)(C)OC(=O)N1CCC(O[C@H]2CC[C@H](Oc3ccc(S(C)(=O)=O)cc3F)CC2)CC1] tert-butyl 4-((1r,4r)-4-(2-fluoro-4- (methylsulfonyl)phenoxy)cyclohexyloxy)piperidine-1-carboxylate |
| 6 | 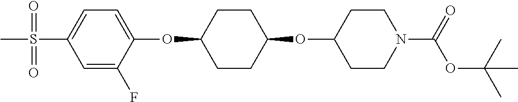 [SMILES: CC(C)(C)OC(=O)N1CCC(O[C@H]2CC[C@@H](Oc3ccc(S(C)(=O)=O)cc3F)CC2)CC1] tert-butyl 4-((1s,4s)-4-(2-fluoro-4- (methylsulfonyl)phenoxy)cyclohexyloxy)piperidine-1-carboxylate |
| 7 | 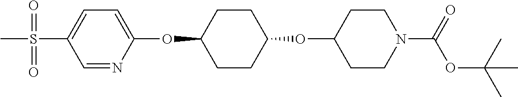 [SMILES: CC(C)(C)OC(=O)N1CCC(O[C@H]2CC[C@H](Oc3ccc(S(C)(=O)=O)cn3)CC2)CC1] tert-butyl 4-((1r,4r)-4-(5-(methylsulfonyl)pyridin-2- yloxy)cyclohexyloxy)piperidine-1-carboxylate |
| 8 | 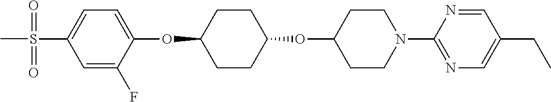 [SMILES: CCc1cnc(N2CCC(O[C@H]3CC[C@H](Oc4ccc(S(C)(=O)=O)cc4F)CC3)CC2)nc1] 5-ethyl-2-(4-((1r,4r)-4-(2-fluoro-4- (methylsulfonyl)phenoxy)cyclohexyloxy)piperidin-1-yl)pyrimidine |
| 9 | 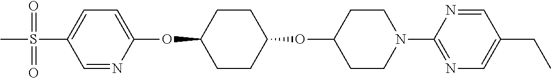 [SMILES: CCc1cnc(N2CCC(O[C@H]3CC[C@H](Oc4ccc(S(C)(=O)=O)cn4)CC3)CC2)nc1] 5-ethyl-2-(4-((1r,4r)-4-(5-(methylsulfonyl)pyridin-2- yloxy)cyclohexyloxy)piperidin-1-yl)pyrimidine |
| 10 | 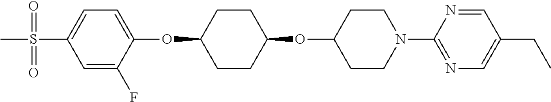 [SMILES: CCc1cnc(N2CCC(O[C@H]3CC[C@@H](Oc4ccc(S(C)(=O)=O)cc4F)CC3)CC2)nc1] 5-ethyl-2-(4-((1s,4s)-4-(2-fluoro-4- (methylsulfonyl)phenoxy)cyclohexyloxy)piperidin-1-yl)pyrimidine |
| 11 | 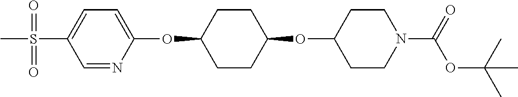 [SMILES: CC(C)(C)OC(=O)N1CCC(O[C@H]2CC[C@@H](Oc3ccc(S(C)(=O)=O)cn3)CC2)CC1] tert-butyl 4-((1s,4s)-4-(5-(methylsulfonyl)pyridin-2- yloxy)cyclohexyloxy)piperidine-1-carboxylate |
| 12 | 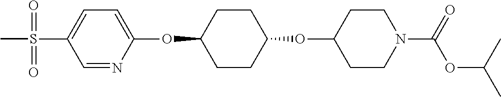 [SMILES: CC(C)OC(=O)N1CCC(O[C@H]2CC[C@H](Oc3ccc(S(C)(=O)=O)cn3)CC2)CC1] isopropyl 4-((1r,4r)-4-(5-(methylsulfonyl)pyridin-2- yloxy)cyclohexyloxy)piperidine-1-carboxylate |
| 13 | 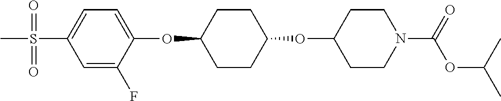 [SMILES: CC(C)OC(=O)N1CCC(O[C@H]2CC[C@H](Oc3ccc(S(C)(=O)=O)cc3F)CC2)CC1] isopropyl 4-((1r,4r)-4-(2-fluoro-4- (methylsulfonyl)phenoxy)cyclohexyloxy)piperidine-1-carboxylate |
| 14 | 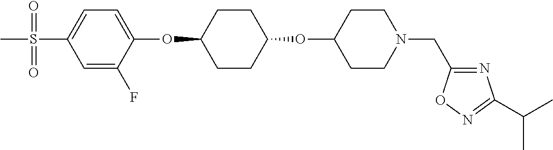 [SMILES: CC(C)c1noc(CN2CCC(O[C@H]3CC[C@H](Oc4ccc(S(C)(=O)=O)cc4F)CC3)CC2)n1] 5-((4-((1r,4r)-4-(2-fluoro-4- (methylsulfonyl)phenoxy)cyclohexyloxy)piperidin-1-yl)methyl)-3- isopropyl-1,2,4-oxadiazole |
| 15 | 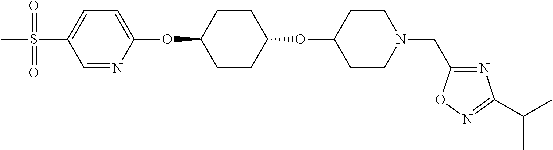 [SMILES: CC(C)c1noc(CN2CCC(O[C@H]3CC[C@H](Oc4ccc(S(C)(=O)=O)cn4)CC3)CC2)n1] 3-isopropyl-5-((4-((1r,4r)-4-(5-(methylsulfonyl)pyridin-2- yloxy)cyclohexyloxy)piperidin-1-yl)methyl)-1,2,4-oxadiazole |
| 16 | 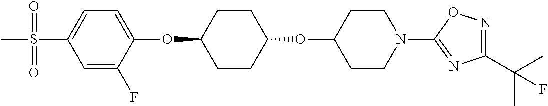 [SMILES: CC(C)(F)c1noc(N2CCC(O[C@H]3CC[C@H](Oc4ccc(S(C)(=O)=O)cc4F)CC3)CC2)n1] 5-(4-((1r,4r)-4-(2-fluoro-4- (methylsulfonyl)phenoxy)cyclohexyloxy)piperidin-1-yl)-3-(2-fluoropropan- 2-yl)-1,2,4-oxadiazole |
| 17 | 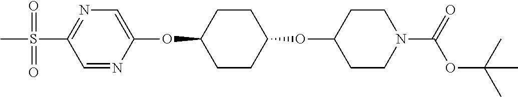 [SMILES: CC(C)(C)OC(=O)N1CCC(O[C@H]2CC[C@H](Oc3cnc(S(C)(=O)=O)cn3)CC2)CC1] tert-butyl 4-((1r,4r)-4-(5-(methylsulfonyl)pyrazin-2- yloxy)cyclohexyloxy)piperidine-1-carboxylate |
| 18 | 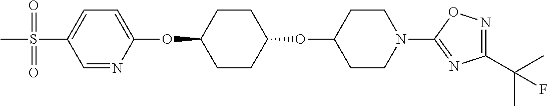 [SMILES: CC(C)(F)c1noc(N2CCC(O[C@H]3CC[C@H](Oc4ccc(S(C)(=O)=O)cn4)CC3)CC2)n1] 3-(2-fluoropropan-2-yl)-5-(4-((1r,4r)-4-(5-(methylsulfonyl)pyridin-2- yloxy)cyclohexyloxy)piperidin-1-yl)-1,2,4-oxadiazole |
| 19 | 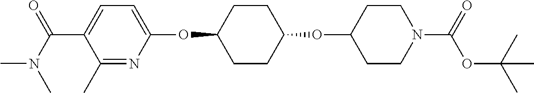 [SMILES: Cc1nc(O[C@H]2CC[C@H](OC3CCN(C(=O)OC(C)(C)C)CC3)CC2)ccc1C(=O)N(C)C] tert-butyl 4-((1r,4r)-4-(5-(dimethylcarbamoyl)-6-methylpyridin-2- yloxy)cyclohexyloxy)piperidine-1-carboxylate |
| 20 | 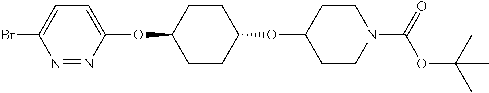 [SMILES: CC(C)(C)OC(=O)N1CCC(O[C@H]2CC[C@H](Oc3ccc(Br)nn3)CC2)CC1] tert-butyl 4-((1r,4r)-4-(6-bromopyridazin-3- yloxy)cyclohexyloxy)piperidine-1-carboxylate |
| 21 | 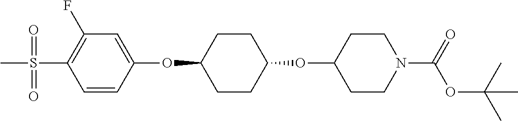 [SMILES: CC(C)(C)OC(=O)N1CCC(O[C@H]2CC[C@H](Oc3ccc(S(C)(=O)=O)c(F)c3)CC2)CC1] tert-butyl 4-((1r,4r)-4-(3-fluoro-4- (methylsulfonyl)phenoxy)cyclohexyloxy)piperidine-1-carboxylate |
| 22 | 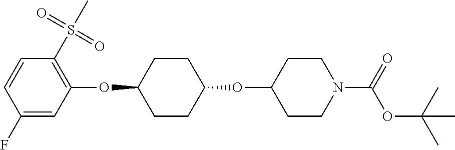 [SMILES: CC(C)(C)OC(=O)N1CCC(O[C@H]2CC[C@H](Oc3cc(F)ccc3S(C)(=O)=O)CC2)CC1] tert-butyl 4-((1r,4r)-4-(5-fluoro-2- (methylsulfonyl)phenoxy)cyclohexyloxy)piperidine-1-carboxylate |
| 23 | 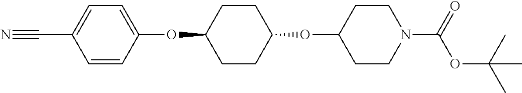 [SMILES: CC(C)(C)OC(=O)N1CCC(O[C@H]2CC[C@H](Oc3ccc(C#N)cc3)CC2)CC1] tert-butyl 4-((1r,4r)-4-(4-cyanophenoxy)cyclohexyloxy)piperidine-1- carboxylate |
| 24 | 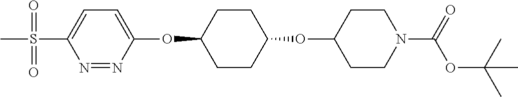 [SMILES: CC(C)(C)OC(=O)N1CCC(O[C@H]2CC[C@H](Oc3ccc(S(C)(=O)=O)nn3)CC2)CC1] tert-butyl 4-((1r,4r)-4-(6-(methylsulfonyl)pyridazin-3- yloxy)cyclohexyloxy)piperidine-1-carboxylate |
| 25 | 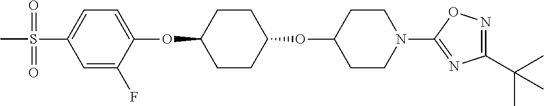 [SMILES: CC(C)(C)c1noc(N2CCC(O[C@H]3CC[C@H](Oc4ccc(S(C)(=O)=O)cc4F)CC3)CC2)n1] 3-tert-butyl-5-(4-((1r,4r)-4-(2-fluoro-4- (methylsulfonyl)phenoxy)cyclohexyloxy)piperidin-1-yl)-1,2,4-oxadiazole |
| 26 | 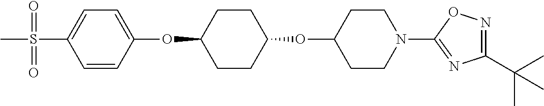 [SMILES: CC(C)(C)c1noc(N2CCC(O[C@H]3CC[C@H](Oc4ccc(S(C)(=O)=O)cc4)CC3)CC2)n1] 3-tert-butyl-5-(4-((1r,4r)-4-(4- (methylsulfonyl)phenoxy)cyclohexyloxy)piperidin-1-yl)-1,2,4-oxadiazole |
| 27 | 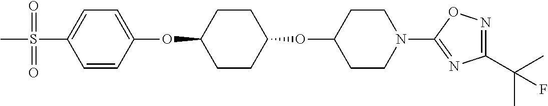 [SMILES: CC(C)(F)c1noc(N2CCC(O[C@H]3CC[C@H](Oc4ccc(S(C)(=O)=O)cc4)CC3)CC2)n1] 3-(2-fluoropropan-2-yl)-5-(4-((1r,4r)-4-(4- (methylsulfonyl)phenoxy)cyclohexyloxy)piperidin-1-yl)-1,2,4-oxadiazole |
| 28 | 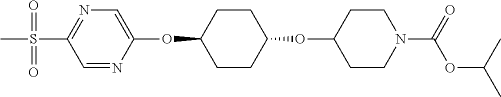 [SMILES: CC(C)OC(=O)N1CCC(O[C@H]2CC[C@H](Oc3cnc(S(C)(=O)=O)cn3)CC2)CC1] isopropyl 4-((1r,4r)-4-(5-(methylsulfonyl)pyrazin-2- yloxy)cyclohexyloxy)piperidine-1-carboxylate |
| 29 | 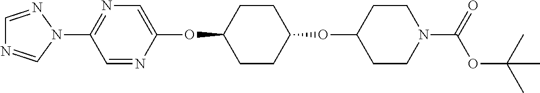 [SMILES: CC(C)(C)OC(=O)N1CCC(O[C@H]2CC[C@H](Oc3cnc(-n4cncn4)cn3)CC2)CC1] tert-butyl 4-((1r,4r)-4-(5-(1H-1,2,4-triazol-1-yl)pyrazin-2- yloxy)cyclohexyloxy)piperidine-1-carboxylate |
| 30 | 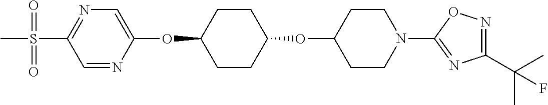 [SMILES: CC(C)(F)c1noc(N2CCC(O[C@H]3CC[C@H](Oc4cnc(S(C)(=O)=O)cn4)CC3)CC2)n1] 3-(2-fluoropropan-2-yl)-5-(4-((1r,4r)-4-(5-(methylsulfonyl)pyrazin-2- yloxy)cyclohexyloxy)piperidin-1-yl)-1,2,4-oxadiazole |
| 31 | 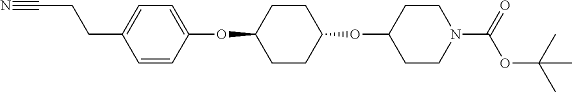 [SMILES: CC(C)(C)OC(=O)N1CCC(O[C@H]2CC[C@H](Oc3ccc(CCC#N)cc3)CC2)CC1] tert-butyl 4-((1r,4r)-4-(4-(2-cyanoethyl)phenoxy)cyclohexyloxy)piperidine- 1-carboxylate |
| 32 | 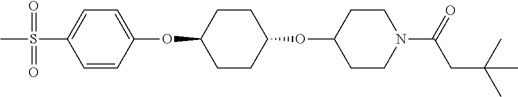 [SMILES: CC(C)(C)CC(=O)N1CCC(O[C@H]2CC[C@H](Oc3ccc(S(C)(=O)=O)cc3)CC2)CC1] 3,3-dimethyl-1-(4-((1r,4r)-4-(4- (methylsulfonyl)phenoxy)cyclohexyloxy)piperidin-1-yl)butan-1-one |
| 33 | 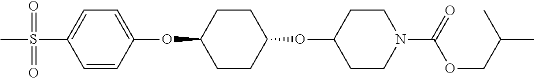 [SMILES: CC(C)COC(=O)N1CCC(O[C@H]2CC[C@H](Oc3ccc(S(C)(=O)=O)cc3)CC2)CC1] isobutyl 4-((1r,4r)-4-(4- (methylsulfonyl)phenoxy)cyclohexyloxy)piperidine-1-carboxylate |
| 34 | 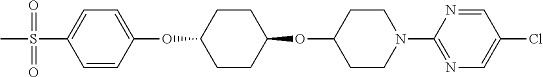 [SMILES: CS(=O)(=O)c1ccc(O[C@H]2CC[C@H](OC3CCN(c4ncc(Cl)cn4)CC3)CC2)cc1] 5-chloro-2-(4-((1r,4r)-4-(4- (methylsulfonyl)phenoxy)cyclohexyloxy)piperidin-1-yl)pyrimidine |
| 35 | 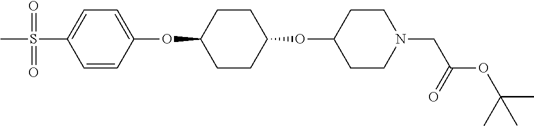 [SMILES: CC(C)(C)OC(=O)CN1CCC(O[C@H]2CC[C@H](Oc3ccc(S(C)(=O)=O)cc3)CC2)CC1] tert-butyl 2-(4-((1r,4r)-4-(4- (methylsulfonyl)phenoxy)cyclohexyloxy)piperidin-1-yl)acetate |
| 36 | 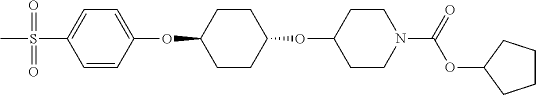 [SMILES: CS(=O)(=O)c1ccc(O[C@H]2CC[C@H](OC3CCN(C(=O)OC4CCCC4)CC3)CC2)cc1] cyclopentyl 4-((1r,4r)-4-(4- (methylsulfonyl)phenoxy)cyclohexyloxy)piperidine-1-carboxylate |
| 37 | 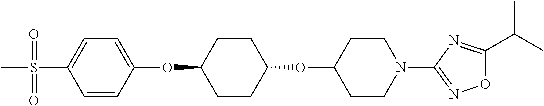 [SMILES: CC(C)c1nc(N2CCC(O[C@H]3CC[C@H](Oc4ccc(S(C)(=O)=O)cc4)CC3)CC2)no1] 5-isopropyl-3-(4-((1r,4r)-4-(4- (methylsulfonyl)phenoxy)cyclohexyloxy)piperidin-1-yl)-1,2,4-oxadiazole |
| 38 | 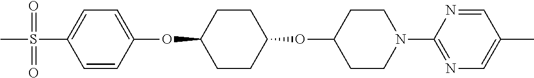 5-methyl-2-(4-((1r,4r)-4-(4- (methylsulfonyl)phenoxy)cyclohexyloxy)piperidin-1-yl)pyrimidine |
| 39 | 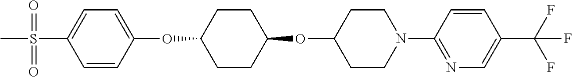 [SMILES: CS(=O)(=O)c1ccc(O[C@H]2CC[C@H](OC3CCN(c4ccc(C(F)(F)F)cn4)CC3)CC2)cc1] 2-(4-((1r,4r)-4-(4-(methylsulfonyl)phenoxy)cyclohexyloxy)piperidin-1-yl)- 5-(trifluoromethyl)pyridine |
| 40 | 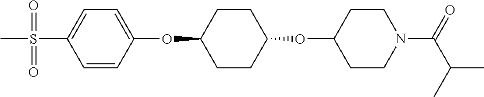 [SMILES: CC(C)C(=O)N1CCC(O[C@H]2CC[C@H](Oc3ccc(S(C)(=O)=O)cc3)CC2)CC1] 2-methyl-1-(4-((1r,4r)-4-(4- (methylsulfonyl)phenoxy)cyclohexyloxy)piperidin-1-yl)propan-1-one |
| 41 | 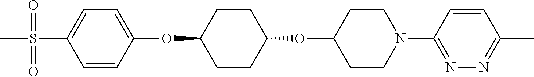 3-methyl-6-(4-((1r,4r)-4-(4- (methylsulfonyl)phenoxy)cyclohexyloxy)piperidin-1-yl)pyridazine |
| 42 | 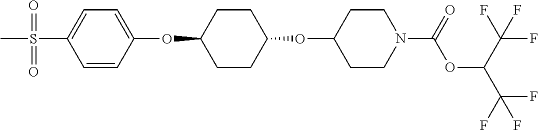 [SMILES: CS(=O)(=O)c1ccc(O[C@H]2CC[C@H](OC3CCN(C(=O)OC(C(F)(F)F)C(F)(F)F)CC3)CC2)cc1] 1,1,1,3,3,3-hexafluoropropan-2-yl 4-((1r,4r)-4-(4- (methylsulfonyl)phenoxy)cyclohexyloxy)piperidine-1-carboxylate |
| 43 | 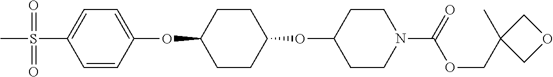 [SMILES: CC1(COC(=O)N2CCC(O[C@H]3CC[C@H](Oc4ccc(S(C)(=O)=O)cc4)CC3)CC2)COC1] (3-methyloxetan-3-yl)methyl4-((1r,4r)-4-(4- (methylsulfonyl)phenoxy)cyclohexyloxy)piperidine-1-carboxylate |
| 44 | 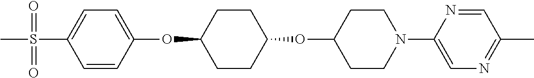 2-methyl-5-(4-((1r,4r)-4-(4- (methylsulfonyl)phenoxy)cyclohexyloxy)piperidin-1-yl)pyrazine |
| 45 | 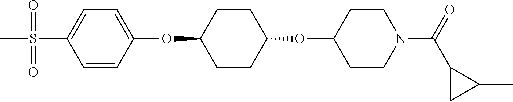 (2-methylcyclopropyl)(4-((1r,4r)-4-(4- (methylsulfonyl)phenoxy)cyclohexyloxy)piperidin-1-yl)methanone |
| 46 | 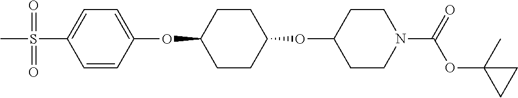 [SMILES: CC1(OC(=O)N2CCC(O[C@H]3CC[C@H](Oc4ccc(S(C)(=O)=O)cc4)CC3)CC2)CC1] 1-methylcyclopropyl4-((1r,4r)-4-(4- (methylsulfonyl)phenoxy)cyclohexyloxy)piperidine-1-carboxylate |
| 47 | 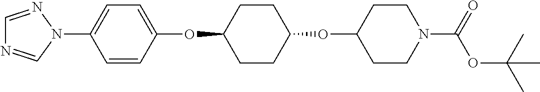 [SMILES: CC(C)(C)OC(=O)N1CCC(O[C@H]2CC[C@H](Oc3ccc(-n4cncn4)cc3)CC2)CC1] tert-butyl 4-((1r,4r)-4-(4-(1H-1,2,4-triazol-1- yl)phenoxy)cyclohexyloxy)piperidine-1-carboxylate |
| 48 | 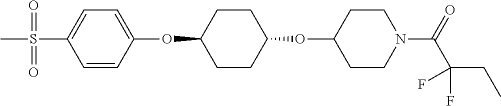 [SMILES: CCC(F)(F)C(=O)N1CCC(O[C@H]2CC[C@H](Oc3ccc(S(C)(=O)=O)cc3)CC2)CC1] 2,2-difluoro-1-(4-((1r,4r)-4-(4- (methylsulfonyl)phenoxy)cyclohexyloxy)piperidin-1-yl)butan-1-one |
| 49 | 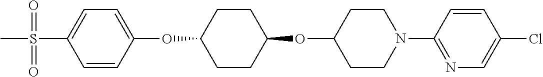 [SMILES: CS(=O)(=O)c1ccc(O[C@H]2CC[C@H](OC3CCN(c4ccc(Cl)cn4)CC3)CC2)cc1] 5-chloro-2-(4-((1r,4r)-4-(4- (methylsulfonyl)phenoxy)cyclohexyloxy)piperidin-1-yl)pyridine |
| 50 | 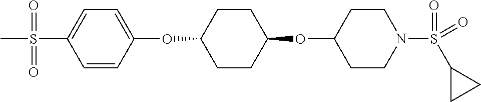 [SMILES: CS(=O)(=O)c1ccc(O[C@H]2CC[C@H](OC3CCN(S(=O)(=O)C4CC4)CC3)CC2)cc1] 1-(cyclopropylsulfonyl)-4-((1r,4r)-4-(4- (methylsulfonyl)phenoxy)cyclohexyloxy)piperidine |
| 51 | 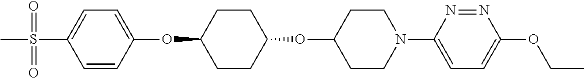 [SMILES: CCOc1ccc(N2CCC(O[C@H]3CC[C@H](Oc4ccc(S(C)(=O)=O)cc4)CC3)CC2)nn1] 3-ethoxy-6-(4-((1r,4r)-4-(4- (methylsulfonyl)phenoxy)cyclohexyloxy)piperidin-1-yl)pyridazine |
| 52 | 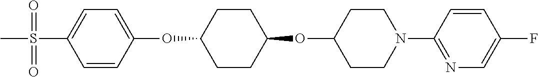 [SMILES: CS(=O)(=O)c1ccc(O[C@H]2CC[C@H](OC3CCN(c4ccc(F)cn4)CC3)CC2)cc1] 5-fluoro-2-(4-((1r,4r)-4-(4- (methylsulfonyl)phenoxy)cyclohexyloxy)piperidin-1-yl)pyridine |
| 53 | 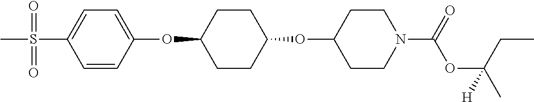 [SMILES: CC[C@@H](C)OC(=O)N1CCC(O[C@H]2CC[C@H](Oc3ccc(S(C)(=O)=O)cc3)CC2)CC1] (R)-sec-butyl 4-((1r,4r)-4-(4- (methylsulfonyl)phenoxy)cyclohexyloxy)piperidine-1-carboxylate |
| 54 | 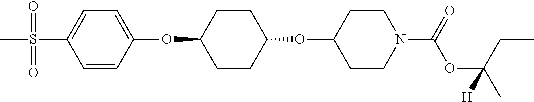 [SMILES: CC[C@H](C)OC(=O)N1CCC(O[C@H]2CC[C@H](Oc3ccc(S(C)(=O)=O)cc3)CC2)CC1] (S)-sec-butyl 4-((1r,4r)-4-(4- (methylsulfonyl)phenoxy)cyclohexyloxy)piperidine-1-carboxylate |
| 55 | 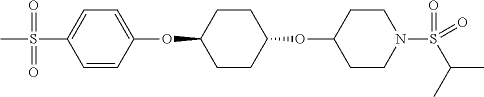 [SMILES: CC(C)S(=O)(=O)N1CCC(O[C@H]2CC[C@H](Oc3ccc(S(C)(=O)=O)cc3)CC2)CC1] 1-(isopropylsulfonyl)-4-((1r,4r)-4-(4- (methylsulfonyl)phenoxy)cyclohexyloxy)piperidine |
| 56 | 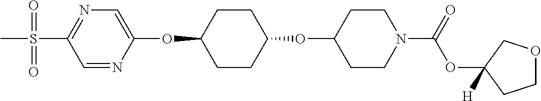 [SMILES: CS(=O)(=O)c1cnc(O[C@H]2CC[C@H](OC3CCN(C(=O)O[C@H]4CCOC4)CC3)CC2)cn1] (S)-tetrahydrofuran-3-yl4-((1r,4r)-4-(5-(methylsulfonyl)pyrazin-2- yloxy)cyclohexyloxy)piperidine-1-carboxylate |
| 57 | 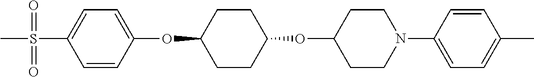 4-((1r,4r)-4-(4-(methylsulfonyl)phenoxy)cyclohexyloxy)-1-p- tolylpiperidine |
| 58 | 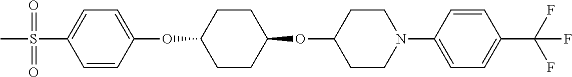 [SMILES: CS(=O)(=O)c1ccc(O[C@H]2CC[C@H](OC3CCN(c4ccc(C(F)(F)F)cc4)CC3)CC2)cc1] 4-((1r,4r)-4-(4-(methylsulfonyl)phenoxy)cyclohexyloxy)-1-(4- (trifluoromethyl)phenyl)piperidine |
| 59 | 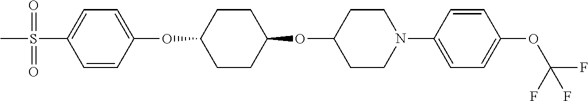 [SMILES: CS(=O)(=O)c1ccc(O[C@H]2CC[C@H](OC3CCN(c4ccc(OC(F)(F)F)cc4)CC3)CC2)cc1] 4-((1r,4r)-4-(4-(methylsulfonyl)phenoxy)cyclohexyloxy)-1-(4- (trifluoromethoxy)phenyl)piperidine |
| 60 | 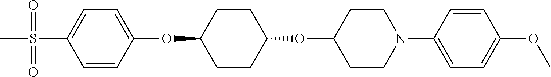 1-(4-methoxyphenyl)-4-((1r,4r)-4-(4- (methylsulfonyl)phenoxy)cyclohexyloxy)piperidine |
| 61 | 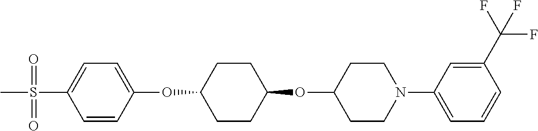 [SMILES: CS(=O)(=O)c1ccc(O[C@H]2CC[C@H](OC3CCN(c4cccc(C(F)(F)F)c4)CC3)CC2)cc1] 4-((1r,4r)-4-(4-(methylsulfonyl)phenoxy)cyclohexyloxy)-1-(3- (trifluoromethyl)phenyl)piperidine |
| 62 | 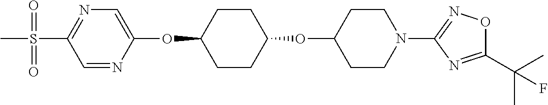 [SMILES: CC(C)(F)c1nc(N2CCC(O[C@H]3CC[C@H](Oc4cnc(S(C)(=O)=O)cn4)CC3)CC2)no1] 5-(2-fluoropropan-2-yl)-3-(4-((1r,4r)-4-(5-(methylsulfonyl)pyrazin-2- yloxy)cyclohexyloxy)piperidin-1-yl)-1,2,4-oxadiazole |
| 63 | 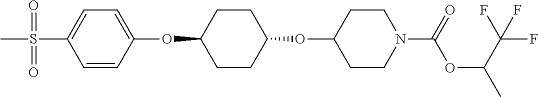 [SMILES: CC(OC(=O)N1CCC(O[C@H]2CC[C@H](Oc3ccc(S(C)(=O)=O)cc3)CC2)CC1)C(F)(F)F] 1,1,1-trifluoropropan-2-yl 4-((1r,4r)-4-(4- (methylsulfonyl)phenoxy)cyclohexyloxy)piperidine-1-carboxylate |
| 64 | 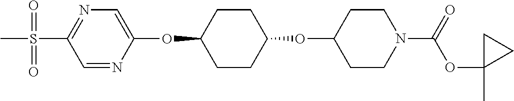 [SMILES: CC1(OC(=O)N2CCC(O[C@H]3CC[C@H](Oc4cnc(S(C)(=O)=O)cn4)CC3)CC2)CC1] 1-methylcyclopropyl 4-((1r,4r)-4-(5-(methylsulfonyl)pyrazin-2- yloxy)cyclohexyloxy)piperidine-1-carboxylate |
| 65 | 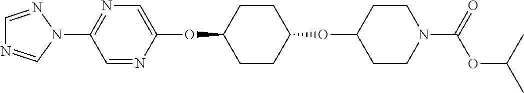 [SMILES: CC(C)OC(=O)N1CCC(O[C@H]2CC[C@H](Oc3cnc(-n4cncn4)cn3)CC2)CC1] isopropyl 4-((1r,4r)-4-(5-(1H-1,2,4-triazol-1-yl)pyrazin-2- yloxy)cyclohexyloxy)piperidine-1-carboxylate |
| 66 | 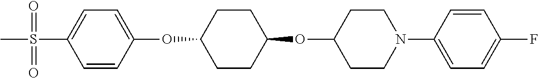 [SMILES: CS(=O)(=O)c1ccc(O[C@H]2CC[C@H](OC3CCN(c4ccc(F)cc4)CC3)CC2)cc1] 1-(4-fluorophenyl)-4-((1r,4r)-4-(4- (methylsulfonyl)phenoxy)cyclohexyloxy)piperidine |
| 67 | 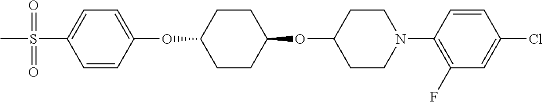 [SMILES: CS(=O)(=O)c1ccc(O[C@H]2CC[C@H](OC3CCN(c4ccc(Cl)cc4F)CC3)CC2)cc1] 1-(4-chloro-2-fluorophenyl)-4-((1r,4r)-4-(4- (methylsulfonyl)phenoxy)cyclohexyloxy)piperidine |
| 68 | 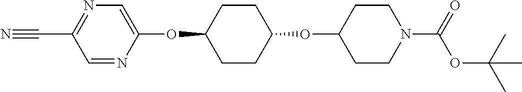 [SMILES: CC(C)(C)OC(=O)N1CCC(O[C@H]2CC[C@H](Oc3cnc(C#N)cn3)CC2)CC1] tert-butyl 4-((1r,4r)-4-(5-cyanopyrazin-2-yloxy)cyclohexyloxy)piperidine- 1-carboxylate |
| 69 | 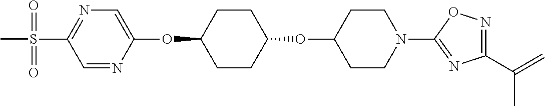 [SMILES: C=C(C)c1noc(N2CCC(O[C@H]3CC[C@H](Oc4cnc(S(C)(=O)=O)cn4)CC3)CC2)n1] 5-(4-((1r,4r)-4-(5-(methylsulfonyl)pyrazin-2- yloxy)cyclohexyloxy)piperidin-1-yl)-3-(prop-1-en-2-yl)-1,2,4-oxadiazole |
| 70 | 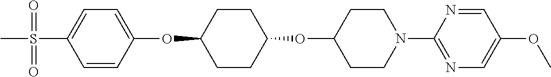 5-methoxy-2-(4-((1r,4r)-4-(4- (methylsulfonyl)phenoxy)cyclohexyloxy)piperidin-1-yl)pyrimidine |
| 71 | 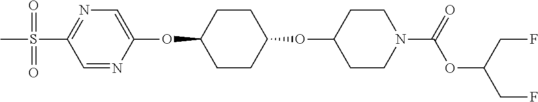 [SMILES: CS(=O)(=O)c1cnc(O[C@H]2CC[C@H](OC3CCN(C(=O)OC(CF)CF)CC3)CC2)cn1] 1,3-difluoropropan-2-yl 4-((1r,4r)-4-(5-(methylsulfonyl)pyrazin-2- yloxy)cyclohexyloxy)piperidine-1-carboxylate |
| 72 | 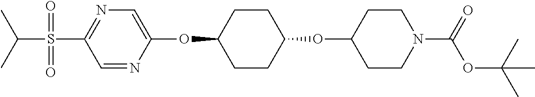 [SMILES: CC(C)S(=O)(=O)c1cnc(O[C@H]2CC[C@H](OC3CCN(C(=O)OC(C)(C)C)CC3)CC2)cn1] tert-butyl 4-((1r,4r)-4-(5-(isopropylsulfonyl)pyrazin-2- yloxy)cyclohexyloxy)piperidine-1-carboxylate |
| 73 | 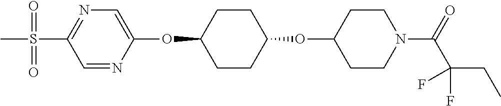 [SMILES: CCC(F)(F)C(=O)N1CCC(O[C@H]2CC[C@H](Oc3cnc(S(C)(=O)=O)cn3)CC2)CC1] 2,2-difluoro-1-(4-((1r,4r)-4-(5-(methylsulfonyl)pyrazin-2- yloxy)cyclohexyloxy)piperidin-1-yl)butan-1-one |
| 74 | 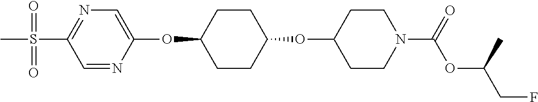 [SMILES: C[C@@H](CF)OC(=O)N1CCC(O[C@H]2CC[C@H](Oc3cnc(S(C)(=O)=O)cn3)CC2)CC1] (S)-1-fluoropropan-2-yl 4-((1r,4r)-4-(5-(methylsulfonyl)pyrazin-2- yloxy)cyclohexyloxy)piperidine-1-carboxylate |
| 75 | 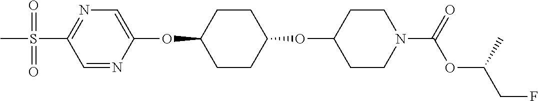 [SMILES: C[C@H](CF)OC(=O)N1CCC(O[C@H]2CC[C@H](Oc3cnc(S(C)(=O)=O)cn3)CC2)CC1] (R)-1-fluoropropan-2-yl 4-((1r,4r)-4-(5-(methylsulfonyl)pyrazin-2- yloxy)cyclohexyloxy)piperidine-1-carboxylate |
| 76 | 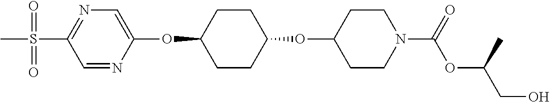 [SMILES: C[C@@H](CO)OC(=O)N1CCC(O[C@H]2CC[C@H](Oc3cnc(S(C)(=O)=O)cn3)CC2)CC1] (S)-1-hydroxypropan-2-yl 4-((1r,4r)-4-(5-(methylsulfonyl)pyrazin-2- yloxy)cyclohexyloxy)piperidine-1-carboxylate |
| 77 | 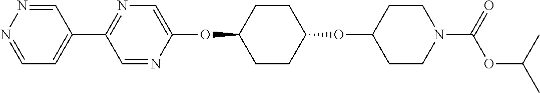 [SMILES: CC(C)OC(=O)N1CCC(O[C@H]2CC[C@H](Oc3cnc(-c4ccnnc4)cn3)CC2)CC1] isopropyl 4-((1r,4r)-4-(5-(pyridazin-4-yl)pyrazin-2-yloxy)cyclohexyloxy)- piperidine-1-carboxylate |
| 78 | 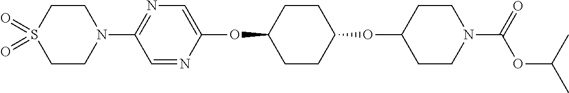 [SMILES: CC(C)OC(=O)N1CCC(O[C@H]2CC[C@H](Oc3cnc(N4CCS(=O)(=O)CC4)cn3)CC2)CC1] isopropyl 4-((1r,4r)-4-(5-(1,1-dioxo-thiomorpholin-4-yl)pyrazin-2- yloxy)cyclohexyloxy)piperidine-1-carboxylate |
| 79 | 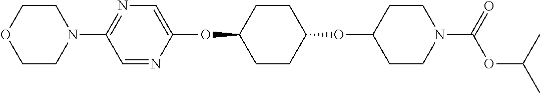 [SMILES: CC(C)OC(=O)N1CCC(O[C@H]2CC[C@H](Oc3cnc(N4CCOCC4)cn3)CC2)CC1] isopropyl 4-((1r,4r)-4-(5-morpholinopyrazin-2- yloxy)cyclohexyloxy)piperidine-1-carboxylate |
| 80 | 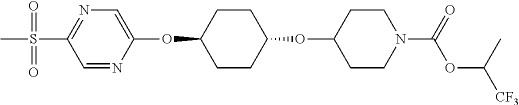 [SMILES: CC(OC(=O)N1CCC(O[C@H]2CC[C@H](Oc3cnc(S(C)(=O)=O)cn3)CC2)CC1)C(F)(F)F] 1,1,1-trifluoropropan-2-yl 4-((1r,4r)-4-(5-(methylsulfonyl)pyrazin-2- yloxy)cyclohexyloxy)piperidine-1-carboxylate |
| 81 | 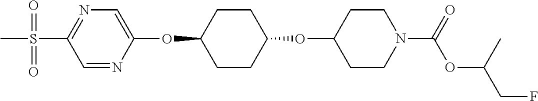 [SMILES: CC(CF)OC(=O)N1CCC(O[C@H]2CC[C@H](Oc3cnc(S(C)(=O)=O)cn3)CC2)CC1] 1-fluoropropan-2-yl 4-((1r,4r)-4-(5-(methylsulfonyl)pyrazin-2- yloxy)cyclohexyloxy)piperidine-1-carboxylate |
| 82 | 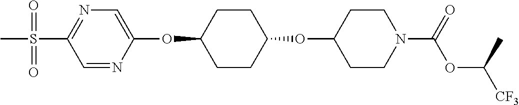 [SMILES: C[C@H](OC(=O)N1CCC(O[C@H]2CC[C@H](Oc3cnc(S(C)(=O)=O)cn3)CC2)CC1)C(F)(F)F] (S)-1,1,1-trifluoropropan-2-yl 4-((1r,4r)-4-(5-(methylsulfonyl)pyrazin-2- yloxy)cyclohexyloxy)piperidine-1-carboxylate |
| 83 | 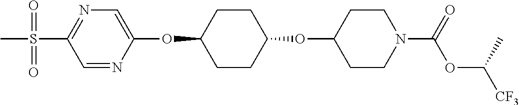 [SMILES: C[C@@H](OC(=O)N1CCC(O[C@H]2CC[C@H](Oc3cnc(S(C)(=O)=O)cn3)CC2)CC1)C(F)(F)F] (R)-1,1,1-trifluoropropan-2-yl 4-((1r,4r)-4-(5-(methylsulfonyl)pyrazin-2- yloxy)cyclohexyloxy)piperidine-1-carboxylate |
| 84 | 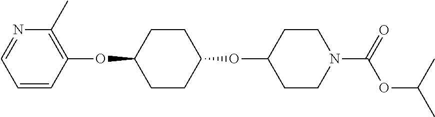 [SMILES: Cc1ncccc1O[C@H]1CC[C@H](OC2CCN(C(=O)OC(C)C)CC2)CC1] isopropyl 4-((1r,4r)-4-(2-methylpyridin-3-yloxy)cyclohexyloxy)piperidine- 1-carboxylate |
| 85 | 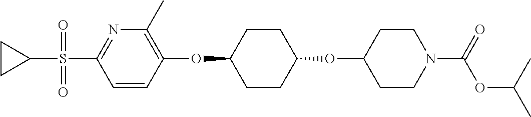 [SMILES: Cc1nc(S(=O)(=O)C2CC2)ccc1O[C@H]1CC[C@H](OC2CCN(C(=O)OC(C)C)CC2)CC1] isopropyl 4-((1r,4r)-4-(6-(cyclopropylsulfonyl)-2-methylpyridin-3- yloxy)cyclohexyloxy)piperidine-1-carboxylate |
| 86 | 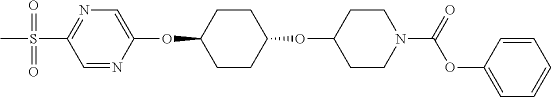 [SMILES: CS(=O)(=O)c1cnc(O[C@H]2CC[C@H](OC3CCN(C(=O)Oc4ccccc4)CC3)CC2)cn1] phenyl 4-((1r,4r)-4-(5-(methylsulfonyl)pyrazin-2- yloxy)cyclohexyloxy)piperidine-1-carboxylate |
| 87 | 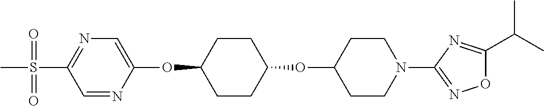 [SMILES: CC(C)c1nc(N2CCC(O[C@H]3CC[C@H](Oc4cnc(S(C)(=O)=O)cn4)CC3)CC2)no1] 5-isopropyl-3-(4-((1r,4r)-4-(5-(methylsulfonyl)pyrazin-2- yloxy)cyclohexyloxy)piperidin-1-yl)-1,2,4-oxadiazole |
| 88 | 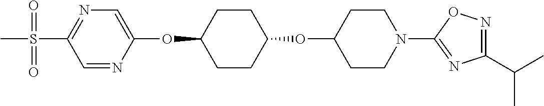 [SMILES: CC(C)c1noc(N2CCC(O[C@H]3CC[C@H](Oc4cnc(S(C)(=O)=O)cn4)CC3)CC2)n1] 3-isopropyl-5-(4-((1r,4r)-4-(5-(methylsulfonyl)pyrazin-2- yloxy)cyclohexyloxy)piperidin-1-yl)-1,2,4-oxadiazole |
| 89 | 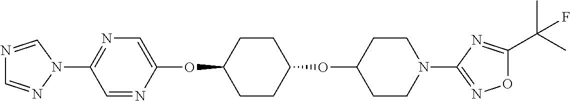 [SMILES: CC(C)(F)c1nc(N2CCC(O[C@H]3CC[C@H](Oc4cnc(-n5cncn5)cn4)CC3)CC2)no1] 3-(4-((1r,4r)-4-(5-(1H-1,2,4-triazol-1-yl)pyrazin-2- yloxy)cyclohexyloxy)piperidin-1-yl)-5-(2-fluoropropan-2-yl)-1,2,4-oxadiazole |
| 90 | 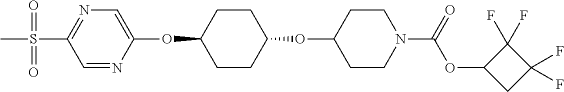 [SMILES: CS(=O)(=O)c1cnc(O[C@H]2CC[C@H](OC3CCN(C(=O)OC4CC(F)(F)C4(F)F)CC3)CC2)cn1] 2,2,3,3-tetrafluorocyclobutyl 4-((1r,4r)-4-(5-(methylsulfonyl)pyrazin-2- yloxy)cyclohexyloxy)piperidine-1-carboxylate |
| 91 | 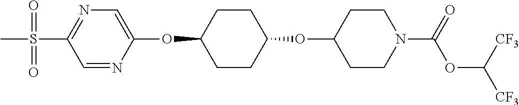 [SMILES: CS(=O)(=O)c1cnc(O[C@H]2CC[C@H](OC3CCN(C(=O)OC(C(F)(F)F)C(F)(F)F)CC3)CC2)cn1] 1,1,1,3,3,3-hexafluoropropan-2-yl 4-((1r,4r)-4-(5-(methylsulfonyl)pyrazin- 2-yloxy)cyclohexyloxy)piperidine-1-carboxylate |
| 92 | 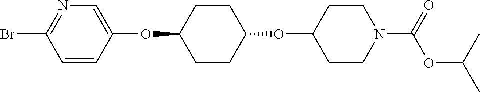 [SMILES: CC(C)OC(=O)N1CCC(O[C@H]2CC[C@H](Oc3ccc(Br)nc3)CC2)CC1] isopropyl 4-((1r,4r)-4-(6-bromopyridin-3-yloxy)cyclohexyloxy)piperidine- 1-carboxylate |
| 93 | 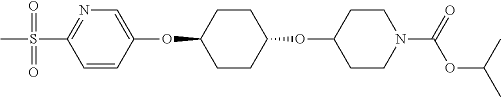 [SMILES: CC(C)OC(=O)N1CCC(O[C@H]2CC[C@H](Oc3ccc(S(C)(=O)=O)nc3)CC2)CC1] isopropyl 4-((1r,4r)-4-(6-(methylsulfonyl)pyridin-3- yloxy)cyclohexyloxy)piperidine-1-carboxylate |
| 94 | 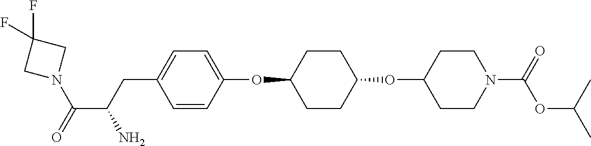 [SMILES: CC(C)OC(=O)N1CCC(O[C@H]2CC[C@H](Oc3ccc(C[C@H](N)C(=O)N4CC(F)(F)C4)cc3)CC2)CC1] isopropyl 4-((1S,4r)-4-(4-((S)-2-amino-3-(3,3-difluoroazetidin-1-yl)-3- oxopropyl)phenoxy)cyclohexyloxy)piperidine-1-carboxylate |
| 95 | 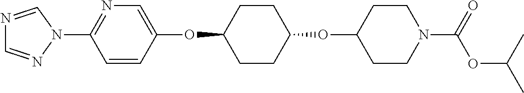 [SMILES: CC(C)OC(=O)N1CCC(O[C@H]2CC[C@H](Oc3ccc(-n4cncn4)nc3)CC2)CC1] isopropyl 4-((1r,4r)-4-(6-(1H-1,2,4-triazol-1-yl)pyridin-3- yloxy)cyclohexyloxy)piperidine-1-carboxylate |
| 96 | 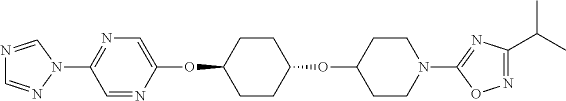 [SMILES: CC(C)c1noc(N2CCC(O[C@H]3CC[C@H](Oc4cnc(-n5cncn5)cn4)CC3)CC2)n1] 5-(4-((1r,4r)-4-(5-(1H-1,2,4-triazol-1-yl)pyrazin-2- yloxy)cyclohexyloxy)piperidin-1-yl)-3-isopropyl-1,2,4-oxadiazole |
| 97 | 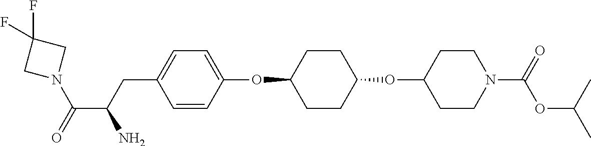 [SMILES: CC(C)OC(=O)N1CCC(O[C@H]2CC[C@H](Oc3ccc(C[C@@H](N)C(=O)N4CC(F)(F)C4)cc3)CC2)CC1] isopropyl 4-((1R,4r)-4-(4-((R)-2-amino-3-(3,3-difluoroazetidin-1-yl)-3- oxopropyl)phenoxy)cyclohexyloxy)piperidine-1-carboxylate |
| 98 | 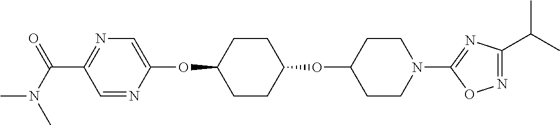 [SMILES: CC(C)c1noc(N2CCC(O[C@H]3CC[C@H](Oc4cnc(C(=O)N(C)C)cn4)CC3)CC2)n1] 5-((1r,4r)-4-(1-(3-isopropyl-1,2,4-oxadiazol-5-yl)piperidin-4- yloxy)cyclohexyloxy)-N,N-dimethylpyrazine-2-carboxamide |
| 99 | 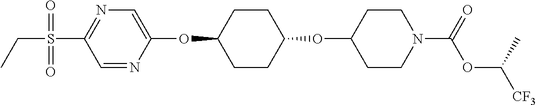 [SMILES: CCS(=O)(=O)c1cnc(O[C@H]2CC[C@H](OC3CCN(C(=O)O[C@H](C)C(F)(F)F)CC3)CC2)cn1] (R)-1,1,1-trifluoropropan-2-yl 4-((1r,4r)-4-(5-(ethylsulfonyl)pyrazin-2- yloxy)cyclohexyloxy)piperidine-1-carboxylate |
| 100 | 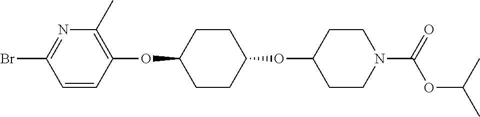 [SMILES: Cc1nc(Br)ccc1O[C@H]1CC[C@H](OC2CCN(C(=O)OC(C)C)CC2)CC1] isopropyl 4-((1r,4r)-4-(6-bromo-2-methylpyridin-3- yloxy)cyclohexyloxy)piperidine-1-carboxylate |
| 101 | 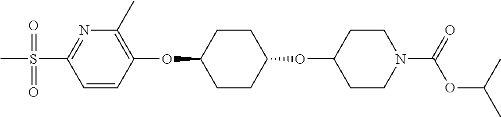 [SMILES: Cc1nc(S(C)(=O)=O)ccc1O[C@H]1CC[C@H](OC2CCN(C(=O)OC(C)C)CC2)CC1] isopropyl 4-((1r,4r)-4-(2-methyl-6-(methylsulfonyl)pyridin-3- yloxy)cyclohexyloxy)piperidine-1-carboxylate |
| 102 | 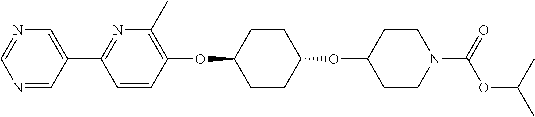 [SMILES: Cc1nc(-c2cncnc2)ccc1O[C@H]1CC[C@H](OC2CCN(C(=O)OC(C)C)CC2)CC1] isopropyl 4-((1r,4r)-4-(2-methyl-6-(pyrimidin-5-yl)pyridin-3- yloxy)cyclohexyloxy)piperidine-1-carboxylate |
| 103 | 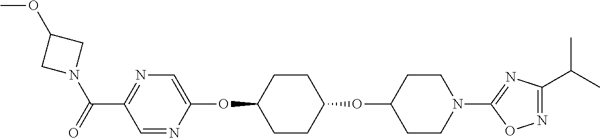 [SMILES: COC1CN(C(=O)c2cnc(O[C@H]3CC[C@H](OC4CCN(c5nc(C(C)C)no5)CC4)CC3)cn2)C1] (5-((1r,4r)-4-(1-(3-isopropyl-1,2,4-oxadiazol-5-yl)piperidin-4- yloxy)cyclohexyloxy)pyrazin-2-yl)(3-methoxyazetidin-1-yl)methanone |
| 104 | 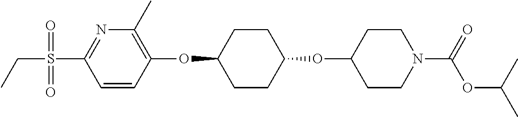 [SMILES: CCS(=O)(=O)c1ccc(O[C@H]2CC[C@H](OC3CCN(C(=O)OC(C)C)CC3)CC2)c(C)n1] isopropyl 4-((1r,4r)-4-(6-(ethylsulfonyl)-2-methylpyridin-3- yloxy)cyclohexyloxy)piperidine-1-carboxylate |
| 105 | 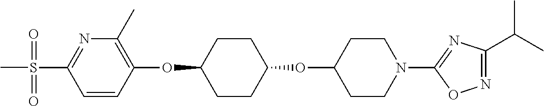 [SMILES: Cc1nc(S(C)(=O)=O)ccc1O[C@H]1CC[C@H](OC2CCN(c3nc(C(C)C)no3)CC2)CC1] 3-isopropyl-5-(4-((1r,4r)-4-(2-methyl-6-(methylsulfonyl)pyridin-3- yloxy)cyclohexyloxy)piperidin-1-yl)-1,2,4-oxadiazole |
| 106 | 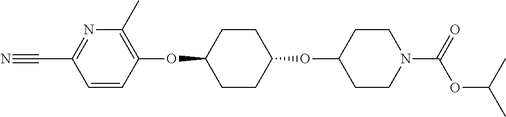 [SMILES: Cc1nc(C#N)ccc1O[C@H]1CC[C@H](OC2CCN(C(=O)OC(C)C)CC2)CC1] isopropyl 4-((1r,4r)-4-(6-cyano-2-methylpyridin-3- yloxy)cyclohexyloxy)piperidine-1-carboxylate |
| 107 | 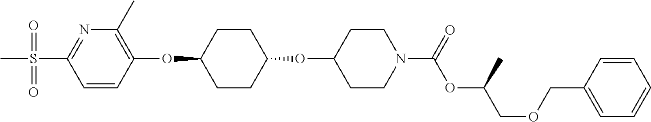 [SMILES: Cc1nc(S(C)(=O)=O)ccc1O[C@H]1CC[C@H](OC2CCN(C(=O)O[C@@H](C)COCc3ccccc3)CC2)CC1] (S)-1-(benzyloxy)propan-2-yl 4-((1r,4r)-4-(2-methyl-6- (methylsulfonyl)pyridin-3-yloxy)cyclohexyloxy)piperidine-1-carboxylate |
| 108 | 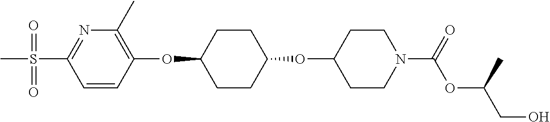 [SMILES: Cc1nc(S(C)(=O)=O)ccc1O[C@H]1CC[C@H](OC2CCN(C(=O)O[C@@H](C)CO)CC2)CC1] (S)-1-hydroxypropan-2-yl 4-((1r,4r)-4-(2-methyl-6- (methylsulfonyl)pyridin-3-yloxy)cyclohexyloxy)piperidine-1-carboxylate |
| 109 | 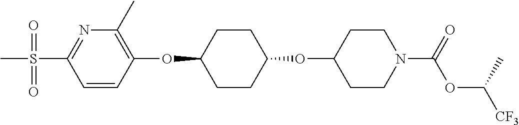 [SMILES: Cc1nc(S(C)(=O)=O)ccc1O[C@H]1CC[C@H](OC2CCN(C(=O)O[C@H](C)C(F)(F)F)CC2)CC1] (R)-1,1,1-trifluoropropan-2-yl 4-((1r,4r)-4-(2-methyl-6- (methylsulfonyl)pyridin-3-yloxy)cyclohexyloxy)piperidine-1-carboxylate |
| 110 | 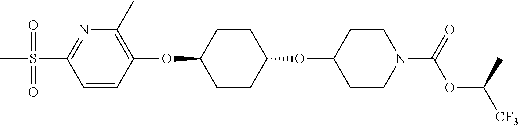 [SMILES: Cc1nc(S(C)(=O)=O)ccc1O[C@H]1CC[C@H](OC2CCN(C(=O)O[C@@H](C)C(F)(F)F)CC2)CC1] (S)-1,1,1-trifluoropropan-2-yl 4-((1r,4r)-4-(2-methyl-6- (methylsulfonyl)pyridin-3-yloxy)cyclohexyloxy)piperidine-1-carboxylate |
| 111 | 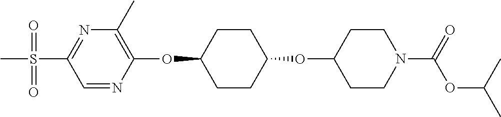 [SMILES: Cc1nc(S(C)(=O)=O)cnc1O[C@H]1CC[C@H](OC2CCN(C(=O)OC(C)C)CC2)CC1] isopropyl 4-((1r,4r)-4-(3-methyl-5-(methylsulfonyl)pyrazin-2- yloxy)cyclohexyloxy)piperidine-1-carboxylate |
| 112 | 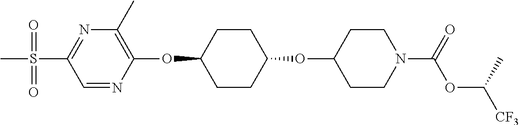 [SMILES: Cc1nc(S(C)(=O)=O)cnc1O[C@H]1CC[C@H](OC2CCN(C(=O)O[C@H](C)C(F)(F)F)CC2)CC1] (R)-1,1,1-trifluoropropan-2-yl 4-((1r,4r)-4-(3-methyl-5- (methylsulfonyl)pyrazin-2-yloxy)cyclohexyloxy)piperidine-1-carboxylate |
| 113 | 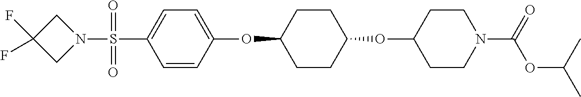 [SMILES: CC(C)OC(=O)N1CCC(O[C@H]2CC[C@H](Oc3ccc(S(=O)(=O)N4CC(F)(F)C4)cc3)CC2)CC1] isopropyl 4-((1r,4r)-4-(4-(3,3-difluoroazetidin-1- ylsulfonyl)phenoxy)cyclohexyloxy)piperidine-1-carboxylate |
| 114 | 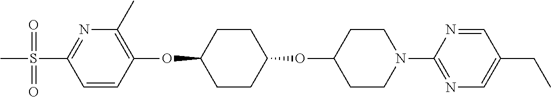 [SMILES: CCc1cnc(N2CCC(O[C@H]3CC[C@H](Oc4ccc(S(C)(=O)=O)nc4C)CC3)CC2)nc1] 5-ethyl-2-(4-((1r,4r)-4-(2-methyl-6-(methylsulfonyl)pyridin-3- yloxy)cyclohexyloxy)piperidin-1-yl)pyrimidine |
| 115 | 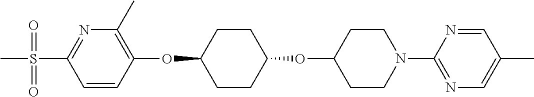 5-methyl-2-(4-((1r,4r)-4-(2-methyl-6-(methylsulfonyl)pyridin-3- yloxy)cyclohexyloxy)piperidin-1-yl)pyrimidine |
| 116 | 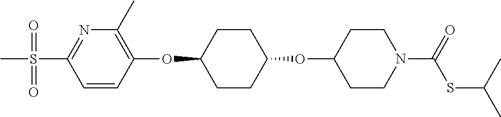 [SMILES: Cc1nc(S(C)(=O)=O)ccc1O[C@H]1CC[C@H](OC2CCN(C(=O)SC(C)C)CC2)CC1] S-isopropyl 4-((1r,4r)-4-(2-methyl-6-(methylsulfonyl)pyridin-3- yloxy)cyclohexyloxy)piperidine-1-carbothioate |
| 117 | 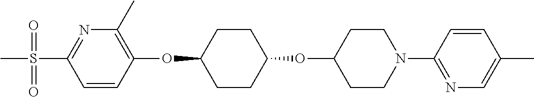 2-methyl-3-((1r,4r)-4-(1-(5-methylpyridin-2-yl)piperidin-4- yloxy)cyclohexyloxy)-6-(methylsulfonyl)pyridine |
| 118 | 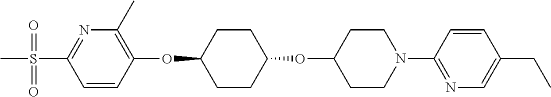 [SMILES: CCc1ccc(N2CCC(O[C@H]3CC[C@H](Oc4ccc(S(C)(=O)=O)nc4C)CC3)CC2)nc1] 3-((1r,4r)-4-(1-(5-ethylpyridin-2-yl)piperidin-4-yloxy)cyclohexyloxy)-2- methyl-6-(methylsulfonyl)pyridine |
| 119 | 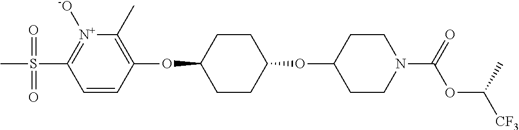 2-methyl-6-(methylsulfonyl)-3-((1r,4r)-4-(1-(((R)-1,1,1-trifluoropropan-2- yloxy)carbonyl)piperidin-4-yloxy)cyclohexyloxy)pyridine 1-oxide |
| 120 | 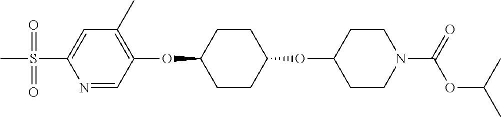 [SMILES: Cc1cc(S(C)(=O)=O)ncc1O[C@H]1CC[C@H](OC2CCN(C(=O)OC(C)C)CC2)CC1] isopropyl 4-((1r,4r)-4-(4-methyl-6-(methylsulfonyl)pyridin-3- yloxy)cyclohexyloxy)piperidine-1-carboxylate |
| 121 | 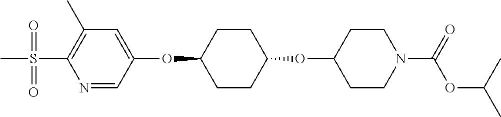 [SMILES: Cc1cc(O[C@H]2CC[C@H](OC3CCN(C(=O)OC(C)C)CC3)CC2)cnc1S(C)(=O)=O] isopropyl 4-((1r,4r)-4-(5-methyl-6-(methylsulfonyl)pyridin-3- yloxy)cyclohexyloxy)piperidine-1-carboxylate |
| 122 | 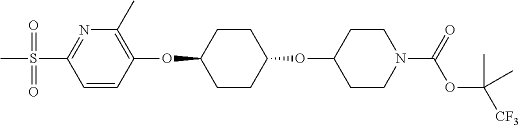 [SMILES: Cc1nc(S(C)(=O)=O)ccc1O[C@H]1CC[C@H](OC2CCN(C(=O)OC(C)(C)C(F)(F)F)CC2)CC1] 1,1,1-trifluoro-2-methylpropan-2-yl 4-((1r,4r)-(2-methyl-6- (methylsulfonyl)pyridin-3-yloxy)cyclohexyloxy)piperidine-1-carboxylate |
| 123 | 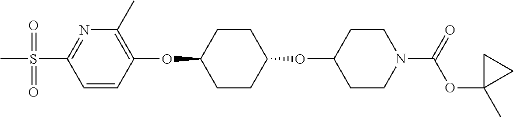 [SMILES: Cc1nc(S(C)(=O)=O)ccc1O[C@H]1CC[C@H](OC2CCN(C(=O)OC3(C)CC3)CC2)CC1] 1-methylcyclopropyl 4-((1r,4r)-4-(2-methyl-6-(methylsulfonyl)pyridin-3- yloxy)cyclohexyloxy)piperidine-1-carboxylate |
| 124 | 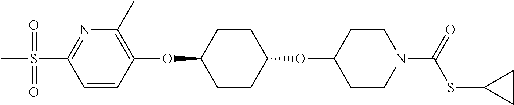 [SMILES: Cc1nc(S(C)(=O)=O)ccc1O[C@H]1CC[C@H](OC2CCN(C(=O)SC3CC3)CC2)CC1] S-cyclopropyl 4-((1r,4r)-4-(2-methyl-6-(methylsulfonyl)pyridin-3- yloxy)cyclohexyloxy)piperidine-1-carbothioate |
| 125 | 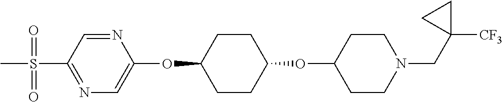 [SMILES: CS(=O)(=O)c1cnc(O[C@H]2CC[C@H](OC3CCN(CC4(C(F)(F)F)CC4)CC3)CC2)cn1] 2-(methylsulfonyl)-5-((1r,4r)-4-(1-((1- (trifluoromethyl)cyclopropyl)methyl)piperidin-4-yloxy)cyclohexyloxy)pyrazine |
| 126 | 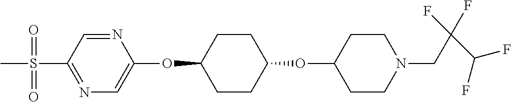 [SMILES: CS(=O)(=O)c1cnc(O[C@H]2CC[C@H](OC3CCN(CC(F)(F)C(F)F)CC3)CC2)cn1] 2-(methylsulfonyl)-5-((1r,4r)-4-(1-(2,2,3,3-tetrafluoropropyl)piperidin-4- yloxy)cyclohexyloxy)pyrazine |
| 127 | 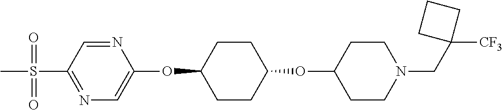 [SMILES: CS(=O)(=O)c1cnc(O[C@H]2CC[C@H](OC3CCN(CC4(C(F)(F)F)CCC4)CC3)CC2)cn1] 2-(methylsulfonyl)-5-((1r,4r)-4-(1-((1- (trifluoromethyl)cyclobutyl)methyl)piperidin-4- yloxy)cyclohexyloxy)pyrazine |
| 128 | 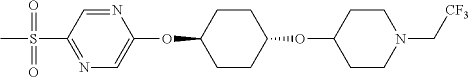 [SMILES: CS(=O)(=O)c1cnc(O[C@H]2CC[C@H](OC3CCN(CC(F)(F)F)CC3)CC2)cn1] 2-(methylsulfonyl)-5-((1r,4r)-4-(1-(2,2,2-trifluoroethyl)piperidin-4- yloxy)cyclohexyloxy)pyrazine |

Additionally, individual compounds and chemical genera of the present invention, for example those compounds found in Table A including, isomers, diastereoisomers and enantiomers thereof, encompass all pharmaceutically acceptable salts, solvates, and hydrates, thereof. Further, mesoisomers of individual compounds and chemical genera of the present invention, for example those compounds found in Table A, encompass all pharmaceutically acceptable salts, solvates and particularly hydrates, thereof.

The compounds of the Formula (Ia) of the present invention may be prepared according to relevant published literature procedures that are used by one skilled in the art. Exemplary reagents and procedures for these reactions appear hereinafter in the working Examples. Protection and deprotection may be carried out by procedures generally known in the art (see, for example, Greene, T. W. and Wuts, P. G. M., Protecting Groups in Organic Synthesis, 3^{rd} Edition, 1999 [Wiley]).

It is understood that the present invention embraces, each isomer, each diastereoisomer, each enantiomer and mixtures thereof of each compound and generic formulae disclosed herein just as if they were each individually disclosed with the specific stereochemical designation for each chiral carbon. Separation of the individual isomers and enantiomers (such as, by chiral HPLC, recrystallization of diastereoisomeric mixtures and the like) or selective synthesis (such as, by enantiomeric selective syntheses and the like) of the individual isomers can be accomplished by application of various methods which are well known to practitioners in the art.

### Certain Embodiments

### Compositions, Methods, Indications, Pharmaceutical Products, Combinations, and Uses of Compounds of the Present Invention

In addition to the foregoing, without limitation, certain other embodiments are described and provided below.

Certain Compositions of the Present Invention:

One aspect of the present invention pertains to compositions comprising a compound of the present invention.

One aspect of the present invention pertains to compositions comprising a compound of the present invention and a pharmaceutically acceptable carrier.

One aspect of the present invention pertains to methods for preparing a composition comprising the step of admixing a compound of the present invention and a pharmaceutically acceptable carrier.

One aspect of the present invention pertains to pharmaceutical products selected from: a pharmaceutical composition, a formulation, a dosage form, a combined preparation, a twin pack, and a kit; comprising a compound of the present invention.

One aspect of the present invention pertains to compositions comprising a compound of the present invention and a second pharmaceutical agent.

In any of the embodiments that recites the terms “a pharmaceutical agent” and “a second pharmaceutical agent”, it is appreciated that these terms in some aspects be further limited to a pharmaceutical agent that is not a compound of Formula (Ia). It is understood that the terms “a pharmaceutical agent” and “a second pharmaceutical agent” may refer to a pharmaceutical agent that is not detectable or has an EC₅₀ that is greater than a value selected from: 50 μM, 10 μM, 1 μM, and 0.1 μM in a GPR119 receptor activity assay as described in Example 4.

One aspect of the present invention pertains to methods for preparing a composition comprising the step of admixing a compound of the present invention and a second pharmaceutical agent.

One aspect of the present invention pertains to compositions comprising a compound of the present invention, a second pharmaceutical agent, and a pharmaceutically acceptable carrier.

One aspect of the present invention pertains to methods for preparing a composition comprising the step of admixing a compound of the present invention, a second pharmaceutical agent, and a pharmaceutically acceptable carrier.

One aspect of the present invention pertains to a pharmaceutical product selected from: a pharmaceutical composition, a formulation, a dosage form, a combined preparation, a twin pack, and a kit; comprising a compound of the present invention and a second pharmaceutical agent.

One aspect of the present invention pertains to compositions obtained by the methods of the present invention as described herein.

Certain Methods, Pharmaceutical Products, Combinations, and Uses of the Present Invention:

One aspect of the present invention pertains to methods for modulating the activity of a GPR119 receptor, comprising administering to an individual in need thereof, a therapeutically effective amount of: a compound of the present invention; a composition of the present invention; or a pharmaceutical product of the present invention.

One aspect of the present invention pertains to the use of a compound of the present invention; a composition of the present invention; or a pharmaceutical product of the present invention; in the manufacture of a medicament for modulating the activity of a GPR119 receptor in an individual.

One aspect of the present invention pertains to a compound of the present invention; a composition of the present invention; or a pharmaceutical product of the present invention; for use in a method of treating the human or animal by therapy.

One aspect of the present invention pertains to a compound of the present invention; a composition of the present invention; or a pharmaceutical product of the present invention; for use in a method of modulating the activity of a GPR119 receptor in an individual.

One aspect of the present invention pertains to a pharmaceutical product selected from: a pharmaceutical composition, a formulation, a dosage form, a combined preparation, a twin pack, and a kit; comprising a compound of the present invention; for use in a method of treating the human or animal by therapy.

One aspect of the present invention pertains to a pharmaceutical product selected from: a pharmaceutical composition, a formulation, a dosage form, a combined preparation, a twin pack, and a kit; comprising a compound of the present invention; for modulating the activity of a GPR119 receptor in an individual.

One aspect of the present invention pertains to methods for modulating the activity of a GPR119 receptor, comprising administering to an individual in need thereof, a therapeutically effective amount of a compound of the present invention in combination with a therapeutically effective amount of a second pharmaceutical agent.

One aspect of the present invention pertains to methods for agonizing a GPR119 receptor, comprising administering to an individual in need thereof, a therapeutically effective amount of a compound of the present invention in combination with a therapeutically effective amount of a second pharmaceutical agent.

One aspect of the present invention pertains to methods for the treatment of a disorder selected from: a GPR119-receptor-related disorder; a condition ameliorated by increasing a blood incretin level, a condition characterized by low bone mass; a neurological disorder; a metabolic-related disorder; and obesity; in an individual; comprising administering to said individual in need thereof, a therapeutically effective amount of a compound of the present invention in combination with a therapeutically effective amount of a second pharmaceutical agent.

One aspect of the present invention pertains to the use of a compound of the present invention in combination with a second pharmaceutical agent in the manufacture of a medicament for modulating the activity of a GPR119 receptor in an individual.

One aspect of the present invention pertains to the use of a compound of the present invention in combination with a second pharmaceutical agent in the manufacture of a medicament for agonizing a GPR119 receptor in an individual.

One aspect of the present invention pertains to the use of a compound of the present invention in combination with a second pharmaceutical agent, in the manufacture of a medicament for the treatment of a disorder selected from: a GPR119-receptor-related disorder; a condition ameliorated by increasing a blood incretin level, a condition characterized by low bone mass; a neurological disorder; a metabolic-related disorder; and obesity.

One aspect of the present invention pertains to a compound of the present invention for use in combination with a second pharmaceutical agent for use in a method of treating the human or animal by therapy.

One aspect of the present invention pertains to a compound of the present invention for use in combination with a second pharmaceutical agent for modulating the activity of a GPR119 receptor in an individual.

One aspect of the present invention pertains to a compound of the present invention for use in combination with a second pharmaceutical agent for agonizing a GPR119 receptor in an individual.

One aspect of the present invention pertains to a compound of the present invention for use in combination with a second pharmaceutical agent for the treatment of a disorder selected from: a GPR119-receptor-related disorder; a condition ameliorated by increasing a blood incretin level, a condition characterized by low bone mass; a neurological disorder; a metabolic-related disorder; and obesity; in an individual.

In some embodiments, the second pharmaceutical agent is selected from: an inhibitor of DPP-IV, a biguanide, an alpha-glucosidase inhibitor, a sulfonylurea, a SGLT2 inhibitor, and a meglitinide. In some embodiments, the second pharmaceutical agent is selected from: sitagliptin, vildagliptin, saxagliptin, alogliptin, linagliptin, phenformin, metformin, buformin, acarbose, miglitol, voglibose, tolbutamide, acetohexamide, tolazamide, chlorpropamide, glipizide, glibenclamide, glimepiride, gliclazide, dapagliflozin, remigliflozin, and sergliflozin.

In some embodiments, the disorder is type 2 diabetes. In some embodiments, the disorder is hyperglycemia. In some embodiments, the disorder is hyperlipidemia. In some embodiments, the disorder is hypertriglyceridemia. In some embodiments, the disorder is type 1 diabetes. In some embodiments, the disorder is dyslipidemia. In some embodiments, the disorder is syndrome X. In some embodiments, the disorder is obesity.

One aspect of the present invention pertains to the use of a pharmaceutical agent in combination with a compound of the present invention, in the manufacture of a medicament for modulating the activity of a GPR119 receptor in an individual.

One aspect of the present invention pertains to the use of a pharmaceutical agent in combination with a compound of the present invention, in the manufacture of a medicament for increasing the secretion of an incretin level in an individual.

One aspect of the present invention pertains to the use of a pharmaceutical agent in combination with a compound of the present invention, in the manufacture of a medicament for increasing a blood incretin level in an individual.

One aspect of the present invention pertains to the use of a pharmaceutical agent in combination with a compound of the present invention, in the manufacture of a medicament for the treatment of a disorder selected from: a GPR119-receptor-related disorder; a condition ameliorated by increasing a blood incretin level, a condition characterized by low bone mass; a neurological disorder; a metabolic-related disorder; and obesity.

One aspect of the present invention pertains to a pharmaceutical agent for use in combination with a compound of the present invention, for use in a method of treating the human or animal by therapy.

One aspect of the present invention pertains to a pharmaceutical agent for use in combination with a compound of the present invention, for use in combination with a pharmaceutical agent for modulating the activity of a GPR119 receptor in an individual.

One aspect of the present invention pertains to a pharmaceutical agent for use in combination with a compound of the present invention, for increasing the secretion of an incretin level in an individual.

One aspect of the present invention pertains to a pharmaceutical agent for use in combination with a compound of the present invention, for use in a method for increasing a blood incretin level in an individual.

One aspect of the present invention pertains to a pharmaceutical agent for use in combination with a compound of the present invention, for the treatment of a disorder selected from: a GPR119-receptor-related disorder; a condition ameliorated by increasing a blood incretin level, a condition characterized by low bone mass; a neurological disorder; a metabolic-related disorder; and obesity; in an individual.

In some embodiments, the pharmaceutical agent is selected from: an inhibitor of DPP-IV, a biguanide, an alpha-glucosidase inhibitor, a sulfonylurea, a SGLT2 inhibitor, and a meglitinide. In some embodiments, the pharmaceutical agent is selected from: sitagliptin, vildagliptin, saxagliptin, alogliptin, linagliptin, phenformin, metformin, buformin, acarbose, miglitol, voglibose, tolbutamide, acetohexamide, tolazamide, chlorpropamide, glipizide, glibenclamide, glimepiride, gliclazide, dapagliflozin, remigliflozin, and sergliflozin.

In some embodiments, the disorder is type 2 diabetes. In some embodiments, the disorder is hyperglycemia. In some embodiments, the disorder is hyperlipidemia. In some embodiments, the disorder is hypertriglyceridemia. In some embodiments, the disorder is type 1 diabetes. In some embodiments, the disorder is dyslipidemia. In some embodiments, the disorder is syndrome X. In some embodiments, the disorder is obesity.

One aspect of the present invention pertains to a pharmaceutical product selected from: a pharmaceutical composition, a formulation, a dosage form, a combined preparation, a twin pack, and a kit; comprising a compound of the present invention and a second pharmaceutical agent; for use in a method of treating the human or animal by therapy.

One aspect of the present invention pertains to a pharmaceutical product selected from: a pharmaceutical composition, a formulation, a dosage form, a combined preparation, a twin pack, and a kit; comprising a compound of the present invention and a second pharmaceutical agent; for modulating the activity of a GPR119 receptor in an individual.

One aspect of the present invention pertains to methods for modulating the activity of a GPR119 receptor, comprising administering to an individual in need thereof, a therapeutically effective amount of a compound of the present invention and an inhibitor of DPP-IV.

One aspect of the present invention pertains to compounds of the present invention for use in combination with an inhibitor of DPP-IV for modulating the activity of a GPR119 receptor in an individual.

One aspect of the present invention pertains to inhibitors of DPP-IV in combination with a compound of the present invention, for use in modulating the activity of a GPR119 receptor.

One aspect of the present invention pertains to a pharmaceutical product selected from: a pharmaceutical composition, a formulation, a dosage form, a combined preparation, a twin pack, and a kit; comprising a compound of the present invention and an inhibitor of DPP-IV; for modulating the activity of a GPR119 receptor in an individual.

One aspect of the present invention pertains to the use of a compound of the present invention and an inhibitor of DPP-IV in the manufacture of a medicament for modulating the activity of a GPR119 receptor in an individual.

One aspect of the present invention pertains to compounds, methods, compositions, uses of compounds, pharmaceutical agents, pharmaceutical products, and inhibitors of DPP-IV, as described herein, wherein modulating the activity of a GPR119 receptor is agonizing the GPR119 receptor in an individual.

One aspect of the present invention pertains to compounds, methods, compositions, uses of compounds, pharmaceutical agents, pharmaceutical products, and inhibitors of DPP-IV, as described herein, wherein modulating the activity of a GPR119 receptor is increasing the secretion of an incretin in an individual.

One aspect of the present invention pertains to compounds, methods, compositions, uses of compounds, pharmaceutical agents, pharmaceutical products, and inhibitors of DPP-IV, as described herein, wherein modulating the activity of a GPR119 receptor is increasing a blood incretin level in an individual.

One aspect of the present invention pertains to compounds, methods, compositions, uses of compounds, pharmaceutical agents, pharmaceutical products, and inhibitors of DPP-IV, as described herein, wherein modulating the activity of a GPR119 receptor treating a disorder, wherein the disorder is selected from: a GPR119-receptor-related disorder; a condition ameliorated by increasing a blood incretin level; a condition characterized by low bone mass; a neurological disorder; a metabolic-related disorder; and obesity.

In some embodiments, the pharmaceutical product comprises a pharmaceutical composition. In some embodiments, the pharmaceutical product comprises a formulation. In some embodiments, the pharmaceutical product comprises a dosage form. In some embodiments, the pharmaceutical product comprises a combined preparation. In some embodiments, the pharmaceutical product comprises a twin pack. In some embodiments, the pharmaceutical product comprises a kit.

In some embodiments, the compound and the pharmaceutical agent or second pharmaceutical agent are administered simultaneously. In some embodiments, the compound and the pharmaceutical agent or second pharmaceutical agent are administered separately. In some embodiments, the compound and the pharmaceutical agent or second pharmaceutical agent are administered sequentially.

In some embodiments, the incretin is GLP-1. In some embodiments, the incretin is GIP. In some embodiments, the incretin is PYY.

In some embodiments, the compound and the pharmaceutical agent or second pharmaceutical agent are provided in amounts which give a synergistic effect in treating the disorder.

In some embodiments, the amount of the compound alone is substantially therapeutically ineffective at treating the disorder.

In some embodiments, the amount of the pharmaceutical agent alone is substantially therapeutically ineffective at treating the disorder.

One aspect of the present invention pertains to methods for preparing a pharmaceutical product, as described herein, comprising: mixing the compound of the present invention with a first pharmaceutically acceptable carrier to prepare a compound dosage form, mixing the second pharmaceutical agent with a second pharmaceutically acceptable carrier to prepare a second pharmaceutical agent dosage form, and providing the compound dosage form and the second pharmaceutical agent dosage form in a combined dosage form for simultaneous, separate, or sequential use.

In some embodiments, the first pharmaceutically acceptable carrier and the second pharmaceutically acceptable carrier are different. In some embodiments, the different pharmaceutically acceptable carriers are suitable for administration by the same route or different routes. In some embodiments, the first pharmaceutically acceptable carrier and the second pharmaceutically acceptable carrier are substantially the same. In some embodiments, the substantially the same pharmaceutically acceptable carriers are suitable for administration by the same route. In some embodiments, the substantially the same pharmaceutically acceptable carriers are suitable for oral administration.

In some embodiments, the pharmaceutical agent or the second pharmaceutical agent is selected from: an inhibitor of DPP-IV, a biguanide, an alpha-glucosidase inhibitor, an insulin analogue, a sulfonylurea, a SGLT2 inhibitor, a meglitinide, a thiazolidinedione, and an anti-diabetic peptide analogue. In some embodiments, the pharmaceutical agent or the second pharmaceutical agent is selected from: an inhibitor of DPP-IV, a biguanide, an alpha-glucosidase inhibitor, a sulfonylurea, a SGLT2 inhibitor, and a meglitinide. In some embodiments, the pharmaceutical agent or the second pharmaceutical agent is selected from: an inhibitor of DPP-IV, a biguanide, and an alpha-glucosidase inhibitor. In some embodiments, the pharmaceutical agent or the second pharmaceutical agent is an inhibitor of DPP-IV. In some embodiments, the pharmaceutical agent or the second pharmaceutical agent is a biguanide. In some embodiments, the pharmaceutical agent or the second pharmaceutical agent is an alpha-glucosidase inhibitor. In some embodiments, the pharmaceutical agent or the second pharmaceutical agent is a sulfonylurea. In some embodiments, the pharmaceutical agent or the second pharmaceutical agent is a SGLT2 inhibitor. In some embodiments, the pharmaceutical agent or the second pharmaceutical agent is a meglitinide. In some embodiments, the pharmaceutical agent or the second pharmaceutical agent is a biguanide selected from the following compounds and pharmaceutically acceptable salts, solvates, and hydrates thereof: metformin, phenformin, buformin, and proguanil. In some embodiments, the pharmaceutical agent or the second pharmaceutical agent is an alpha-glucosidase inhibitor selected from the following compounds and pharmaceutically acceptable salts, solvates, and hydrates thereof: acarbose, miglitol, and voglibose.

In some embodiments, the pharmaceutical agent or the second pharmaceutical agent is a sulfonylurea selected from the following compounds and pharmaceutically acceptable salts, solvates, and hydrates thereof: here here

In some embodiments, the pharmaceutical agent or the second pharmaceutical agent is a SGLT2 inhibitor selected from the following compounds and pharmaceutically acceptable salts, solvates, and hydrates thereof:

In some embodiments, the pharmaceutical agent or the second pharmaceutical agent is a meglitinide selected from the following compounds and pharmaceutically acceptable salts, solvates, and hydrates thereof:

One aspect of the present invention pertains to methods for weight management, comprising administering to an individual in need thereof, a therapeutically effective amount of a compound of the present invention in combination with a therapeutically effective amount of a pharmaceutical agent, such as any agent described herein; wherein the compound and the pharmaceutical agent.

In some embodiments, the weight management comprises weight loss. In some embodiments, the weight management comprises maintenance of weight loss. In some embodiments, the weight management further comprises a reduced-calorie diet. In some embodiments, the weight management further comprises a program of regular exercise. In some embodiments, the weight management further comprises both a reduced-calorie diet and a program of regular exercise.

In some embodiments, the individual in need of weight management is a patient with an initial body mass of index ≧40 kg/m²; ≧39 kg/m²; ≧38 kg/m²; ≧37 kg/m²; ≧36 kg/m²; ≧35 kg/m²; ≧34 kg/m²; ≧33 kg/m²; ≧32 kg/m²; ≧31 kg/m²; ≧30 kg/m²; ≧29 kg/m²; ≧28 kg/m²; ≧27 kg/m²; ≧26 kg/m²; ≧25 kg/m²; ≧24 kg/m²; ≧23 kg/m²; ≧22 kg/m²; ≧21 kg/m²; or ≧20 kg/m²; and the patient optionally has at least one or at least two weight related comorbid condition(s).

In some embodiments, the comorbid condition(s) when present are selected from: hypertension, dyslipidemia, cardiovascular disease, glucose intolerance, and sleep apnea.

Certain Indications of the Present Invention:

In the context of the present invention, a compound as described herein or a pharmaceutical composition thereof can be utilized for modulating the activity of GPR119-receptor-related diseases, conditions and/or disorders as described herein.

In some embodiments, modulating the activity includes the treatment of a GPR119-receptor-related disorder. In some embodiments, a GPR119-receptor-related disorder is a condition ameliorated by increasing a blood incretin level. In some embodiments, a GPR119-receptor-related disorder is a condition characterized by low bone mass. In some embodiments, a GPR119-receptor-related disorder is a neurological disorder. In some embodiments, a GPR119-receptor-related disorder is a metabolic-related disorder. In some embodiments, a GPR119-receptor-related disorder is obesity

Some embodiments of the present invention include every combination of one or more conditions characterized by low bone mass selected from: osteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine, and loss of height.

In some embodiments, the neurological disorder selected from: stroke and Parkinsonism.

Some embodiments of the present invention include every combination of one or more metabolic-related disorders selected from: type 1 diabetes, type 2 diabetes mellitus, and conditions associated therewith, such as, but not limited to, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertriglyceridemia, insulin resistance, impaired glucose metabolism, erectile dysfunction, skin and connective tissue disorders, foot ulcerations and ulcerative colitis, endothelial dysfunction and impaired vascular compliance.

Some embodiments of the present invention include every combination of one or more metabolic-related disorders selected from: diabetes, type 1 diabetes, type 2 diabetes, inadequate glucose tolerance, impaired glucose tolerance, insulin resistance, hyperglycemia, hyperlipidemia, hypertriglyceridemia, hypercholesterolemia, dyslipidemia, atherosclerosis, stroke, syndrome X, hypertension, pancreatic beta-cell insufficiency, enteroendocrine cell insufficiency, glucosuria, metabolic acidosis, cataracts, diabetic nephropathy, diabetic neuropathy, peripheral neuropathy, diabetic coronary artery disease, diabetic cerebrovascular disease, diabetic peripheral vascular disease, diabetic retinopathy, metabolic syndrome, a condition related to diabetes, myocardial infarction, learning impairment, memory impairment, a neurodegenerative disorder, a condition ameliorated by increasing a blood GLP-1 level in an individual with a neurodegenerative disorder, excitotoxic brain damage caused by severe epileptic seizures, Alzheimer's disease, Parkinson's disease, Huntington's disease, prion-associated disease, stroke, motor-neuron disease, traumatic brain injury, spinal cord injury, and obesity.

In some embodiments, the disorder is type 2 diabetes. In some embodiments, the disorder is hyperglycemia. In some embodiments, the disorder is hyperlipidemia. In some embodiments, the disorder is hypertriglyceridemia. In some embodiments, the disorder is type 1 diabetes. In some embodiments, the disorder is dyslipidemia. In some embodiments, the disorder is syndrome X. In some embodiments, the disorder is obesity.

### Formulations and Compositions

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a solvate, hydrate or physiologically functional derivative thereof can be used as active ingredients in pharmaceutical compositions, specifically as GPR119 receptor modulators. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds of the present invention can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis conducted or on whether further active compounds are administered in addition to the compounds of the present invention. Representative doses of the present invention include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt, solvate, or hydrate of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size. The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” refers to the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well known to the person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain compounds of the present invention which contain a carboxylic acid functional group may optionally exist as pharmaceutically acceptable salts containing non-toxic, pharmaceutically acceptable metal cations and cations derived from organic bases. Representative metals include, but are not limited to, aluminium, calcium, lithium, magnesium, potassium, sodium, zinc and the like. In some embodiments the pharmaceutically acceptable metal is sodium. Representative organic bases include, but are not limited to, benzathine (N¹,N²-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et al., Journal of Pharmaceutical Sciences, 66:1-19 (1977).

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

It is noted that when the GPR119 receptor modulators are utilized as active ingredients in pharmaceutical compositions, these are not intended for use in humans only, but in non-human mammals as well. Recent advances in the area of animal health-care mandate that consideration be given for the use of active agents, such as GPR119 receptor modulators, for the treatment of a GPR119 receptor-associated disease or disorder in companionship animals (e.g., cats, dogs, etc.) and in livestock animals (e.g., horses, cows, etc.) Those of ordinary skill in the art are readily credited with understanding the utility of such compounds in such settings.

### Hydrates and Solvates

It is understood that when the phrase “pharmaceutically acceptable salts, solvates, and hydrates” or the phrase “pharmaceutically acceptable salt, solvate, or hydrate” is used when referring to compounds described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used when referring to salts described herein, it embraces pharmaceutically acceptable solvates and/or hydrates of such salts.

It will be apparent to those skilled in the art that the dosage forms described herein may comprise, as the active component, either a compound described herein or a pharmaceutically acceptable salt or as a pharmaceutically acceptable solvate or hydrate thereof. Moreover, various hydrates and solvates of the compounds described herein and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present invention pertains to methods of administering hydrates and solvates of compounds described herein and/or their pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

### Combination Therapy

A compound of the invention can be administered as the sole active pharmaceutical agent (i.e., mono-therapy), or it can be used in combination with one or more pharmaceutical agents (i.e., combination-therapy), such as pharmaceutical agents, such as, known anti-diabetic agents, either administered together or separately for the treatment of the diseases, conditions, and disorders described herein. Therefore, another aspect of the present invention includes methods of treatment of a metabolic related disorder, including a weight-related disorder, such as obesity, comprising administering to an individual in need thereof a therapeutically effective amount of a compound of Formula (Ia) and pharmaceutically acceptable salts, solvates and hydrates thereof, in combination with one or more pharmaceutical agents, such as anti-diabetic agents, as described herein.

In accordance with the present invention, the combination can be used by mixing the respective active components, a compound of Formula (Ia) and a pharmaceutical agent, either together or independently optionally with a physiologically acceptable carrier, excipient, binder, diluent, etc., as described herein, and administering the mixture or mixtures either orally or non-orally as a pharmaceutical composition(s). When a compound of Formula (Ia) is administered as a combination therapy with another active compound the compound of Formula (Ia) and the pharmaceutical agent can be formulated as separate pharmaceutical compositions given at the same time or at different times; or the compound of Formula (Ia) and the pharmaceutical agent can be formulated together as a single unit dosage.

Suitable pharmaceutical agents that can be used in combination with the compounds of the present invention include anti-obesity agents such as apolipoprotein-B secretion/microsomal triglyceride transfer protein (apo-B/MTP) inhibitors; MCR-4 agonists, cholescystokinin-A (CCK-A) agonists; serotonin and norepinephrine reuptake inhibitors (for example, sibutramine); sympathomimetic agents; β3 adrenergic receptor agonists; dopamine agonists (for example, bromocriptine); melanocyte-stimulating hormone receptor analogues; cannabinoid 1 receptor antagonists [for example, SR141716: N-(piperidin-1-yl)-5-(4-chlorophenyl)-1-(2,4-dichlorophenyl)-4-methyl-1H-pyrazole-3-carboxamide]; melanin concentrating hormone antagonists; leptin (the OB protein); leptin analogues; leptin receptor agonists; galanin antagonists; lipase inhibitors (such as tetrahydrolipstatin, i.e., Orlistat); anorectic agents (such as a bombesin agonist); neuropeptide-Y antagonists; thyromimetic agents; dehydroepiandrosterone or an analogue thereof; glucocorticoid receptor agonists or antagonists; orexin receptor antagonists; urocortin binding protein antagonists; glucagon-like peptide-1 (GLP-1) receptor agonists; ciliary neutrotrophic factors (such as Axokine™ available from Regeneron Pharmaceuticals, Inc., Tarrytown, N.Y. and Procter & Gamble Company, Cincinnati, Ohio); human agouti-related proteins (AGRP); ghrelin receptor antagonists; histamine 3 receptor (H3R) antagonists or inverse agonists; neuromedin U receptor agonists; noradrenergic anorectic agents (for example, phentermine, mazindol and the like); and appetite suppressants (for example, bupropion).

Other anti-obesity agents, including the agents set forth infra, are well known, or will be readily apparent in light of the instant disclosure, to one of ordinary skill in the art. In some embodiments, the anti-obesity agents are selected from the group consisting of orlistat, sibutramine, bromocriptine, ephedrine, leptin, and pseudoephedrine. In a further embodiment, compounds of the present invention and combination therapies are administered in conjunction with exercise and/or a calorie-controlled diet.

It is understood that the scope of combination-therapy of the compounds of the present invention with anti-obesity agents, anorectic agents, appetite suppressant and related agents is not limited to those listed above, but includes in principle any combination with any pharmaceutical agent or pharmaceutical composition useful for the treatment of overweight and obese individuals.

It is understood that the scope of combination-therapy of the compounds of the present invention with other pharmaceutical agents is not limited to those listed herein, supra or infra, but includes in principle any combination with any pharmaceutical agent or pharmaceutical composition useful for the treatment of diseases, conditions or disorders that are linked to metabolic related disorders.

Some embodiments of the present invention include methods of treatment of a disease, disorder, condition or complication thereof as described herein, comprising administering to an individual in need of such treatment a therapeutically effective amount or dose of a compound of Formula (Ia) in combination with at least one pharmaceutical agent selected from the group consisting of: sulfonylureas (for example, tolbutamide (Orinase); acetohexamide (Dymelor); tolazamide (Tolinase); chlorpropamide (Diabinese); glipizide (Glucotrol); glyburide (Diabeta, Micronase, Glynase); glimepiride (Amaryl); gliclazide (Diamicron); and sulfonylureas known in the art); meglitinides (for example, repaglinide (Prandin), nateglinide (Starlix), mitiglinide, and other meglitinides known in the art); biguanides (for example, phenformin, metformin, buformin, and biguanides known in the art); α-glucosidase inhibitors (for example, acarbose, miglitol, and α-glucosidase inhibitors known in the art); thiazolidinediones-peroxisome proliferators-activated receptor-γ (i.e., PPAR-γ) agonists (for example, rosiglitazone (Avandia), pioglitazone (Actos), troglitazone (Rezulin), rivoglitazone, ciglitazone, and thiazolidinediones known in the art); insulin and insulin analogues; anti-diabetic peptide analogues (for example, exenatide, liraglutide, taspoglutide, and anti-diabetic peptides analogues know in the art); HMG-CoA reductase inhibitors (for example, rosuvastatin, pravastatin and its sodium salt, simvastatin, lovastatin, atorvastatin, fluvastatin, cerivastatin, rosuvastatin, pitavastatin, pravastatin, and other HMG-CoA reductase inhibitors known in the art); cholesterol-lowering drugs (for example, fibrates that include: bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, theofibrate, and other fibrates known in the art; bile acid sequestrants which include: cholestyramine, colestipol and the like; and niacin); antiplatelet agents (for example, aspirin and adenosine diphosphate receptor antagonists that include: clopidogrel, ticlopidine and the like); angiotensin-converting enzyme inhibitors (for example, captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and other angiotensin converting enzyme inhibitors known in the art); angiotensin II receptor antagonists [for example, losartan (and the potassium salt form), and other angiotensin II receptor antagonists known in the art; adiponectin; squalene synthesis inhibitors {for example, (S)-α-[bis[2,2-dimethyl-1-oxopropoxy)methoxy]phosphinyl]-3-phenoxybenzenebutanesulfonic acid, mono potassium salt (BMS-188494) and other squalene synthesis inhibitors known in the art}; and the like. In some embodiments, compounds of the present invention and the pharmaceutical agents are administered separately. In further embodiments, compounds of the present invention and the pharmaceutical agents are administered simultaneously.

Suitable pharmaceutical agents that can be used in conjunction with compounds of the present invention include, but are not limited to: amylin agonists (for example, pramlintide); insulin secretagogues (for example, GLP-1 agonists, exendin-4, and insulinotropin (NN2211)); acyl CoA cholesterol acetyltransferase inhibitors (for example, ezetimibe, eflucimibe, and other acyl CoA cholesterol acetyltransferase inhibitors known in the art); cholesterol absorption inhibitors (for example, ezetimibe, pamaqueside and other cholesterol absorption inhibitors known in the art); cholesterol ester transfer protein inhibitors (for example, CP-529414, JTT-705, CETi-1, and other cholesterol ester transfer protein inhibitors known in the art); microsomal triglyceride transfer protein inhibitors (for example, implitapide, and other microsomal triglyceride transfer protein inhibitors known in the art); cholesterol modulators (for example, NO-1886, and other cholesterol modulators known in the art); bile acid modulators (for example, GT103-279 and other bile acid modulators known in the art); insulin signaling pathway modulators; inhibitors of protein tyrosine phosphatases (PTPases); non-small molecule mimetics and inhibitors of glutamine-fructose-6-phosphate amidotransferase (GFAT); compounds influencing a dysregulated hepatic glucose production; inhibitors of glucose-6-phosphatase (G6Pase); inhibitors of fructose-1,6-bisphosphatase (F-1,6-BPase); inhibitors of glycogen phosphorylase (GP); glucagon receptor antagonists; inhibitors of phosphoenolpyruvate carboxykinase (PEPCK); pyruvate dehydrogenase kinase (PDHK) inhibitors; insulin sensitivity enhancers; insulin secretion enhancers; inhibitors of gastric emptying; α₂-adrenergic antagonists; retinoid X receptor (RXR) agonists; and dipeptidyl peptidase-4 (DPP-IV) inhibitors; and the like.

### Tripartite Combinations

Some aspects of the present invention include compounds of Formula (Ia) that can be employed in any of the methods, pharmaceutical products, uses, compounds, and pharmaceutical agents, as described herein, in combination with two distinct pharmaceutical agents.

In some embodiments, the two distinct pharmaceutical agents are selected from any of the pharmaceutical agents, or classes of pharmaceutical agents described herein. In some embodiments, the two distinct pharmaceutical agents are selected from: an inhibitor of DPP-IV, a biguanide, an alpha-glucosidase inhibitor, an insulin analogue, a sulfonylurea, a SGLT2 inhibitor, a meglitinide, a thiazolidinedione, and an anti-diabetic peptide analogue. In some embodiments, the two distinct pharmaceutical agents include every combination selected from pharmaceutical agents of the following group: an inhibitor of DPP-IV, a biguanide, an alpha-glucosidase inhibitor, a sulfonylurea, and a SGLT2 inhibitor.

Some embodiments of the present invention include every combination of one or more compounds selected from compounds of the following group and pharmaceutically acceptable salts, solvates, and hydrates thereof: an inhibitor of DPP-IV selected from: 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one; 1-[2-(3-hydroxyadamant-1-ylamino)acetyl]pyrrolidine-2(S)-carbonitrile; (1S,3S,5S)-2-[2(S)-amino-2-(3-hydroxyadamantan-1-yl)acetyl]-2-azabicyclo[3.1.0]hexane-3-carbonitrile; 2-[6-[3(R)-aminopiperidin-1-yl]-3-methyl-2,4-dioxo-1,2,3,4-tetrahydropyrimidin-1-ylmethyl]benzonitrile; 8-[3(R)-aminopiperidin-1-yl]-7-(2-butynyl)-3-methyl-1-(4-methylquinazolin-2-ylmethyl)xanthine; 1-[N-[3(R)-pyrrolidinyl]glycyl]pyrrolidin-2(R)-yl boronic acid; 4(S)-fluoro-1-[2-[(1R,3S)-3-(1H-1,2,4-triazol-1-ylmethyl)cyclopentylamino]acetyl]pyrrolidine-2(S)-carbonitrile; 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one; (2S,4S)-2-cyano-4-fluoro-1-[(2-hydroxy-1,1-dimethyl)ethylamino]acetylpyrrolidine; 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione; 1-((3S,4S)-4-amino-1-(4-(3,3-difluoropyrrolidin-1-yl)-1,3,5-triazin-2-yl)pyrrolidin-3-yl)-5,5-difluoropiperidin-2-one; (R)-2-((6-(3-aminopiperidin-1-yl)-3-methyl-2,4-dioxo-3,4-dihydropyrimidin-1(2H)-yl)methyl)-4-fluorobenzonitrile; 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide; ((2S,4S)-4-(4-(3-methyl-1-phenyl-1H-pyrazol-5-yl)piperazin-1-yl)pyrrolidin-2-yl)(thiazolidin-3-yl)methanone; (2S,4S)-1-[2-[(4-ethoxycarbonylbicyclo[2.2.2]oct-1-yl)amino]acetyl]-4-fluoropyrrolidine-2-carbonitrile; 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione; 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile; (2S)-1-{[2-(5-methyl-2-phenyl-oxazol-4-yl)-ethylamino]-acetyl}-pyrrolidine-2-carbonitrile; (2S)-1-{[1,1-dimethyl-3-(4-pyridin-3-yl-imidazol-1-yl)-propylamino]-acetyl}-pyrrolidine-2-carbonitrile; (3,3-difluoropyrrolidin-1-yl)-((2S,4S)-4-(4-(pyrimidin-2-yl)piperazin-1-yl)pyrrolidin-2-yl)methanone; (2S,4S)-1-[(2S)-2-amino-3,3-bis(4-fluorophenyl)propanoyl]-4-fluoropyrrolidine-2-carbonitrile; (2S,5R)-5-ethynyl-1-{N-(4-methyl-1-(4-carboxy-pyridin-2-yl)piperidin-4-yl)glycyl}pyrrolidine-2-carbonitrile; and (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine; a biguanide selected from: phenformin ((phenylethyl)biguanide); metformin (dimethylbiguanide); buformin (butylbiguanide); and proguanil (1-(p-chlorophenyl)-5-isopropylbiguanide); an α-glucosidase inhibitor selected from: acarbose ((2R,3R,4R,5R)-4-((2R,3R,4R,5S,6R)-5-(2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal); miglitol ((2R,3R,4R,5S)-1-(2-hydroxyethyl)-2-(hydroxymethyl)piperidine-3,4,5-triol); and voglibose ((1S,2S,3R,4S,5S)-5-(1,3-dihydroxypropan-2-ylamino)-1-(hydroxymethyl)cyclohexane-1,2,3,4-tetraol); an insulin analogue selected from: NPH insulin (also known as Humulin N, Novolin N, NPH Lletin II, and insulin isophane); insulin lispro (28B-L-lysine-29B-L-proline-insulin, wherein insulin is human insulin); insulin aspart (28B-L-aspartic acid-insulin, wherein insulin is human insulin); and insulin glulisine (3B-L-lysine-29B-L-glutamic acid-insulin, wherein insulin is human insulin); a sulfonylurea selected from: tolbutamide (Orinase, N-(butylcarbamoyl)-4-methylbenzenesulfonamide); acetohexamide (Dymelor, 4-acetyl-N-(cyclohexylcarbamoyl)benzenesulfonamide); tolazamide (Tolinase, N-(azepan-1-ylcarbamoyl)-4-methylbenzenesulfonamide); chlorpropamide (Diabinese, 4-chloro-N-(propylcarbamoyl)benzenesulfonamide); glipizide (Glucotrol, N-(4-(N-(cyclohexylcarbamoyl)sulfamoyl)phenethyl)-5-methylpyrazine-2-carboxamide); glibenclamide, also known as glyburide (Diabeta, Micronase, Glynase, 5-chloro-N-(4-(N-(cyclohexylcarbamoyl)sulfamoyl)phenethyl)-2-methoxybenzamide); glimepiride (Amaryl, 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide); and gliclazide (Diamicron, N-(hexahydrocyclopenta[c]pyrrol-2(1H)-ylcarbamoyl)-4-methylbenzenesulfonamide); a SGLT2 inhibitor selected from: dapagliflozin ((2S,3R,4R,5S,6R)-2-(4-chloro-3-(4-ethoxybenzyl)phenyl)-6-(hydroxymethyl)tetrahydro-2H-pyran-3,4,5-triol); remogliflozin (ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate); ASP1941, canagliflozin ((2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol); ISIS 388626; sergliflozin (ethyl ((2R,3S,4S,5R,6S)-3,4,5-trihydroxy-6-(2-(4-methoxybenzyl)phenoxy)tetrahydro-2H-pyran-2-yl)methyl carbonate), AVE2268 ((2R,3S,4S,5R,6S)-2-(hydroxymethyl)-6-(2-(4-methoxybenzyl)thiophen-3-yloxy)tetrahydro-2H-pyran-3,4,5-triol), BI10773, CSG453; and LX4211; a meglitinide selected from: repaglinide (Prandin, (S)-2-ethoxy-4-(2-(3-methyl-1-(2-(piperidin-1-yl)phenyl)butylamino)-2-oxoethyl)benzoic acid); nateglinide (Starlix, (R)-2-((1r,4R)-4-isopropylcyclohexanecarboxamido)-3-phenylpropanoic acid); and mitiglinide ((S)-2-benzyl-4-((3aR,7aS)-1H-isoindol-2(3H,3aH,4H,5H,6H,7H,7aH)-yl)-4-oxobutanoic acid); a thiazolidinedione selected from: rosiglitazone (Avandia, 5-(4-(2-(methyl(pyridin-2-yl)amino)ethoxy)benzyl)thiazolidine-2,4-dione); pioglitazone (Actos, 5-(4-(2-(5-ethylpyridin-2-yl)ethoxy)benzyl)thiazolidine-2,4-dione); troglitazone (Rezulin, 5-(4-((6-hydroxy-2,5,7,8-tetramethylchroman-2-yl)methoxy)benzyl)thiazolidine-2,4-dione); rivoglitazone (5-(4-((6-methoxy-1-methyl-1H-benzo[d]imidazol-2-yl)methoxy)benzyl)thiazolidine-2,4-dione); and ciglitazone (5-(4-((1-methylcyclohexyl)methoxy)benzyl)thiazolidine-2,4-dione); and an anti-diabetic peptide analogue selected from: exenatide; liraglutide; and taspoglutide.

In some embodiments, the two distinct pharmaceutical agents include every combination selected from pharmaceutical agents of the following group: sitagliptin, vildagliptin, saxagliptin, alogliptin, linagliptin, phenformin, metformin, buformin, acarbose, miglitol, voglibose, tolbutamide, acetohexamide, tolazamide, chlorpropamide, glipizide, glibenclamide, glimepiride, gliclazide, dapagliflozin, remigliflozin, and sergliflozin.

### Dipeptidyl Peptidase IV Inhibitors

Dipeptidyl peptidase IV (DPP-IV, EC 3.4.14.5) exhibits catalytic activity against a broad range of peptide substrates that includes peptide hormones, neuropeptides, and chemokines. The incretins glucagon-like peptide 1 (GLP-1), and glucose-dependent insulinotropic polypeptide (GIP), which stimulate glucose-dependent insulin secretion and otherwise promote blood glucose homeostasis, are rapidly cleaved by DPP-IV at the position-2 alanine leading to inactivation of their biological activity. Peptide YY (PYY) is a gut peptide that has been implicated in modulating satiety (Chaudhri et al, Annu Rev Physiol (2008) 70:239-255). PYY is released into the circulation as PYY₁₋₃₆ and PYY₃₋₃₆ (Eberlein et al, Peptides (1989) 10:797-803). PYY₃₋₃₆ is generated from PYY₁₋₃₆ by cleavage of the N-terminal Tyr and Pro residues by DPP-IV. Both pharmacological and genetic attenuation of DPP-IV activity is associated with enhanced incretin action, increased insulin, and lower blood glucose in vivo. Genetic attenuation of DPP-IV activity has been shown to provide resistance to obesity and to improve insulin sensitivity. Inhibitors of DPP-IV have shown to be useful as therapeutics, for example, oral administration of vildagliptin (1-[2-(3-hydroxyadamant-1-ylamino)acetyl]pyrrolidine-2(S)-carbonitrile) or sitagliptin (3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one) to human patients suffering with type 2 diabetes has been found to reduce fasting glucose and postprandial glucose excursion in association with significantly reduced HbA_{1c} levels. For reviews on the application of DPP-IV inhibitors for the treatment of type 2 diabetes, reference is made to the following publications: (1) H.-U. Demuth, et al., “Type 2 diabetes-therapy with DPP-IV inhibitors,” Biochim. Biophys. Acta, 1751: 33-44 (2005), and (2) K. Augustyns, et al., “Inhibitors of proline-specific dipeptidyl peptidases: DPP-IV inhibitors as a novel approach for the treatment of type 2 diabetes,” Expert Opin. Ther. Patents, 15: 1387-1407 (2005).

Accordingly, suitable pharmaceutical agents include inhibitors of DPP-IV that can be used in conjunction with compounds of the present invention either dosed separately or together. Inhibitors of DPP-IV are well-known in the art or can be readily identified and their in vitro biological activity determined using any number of methods available, for example, O'Brien, M., Daily, B., Schurria, M., “Assay for DPPIV activity using a homogeneous, luminescent method,” Cell Notes, Issue 11, 2005; see also the DPPIV-Glo™ Protease Assay Technical Bulletin #TB339.

Examples of DPP-IV inhibitors are described in Villhauer et al., J. Med. Chem. (2003) 46:2774-2789, for LAF237; Ahren et al., J. Clin. Endocrinol. Metab. (2004) 89:2078-2084; Villhauer et al., J. Med. Chem. (2002) 45:2362-2365 for NVP-DPP728; Ahren et al., Diabetes Care (2002) 25:869-875 for NVP-DPP728; Peters et al., Bioorg. Med. Chem. Lett. (2004) 14:1491-1493; Caldwell et al., Bioorg. Med. Chem. Lett. (2004) 14:1265-1268; Edmondson et al., Bioorg. Med. Chem. Lett. (2004) 14:5151-5155; and Abe et al., J. Na.t Prod. (2004) 67:999-1004.

Specific examples of DPP-IV inhibitors include, but are not limited to, dipeptide derivatives or dipeptide mimetics such as alanine-pyrrolidide, isoleucine-thiazolidide, and the pseudosubstrate N-valyl prolyl, O-benzoyl hydroxylamine, as described, for example, in U.S. Pat. No. 6,303,661.

Some embodiments of the present invention include every combination of one or more DPP-IV inhibitors selected from the DPP-IV inhibitors found in U.S. Pat. Nos. 6,869,947, 6,867,205, 6,861,440, 6,849,622, 6,812,350, 6,803,357, 6,800,650, 6,727,261, 6,716,843, 6,710,040, 6,706,742, 6,645,995, 6,617,340, 6,699,871, 6,573,287, 6,432,969, 6,395,767, 6,380,398, 6,303,661, 6,242,422, 6,166,063, 6,100,234, and 6,040,145.

Some embodiments of the present invention include every combination of one or more DPP-IV inhibitors selected from the DPP-IV inhibitors found in U.S. Pat. Nos. 2005059724, 2005059716, 2005043292, 2005038020, 2005032804, 2005004205, 2004259903, 2004259902, 2004259883, 2004254226, 2004242898, 2004229926, 2004180925, 2004176406, 2004138214, 2004116328, 2004110817, 2004106656, 2004097510, 2004087587, 2004082570, 2004077645, 2004072892, 2004063935, 2004034014, 2003232788, 2003225102, 2003216450, 2003216382, 2003199528, 2003195188, 2003162820, 2003149071, 2003134802, 2003130281, 2003130199, 2003125304, 2003119750, 2003119738, 2003105077, 2003100563, 2003087950, 2003078247, 2002198205, 2002183367, 2002103384, 2002049164, and 2002006899.

Some embodiments of the present invention include every combination of one or more DPP-IV inhibitors selected from the DPP-IV inhibitors found in International Patent Application Publication Nos. WO 2005/087235, WO 2005/082348, WO 2005/082849, WO 2005/079795, WO 2005/075426, WO 2005/072530, WO 2005/063750, WO 2005/058849, WO 2005/049022, WO 2005/047297, WO 2005/044195, WO 2005/042488, WO 2005/040095, WO 2005/037828, WO 2005/037779, WO 2005/034940, WO 2005/033099, WO 2005/032590, WO 2005/030751, WO 2005/030127, WO 2005/026148, WO 2005/025554, WO 2005/023762, WO 2005/020920, WO 05/19168, WO 05/12312, WO 05/12308, WO 05/12249, WO 05/11581, WO 05/09956, WO 05/03135, WO 05/00848, WO 05/00846, WO 04/112701, WO 04/111051, WO 04/111041, WO 04/110436, WO 04/110375, WO 04/108730, WO 04/104216, WO 04/104215, WO 04/103993, WO 04/103276, WO 04/99134, WO 04/96806, WO 04/92128, WO 04/87650, WO 04/87053, WO 04/85661, WO 04/85378, WO 04/76434, WO 04/76433, WO 04/71454, WO 04/69162, WO 04/67509, WO 04/64778, WO 04/58266, WO 04/52362, WO 04/52850, WO 04/50022, WO 04/50658, WO 04/48379, WO 04/46106, WO 04/43940, WO 04/41820, WO 04/41795, WO 04/37169, WO 04/37181, WO 04/33455, WO 04/32836, WO 04/20407, WO 04/18469, WO 04/18468, WO 04/18467, WO 04/14860, WO 04/09544, WO 04/07468, WO 04/07446, WO 04/04661, WO 04/00327, WO 03/106456, WO 03/104229, WO 03/101958, WO 03/101448, WO 03/99279, WO 03/95425, WO 03/84940, WO 03/82817, WO 03/80633, WO 03/74500, WO 03/72556, WO 03/72528, WO 03/68757, WO 03/68748, WO 03/57666, WO 03/57144, WO 03/55881, WO 03/45228, WO 03/40174, WO 03/38123, WO 03/37327, WO 03/35067, WO 03/35057, WO 03/24965, WO 03/24942, WO 03/22871, WO 03/15775, WO 03/04498, WO 03/04496, WO 03/02530, WO 03/02596, WO 03/02595, WO 03/02593, WO 03/02553, WO 03/02531, WO 03/00181, WO 03/00180, WO 03/00250, WO 02/83109, WO 02/83128, WO 02/76450, WO 02/68420, WO 02/62764, WO 02/55088, WO 02/51836, WO 02/38541, WO 02/34900, WO 02/30891, WO 02/30890, WO 02/14271, WO 02/02560, WO 01/97808, WO 01/96295, WO 01/81337, WO 01/81304, WO 01/68603, WO 01/55105, WO 01/52825, WO 01/34594, WO 00/71135, WO 00/69868, WO 00/56297, WO 00/56296, WO 00/34241, WO 00/23421, WO 00/10549, WO 99/67278, WO 99/62914, WO 99/61431, WO 99/56753, WO 99/25719, WO 99/16864, WO 98/50066, WO 98/50046, WO 98/19998, WO 98/18763, WO 97/40832, WO 95/29691, WO 95/15309, WO 93/10127, WO 93/08259, and WO 91/16339.

Some embodiments of the present invention include every combination of one or more DPP-IV inhibitors selected from the DPP-IV inhibitors found in Patent Publication Nos. EP 1517907, EP 1513808, EP 1492777, EP 1490335, EP 1489088, EP 1480961, EP 1476435, EP 1476429, EP 1469873, EP 1465891, EP 1463727, EP 1461337, EP 1450794, EP 1446116, EP 1442049, EP 1441719, EP 1426366, EP 1412357, EP1406873, EP 1406872, EP 1406622, EP 1404675, EP 1399420, EP 1399471, EP 1399470, EP 1399469, EP 1399433, EP 1399154, EP 1385508, EP 1377288, EP 1355886, EP 1354882, EP 1338592, EP 1333025, EP 1304327, EP 1301187, EP 1296974, EP 1280797, EP 1282600, EP 1261586, EP 1258476, EP 1254113, EP 1248604, EP 1245568, EP 1215207, EP 1228061, EP 1137635, EP 1123272, EP 1104293, EP 1082314, EP 1050540, EP 1043328, EP 0995440, EP 0980249, EP 0975359, EP 0731789, EP 0641347, EP 0610317, EP 0528858, CA 2466870, CA 2433090, CA 2339537, CA 2289125, CA 2289124, CA 2123128, DD 296075, DE 19834591, DE 19828113, DE 19823831, DE 19616486, DE 10333935, DE 10327439, DE 10256264, DE 10251927, DE 10238477, DE 10238470, DE 10238243, DE 10143840, FR 2824825, FR 2822826, JP2005507261, JP 2005505531, JP 2005502624, JP 2005500321, JP 2005500308, JP2005023038, JP 2004536115, JP 2004535445, JP 2004535433, JP 2004534836, JP 2004534815, JP 2004532220, JP 2004530729, JP 2004525929, JP 2004525179, JP 2004522786, JP 2004521149, JP 2004503531, JP 2004315496, JP 2004244412, JP 2004043429, JP 2004035574, JP 2004026820, JP 2004026678, JP 2004002368, JP 2004002367, JP 2003535898, JP 2003535034, JP 2003531204, JP 2003531191, JP 2003531118, JP 2003524591, JP 2003520849, JP 2003327532, JP 2003300977, JP 2003238566, JP 2002531547, JP 2002527504, JP 2002517401, JP 2002516318, JP 2002363157, JP 2002356472, JP 2002356471, JP 2002265439, JP 2001510442, JP 2000511559, JP 2000327689, JP 2000191616, JP 1998182613, JP 1998081666, JP 1997509921, JP 1995501078, and JP 1993508624.

In some embodiments, the DPP-IV inhibitor has an IC₅₀ of less than about 10 μM, less than about 1 μM, less than about 100 nM, less than about 75 nM, less than about 50 nM, less than about 25 nM, less than about 20 nM, less than about 15 nM, less than about 10 nM, less than about 5 nM, less than about 4 nM, less than about 3 nM, less than about 2 nM, or less than about 1 nM. In some embodiments, the DPP-IV inhibitor has an IC₅₀ of less than about 50 nM, less than about 25 nM, less than about 20 nM, less than about 15 nM, less than about 10 nM, less than about 5 nM, less than about 4 nM, less than about 3 nM, less than about 2 nM, or less than about 1 nM.

In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 10-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 100-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 10-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 1000-fold.

In some embodiments, the DPP-IV inhibitor is orally active.

In some embodiments, the DPP-IV inhibitor is an inhibitor of human DPP-IV.

Some embodiments of the present invention include every combination of one or more compounds selected from compounds of the following group and pharmaceutically acceptable salts, solvates, and hydrates thereof: 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one; 1-[2-(3-hydroxyadamant-1-ylamino)acetyl]pyrrolidine-2(S)-carbonitrile; (1S,3S,5S)-2-[2(S)-amino-2-(3-hydroxyadamantan-1-yl)acetyl]-2-azabicyclo[3.1.0]hexane-3-carbonitrile; 2-[6-[3 (R)-aminopiperidin-1-yl]-3-methyl-2,4-dioxo-1,2,3,4-tetrahydropyrimidin-1-ylmethyl]benzonitrile; 8-[3(R)-aminopiperidin-1-yl]-7-(2-butynyl)-3-methyl-1-(4-methylquinazolin-2-ylmethyl)xanthine; 1-[N-[3(R)-pyrrolidinyl]glycyl]pyrrolidin-2(R)-yl boronic acid; 4(S)-fluoro-1-[2-[(1R,3S)-3-(1H-1,2,4-triazol-1-ylmethyl)cyclopentylamino]acetyl]pyrrolidine-2(S)-carbonitrile; 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one; (2S,4S)-2-cyano-4-fluoro-1-[(2-hydroxy-1,1-dimethyl)ethylamino]acetylpyrrolidine; 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione; 1-((3S,4S)-4-amino-1-(4-(3,3-difluoropyrrolidin-1-yl)-1,3,5-triazin-2-yl)pyrrolidin-3-yl)-5,5-difluoropiperidin-2-one; (R)-2-((6-(3-aminopiperidin-1-yl)-3-methyl-2,4-dioxo-3,4-dihydropyrimidin-1(2H)-yl)methyl)-4-fluorobenzonitrile; 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide; ((2S,4S)-4-(4-(3-methyl-1-phenyl-1H-pyrazol-5-yl)piperazin-1-yl)pyrrolidin-2-yl)(thiazolidin-3-yl)methanone; (2S,4S)-1-[2-[(4-ethoxycarbonylbicyclo[2.2.2]oct-1-yl)amino]acetyl]-4-fluoropyrrolidine-2-carbonitrile; 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione; 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile; (2S)-1-{[2-(5-methyl-2-phenyl-oxazol-4-yl)-ethylamino]-acetyl}-pyrrolidine-2-carbonitrile; (2S)-1-{[1,1-dimethyl-3-(4-pyridin-3-yl-imidazol-1-yl)-propylamino]-acetyl}-pyrrolidine-2-carbonitrile; (3,3-difluoropyrrolidin-1-yl)-((2S,4S)-4-(4-(pyrimidin-2-yl)piperazin-1-yl)pyrrolidin-2-yl)methanone; (2S,4S)-1-[(2S)-2-amino-3,3-bis(4-fluorophenyl)propanoyl]-4-fluoropyrrolidine-2-carbonitrile; (2S,5R)-5-ethynyl-1-{N-(4-methyl-1-(4-carboxy-pyridin-2-yl)piperidin-4-yl)glycyl}pyrrolidine-2-carbonitrile; and (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine

Sitagliptin phosphate (Januvia®, MK-0431, dihydrogenphosphate salt of 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one) is marketed by Merck & Co. for once-daily oral treatment of type 2 diabetes. Januvia was first launched in Mexico followed by commercialization in the U.S. In 2007, the product was approved by the European Medicines Evaluation Agency (EMEA) and is currently available in the U.K., Germany and Spain. In 2009, Januvia was approved and launched in Japan. In addition, Merck has also filed for approval of Januvia in the U.S. as an adjunct to diet and exercise and in combination with other therapies to improve glycemic control in the treatment of diabetes. The compound, 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one, and pharmaceutically acceptable salts thereof are disclosed in international patent publication WO2003/004498. Some embodiments of the present invention include every combination of one or more compounds selected from compounds disclosed in WO2003/004498 and pharmaceutically acceptable salts, solvates, and hydrates thereof. In some embodiments, the DPP-IV inhibitor is selected from 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one, and pharmaceutically acceptable salts, solvates, and hydrates thereof:

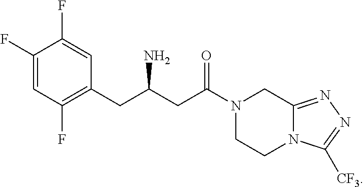 [SMILES: N[C@@H](CC(=O)N1CCn2c(nnc2C(F)(F)F)C1)Cc1cc(F)c(F)cc1F]

In some embodiments, the DPP-IV inhibitor is (R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate:

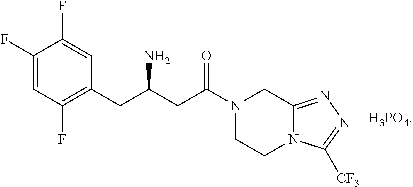

The crystalline form of (R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate salt monohydrate is disclosed in international patent publication WO2005/003135. In some embodiments, the DPP-IV inhibitor is crystalline (R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate monohydrate.

Vildagliptin (Galvus®, LAF-237, 1-[2-(3-hydroxyadamant-1-ylamino)acetyl]pyrrolidine-2(S)-carbonitrile) is another DPP-IV inhibitor and was first commercialized in Brazil and Mexico by Novartis for oral, once-daily treatment of type 2 diabetes. In 2008, a marketing authorization application (MAA) was approved in the E.U. for this indication and launch took place in the U.K. in March, 2008. An approvable letter has been received for the regulatory application filed in the U.S. Vildagliptin was approved in Japan in 2010. The compound, 1-[2-(3-hydroxyadamant-1-ylamino)acetyl]pyrrolidine-2(S)-carbonitrile, is disclosed in international patent publication WO2000/034241. Some embodiments of the present invention include every combination of one or more compounds selected from compounds disclosed in WO2000/034241 and pharmaceutically acceptable salts, solvates, and hydrates thereof. In some embodiments, the DPP-IV inhibitor is selected from 1-[2-(3-hydroxyadamant-1-ylamino)acetyl]pyrrolidine-2(S)-carbonitrile, and pharmaceutically acceptable salts, solvates, and hydrates thereof:

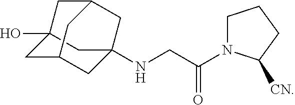 [SMILES: N#C[C@@H]1CCCN1C(=O)CNC12CC3CC(CC(O)(C3)C1)C2]

Certain salts of the compound, 1-[2-(3-hydroxyadamant-1-ylamino)acetyl]pyrrolidine-2(S)-carbonitrile, are disclosed in international patent publication WO2007/019255. In some embodiments, the DPP-IV inhibitor is 1-[2-(3-hydroxyadamant-1-ylamino)acetyl]pyrrolidine-2(S)-carbonitrile HCl:

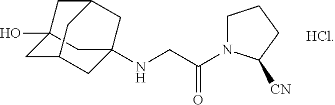 [SMILES: N#C[C@@H]1CCCN1C(=O)CNC12CC3CC(CC(O)(C3)C1)C2]

Saxagliptin (Onglyza™, BMS-477118, (1S,3S,5S)-2-[2(S)-amino-2-(3-hydroxyadamantan-1-yl)acetyl]-2-azabicyclo[3.1.0]hexane-3-carbonitrile) is another DPP-IV inhibitor, which was launched in 2009 by AstraZeneca and Bristol-Myers Squibb in the U.S. for the treatment of type 2 diabetes. In 2009, the product was approved in the E.U. for the treatment of type 2 diabetes independently or in combination with metformin. Phase 3 clinical studies are ongoing in Japan for the treatment of type 2 diabetes. The compound, (1S,3S,5S)-2-[2(S)-amino-2-(3-hydroxyadamantan-1-yl)acetyl]-2-azabicyclo[3.1.0]hexane-3-carbonitrile, is disclosed in international patent publication WO2001/068603. Some embodiments of the present invention include every combination of one or more compounds selected from compounds disclosed in WO2001/068603 and pharmaceutically acceptable salts, solvates, and hydrates thereof. In some embodiments, the DPP-IV inhibitor is selected from (1S,3S,5S)-2-[2(S)-amino-2-(3-hydroxyadamantan-1-yl)acetyl]-2-azabicyclo[3.1.0]hexane-3-carbonitrile, and pharmaceutically acceptable salts, solvates, and hydrates thereof:

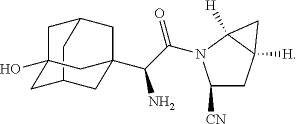

Takeda has filed for regulatory approval of the DPP-IV inhibitor, alogliptin (SYR-322, 2-[6-[3(R)-aminopiperidin-1-yl]-3-methyl-2,4-dioxo-1,2,3,4-tetrahydropyrimidin-1-ylmethyl]benzonitrile) in Japan and the U.S for the once-daily, oral treatment of type 2 diabetes. The compound, 2-[6-[3(R)-aminopiperidin-1-yl]-3-methyl-2,4-dioxo-1,2,3,4-tetrahydropyrimidin-1-ylmethyl]benzonitrile, and pharmaceutically acceptable salts thereof are disclosed in international patent publication WO 2005/095381. Some embodiments of the present invention include every combination of one or more compounds selected from compounds disclosed in WO 2005/095381 and pharmaceutically acceptable salts, solvates, and hydrates thereof. In some embodiments, the DPP-IV inhibitor is selected from 2-[6-[3(R)-aminopiperidin-1-yl]-3-methyl-2,4-dioxo-1,2,3,4-tetrahydropyrimidin-1-ylmethyl]benzonitrile, and pharmaceutically acceptable salts, solvates, and hydrates thereof:

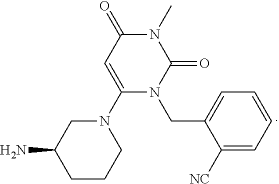 [SMILES: Cn1c(=O)cc(N2CCC[C@@H](N)C2)n(Cc2ccccc2C#N)c1=O]

The crystalline form of 2-[6-[3(R)-aminopiperidin-1-yl]-3-methyl-2,4-dioxo-1,2,3,4-tetrahydropyrimidin-1-ylmethyl]benzonitrile is disclosed in international patent publication WO2007/035372. In some embodiments, the DPP-IV inhibitor is 2-[6-[3(R)-aminopiperidin-1-yl]-3-methyl-2,4-dioxo-1,2,3,4-tetrahydropyrimidin-1-ylmethyl]benzonitrile benzoate:

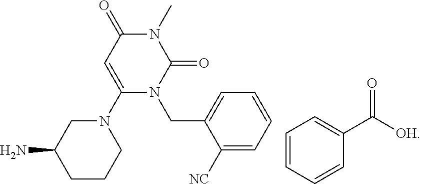

Linagliptin (BI-1356, Ondero®, 8-[3(R)-aminopiperidin-1-yl]-7-(2-butynyl)-3-methyl-1-(4-methylquinazolin-2-ylmethyl)xanthine) is a DPP-IV inhibitor in phase 3 clinical development at Boehringer Ingelheim to evaluate its potential as add-on therapy to metformin for the treatment of type 2 diabetes. The compound, 8-[3(R)-aminopiperidin-1-yl]-7-(2-butynyl)-3-methyl-1-(4-methylquinazolin-2-ylmethyl)xanthine, is disclosed in international patent publication WO2004/018468. Some embodiments of the present invention include every combination of one or more compounds selected from compounds disclosed in WO2004/018468 and pharmaceutically acceptable salts, solvates, and hydrates thereof. In some embodiments, the DPP-IV inhibitor is selected from 8-[3(R)-aminopiperidin-1-yl]-7-(2-butynyl)-3-methyl-1-(4-methylquinazolin-2-ylmethyl)xanthine, and pharmaceutically acceptable salts, solvates, and hydrates thereof:

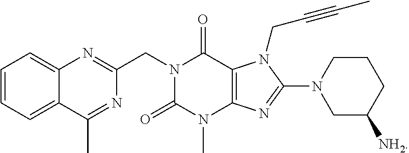 [SMILES: CC#CCn1c(N2CCC[C@@H](N)C2)nc2c1c(=O)n(Cc1nc(C)c3ccccc3n1)c(=O)n2C]

Certain polymorphs of the compound, 8-[3(R)-aminopiperidin-1-yl]-7-(2-butynyl)-3-methyl-1-(4-methylquinazolin-2-ylmethyl)xanthine, are disclosed in international patent publication WO 2007/128721. In some embodiments, the DPP-IV inhibitor is a crystalline form of 8-[3(R)-aminopiperidin-1-yl]-7-(2-butynyl)-3-methyl-1-(4-methylquinazolin-2-ylmethyl)xanthine.

Dutogliptin (PHX-1149, 1-[N-[3(R)-pyrrolidinyl]glycyl]pyrrolidin-2(R)-yl boronic acid) is a DPP-IV inhibitor in phase 3 clinical trials by Phenomix and Forest for the oral, once-daily treatment of type 2 diabetes. The compound, 1-[N-[3(R)-pyrrolidinyl]glycyl]pyrrolidin-2(R)-yl boronic acid, and pharmaceutically acceptable salts thereof are disclosed in international patent publication WO2005/047297. Some embodiments of the present invention include every combination of one or more compounds selected from compounds disclosed in WO2005/047297 and pharmaceutically acceptable salts, solvates, and hydrates thereof. In some embodiments, the DPP-IV inhibitor is selected from 1-[N-[3(R)-pyrrolidinyl]glycyl]pyrrolidin-2(R)-yl boronic acid, and pharmaceutically acceptable salts, solvates, and hydrates thereof:

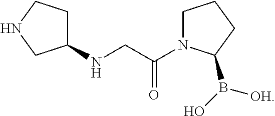 [SMILES: O=C(CN[C@@H]1CCNC1)N1CCC[C@H]1B(O)O]

The crystalline form of 1-[N-[3(R)-pyrrolidinyl]glycyl]pyrrolidin-2(R)-yl boronic acid tartrate is disclosed in international patent publication WO2008/027273. In some embodiments, the DPP-IV inhibitor is 1-[N-[3(R)-pyrrolidinyl]glycyl]pyrrolidin-2(R)-yl boronic acid tartrate:

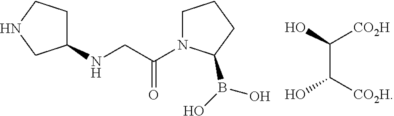

Melogliptin (GRC-8200, 4(S)-fluoro-1-[2-[(1R,3S)-3-(1H-1,2,4-triazol-1-ylmethyl)cyclopentylamino]acetyl]pyrrolidine-2(S)-carbonitrile) is a DPP-IV inhibitor currently undergoing phase 2 clinical trials by Glenmark Pharmaceuticals and Merck KGaA for the treatment of type 2 diabetes. The compound, 4(S)-fluoro-1-[2-[(1R,3S)-3-(1H-1,2,4-triazol-1-ylmethyl)cyclopentylamino]acetyl]pyrrolidine-2(S)-carbonitrile, is disclosed in international patent publication WO2006/040625. Some embodiments of the present invention include every combination of one or more compounds selected from compounds disclosed in WO2006/040625 and pharmaceutically acceptable salts, solvates, and hydrates thereof. In some embodiments, the DPP-IV inhibitor is selected from 4(S)-fluoro-1-[2-[(1R,3S)-3-(1H-1,2,4-triazol-1-ylmethyl)cyclopentylamino]acetyl]pyrrolidine-2(S)-carbonitrile, and pharmaceutically acceptable salts, solvates, and hydrates thereof:

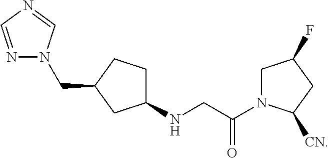 [SMILES: N#C[C@@H]1C[C@H](F)CN1C(=O)CN[C@@H]1CC[C@H](Cn2cncn2)C1]

Carmegliptin (R-1579, 1-[2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one) is a DPP-IV inhibitor. The compound, 1-[2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one, is disclosed in international patent publication WO2005/000848. Some embodiments of the present invention include every combination of one or more compounds selected from compounds disclosed in WO2005/000848 and pharmaceutically acceptable salts, solvates, and hydrates thereof. In some embodiments, the DPP-IV inhibitor is selected from 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one, and pharmaceutically acceptable salts, solvates, and hydrates thereof:

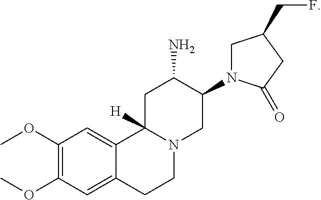 [SMILES: COc1cc2c(cc1OC)[C@@H]1C[C@H](N)[C@@H](N3C[C@@H](CF)CC3=O)CN1CC2]

Taisho disclosed (2S,4S)-2-cyano-4-fluoro-1-[(2-hydroxy-1,1-dimethyl)ethylamino]acetylpyrrolidine, a DPP-IV inhibitor in US patent publication US 2007/0112059. Some embodiments of the present invention include every combination of one or more compounds selected from compounds disclosed in US 2007/0112059 and pharmaceutically acceptable salts, solvates, and hydrates thereof. In some embodiments, the DPP-IV inhibitor is selected from (2S,4S)-2-cyano-4-fluoro-1-[(2-hydroxy-1,1-dimethyl)ethylamino]acetylpyrrolidine, and pharmaceutically acceptable salts, solvates, and hydrates thereof:

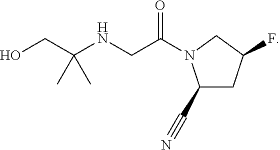 [SMILES: CC(C)(CO)NCC(=O)N1C[C@@H](F)C[C@H]1C#N]

Sanofi-Aventis disclosed a series of substituted bicyclic 8-pyrrolidineoxanthine derivatives as DPP-IV inhibitors in US publication US 2007/0167468. Some embodiments of the present invention include every combination of one or more compounds selected from compounds disclosed in US publication US 2007/0167468 and pharmaceutically acceptable salts, solvates, and hydrates thereof. In some embodiments, the DPP-IV inhibitor is selected from 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione, and pharmaceutically acceptable salts, solvates, and hydrates thereof:

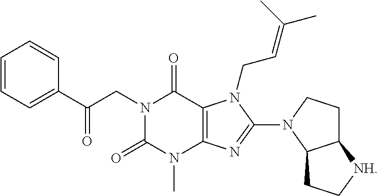 [SMILES: CC(C)=CCn1c(N2CC[C@H]3NCC[C@H]32)nc2c1c(=O)n(CC(=O)c1ccccc1)c(=O)n2C]

Pfizer disclosed a series of 3-amino-pyrrolidine-4-lactam derivatives as DPP-IV inhibitors in international patent publication WO2007/148185. Some embodiments of the present invention include every combination of one or more compounds selected from compounds disclosed in WO2007/148185 and pharmaceutically acceptable salts, solvates, and hydrates thereof. One such compound is 1-((3S,4S)-4-amino-1-(4-(3,3-difluoropyrrolidin-1-yl)-1,3,5-triazin-2-yl)pyrrolidin-3-yl)-5,5-difluoropiperidin-2-one. In some embodiments, the DPP-IV inhibitor is selected from 1-((3S,4S)-4-amino-1-(4-(3,3-difluoropyrrolidin-1-yl)-1,3,5-triazin-2-yl)pyrrolidin-3-yl)-5,5-difluoropiperidin-2-one, and pharmaceutically acceptable salts, solvates, and hydrates thereof:

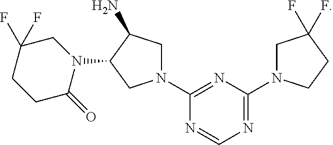 [SMILES: N[C@H]1CN(c2ncnc(N3CCC(F)(F)C3)n2)C[C@@H]1N1CC(F)(F)CCC1=O]

Syrrx disclosed a series of substituted pyrimidine-2,4(1H,3H)-dione derivatives as DPP-IV inhibitors in international patent publication WO2005/095381. Some embodiments of the present invention include every combination of one or more compounds selected from compounds disclosed in WO2005/095381 and pharmaceutically acceptable salts, solvates, and hydrates thereof. One such compound is (R)-2-((6-(3-aminopiperidin-1-yl)-3-methyl-2,4-dioxo-3,4-dihydropyrimidin-1(2H)-yl)methyl)-4-fluorobenzonitrile. In some embodiments, the DPP-IV inhibitor is selected from (R)-2-((6-(3-aminopiperidin-1-yl)-3-methyl-2,4-dioxo-3,4-dihydropyrimidin-1(2H)-yl)methyl)-4-fluorobenzonitrile, and pharmaceutically acceptable salts, solvates, and hydrates thereof:

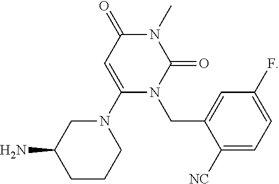 [SMILES: Cn1c(=O)cc(N2CCC[C@@H](N)C2)n(Cc2cc(F)ccc2C#N)c1=O]

Various crystalline forms of (R)-2-((6-(3-aminopiperidin-1-yl)-3-methyl-2,4-dioxo-3,4-dihydropyrimidin-1(2H)-yl)methyl)-4-fluorobenzonitrile succinic acid salt are disclosed in international patent publication WO2008/067465. One embodiment of the present invention pertains to any one or more crystalline forms of (R)-2-((6-(3-aminopiperidin-1-yl)-3-methyl-2,4-dioxo-3,4-dihydropyrimidin-1(2H)-yl)methyl)-4-fluorobenzonitrile succinic acid salt as described in international patent publication WO2008/067465. In some embodiments, the DPP-IV inhibitor is crystalline (R)-2-((6-(3-aminopiperidin-1-yl)-3-methyl-2,4-dioxo-3,4-dihydropyrimidin-1(2H)-yl)methyl)-4-fluorobenzonitrile succinic acid salt:

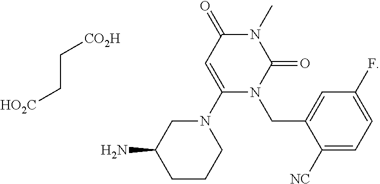 [SMILES: O=C(O)CCC(=O)O ; Cn1c(=O)cc(N2CCC[C@@H](N)C2)n(Cc2cc(F)ccc2C#N)c1=O]

Alantos disclosed a series of substituted 2-cyano-pyrrolidine derivatives as DPP-IV inhibitors in international patent publication WO2006/116157. Some embodiments of the present invention include every combination of one or more compounds selected from compounds disclosed in WO2006/116157 and pharmaceutically acceptable salts, solvates, and hydrates thereof. One such compound is 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide. In some embodiments, the DPP-IV inhibitor is selected from 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide, and pharmaceutically acceptable salts, solvates, and hydrates thereof:

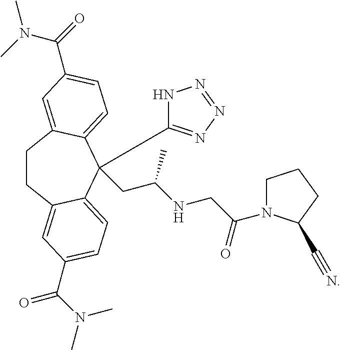 [SMILES: C[C@@H](CC1(c2nnn[nH]2)c2ccc(C(=O)N(C)C)cc2CCc2cc(C(=O)N(C)C)ccc21)NCC(=O)N1CCC[C@H]1C#N]

Mitsubishi disclosed a series of 2,4-disubstituted pyrrolidine derivatives as DPP-IV inhibitors in international patent publication WO2002/0014271. Some embodiments of the present invention include every combination of one or more compounds selected from compounds disclosed in WO2002/0014271 and pharmaceutically acceptable salts, solvates, and hydrates thereof. One such compound is ((2S,4S)-4-(4-(3-methyl-1-phenyl-1H-pyrazol-5-yl)piperazin-1-yl)pyrrolidin-2-yl)(thiazolidin-3-yl)methanone. In some embodiments, the DPP-IV inhibitor is selected from ((2S,4S)-4-(4-(3-methyl-1-phenyl-1H-pyrazol-5-yl)piperazin-1-yl)pyrrolidin-2-yl)(thiazolidin-3-yl)methanone, and pharmaceutically acceptable salts, solvates, and hydrates thereof:

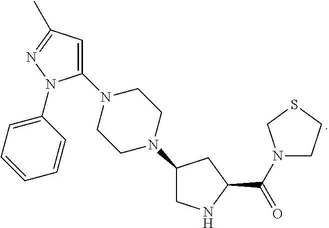 [SMILES: Cc1cc(N2CCN([C@@H]3CN[C@H](C(=O)N4CCSC4)C3)CC2)n(-c2ccccc2)n1 ; "."]

Various crystalline forms of ((2S,4S)-4-(4-(3-methyl-1-phenyl-1H-pyrazol-5-yl)piperazin-1-yl)pyrrolidin-2-yl)(thiazolidin-3-yl)methanone salts are disclosed in international patent publication WO2006/088129 and US publication 2009/0216016. One embodiment of the present invention pertains to any one or more crystalline forms of ((2S,4S)-4-(4-(3-methyl-1-phenyl-1H-pyrazol-5-yl)piperazin-1-yl)pyrrolidin-2-yl)(thiazolidin-3-yl)methanone salt as described in international patent publication WO2006/088129 and US publication 2009/0216016. In some embodiments, the DPP-IV inhibitor is crystalline ((2S,4S)-4-(4-(3-methyl-1-phenyl-1H-pyrazol-5-yl)piperazin-1-yl)pyrrolidin-2-yl)(thiazolidin-3-yl)methanone 2.5 hydrobromide salt:

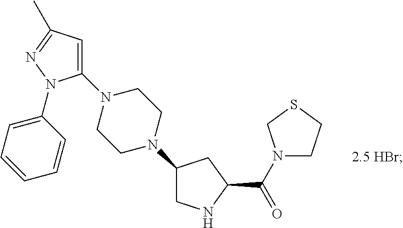

or a mono or a dihydrate thereof. In some embodiments, the DPP-IV inhibitor is crystalline ((2S,4S)-4-(4-(3-methyl-1-phenyl-1H-pyrazol-5-yl)piperazin-1-yl)pyrrolidin-2-yl)(thiazolidin-3-yl)methanone di-hydrobromide salt.

Kyorin disclosed a series of pyrrolidinecarbonitrile derivatives as DPP-IV inhibitors in international patent publication WO2008/114857 and US publication US 2008/0146818. Some embodiments of the present invention include every combination of one or more compounds selected from compounds disclosed in WO2008/114857 and US publication US 2008/0146818, and pharmaceutically acceptable salts, solvates, and hydrates thereof. One such compound is (2S,4S)-1-[2-[(4-ethoxycarbonylbicyclo[2.2.2]oct-1-yl)amino]acetyl]-4-fluoropyrrolidine-2-carbonitrile. In some embodiments, the DPP-IV inhibitor is selected from (2S,4S)-1-[2-[(4-ethoxycarbonylbicyclo[2.2.2]oct-1-yl)amino]acetyl]-4-fluoropyrrolidine-2-carbonitrile, and pharmaceutically acceptable salts, solvates, and hydrates thereof:

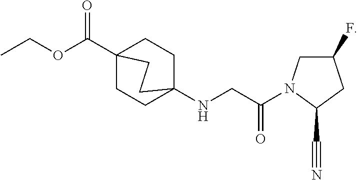 [SMILES: CCOC(=O)C12CCC(NCC(=O)N3C[C@@H](F)C[C@H]3C#N)(CC1)CC2]

Dainippon Sumitomo disclosed a series of bicyclic pyrrole derivatives as DPP-IV inhibitors in international patent publication WO2006/068163 and US publication US 2009/0192129. Some embodiments of the present invention include every combination of one or more compounds selected from compounds disclosed in WO2006/068163 and US publication US 2009/0192129 and pharmaceutically acceptable salts, solvates, and hydrates thereof. One such compound is (6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione. In some embodiments, the DPP-IV inhibitor is selected from (6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione, and pharmaceutically acceptable salts, solvates, and hydrates thereof:

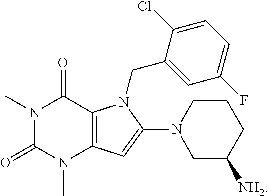 [SMILES: Cn1c(=O)c2c(cc(N3CCC[C@@H](N)C3)n2Cc2cc(F)ccc2Cl)n(C)c1=O]

Dainippon Sumitomo disclosed 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile as a DPP-IV inhibitor in international patent publication WO2009/084497. In some embodiments, the DPP-IV inhibitor is selected from 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile, and pharmaceutically acceptable salts, solvates, and hydrates thereof:

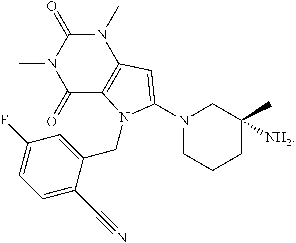 [SMILES: Cn1c(=O)c2c(cc(N3CCC[C@@](C)(N)C3)n2Cc2cc(F)ccc2C#N)n(C)c1=O]

Hoffmann-La Roche disclosed a series of N-substituted pyrrolidine derivatives as DPP-IV inhibitors in international patent publication WO 03/037327. Some embodiments of the present invention include every combination of one or more compounds selected from compounds disclosed in WO 03/037327 and pharmaceutically acceptable salts, solvates, and hydrates thereof. One such compound is (2S)-1-{[2-(5-methyl-2-phenyl-oxazol-4-yl)-ethylamino]-acetyl}-pyrrolidine-2-carbonitrile. In some embodiments, the DPP-IV inhibitor is selected from (2S)-1-{[2-(5-methyl-2-phenyl-oxazol-4-yl)-ethylamino]-acetyl}-pyrrolidine-2-carbonitrile, and pharmaceutically acceptable salts, solvates, and hydrates thereof:

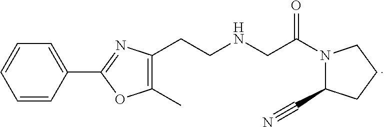 [SMILES: Cc1oc(-c2ccccc2)nc1CCNCC(=O)N1CCC[C@H]1C#N ; "."]

Various crystalline forms of (2S)-1-{[2-(5-methyl-2-phenyl-oxazol-4-yl)-ethylamino]-acetyl}-pyrrolidine-2-carbonitrile methansulfonic acid salt are disclosed in international patent publication WO2006/100181. In some embodiments, the DPP-IV inhibitor is (2S)-1-{[2-(5-methyl-2-phenyl-oxazol-4-yl)-ethylamino]-acetyl}-pyrrolidine-2-carbonitrile methansulfonic acid salt (i.e., mesylate):

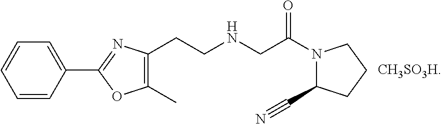 [SMILES: Cc1oc(-c2ccccc2)nc1CCNCC(=O)N1CCC[C@H]1C#N]

Other compounds disclosed by Hoffmann-La Roche in international patent publication WO 03/037327 include (2S)-1-{[1,1-dimethyl-3-(4-pyridin-3-yl-imidazol-1-yl)-propylamino]-acetyl}-pyrrolidine-2-carbonitrile, and pharmaceutically acceptable salts thereof, such as the methansulfonic acid salt. In some embodiments, the DPP-IV inhibitor is selected from (2S)-1-{[1,1-dimethyl-3-(4-pyridin-3-yl-imidazol-1-yl)-propylamino]-acetyl}-pyrrolidine-2-carbonitrile, and pharmaceutically acceptable salts, solvates, and hydrates thereof:

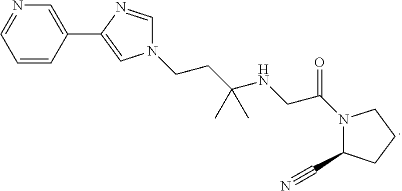 [SMILES: CC(C)(CCn1cnc(-c2cccnc2)c1)NCC(=O)N1CCC[C@H]1C#N]

In some embodiments, the DPP-IV inhibitor is (2S)-1-{[1,1-dimethyl-3-(4-pyridin-3-yl-imidazol-1-yl)-propylamino]-acetyl}-pyrrolidine-2-carbonitrile methansulfonic acid:

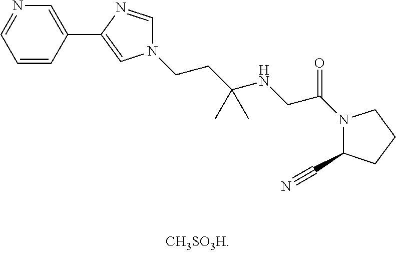 [SMILES: CC(C)(CCn1cnc(-c2cccnc2)c1)NCC(=O)N1CCC[C@H]1C#N]

Various crystalline forms of (2S)-1-{[1,1-dimethyl-3-(4-pyridin-3-yl-imidazol-1-yl)-propylamino]-acetyl}-pyrrolidine-2-carbonitrile fumaric acid salt are disclosed in international patent publication WO2007/071576. In some embodiments, the DPP-IV inhibitor is (2S)-1-{[1,1-dimethyl-3-(4-pyridin-3-yl-imidazol-1-yl)-propylamino]-acetyl}-pyrrolidine-2-carbonitrile fumaric acid salt (i.e., fumarate):

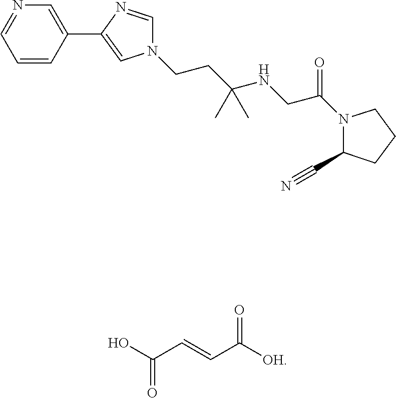 [SMILES: CC(C)(CCn1cnc(-c2cccnc2)c1)NCC(=O)N1CCC[C@H]1C#N ; O=C(O)/C=C/C(=O)O]

Pfizer disclosed a series of proline derivatives as DPP-IV inhibitors in international patent publication WO2005/116014. Some embodiments of the present invention include every combination of one or more compounds selected from compounds disclosed in WO2005/116014 and pharmaceutically acceptable salts, solvates, and hydrates thereof. One such compound is (3,3-difluoropyrrolidin-1-yl)-((2S,4S)-4-(4-(pyrimidin-2-yl)piperazin-1-yl)pyrrolidin-2-yl)methanone. In some embodiments, the DPP-IV inhibitor is selected from (3,3-difluoropyrrolidin-1-yl)-((2S,4S)-4-(4-(pyrimidin-2-yl)piperazin-1-yl)pyrrolidin-2-yl)methanone, and pharmaceutically acceptable salts, solvates, and hydrates thereof:

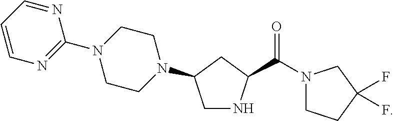 [SMILES: O=C([C@@H]1C[C@H](N2CCN(c3ncccn3)CC2)CN1)N1CCC(F)(F)C1]

GlaxoSmithKline disclosed a series of fluoropyrrolidine derivatives as DPP-IV inhibitors in international patent publication WO 03/002531. Some embodiments of the present invention include every combination of one or more compounds selected from the DPP-IV inhibitors disclosed in WO 03/037327 and pharmaceutically acceptable salts, solvates, and hydrates thereof. One such compound is (2S,4S)-1-[2S)-2-amino-3,3-bis(4-fluorophenyl)propanoyl]-4-fluoropyrrolidine-2-carbonitrile (Denagliptin). In some embodiments, the DPP-IV inhibitor is selected from (2S,4S)-1-[(2S)-2-amino-3,3-bis(4-fluorophenyl)propanoyl]-4-fluoropyrrolidine-2-carbonitrile, and pharmaceutically acceptable salts, solvates, and hydrates thereof:

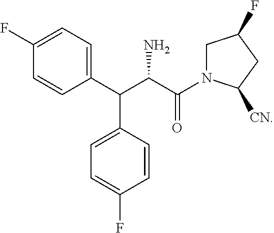 [SMILES: N#C[C@@H]1C[C@H](F)CN1C(=O)[C@@H](N)C(c1ccc(F)cc1)c1ccc(F)cc1]

Various crystalline forms of (2S,4S)-1-[(2S)-2-amino-3,3-bis(4-fluorophenyl)propanoyl]-4-fluoropyrrolidine-2-carbonitrile and salts have been disclosed in international patent publication WO 2005/009956. One salt disclosed is (2S,4S)-1-[2S)-2-amino-3,3-bis(4-fluorophenyl)propanoyl]-4-fluoropyrrolidine-2-carbonitrile p-toluenesulfonic acid salt (also referred to as (2S,4S)-4-fluoro-1-[4-fluoro-β-(4-fluorophenyl)-L-phenylalanyl]-2-pyrrolidinecarbonitrile p-toluenesulfonic acid salt, or Denagliptin tosylate). In some embodiments, the DPP-IV inhibitor is (2S,4S)-1-[(2S)-2-amino-3,3-bis(4-fluorophenyl)propanoyl]-4-fluoropyrrolidine-2-carbonitrile p-toluenesulfonic acid salt:

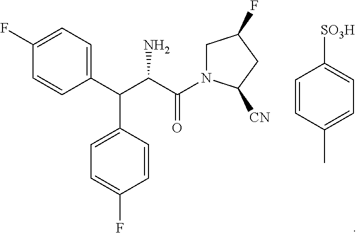

Abbott disclosed a series of substituted pyrrolidinyl derivatives as DPP-IV inhibitors in international patent publication WO 2004/026822. Some embodiments of the present invention include every combination of one or more compounds selected from the DPP-IV inhibitors disclosed in WO 2004/026822 and pharmaceutically acceptable salts, solvates, and hydrates thereof. One such compound is (2S,5R)-5-ethynyl-1-{N-(4-methyl-1-(4-carboxy-pyridin-2-yl)piperidin-4-yl)glycyl}pyrrolidine-2-carbonitrile. In some embodiments, the DPP-IV inhibitor is selected from (2S,5R)-5-ethynyl-1-{N-(4-methyl-1-(4-carboxy-pyridin-2-yl)piperidin-4-yl)glycyl}pyrrolidine-2-carbonitrile, and pharmaceutically acceptable salts, solvates, and hydrates thereof:

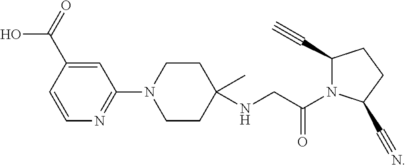 [SMILES: C#C[C@H]1CC[C@@H](C#N)N1C(=O)CNC1(C)CCN(c2cc(C(=O)O)ccn2)CC1]

Abbott has further disclosed a series of substituted cyclohexanyl/cyclohexenyl derivatives as DPP-IV inhibitors in international patent publication WO 2007/027651. Some embodiments of the present invention include every combination of one or more compounds selected from the DPP-IV inhibitors disclosed in WO 2007/027651 and pharmaceutically acceptable salts, solvates, and hydrates thereof. One such compound is (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine. In some embodiments, the DPP-IV inhibitor is selected from (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine, and pharmaceutically acceptable salts, solvates, and hydrates thereof:

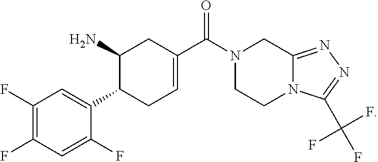 [SMILES: N[C@H]1CC(C(=O)N2CCn3c(nnc3C(F)(F)F)C2)=CC[C@@H]1c1cc(F)c(F)cc1F]

### Biguanides

The biguanides are a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin ((phenylethyl)biguanide), metformin (dimethylbiguanide), buformin (butylbiguanide), proguanil (1-(p-chlorophenyl)-5-isopropylbiguanide), and biguanides known in the art.

In some embodiments, the pharmaceutical agent or said second pharmaceutical agent is a biguanide selected from the following biguanide:

(phenylethyl)biguanide, dimethylbiguanide, butylbiguanide, 1-(p-chlorophenyl)-5-isopropylbiguanide, and pharmaceutically acceptable salts, solvates, and hydrates thereof.

In some embodiments, the pharmaceutical agent or the second pharmaceutical agent is a biguanide selected from (phenylethyl)biguanide (chemical structure shown below) and pharmaceutically acceptable salts, solvates, and hydrates thereof:

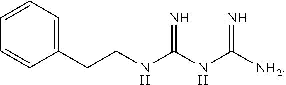 [SMILES: N=C(N)NC(=N)NCCc1ccccc1]

In some embodiments, the pharmaceutical agent or the second pharmaceutical agent is a biguanide selected from dimethylbiguanide (chemical structure shown below) and pharmaceutically acceptable salts, solvates, and hydrates thereof; the chemical structure is as follows:

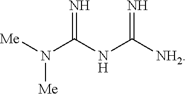

In some embodiments, the pharmaceutical agent or the second pharmaceutical agent is a biguanide selected from butylbiguanide (chemical structure shown below) and pharmaceutically acceptable salts, solvates, and hydrates thereof; the chemical structure is as follows:

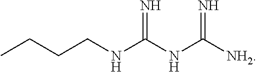

In some embodiments, the pharmaceutical agent or the second pharmaceutical agent is a biguanide selected from 1-(p-chlorophenyl)-5-isopropylbiguanide (chemical structure shown below) and pharmaceutically acceptable salts, solvates, and hydrates thereof; the chemical structure is as follows:

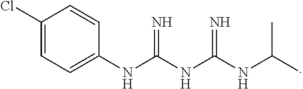

In some embodiments, the pharmaceutical agent or said second pharmaceutical agent is a biguanide selected from the following biguanides: metformin, phenformin, buformin, and proguanil. In some embodiments, the pharmaceutical agent or the second pharmaceutical agent is metformin. In some embodiments, the pharmaceutical agent or the second pharmaceutical agent is phenformin. In some embodiments, the pharmaceutical agent or the second pharmaceutical agent is buformin. In some embodiments, the pharmaceutical agent or the second pharmaceutical agent is proguanil.

### Alpha-Gluocosidase Inhibitors

α-Glucosidase inhibitors belong to the class of drugs which competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Some representative examples of α-glucosidase inhibitors include acarbose ((2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal), miglitol ((2R,3R,4R,5S)-1-(2-hydroxyethyl)-2-(hydroxymethyl)piperidine-3,4,5-triol), voglibose ((1S,2S,3R,4S,5S)-5-(1,3-dihydroxypropan-2-ylamino)-1-(hydroxymethyl)cyclohexane-1,2,3,4-tetraol), and α-glucosidase inhibitors known in the art.

In some embodiments, the pharmaceutical agent or said second pharmaceutical agent is a α-glucosidase inhibitor selected from the following α-glucosidase inhibitors:

(2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal; (2R,3R,4R,5S)-1-(2-hydroxyethyl)-2-(hydroxymethyl)piperidine-3,4,5-triol; (1S,2S,3R,4S,5S)-5-(1,3-dihydroxypropan-2-ylamino)-1-(hydroxymethyl)cyclohexane-1,2,3,4-tetraol; and pharmaceutically acceptable salts, solvates, and hydrates thereof.

In some embodiments, the pharmaceutical agent or the second pharmaceutical agent is a α-glucosidase inhibitor selected from (2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal (chemical structure shown below) and pharmaceutically acceptable salts, solvates, and hydrates thereof:

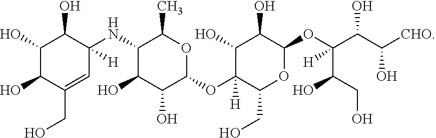 [SMILES: C[C@H]1O[C@H](O[C@H]2[C@H](O)[C@@H](O)[C@@H](O[C@@H]([C@H](O)[C@@H](O)C=O)[C@H](O)CO)O[C@@H]2CO)[C@H](O)[C@@H](O)[C@@H]1N[C@H]1C=C(CO)[C@@H](O)[C@H](O)[C@H]1O]

In some embodiments, the pharmaceutical agent or the second pharmaceutical agent is a α-glucosidase inhibitor selected from (2R,3R,4R,5S)-1-(2-hydroxyethyl)-2-(hydroxymethyl)piperidine-3,4,5-triol (chemical structure shown below) and pharmaceutically acceptable salts, solvates, and hydrates thereof:

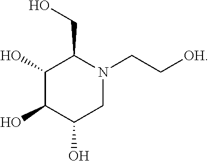 [SMILES: OCCN1C[C@H](O)[C@@H](O)[C@H](O)[C@H]1CO]

In some embodiments, the pharmaceutical agent or the second pharmaceutical agent is a α-glucosidase inhibitor selected from (1S,2S,3R,4S,5S)-5-(1,3-dihydroxypropan-2-ylamino)-1-(hydroxymethyl)cyclohexane-1,2,3,4-tetraol (chemical structure shown below) and pharmaceutically acceptable salts, solvates, and hydrates thereof:

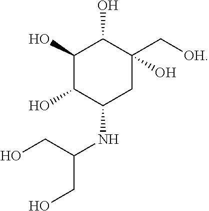 [SMILES: OCC(CO)N[C@H]1C[C@](O)(CO)[C@@H](O)[C@H](O)[C@H]1O]

In some embodiments, the pharmaceutical agent or the second pharmaceutical agent is an alpha-glucosidase inhibitor selected from: acarbose, miglitol, and voglibose. In some embodiments, the pharmaceutical agent or the second pharmaceutical agent is acarbose. In some embodiments, the pharmaceutical agent or the second pharmaceutical agent is miglitol. In some embodiments, the pharmaceutical agent or the second pharmaceutical agent is voglibose.

### Insulin and Insulin Analogues

The term “insulin analogue” refers to the naturally occurring human hormone and insulin receptor ligands (i.e., synthetic insulin analogues). Insulin receptor ligands are structurally different from the natural human hormone, but have substantially the same activity as human insulin in terms of glycemic control. Examples of an insulin analogue include, NPH insulin (also known as Humulin N, Novolin N, NPH Lletin II, and insulin isophane), insulin lispro (28B-L-lysine-29B-L-proline-insulin, wherein insulin is human insulin), insulin aspart (28B-L-aspartic acid-insulin, wherein insulin is human insulin), insulin glulisine (3B-L-lysine-29B-L-glutamic acid-insulin, wherein insulin is human insulin), and insulin analogues known in the art.

NPH insulin is marketed by Eli Lilly and Company under the name Humulin N, and is considered as an intermediate-acting insulin analogue given to help control the blood sugar level of those with diabetes. Insulin lispro is marketed by Eli Lilly and Company under the name Humalog, and is considered a rapid acting insulin analogue. Insulin aspart is marketed by Novo Nordisk and sold as NovoRapid. Insulin aspart is considered a fast acting insulin analogue. Insulin glulisine was developed by Sanofi-Aventis and is sold under the trade name Apidra. Insulin glulisine is considered a rapid acting insulin analogue but shorter duration of action compared to human insulin.

In some embodiments, the pharmaceutical agent or the second pharmaceutical agent is an insulin analogue selected from NPH insulin and pharmaceutically acceptable salts, solvates, and hydrates thereof. In some embodiments, the pharmaceutical agent or the second pharmaceutical agent is an insulin analogue selected from insulin lispro and pharmaceutically acceptable salts, solvates, and hydrates thereof. In some embodiments, the pharmaceutical agent or the second pharmaceutical agent is an insulin analogue selected from insulin aspart and pharmaceutically acceptable salts, solvates, and hydrates thereof. In some embodiments, the pharmaceutical agent or the second pharmaceutical agent is an insulin analogue selected from insulin glulisine and pharmaceutically acceptable salts, solvates, and hydrates thereof.

### Sulfonylureas

The sulfonylureas are drugs which promote secretion of insulin from pancreatic beta cells by transmitting signals of insulin secretion via receptors in the cell membranes. Examples of a sulfonylurea include tolbutamide (Orinase, N-(butylcarbamoyl)-4-methylbenzenesulfonamide); acetohexamide (Dymelor, 4-acetyl-N-(cyclohexylcarbamoyl)benzenesulfonamide); tolazamide (Tolinase, N-(azepan-1-ylcarbamoyl)-4-methylbenzenesulfonamide); chlorpropamide (Diabinese, 4-chloro-N-(propylcarbamoyl)benzenesulfonamide); glipizide (Glucotrol, N-(4-(N-(cyclohexylcarbamoyl)sulfamoyl)phenethyl)-5-methylpyrazine-2-carboxamide); glibenclamide, also known as glyburide (Diabeta, Micronase, Glynase, 5-chloro-N-(4-(N-(cyclohexylcarbamoyl)sulfamoyl)phenethyl)-2-methoxybenzamide); glimepiride (Amaryl, 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide); gliclazide (Diamicron, N-(hexahydrocyclopenta[c]pyrrol-2(1H)-ylcarbamoyl)-4-methylbenzenesulfonamide); and sulfonylureas known in the art.

In some embodiments, the pharmaceutical agent or the second pharmaceutical agent is a sulfonylurea selected from sulfonylureas:

N-(4-(N-(cyclohexylcarbamoyl)sulfamoyl)phenethyl)-5-methylpyrazine-2-carboxamide); 5-chloro-N-(4-(N-(cyclohexylcarbamoyl)sulfamoyl)phenethyl)-2-methoxybenzamide; 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide; and pharmaceutically acceptable salts, solvates, and hydrates thereof.

In some embodiments, the pharmaceutical agent or the second pharmaceutical agent is a sulfonylurea selected from N-(butylcarbamoyl)-4-methylbenzenesulfonamide (chemical structure shown below) and pharmaceutically acceptable salts, solvates, and hydrates thereof:

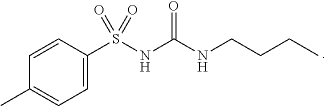 [SMILES: CCCCNC(=O)NS(=O)(=O)c1ccc(C)cc1]

In some embodiments, the pharmaceutical agent or the second pharmaceutical agent is a sulfonylurea selected from 4-acetyl-N-(cyclohexylcarbamoyl)benzenesulfonamide (chemical structure shown below) and pharmaceutically acceptable salts, solvates, and hydrates thereof:

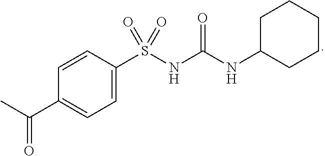 [SMILES: CC(=O)c1ccc(S(=O)(=O)NC(=O)NC2CCCCC2)cc1]

In some embodiments, the pharmaceutical agent or the second pharmaceutical agent is a sulfonylurea selected from N-(azepan-1-ylcarbamoyl)-4-methylbenzenesulfonamide (chemical structure shown below) and pharmaceutically acceptable salts, solvates, and hydrates thereof:

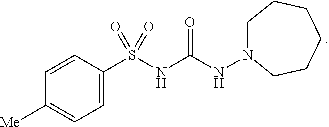 [SMILES: Cc1ccc(S(=O)(=O)NC(=O)NN2CCCCCC2)cc1]

In some embodiments, the pharmaceutical agent or the second pharmaceutical agent is a sulfonylurea selected from 4-chloro-N-(propylcarbamoyl)benzenesulfonamide (chemical structure shown below) and pharmaceutically acceptable salts, solvates, and hydrates thereof:

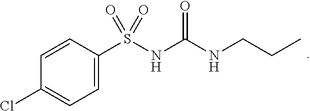 [SMILES: CCCNC(=O)NS(=O)(=O)c1ccc(Cl)cc1]

In some embodiments, the pharmaceutical agent or the second pharmaceutical agent is a sulfonylurea selected from N-(4-(N-(cyclohexylcarbamoyl)sulfamoyl)phenethyl)-5-methylpyrazine-2-carboxamide (chemical structure shown below) and pharmaceutically acceptable salts, solvates, and hydrates thereof:

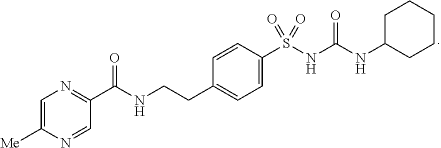 [SMILES: Cc1cnc(C(=O)NCCc2ccc(S(=O)(=O)NC(=O)NC3CCCCC3)cc2)cn1]

In some embodiments, the pharmaceutical agent or the second pharmaceutical agent is a sulfonylurea selected from 5-chloro-N-(4-(N-(cyclohexylcarbamoyl)sulfamoyl)phenethyl)-2-methoxybenzamide (chemical structure shown below) and pharmaceutically acceptable salts, solvates, and hydrates thereof:

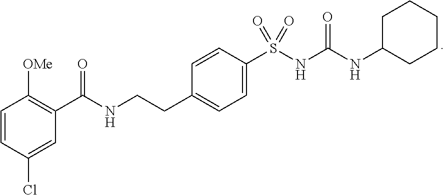 [SMILES: COc1ccc(Cl)cc1C(=O)NCCc1ccc(S(=O)(=O)NC(=O)NC2CCCCC2)cc1]

In some embodiments, the pharmaceutical agent or the second pharmaceutical agent is a sulfonylurea selected from 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide (chemical structure shown below) and pharmaceutically acceptable salts, solvates, and hydrates thereof:

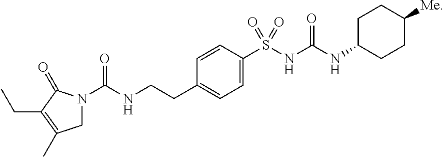 [SMILES: CCC1=C(C)CN(C(=O)NCCc2ccc(S(=O)(=O)NC(=O)N[C@H]3CC[C@H](C)CC3)cc2)C1=O]

In some embodiments, the pharmaceutical agent or the second pharmaceutical agent is a sulfonylurea selected from N-(hexahydrocyclopenta[c]pyrrol-2(1H)-ylcarbamoyl)-4-methylbenzenesulfonamide (chemical structure shown below) and pharmaceutically acceptable salts, solvates, and hydrates thereof:

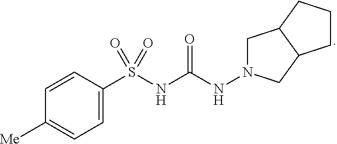 [SMILES: Cc1ccc(S(=O)(=O)NC(=O)NN2CC3CCCC3C2)cc1]

In some embodiments, the pharmaceutical agent or the second pharmaceutical agent is a sulfonylurea selected from the following sulfonylureas and pharmaceutically acceptable salts, solvates, and hydrates thereof: glipizide, glimepiride, and glibenclamide. In some embodiments, the pharmaceutical agent or the second pharmaceutical agent is tolbutamide. In some embodiments, the pharmaceutical agent or the second pharmaceutical agent is acetohexamide. In some embodiments, the pharmaceutical agent or the second pharmaceutical agent is tolazamide. In some embodiments, the pharmaceutical agent or the second pharmaceutical agent is chlorpropamide In some embodiments, the pharmaceutical agent or the second pharmaceutical agent is glipizide. In some embodiments, the pharmaceutical agent or the second pharmaceutical agent is glyburide. In some embodiments, the pharmaceutical agent or the second pharmaceutical agent is glimepiride. In some embodiments, the pharmaceutical agent or the second pharmaceutical agent is gliclazide.

### SGLT2 Inhibitors

Sodium-glucose transporter-2 (SGLT2) inhibitors belong to the class of drugs which inhibit the protein SGLT2 and the reabsorption of glucose in the kidney. The inhibition by SGLT2 inhibitors retard, diminish, or otherwise reduce the amount of glucose that is reabsorbed and therefore is eliminated in the urine. Some representative examples of SGLT2 inhibitors include dapagliflozin ((2S,3R,4R,5S,6R)-2-(4-chloro-3-(4-ethoxybenzyl)phenyl)-6-(hydroxymethyl)tetrahydro-2H-pyran-3,4,5-triol, Bristol-Myers Squibb and AstraZeneca), remogliflozin (ethyl((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate, GlaxoSmithKline), ASP1941 (Kotobuki/Astellas), canagliflozin ((2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol, Johnson & Johnson/Mitsubishi/Tanabe), ISIS 388626 (an antisense oligonucleotide, Isis Pharmaceuticals), sergliflozin (ethyl((2R,3S,4S,5R,6S)-3,4,5-trihydroxy-6-(2-(4-methoxybenzyl)phenoxy)tetrahydro-2H-pyran-2-yl)methyl carbonate, GlaxoSmithKline), AVE2268 ((2R,3S,4S,5R,6S)-2-(hydroxymethyl)-6-(2-(4-methoxybenzyl)thiophen-3-yloxy)tetrahydro-2H-pyran-3,4,5-triol, Sanofi-Aventis), BI10773 (Boehringer Ingelheim), CSG453 (Chugai/Roche), LX4211 (Lexicon), and SGLT2 inhibitors known in the art.

In some embodiments, the pharmaceutical agent or the second pharmaceutical agent is a SGLT2 inhibitor selected from the following SGLT2 inhibitors:

(2S,3R,4R,5S,6R)-2-(4-chloro-3-(4-ethoxybenzyl)phenyl)-6-(hydroxymethyl)tetrahydro-2H-pyran-3,4,5-triol; ethyl((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; ethyl ((2R,3S,4S,5R,6S)-3,4,5-trihydroxy-6-(2-(4-methoxybenzyl)phenoxy)tetrahydro-2H-pyran-2-yl)methyl carbonate; (2R,3S,4S,5R,6S)-2-(hydroxymethyl)-6-(2-(4-methoxybenzyl)thiophen-3-yloxy)tetrahydro-2H-pyran-3,4,5-triol; (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol; and pharmaceutically acceptable salts, solvates, and hydrates thereof.

In some embodiments, the pharmaceutical agent or the second pharmaceutical agent is a sulfonylurea selected from (2S,3R,4R,5S,6R)-2-(4-chloro-3-(4-ethoxybenzyl)phenyl)-6-(hydroxymethyl)tetrahydro-2H-pyran-3,4,5-triol (chemical structure shown below) and pharmaceutically acceptable salts, solvates, and hydrates thereof:

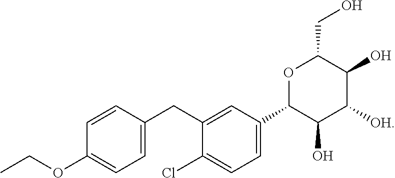 [SMILES: CCOc1ccc(Cc2cc([C@@H]3O[C@H](CO)[C@@H](O)[C@H](O)[C@H]3O)ccc2Cl)cc1]

In some embodiments, the pharmaceutical agent or the second pharmaceutical agent is a sulfonylurea selected from ethyl((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate (chemical structure shown below) and pharmaceutically acceptable salts, solvates, and hydrates thereof:

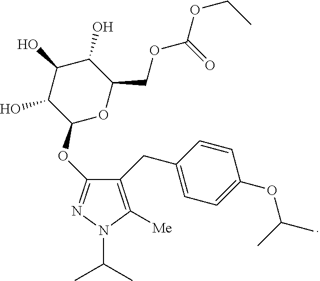 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

In some embodiments, the pharmaceutical agent or the second pharmaceutical agent is a sulfonylurea selected from ethyl((2R,3S,4S,5R,6S)-3,4,5-trihydroxy-6-(2-(4-methoxybenzyl)phenoxy)tetrahydro-2H-pyran-2-yl)methyl carbonate (chemical structure shown below) and pharmaceutically acceptable salts, solvates, and hydrates thereof:

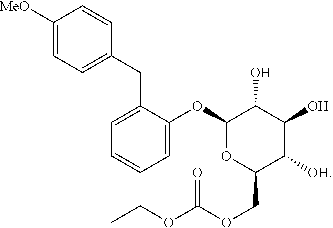 [SMILES: CCOC(=O)OC[C@H]1O[C@@H](Oc2ccccc2Cc2ccc(OC)cc2)[C@H](O)[C@@H](O)[C@@H]1O]

In some embodiments, the pharmaceutical agent or the second pharmaceutical agent is a SGLT2 inhibitor selected from: dapagliflozin, remigliflozin, and sergliflozin. In some embodiments, the pharmaceutical agent or the second pharmaceutical agent is dapagliflozin. In some embodiments, the pharmaceutical agent or the second pharmaceutical agent is remigliflozin. In some embodiments, the pharmaceutical agent or the second pharmaceutical agent is sergliflozin.

Astellas and Kotobuki disclosed a series of SGLT2 inhibitors in international patent publication WO2004/080990. Some embodiments of the present invention include every combination of one or more compounds selected from compounds disclosed in WO2004/080990 and pharmaceutically acceptable salts, solvates, and hydrates thereof.

Aventis disclosed a series of SGLT2 inhibitors in international patent publication WO2004/007517. Some embodiments of the present invention include every combination of one or more compounds selected from compounds disclosed in WO2004/007517 and pharmaceutically acceptable salts, solvates, and hydrates thereof. One such compound is (2R,3S,4S,5R,6S)-2-(hydroxymethyl)-6-(2-(4-methoxybenzyl)thiophen-3-yloxy)tetrahydro-2H-pyran-3,4,5-triol. In some embodiments, the SGLT2 inhibitor is selected from (2R,3S,4S,5R,6S)-2-(hydroxymethyl)-6-(2-(4-methoxybenzyl)thiophen-3-yloxy)tetrahydro-2H-pyran-3,4,5-triol, and pharmaceutically acceptable salts, solvates, and hydrates thereof:

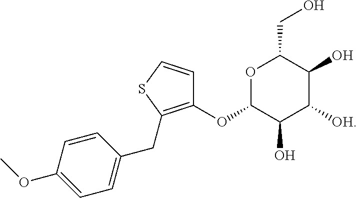 [SMILES: COc1ccc(Cc2sccc2O[C@@H]2O[C@H](CO)[C@@H](O)[C@H](O)[C@H]2O)cc1]

Tanabe disclosed a series of SGLT2 inhibitors in international patent publication WO2005/012326. Some embodiments of the present invention include every combination of one or more compounds selected from compounds disclosed in WO2005/012326 and pharmaceutically acceptable salts, solvates, and hydrates thereof. One such compound is (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol. In some embodiments, the SGLT2 inhibitor is selected from (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol, and pharmaceutically acceptable salts, solvates, and hydrates thereof:

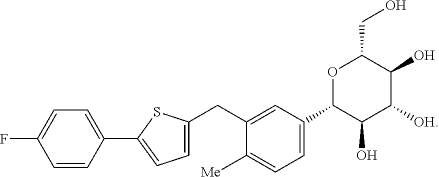 [SMILES: Cc1ccc([C@@H]2O[C@H](CO)[C@@H](O)[C@H](O)[C@H]2O)cc1Cc1ccc(-c2ccc(F)cc2)s1]

Boehringer Ingelheim disclosed a series of SGLT2 inhibitors in international patent publication WO2005/092877. Some embodiments of the present invention include every combination of one or more compounds selected from compounds disclosed in WO2005/092877 and pharmaceutically acceptable salts, solvates, and hydrates thereof.

Chugai disclosed a series of SGLT2 inhibitors in international patent publication WO2006/080421. Some embodiments of the present invention include every combination of one or more compounds selected from compounds disclosed in WO2006/080421 and pharmaceutically acceptable salts, solvates, and hydrates thereof.

Lexicon disclosed a series of SGLT2 inhibitors in international patent publication WO2008/109591. Some embodiments of the present invention include every combination of one or more compounds selected from compounds disclosed in WO2008/109591 and pharmaceutically acceptable salts, solvates, and hydrates thereof.

### Meglitinides

The meglitinides promote secretion of insulin by binding to the pancreatic beta cells in a similar manner as sulfonylureas but at an alternative binding site. Examples of meglitinides include Novo Nordisk's repaglinide (Prandin, (S)-2-ethoxy-4-(2-(3-methyl-1-(2-(piperidin-1-yl)phenyl)butylamino)-2-oxoethyl)benzoic acid), nateglinide (Starlix, (R)-2-((1r,4R)-4-isopropylcyclohexanecarboxamido)-3-phenylpropanoic acid), mitiglinide ((S)-2-benzyl-4-((3aR,7aS)-1H-isoindol-2(3H,3aH,4H,5H,6H,7H,7aH)-yl)-4-oxobutanoic acid), and the like.

In some embodiments, the pharmaceutical agent or the second pharmaceutical agent is a meglitinide selected from the following meglitinides: (S)-2-ethoxy-4-(2-(3-methyl-1-(2-(piperidin-1-yl)phenyl)butylamino)-2-oxoethyl)benzoic acid; (R)-2-((1r,4R)-4-isopropylcyclohexanecarboxamido)-3-phenylpropanoic acid; (S)-2-benzyl-4-((3aR,7aS)-1H-isoindol-2(3H,3aH,4H,5H,6H,7H,7aH)-yl)-4-oxobutanoic acid; and pharmaceutically acceptable salts, solvates, and hydrates thereof.

In some embodiments, the pharmaceutical agent or the second pharmaceutical agent is (S)-2-ethoxy-4-(2-(3-methyl-1-(2-(piperidin-1-yl)phenyl)butylamino)-2-oxoethyl)benzoic acid (chemical structure shown below) and pharmaceutically acceptable salts, solvates, and hydrates thereof:

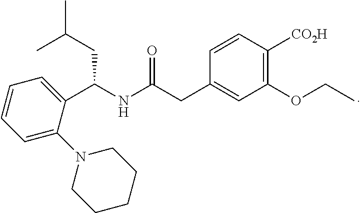 [SMILES: CCOc1cc(CC(=O)N[C@@H](CC(C)C)c2ccccc2N2CCCCC2)ccc1C(=O)O]

In some embodiments, the pharmaceutical agent or the second pharmaceutical agent is a sulfonylurea selected from (R)-2-((1r,4R)-4-isopropylcyclohexanecarboxamido)-3-phenylpropanoic acid (chemical structure shown below) and pharmaceutically acceptable salts, solvates, and hydrates thereof:

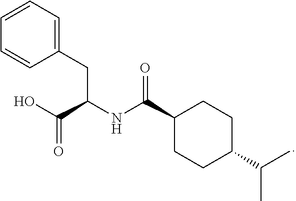 [SMILES: CC(C)[C@H]1CC[C@H](C(=O)N[C@H](Cc2ccccc2)C(=O)O)CC1]

In some embodiments, the pharmaceutical agent or the second pharmaceutical agent is a sulfonylurea selected from (S)-2-benzyl-4-((3aR,7aS)-1H-isoindol-2(3H,3aH,4H,5H,6H,7H,7aH)-yl)-4-oxobutanoic acid (chemical structure shown below) and pharmaceutically acceptable salts, solvates, and hydrates thereof:

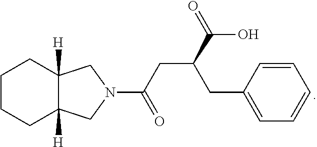 [SMILES: O=C(O)[C@H](CC(=O)N1C[C@H]2CCCC[C@H]2C1)Cc1ccccc1]

In some embodiments, the pharmaceutical agent or the second pharmaceutical agent is a meglitinide selected from the following meglitinides: repaglinide, nateglinide, mitiglinide, and pharmaceutically acceptable salts, solvates, and hydrates thereof. In some embodiments, the pharmaceutical agent or the second pharmaceutical agent is a meglitinide selected from repaglinide and pharmaceutically acceptable salts, solvates, and hydrates thereof. In some embodiments, the pharmaceutical agent or the second pharmaceutical agent is a meglitinide selected from nateglinide and pharmaceutically acceptable salts, solvates, and hydrates thereof. In some embodiments, the pharmaceutical agent or the second pharmaceutical agent is a meglitinide selected from mitiglinide and pharmaceutically acceptable salts, solvates, and hydrates thereof.

### Thiazolidinediones

Thiazolidinediones belong to the class of drugs more commonly known as TZDs. These drugs act by binding to the nuclear receptor peroxisome proliferator-activated receptor gamma (PPARγ) activate transcription of a number of specific genes leading to a decrease in insulin resistance. Examples of thiazolidinediones include rosiglitazone (Avandia, 5-(4-(2-(methyl(pyridin-2-yl)amino)ethoxy)benzyl)thiazolidine-2,4-dione), pioglitazone (Actos, 5-(4-(2-(5-ethylpyridin-2-yl)ethoxy)benzyl)thiazolidine-2,4-dione), troglitazone (Rezulin, 5-(4-((6-hydroxy-2,5,7,8-tetramethylchroman-2-yl)methoxy)benzyl)thiazolidine-2,4-dione), rivoglitazone (5-(4-((6-methoxy-1-methyl-1H-benzo[d]imidazol-2-yl)methoxy)benzyl)thiazolidine-2,4-dione), ciglitazone(5-(4-((1-methylcyclohexyl)methoxy)benzyl)thiazolidine-2,4-dione), and thiazolidinediones known in the art.

In some embodiments, the pharmaceutical agent or the second pharmaceutical agent is a meglitinide selected from: 5-(4-(2-(methyl(pyridin-2-yl)amino)ethoxy)benzyl)thiazolidine-2,4-dione; 5-(4-(2-(5-ethylpyridin-2-yl)ethoxy)benzyl)thiazolidine-2,4-dione; 5-(4-((6-methoxy-1H-benzo[d]imidazol-2-yl)methoxy)benzyl)thiazolidine-2,4-dione; 5-(4-((1-methylcyclohexyl)methoxy)benzyl)thiazolidine-2,4-dione; and pharmaceutically acceptable salts, solvates, and hydrates thereof.

In some embodiments, the pharmaceutical agent or the second pharmaceutical agent is 5-(4-(2-(methyl(pyridin-2-yl)amino)ethoxy)benzyl)thiazolidine-2,4-dione (chemical structure shown below) and pharmaceutically acceptable salts, solvates, and hydrates thereof:

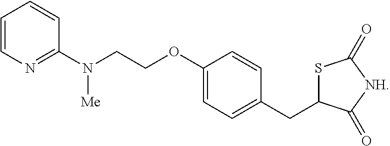 [SMILES: CN(CCOc1ccc(CC2SC(=O)NC2=O)cc1)c1ccccn1]

In some embodiments, the pharmaceutical agent or the second pharmaceutical agent is 5-(4-(2-(5-ethylpyridin-2-yl)ethoxy)benzyl)thiazolidine-2,4-dione (chemical structure shown below) and pharmaceutically acceptable salts, solvates, and hydrates thereof:

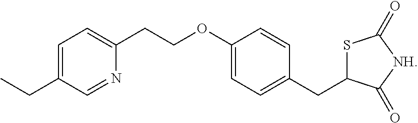 [SMILES: CCc1ccc(CCOc2ccc(CC3SC(=O)NC3=O)cc2)nc1]

In some embodiments, the pharmaceutical agent or the second pharmaceutical agent is 5-(4-((6-hydroxy-2,5,7,8-tetramethylchroman-2-yl)methoxy)benzyl)thiazolidine-2,4-dione (chemical structure shown below) and pharmaceutically acceptable salts, solvates, and hydrates thereof:

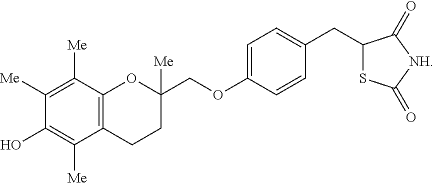 [SMILES: Cc1c(C)c2c(c(C)c1O)CCC(C)(COc1ccc(CC3SC(=O)NC3=O)cc1)O2]

In some embodiments, the pharmaceutical agent or the second pharmaceutical agent is 5-(4-((6-methoxy-1-methyl-1H-benzo[d]imidazol-2-yl)methoxy)benzyl)thiazolidine-2,4-dione (chemical structure shown below) and pharmaceutically acceptable salts, solvates, and hydrates thereof:

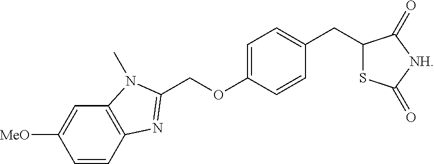 [SMILES: COc1ccc2nc(COc3ccc(CC4SC(=O)NC4=O)cc3)n(C)c2c1]

In some embodiments, the pharmaceutical agent or the second pharmaceutical agent is 5-(4-((1-methylcyclohexyl)methoxy)benzyl)thiazolidine-2,4-dione (chemical structure shown below) and pharmaceutically acceptable salts, solvates, and hydrates thereof:

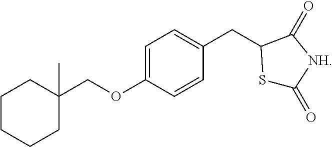 [SMILES: CC1(COc2ccc(CC3SC(=O)NC3=O)cc2)CCCCC1]

In some embodiments, the pharmaceutical agent or the second pharmaceutical agent is a thiazolidinedione selected from rosiglitazone and pharmaceutically acceptable salts, solvates, and hydrates thereof. In some embodiments, the pharmaceutical agent or the second pharmaceutical agent is a thiazolidinedione selected from pioglitazone and pharmaceutically acceptable salts, solvates, and hydrates thereof. In some embodiments, the pharmaceutical agent or the second pharmaceutical agent is a thiazolidinedione selected from troglitazone and pharmaceutically acceptable salts, solvates, and hydrates thereof. In some embodiments, the pharmaceutical agent or the second pharmaceutical agent is a thiazolidinedione selected from rivoglitazone and pharmaceutically acceptable salts, solvates, and hydrates thereof. In some embodiments, the pharmaceutical agent or the second pharmaceutical agent is a thiazolidinedione selected from ciglitazone and pharmaceutically acceptable salts, solvates, and hydrates thereof.

### Anti-Diabetic Peptide Analogues

Anti-diabetic peptide analogues are peptides that promote secretion of insulin by acting as an incretin mimetic, such as, GLP-1 and GIP. Examples of an anti-diabetic peptide analog include, exenatide, liraglutide, taspoglutide, and anti-diabetic peptides analogues know in the art.

In some embodiments, the pharmaceutical agent or the second pharmaceutical agent is an anti-diabetic peptide analogue selected from: exenatide; liraglutide; and taspoglutide. In some embodiments, the pharmaceutical agent or the second pharmaceutical agent is exenatide. In some embodiments, the pharmaceutical agent or the second pharmaceutical agent is liraglutide. In some embodiments, the pharmaceutical agent or the second pharmaceutical agent is taspoglutide.

In some embodiments, the pharmaceutical agent or the second pharmaceutical agent is L-histidylglycyl-L-α-glutamylglycyl-L-threonyl-L-phenylalanyl-L-threonyl-L-seryl-L-α-aspartyl-L-leucyl-L-seryl-L-lysyl-L-glutaminyl-L-methionyl-L-α-glutamyl-L-α-glutamyl-L-α-glutamyl-L-alanyl-L-valyl-L-arginyl-L-leucyl-L-phenylalanyl-L-isoleucyl-L-α-glutamyl-L-tryptophyl-L-leucyl-L-lysyl-L-asparaginylglycylglycyl-L-prolyl-L-seryl-L-serylglycyl-L-alanyl-L-prolyl-L-prolyl-L-prolyl-L-serinamide (i.e., exenatide) and pharmaceutically acceptable salts, solvates, and hydrates thereof.

In some embodiments, the pharmaceutical agent or the second pharmaceutical agent is L-histidyl-L-alanyl-L-α-glutamylglycyl-L-threonyl-L-phenylalanyl-L-threonyl-L-seryl-L-α-aspartyl-L-valyl-L-seryl-L-seryl-L-tyrosyl-L-leucyl-L-α-glutamylglycyl-L-glutaminyl-L-alanyl-L-alanyl-N6-[N-(1-oxohexadecyl)-L-α-glutamyl]-L-lysyl-L-α-glutamyl-L-phenylalanyl-L-isoleucyl-L-alanyl-L-tryptophyl-L-leucyl-L-valyl-L-arginylglycyl-L-arginyl-glycine (liraglutide) and pharmaceutically acceptable salts, solvates, and hydrates thereof.

In some embodiments, the pharmaceutical agent or the second pharmaceutical agent is H₂N-His-2-methyl-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-2-methyl-Ala-Arg-CONH₂ (taspoglutide) and pharmaceutically acceptable salts, solvates, and hydrates thereof.

### Other Utilities

Another object of the present invention relates to radio-labeled compounds of the present invention that would be useful not only in radio-imaging but also in assays, both in vitro and in vivo, for localizing and quantitating GPR119 receptors in tissue samples, including human and for identifying GPR119 receptor ligands by inhibition binding of a radio-labeled compound. It is a further object of this invention to develop novel GPR119 receptor assays of which comprise such radio-labeled compounds.

The present disclosure includes all isotopes of atoms occurring in the present compounds, intermediates, salts and crystalline forms thereof. Isotopes include those atoms having the same atomic number but different mass numbers. One aspect of the present invention includes every combination of one or more atoms in the present compounds, intermediates, salts, and crystalline forms thereof that is replaced with an atom having the same atomic number but a different mass number. One such example is the replacement of an atom that is the most naturally abundant isotope, such as ¹H or ¹²C, found in one the present compounds, intermediates, salts, and crystalline forms thereof, with a different atom that is not the most naturally abundant isotope, such as ²H or ³H (replacing ¹H), or ¹¹C, ¹³C, or ¹⁴C (replacing ¹²C). A compound wherein such a replacement has taken place is commonly referred to as being an isotopically-labeled compound. Isotopic-labeling of the present compounds, intermediates, salts, and crystalline forms thereof can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present compounds, intermediates, salts, and crystalline forms thereof, wherein the naturally occurring distribution of the isotopes in the composition is perturbed. Another aspect of the present invention includes compositions and pharmaceutical compositions comprising compounds as described herein wherein the compound is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Drawings and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the scarcer radio-isotope or nonradioactive isotope.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas: This procedure normally yields high specific activity products and requires halogenated or unsaturated precursors.

B. Reduction with Sodium Borohydride [³H]: This procedure is rather inexpensive and requires precursors containing reducible functional groups such as aldehydes, ketones, lactones, esters and the like.

C. Reduction with Lithium Aluminum Hydride [³H]: This procedure offers products at almost theoretical specific activities. It also requires precursors containing reducible functional groups such as aldehydes, ketones, lactones, esters and the like.

D. Tritium Gas Exposure Labeling: This procedure involves exposing precursors containing exchangeable protons to tritium gas in the presence of a suitable catalyst.

E. N-Methylation using Methyl Iodide [³H]: This procedure is usually employed to prepare O-methyl or N-methyl (3H) products by treating appropriate precursors with high specific activity methyl iodide (3H). This method in general allows for higher specific activity, such as for example, about 70-90 Ci/mmol

Synthetic methods for incorporating activity levels of ¹²⁵I into target molecules include:

A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A represented procedure was reported by Zhu, G-D. and co-workers in J. Org. Chem., 2002, 67, 943-948.

B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.

C. Aryl and heteroaryl bromide exchange with ¹²⁵I: This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm. 2001, 44, S280-S282.

A radiolabeled GPR119 receptor compound of Formula (Ia) can be used in a screening assay to identify/evaluate compounds. In general terms, a newly synthesized or identified compound (i.e., test compound) can be evaluated for its ability to reduce binding of the “radio-labeled compound of Formula (Ia)” to a GPR119 receptor. Accordingly, the ability of a test compound to compete with the “radio-labeled compound of Formula (Ia)” for the binding to a GPR119 receptor directly correlates to its binding affinity.

Certain labeled compounds of the present invention bind to certain GPR119 receptors. In one embodiment the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM and in still yet another embodiment the labeled inhibitor has an IC₅₀ less than about 0.1 μM.

Other uses of the disclosed receptors and methods will become apparent to those skilled in the art based upon, inter alia, a review of this disclosure.

As will be recognized, the steps of the methods of the present invention need not be performed any particular number of times or in any particular sequence. Additional objects, advantages and novel features of this invention will become apparent to those skilled in the art upon examination of the following examples thereof, which are intended to be illustrative and not intended to be limiting.

### EXAMPLES

### Example 1

### Syntheses of Compounds of the Present Invention

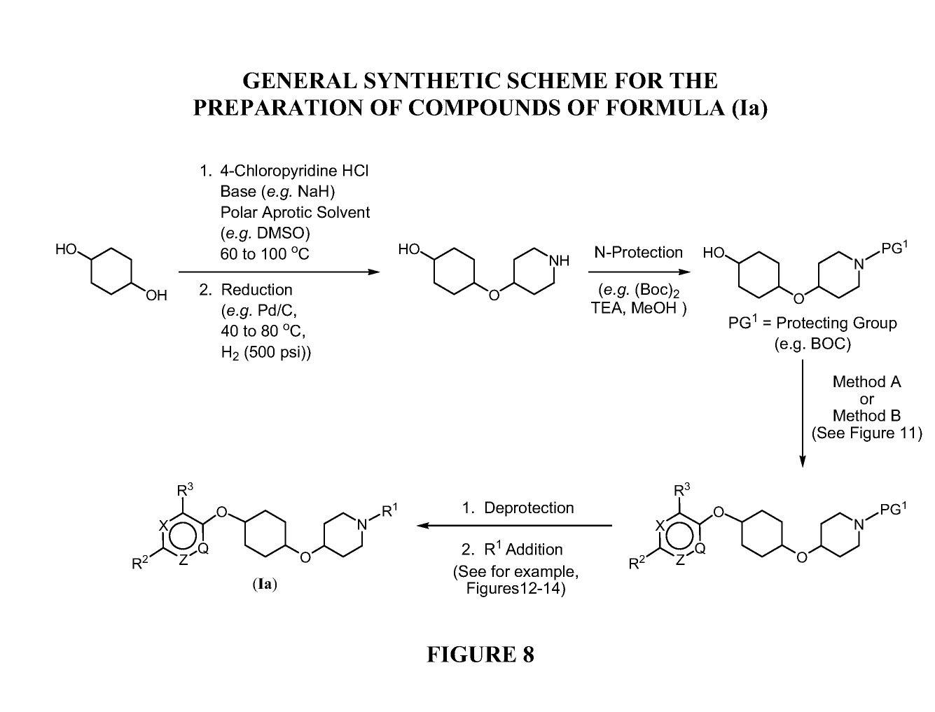

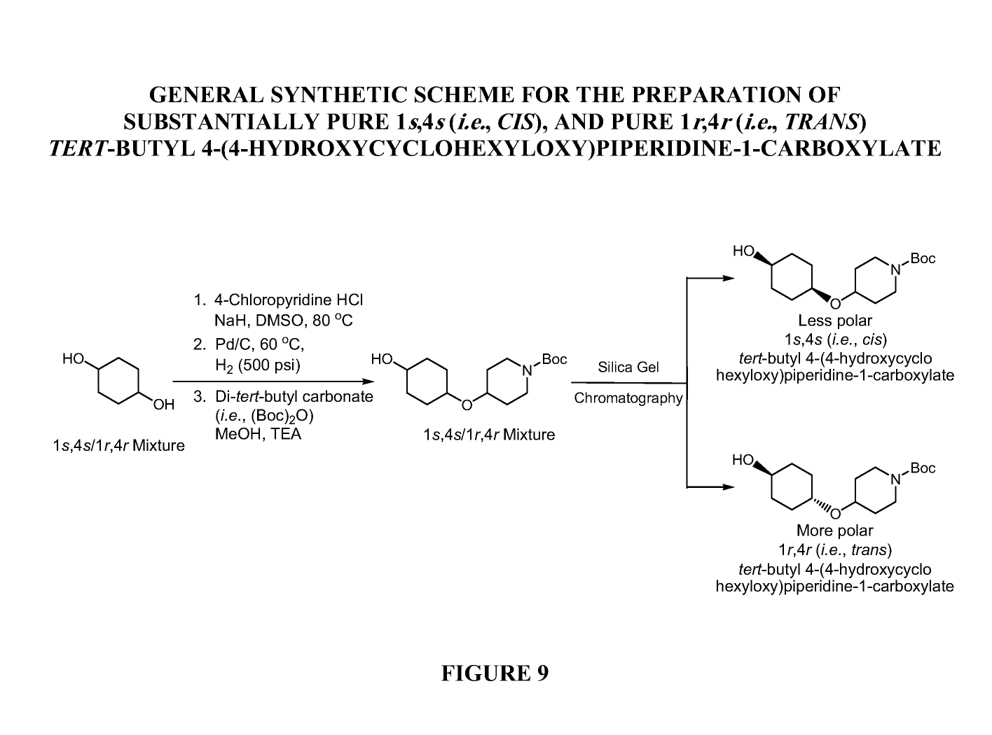

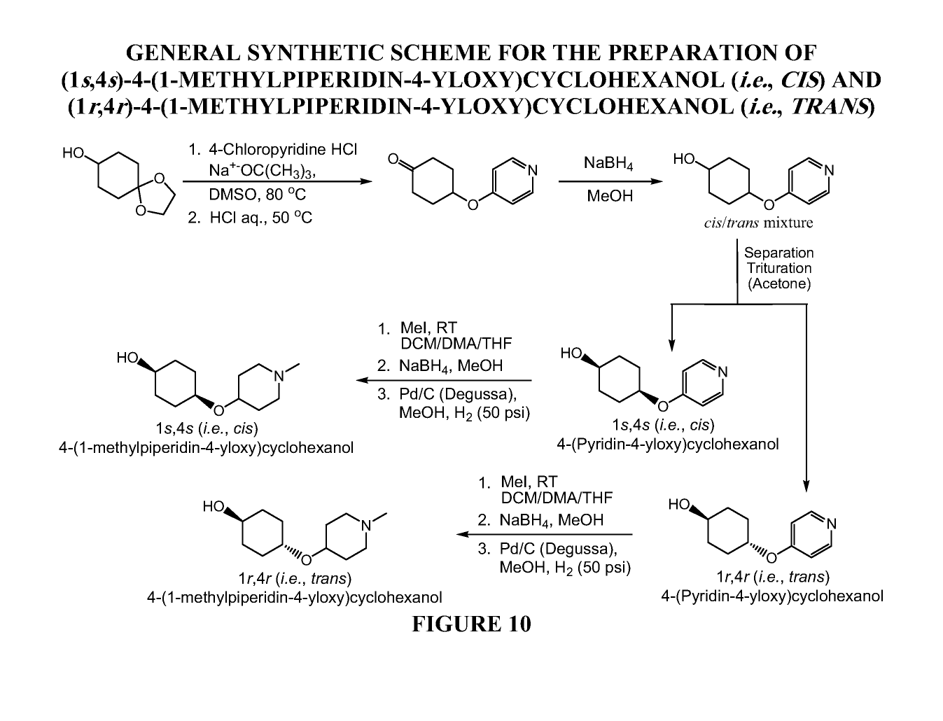

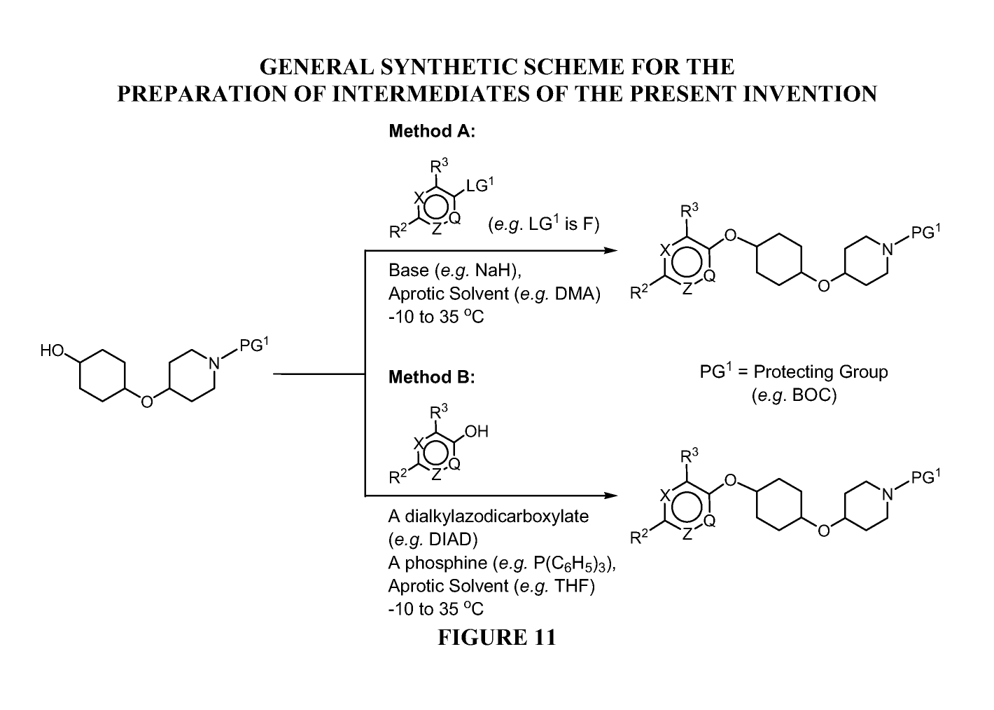

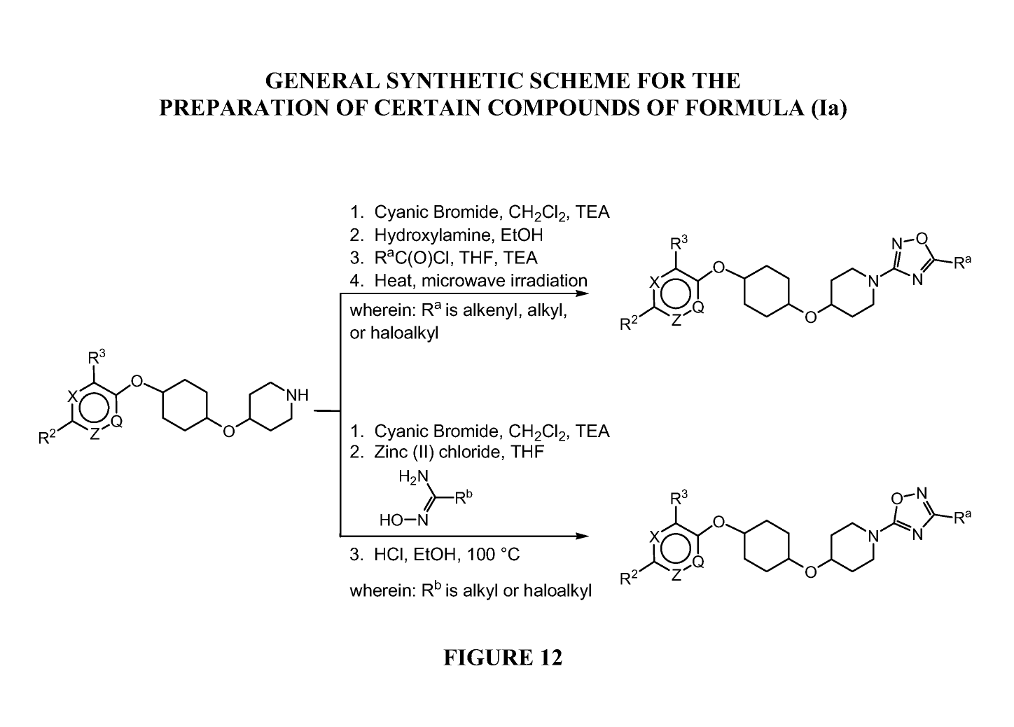

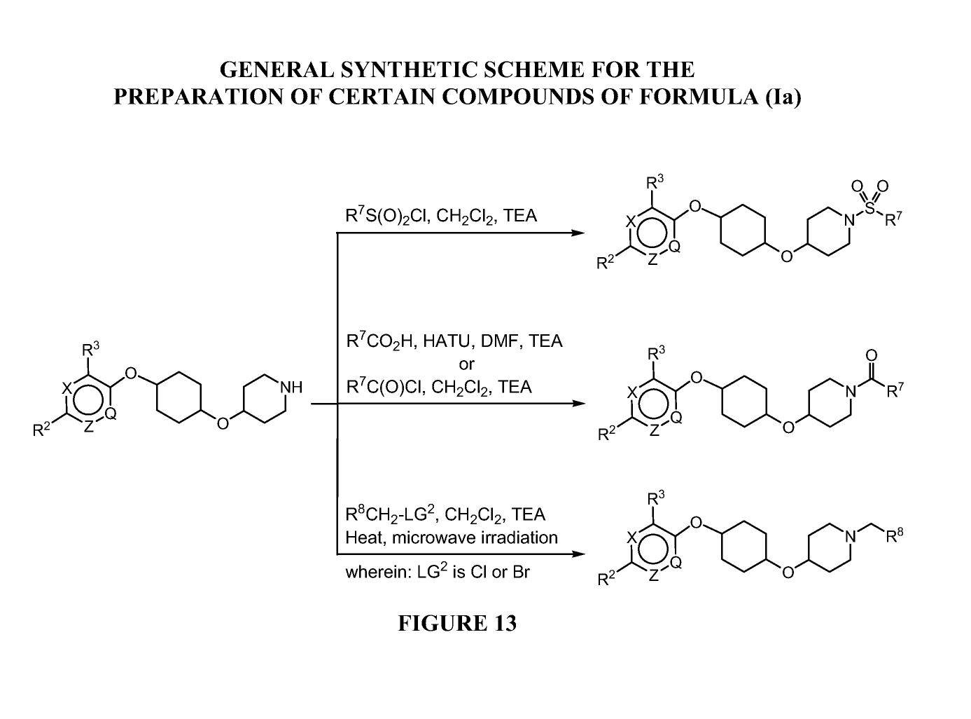

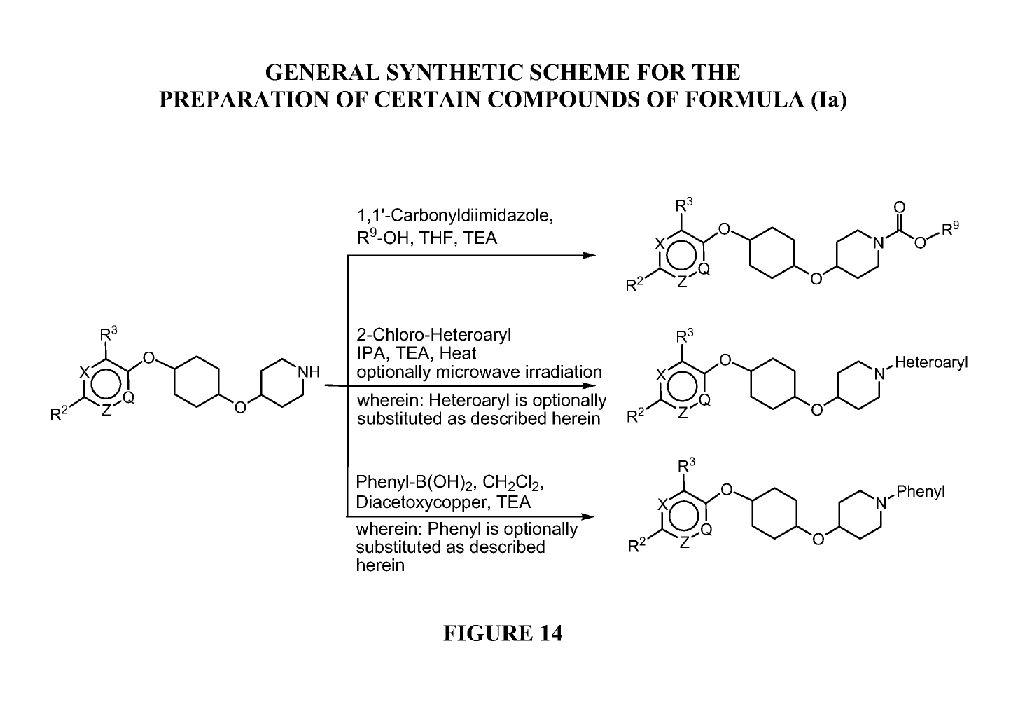

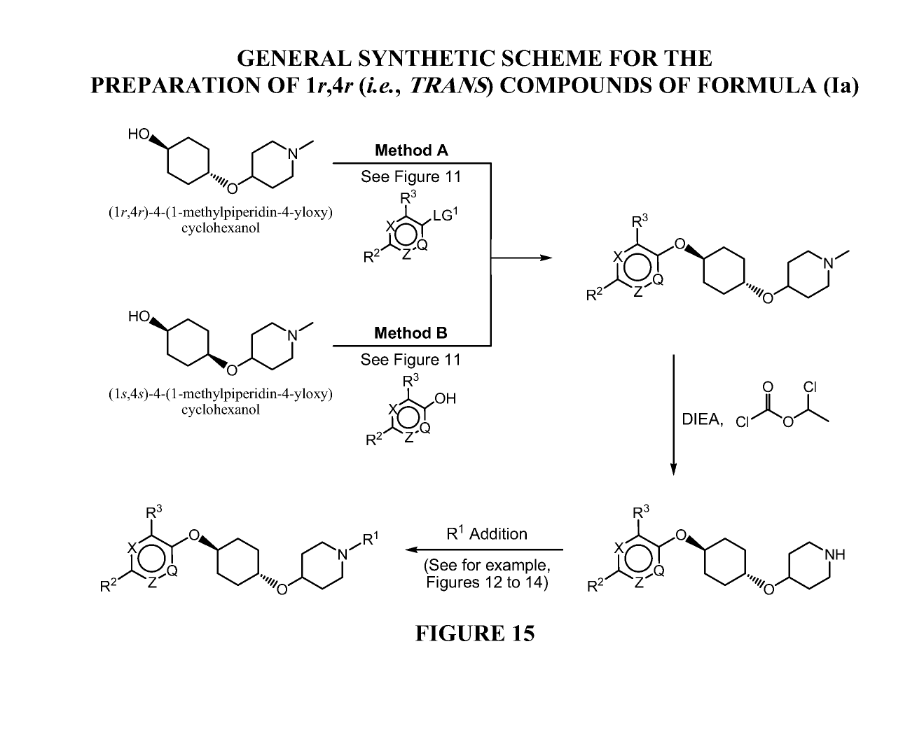

Illustrated syntheses for compounds of the present invention are shown in FIGS. 8 through 15 where the variables have the same definitions as used throughout this disclosure.

The compounds of the invention and their syntheses are further illustrated by the following examples. The following examples are provided to further define the invention without, however, limiting the invention to the particulars of these examples. The compounds described herein, supra and infra, are named according to AutoNom version 2.2, AutoNom 2000, CS ChemDraw Ultra Version 7.0.1, or CS ChemDraw Ultra Version 9.0.7. In certain instances common names are used and it is understood that these common names would be recognized by those skilled in the art.

Chemistry:

Proton nuclear magnetic resonance (¹H NMR) spectra were recorded on a Bruker Avance-400 equipped with a QNP (Quad Nucleus Probe) or a BBI (Broad Band Inverse) and z-gradient. Chemical shifts are given in parts per million (ppm) with the residual solvent signal used as reference. NMR abbreviations are used as follows: s=singlet, d=doublet, dd=doublet of doublets, ddd=doublet of doublet of doublets, dt=doublet of triplets, t=triplet, td=triplet of doublets, tt=triplet of triplets, q=quartet, m=multiplet, bs=broad singlet, bt=broad triplet. Microwave irradiations were carried out using a Smith Synthesizer™ or an Emrys Optimizer™ (Biotage). Thin-layer chromatography (TLC) was performed on silica gel 60 F₂₅₄ (Merck), preparatory thin-layer chromatography (prep TLC) was preformed on PK6F silica gel 60 A 1 mm plates (Whatman) and column chromatography was carried out on a silica gel column using Kieselgel 60, 0.063-0.200 mm (Merck). Evaporation was done under reduced pressure on a Büchi rotary evaporator.

LCMS Spec:

HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: API 150EX with Turbo Ion Spray source, AB/MDS Sciex; Software: Analyst 1.2.

### Example 1.1

### Preparation of tert-Butyl 4-((1s,4s)-4-Hydroxycyclohexyloxy)piperidine-1-carboxylate (Intermediate 1) and tert-Butyl 4-((1r,4r)-4-Hydroxycyclohexyloxy)piperidine-1-carboxylate (Intermediate 2)

### Step A: Preparation of 4-(Pyridin-4-yloxy)cyclohexanol

To a solution of sodium hydride (13.77 g, 344 mmol) in DMSO (250 mL) at room temperature, was added cyclohexane-1,4-diol (20 g, 172 mmol) portion wise. The mixture was stirred at room temperature for 1 h and 4-chloropyridine hydrochloride (25.8 g, 172 mmol) was then added portion wise. The mixture was stirred at room temperature for 2 h until H₂ evolution ceased and then heated at 80° C. for 8 h. The mixture was diluted with icy water and extracted with DCM (6 times). The organic layer was combined and washed with water and brine and dried over MgSO₄. The solvent was removed and the resulting solid was dried under high vacuum at a temperature of 80° C. for 2 h to further remove any remaining DMSO. The solid was then dissolved in minimum amount of 10% MeOH/DCM and purified by silica gel column chromatography (MeOH/EtOAc) to give the title compound (10 g).

### Step B: Preparation of 4-(Piperidin-4-yloxy)cyclohexanol

To a solution of 4-(pyridin-4-yloxy)cyclohexanol (13 g, 67.3 mmol) in anhydrous EtOH (336 mL) in a stainless steel vessel, was added palladium on carbon (12 g, 11.28 mmol). The above mixture was placed under vacuum and charged with H₂ at 500 psi. The reaction vessel was then stirred at 60° C. for 144 h. The palladium was filtered off and the filtrate was concentrated to give the title compound.

### Step C: Preparation of tert-Butyl 4-((1s,4s)-4-Hydroxycyclohexyloxy)piperidine-1-carboxylate (Intermediate 1) and tert-Butyl 4-((1r,4r)-4-Hydroxycyclohexyloxy)piperidine-1-carboxylate (Intermediate 2)

To a solution of 4-(piperidin-4-yloxy)cyclohexanol (9 g, 45.2 mmol) in MeOH (125 mL), was added triethylamine (6.35 mL, 45.2 mmol) followed by di-tert-butyl dicarbonate (24.64 g, 113 mmol). The mixture was heated at 50° C. for 4 h. The solvent was removed and the residue was purified by silica gel column chromatography (40% to 100% EtOAc/Hexane) to give Intermediate 1 (5.6 g) as colorless oil and Intermediate 2 (5.2 g) as a white solid.

Intermediate 1: LCMS m/z=300.2 [M+H]⁺; ¹H NMR (400 MHz, CDCl₃) δ 1.45 (s, 9H), 1.47-1.56 (m, 6H), 1.60-1.70 (m, 3H), 1.70-1.82 (m, 4H), 3.07-3.14 (m, 2H), 3.47-3.56 (m, 2H), 3.71-3.77 (m, 3H).

Intermediate 2: LCMS m/z=300.0 [M+H]⁺; ¹H NMR (400 MHz, CDCl₃) δ 1.30-1.37 (m, 4H), 1.43 (s, 9H), 1.43-1.52 (m, 2H), 1.75-1.79 (m, 2H), 1.93-1.98 (m, 4H), 3.02-3.09 (m, 2H), 3.05-3.40 (m, 1H), 3.51-3.55 (m, 1H), 3.66-3.71 (m, 1H), 3.74-3.80 (m, 2H).

### Example 1.2

### Preparation of tert-Butyl 4-((1r,4r)-4-(4-(Methylsulfonyl)phenoxy)cyclohexyloxy)piperidine-1-carboxylate (Compound 2)

### Method A

To a mixture of NaH (15 mg, 0.376 mmol) in anhydrous DMA (0.3 mL) under N₂ atmosphere at 0° C. was added dropwise Intermediate 2 (75 mg, 0.250 mmol) in DMA (0.5 mL). The reaction was stirred at 0° C. for 20 min, 1-fluoro-4-(methylsulfonyl)benzene (44 mg, 0.250 mmol) in DMA (0.4 mL) was added dropwise and the reaction was allowed to warm up to room temperature, and stirred for 16 h. The reaction was quenched with ice water and extracted with ethylacetate. The organic layer was separated, dried over MgSO₄, and concentrated. The residue was purified by flash silica gel column chromatography to give the title compound (0.10 g) as a white solid. LCMS m/z=454.2 [M+H]⁺; ¹H NMR (400 MHz, CDCl₃) δ 1.45 (s, 9H), 1.47-1.64 (m, 6H), 1.76-1.81 (m, 2H), 1.96-2.00 (m, 2H), 2.08-2.13 (m, 2H), 3.02 (s, 3H), 3.05-3.12 (m, 2H), 3.49-3.58 (m, 2H), 3.74-3.79 (m, 2H), 4.37-4.43 (m, 1H), 6.99 (d, J=9.1 Hz, 2H), 7.84 (d, J=9.1 Hz, 2H).

### Method B

To a solution of 4-(methylsulfonyl)phenol (0.949 g, 5.51 mmol), Intermediate 1 (1.5 g, 5.01 mmol) and triphenylphosphine (1.445 g, 5.51 mmol) in THF (25 mL) at 0° C., was added dropwise F (1.071 mL, 5.51 mmol). The mixture was allowed to warm up to room temperature during 1 h and stirred overnight. After removal of the solvent the residue was purified by silica gel column chromatography (EtOAc/Hexane) to give the title compound (590 mg) as a white solid.

### Example 1.3

### Preparation of tert-Butyl 4-((1r,4r)-4-(5-(Methylsulfonyl)pyridin-2-yloxy)cyclohexyloxy)piperidine-1-carboxylate (Compound 7)

The title compound was prepared in a similar manner as described in Example 1.2 (Method A) using Intermediate 2 and 2-bromo-5-(methylsulfonyl)pyridine. LCMS m/z=455.3 [M+H]⁺; ¹H NMR (400 MHz, CDCl₃) δ 1.46 (s, 9H), 1.46-1.61 (m, 6H), 1.77-1.82 (m, 2H), 1.96-2.00 (m, 2H), 2.12-2.17 (m, 2H), 3.06 (s, 3H), 3.15-3.12 (m, 2H), 3.47-3.59 (m, 2H), 3.75-3.81 (m, 2H), 5.13-5.19 (m, 1H), 6.78-6.80 (d, J=8.8 Hz, 1H), 7.99-8.02 (dd, J=8.8, 2.8 Hz, 1H), 8.69-8.70 (d, J=2.8 Hz, 1H).

### Example 1.4

### Preparation of tert-Butyl 4-((1s,4s)-4-(5-(Methylsulfonyl)pyridin-2-yloxy)cyclohexyloxy)piperidine-1-carboxylate (Compound 11)

The title compound was prepared in a similar manner as described in Example 1.2 (Method A) using Intermediate 1 and 2-bromo-5-(methylsulfonyl)pyridine. LCMS m/z=455.4 [M+H]⁺; ¹H NMR (400 MHz, CDCl₃) δ 1.46 (s, 9H), 1.50-1.59 (m, 2H), 1.66-1.85 (m, 7H), 1.89-2.04 (m, 3H), 3.08 (s, 3H), 3.13-3.20 (m, 2H), 3.55-3.63 (m, 2H), 3.71-3.77 (m, 2H), 5.20-5.25 (m, 1H), 6.82-6.84 (d, J=8.7 Hz, 1H), 8.00-8.03 (dd, J=8.7, 2.6 Hz, 1H), 8.69-8.70 (d, J=2.6 Hz, 1H).

### Example 1.5

### Preparation of tert-Butyl 4-((1s,4s)-4-(2-Fluoro-4-(methylsulfonyl)phenoxy)cyclohexyloxy)piperidine-1-carboxylate (Compound 6)

The title compound was prepared in a similar manner as described in Example 1.2 (Method A) using Intermediate 1 and 1,2-difluoro-4-(methylsulfonyl)benzene. LCMS m/z=471.9 [M+H]⁺; ¹H NMR (400 MHz, CDCl₃) δ 1.46 (s, 9H), 1.49-1.56 (m, 2H), 1.61-1.70 (m, 2H), 1.71-1.88 (m, 6H), 1.98-2.07 (m, 2H), 3.05 (s, 3H), 3.09-3.15 (m, 2H), 3.53-3.60 (m, 2H), 3.72-3.78 (m, 2H), 4.47-4.52 (m, 1H), 7.09-7.13 (t, J=8.5 Hz, 1H), 7.62-7.67 (m, 2H).

### Example 1.6

### Preparation of tert-Butyl 4-((1r,4r)-4-(2-Fluoro-4-(methylsulfonyl)phenoxy)cyclohexyloxy)piperidine-1-carboxylate (Compound 5)

The title compound was prepared in a similar manner as described in Example 1.2 (Method A) using Intermediate 2 and 1,2-difluoro-4-(methylsulfonyl)benzene. LCMS m/z=472.4 [M+H]⁺; ¹H NMR (400 MHz, CDCl₃) δ 1.46 (s, 9H), 1.46-1.54 (m, 4H), 1.61-1.70 (m, 2H), 1.75-1.82 (m, 2H), 1.96-2.03 (m, 2H), 2.08-2.15 (m, 2H), 3.04 (s, 3H), 3.06-3.13 (m, 2H), 3.52-3.58 (m, 2H), 3.74-3.80 (m, 2H), 4.43-4.49 (m, 1H), 7.07-7.11 (t, J=8.8 Hz, 1H), 7.63-7.68 (m, 2H).

### Example 1.7

### Preparation of tert-Butyl 4-((1r,4r)-4-(6-Bromopyridazin-3-yloxy)cyclohexyloxy)piperidine-1-carboxylate (Compound 20)

The title compound was prepared in a similar manner as described in Example 1.2 (Method A) using Intermediate 2 and 3,6-dibromo-pyridazine. LCMS m/z=456.1 [M+H]⁺; ¹H NMR (400 MHz, CDCl₃) δ 1.45 (s, 9H), 1.47-1.61 (m, 6H), 1.76-1.81 (m, 2H), 1.95-1.99 (m, 2H), 2.19-2.24 (m, 2H), 3.05-3.11 (m, 2H), 3.45-3.59 (m, 2H), 3.74-3.80 (m, 2H), 5.22-5.29 (m, 1H), 6.78-6.80 (d, J=9.2 Hz, 1H), 7.44-7.46 (d, J=9.2 Hz, 1H).

### Example 1.8

### Preparation of tert-Butyl 4-((1r,4r)-4-(6-(Methylsulfonyl)pyridazin-3-yloxy)cyclohexyloxy)piperidine-1-carboxylate (Compound 24)

A mixture of tert-butyl 4-((1r,4r)-4-(6-bromopyridazin-3-yloxy)cyclohexyloxy)piperidine-1-carboxylate (0.070 g, 0.153 mmol), sodium methanesulfinate (0.028 g, 0.275 mmol), copper (I) trifluoromethanesulfonate benzene complex (6.95 mg, 0.014 mmol) and N1,N2-dimethylethane-1,2-diamine (3.30 μl, 0.031 mmol) in DMSO (0.9 mL) was heated at 110° C. for 2.5 h. The reaction mixture was diluted with water and extracted with DCM (3×25 mL). The combined organic phases were dried over Na₂SO₄ and concentrated. The residue was purified by flash silica gel column chromatography to give the title compound (0.045 g) as white solid. LCMS m/z=456.2 [M+H]⁺; ¹H NMR (400 MHz, CDCl₃) δ 1.46 (s, 9H), 1.48-1.57 (m, 4H), 1.60-1.69 (m, 2H), 1.77-1.82 (m, 2H), 1.97-2.02 (m, 2H), 2.23-2.28 (m, 2H), 3.05-3.12 (m, 2H), 3.39 (s, 3H), 3.49-3.60 (m, 2H), 3.75-3.81 (m, 2H), 5.39-5.45 (m, 1H), 7.10-7.12 (d, J=9.2 Hz, 1H), 8.01-8.03 (d, J=9.2 Hz, 1H).

### Example 1.9

### Preparation of tert-Butyl 4-((1r,4r)-4-(3-Fluoro-4-(methylsulfonyl)phenoxy)cyclohexyloxy)piperidine-1-carboxylate (Compound 21)

The title compound was prepared in a similar manner as described in Example 1.2 (Method A) using Intermediate 2 and 2,4-difluoro-1-(methylsulfonyl)benzene. LCMS m/z=472.0 [M+H]⁺; ¹H NMR (400 MHz, CDCl₃) δ 1.46 (s, 9H), 1.48-1.66 (m, 6H), 1.77-1.81 (m, 2H), 1.94-1.99 (m, 2H), 2.07-2.12 (m, 2H), 3.06-3.13 (m, 2H), 3.18 (s, 3H), 3.50-3.58 (m, 2H), 3.74-3.80 (m, 2H), 4.35-4.41 (m, 1H), 6.69-6.72 (dd, J=12.0, 2.2 Hz, 1H), 6.76-6.79 (dd, J=8.8, 2.4 Hz, 1H), 7.83 (t, J=8.6 Hz, 1H).

### Example 1.10

### Preparation of tert-Butyl 4-((1r,4r)-4-(5-Fluoro-2-(methylsulfonyl)phenoxy)cyclohexyloxy)piperidine-1-carboxylate (Compound 22)

The title compound was obtained as a side product from Example 1.9. LCMS m/z=471.9 [M+H]⁺; ¹H NMR (400 MHz, CDCl₃) δ 1.46 (s, 9H), 1.48-1.56 (m, 4H), 1.71-1.81 (m, 4H), 1.98-2.04 (m, 2H), 2.10-2.16 (m, 2H), 3.07-3.14 (m, 2H), 3.18 (s, 3H), 3.51-3.57 (m, 1H), 3.59-3.64 (m, 1H), 3.73-3.79 (m, 2H), 4.51-4.56 (m, 1H), 6.71-6.78 (m, 2H), 7.95-7.99 (m, 1H).

### Example 1.11

### Preparation of 5-Isopropyl-3-(4-((1r,4r)-4-(4-(Methylsulfonyl)phenoxy)cyclohexyloxy)piperidin-1-yl)-1,2,4-oxadiazole (Compound 37)

### Step A: Preparation of 4-((1r,4r)-4-(4-(Methylsulfonyl)phenoxy)cyclohexyloxy)piperidine Hydrochloride

To a solution of tert-butyl 4-((1r,4r)-4-(4-(methylsulfonyl)phenoxy)cyclohexyloxy)piperidine-1-carboxylate (3.27 g, 7.21 mmol) in DCM (25 mL) was added 4M HCl in dioxane (4.51 mL, 18.02 mmol) at room temperature. The mixture was stirred at room temperature for 3 h. The resulting white precipitate was filtered and washed with ether to give the title compound (2.66 g).

### Step B: Preparation of 4-((1r,4r)-4-(4-(Methylsulfonyl)phenoxy)cyclohexyloxy)piperidine-1-carbonitrile

To a solution of triethylamine (0.284 mL, 2.052 mmol) in DCM (8 mL) was added 4-((1r,4r)-4-(4-(methylsulfonyl)phenoxy)cyclohexyloxy)piperidine hydrochloride (100 mg, 0.256 mmol). The reaction mixture was cooled to 0° C. and slowly added a solution of cyanic bromide (32.6 mg, 0.308 mmol) in DCM (1 mL). After 20 minutes of stirring at 0° C., the ice bath was removed and the reaction was stirred at room temperature for 2 h. The reaction mixture was then concentrated and filtered through a short silica cartridge to give the title compound (60 mg) as a white solid.

### Step C: Preparation of a Mixture of N-Hydroxy-4-((1r,4r)-4-(4-(methylsulfonyl)phenoxy)cyclohexyloxy)piperidine-1-carboximidamide and 4-((1r,4r)-4-(4-(Methylsulfonyl)phenoxy)cyclohexyloxy)piperidine

To a solution of 4-((1r,4r)-4-(4-(methylsulfonyl)phenoxy)cyclohexyloxy)piperidine-1-carbonitrile (200 mg, 0.528 mmol) in ethanol (1049 μL, 17.97 mmol), was added hydroxylamine (50 wt % in H₂O, 70.5 μL, 1.057 mmol). The reaction mixture was heated at 65° C. for 18 h. The solvent was evaporated in to give the title compounds (200 mg) as an oil which is used without further purification.

### Step D: Preparation of 5-Isopropyl-3-(4-((1r,4r)-4-(4-(methylsulfonyl)phenoxy)cyclohexyloxy)piperidin-1-yl)-1,2,4-oxadiazole

The mixture of N-hydroxy-4-((1r,4r)-4-(4-(methylsulfonyl)phenoxy)cyclohexyloxy)piperidine-1-carboximidamide and 4-((1r,4r)-4-(4-(methylsulfonyl)phenoxy)cyclohexyloxy)piperidine (100 mg, 0.243 mmol) from Step C and triethylamine (29.5 mg, 0.292 mmol) was dissolved in tetrahydrofuran (2 mL, 0.243 mmol). Isobutyryl chloride (28.5 mg, 0.267 mmol) was added slowly until the starting material was consumed. The mixture was then heated under microwave irradiation at 120° C. for 1 h. The mixture was concentrated and purified by silica gel column chromatography to give the title compound (15 mg). LCMS m/z=464.2 [M+H]⁺; ¹H NMR (400 MHz, CDCl₃) δ 1.31-1.32 (d, J=7.0 Hz, 6H), 1.42-1.54 (m, 2H), 1.55-1.65 (m, 4H), 1.83-1.90 (m, 2H), 1.93-2.00 (m, 2H), 2.05-2.12 (m, 2H), 2.99 (s, 3H), 3.00-3.07 (m, 1H), 3.12-3.18 (m, 2H), 3.50-3.61 (m, 2H), 3.72-3.78 (m, 2H), 4.35-4.41 (m, 1H), 6.95-6.97 (d, J=8.8 Hz, 2H), 7.80-7.82 (d, J=8.8 Hz, 2H).

### Example 1.12

### Preparation of 2-Methyl-1-(4-((1r,4r)-4-(4-(Methylsulfonyl)phenoxy)cyclohexyloxy)piperidin-1-yl)propan-1-one (Compound 40)

The title compound was isolated from Example 1.11, Step D during silica gel column chromatography purification. LCMS m/z=424.3 [M+H]⁺; ¹H NMR (400 MHz, CDCl₃) δ 1.13-1.15 (dd, J=6.8, 1.5 Hz, 6H), 1.17-1.26 (m, 2H), 1.47-1.66 (m, 4H), 1.82-1.87 (m, 2H), 1.99-2.04 (m, 2H), 2.11-2.16 (m, 2H), 2.80-2.86 (m, 1H), 3.04 (s, 3H), 3.26-3.33 (m, 2H), 3.53-3.59 (m, 1H), 3.63-3.69 (m, 1H), 3.82-3.92 (m, 2H), 4.41-4.46 (m, 1H), 7.00-7.02 (d, J=8.8 Hz, 2H), 7.85-7.87 (d, J=8.8 Hz, 2H).

### Example 1.13

### Preparation of 3-tert-Butyl-5-(4-((1r,4r)-4-(4-(Methylsulfonyl)phenoxy)cyclohexyloxy)piperidin-1-yl)-1,2,4-oxadiazole (Compound 26)

To a solution of 4-((1r,4r)-4-(4-(methylsulfonyl)phenoxy)cyclohexyloxy)piperidine-1-carbonitrile (30 mg, 0.08 mmol) and N′-hydroxypivalimidamide (13.8 mg, 0.12 mmol) in DCM (3.5 mL) was added 0.5 M zinc(II) chloride in THF (0.476 mL, 0.24 mmol). The mixture was stirred at room temperature for 1 h until complete disappearance of the starting material. The solvent was removed under vacuum. To the residue was added 1.25 N hydrogen chloride in EtOH (0.64 mL, 0.79 mmol). The mixture was heated at 100° C. for 1 h, cooled down to room temperature, diluted with EtOAc, and washed with water. The organic layer was separated, dried over MgSO₄ and concentrated. The residue was purified by silica gel column chromatography to give the title compound (15 mg) as a white solid. LCMS m/z=478.2 [M+H]⁺; ¹H NMR (400 MHz, CDCl₃) δ 1.23 (s, 9H), 1.38-1.48 (m, 2H), 1.50-1.64 (m, 4H), 1.77-1.85 (m, 2H), 1.89-1.95 (m, 2H), 2.01-2.08 (m, 2H), 2.95 (s, 3H), 3.32-3.38 (m, 2H), 3.45-3.51 (m, 1H), 3.56-3.62 (m, 1H), 3.73-3.79 (m, 2H), 4.32-4.38 (m, 1H), 6.91-6.93 (d, J=8.8 Hz, 2H), 7.76-7.78 (d, J=8.8 Hz, 2H).

### Example 1.14

### Preparation of 3-(2-Fluoropropan-2-yl)-5-(4-((1r,4r)-4-(5-(Methylsulfonyl)pyridin-2-yloxy)cyclohexyloxy)piperidin-1-yl)-1,2,4-oxadiazole (Compound 18)

The title compound was prepared in a similar manner as described in Example 1.13 using 4-((1r,4r)-4-(5-(methylsulfonyl)pyridin-2-yloxy)cyclohexyloxy)piperidine-1-carbonitrile and 2-fluoro-N′-hydroxy-2-methylpropanimidamide. LCMS m/z=483.1 [M+H]⁺; ¹H NMR (400 MHz, CDCl₃) δ 1.48-1.63 (m, 4H), 1.67-1.74 (m, 2H), 1.71 (s, 3H), 1.76 (s, 3H), 1.86-1.92 (m, 2H), 1.97-2.01 (m, 2H), 2.12-2.17 (m, 2H), 3.07 (s, 3H), 3.48-3.55 (m, 3H), 3.68-3.73 (m, 1H), 3.82-3.88 (m, 2H), 5.14-5.19 (m, 1H), 6.79-6.81 (d, J=9.0 Hz, 1H), 8.00-8.03 (dd, J=9.0, 2.7 Hz, 1H), 8.70-8.71 (d, J=2.7 Hz, 1H).

### Example 1.15

### Preparation of 5-(4-((1r,4r)-4-(2-Fluoro-4-(methylsulfonyl)phenoxy)cyclohexyloxy)piperidin-1-yl)-3-(2-fluoropropan-2-yl)-1,2,4-oxadiazole (Compound 16)

The title compound was prepared in a similar manner as described in Example 1.13 using 4-((1r,4r)-4-(2-fluoro-4-(methylsulfonyl)phenoxy)cyclohexyloxy)piperidine-1-carbonitrile and 2-fluoro-N′-hydroxy-2-methylpropanimidamide. LCMS m/z=500.3 [M+H]⁺; ¹H NMR (400 MHz, CDCl₃) δ 1.47-1.59 (m, 2H), 1.63-1.73 (m, 4H), 1.71 (s, 3H), 1.76 (s, 3H), 1.85-1.92 (m, 2H), 1.97-2.04 (m, 2H), 2.08-2.15 (m, 2H), 3.04 (s, 3H), 3.47-3.53 (m, 2H), 3.55-3.60 (m, 1H), 3.66-3.71 (m, 1H), 3.82-3.88 (m, 2H), 4.45-4.51 (m, 1H), 7.07-7.11 (t, J=8.3 Hz, 1H), 7.63-7.68 (m, 2H).

### Example 1.16

### Preparation of 3-tert-Butyl-5-(4-((1r,4r)-4-(2-Fluoro-4-(methylsulfonyl)phenoxy)cyclohexyloxy)piperidin-1-yl)-1,2,4-oxadiazole (Compound 25)

The title compound was prepared in a similar manner as described in Example 1.13 using 4-((1r,4r)-4-(2-fluoro-4-(methylsulfonyl)phenoxy)cyclohexyloxy)piperidine-1-carbonitrile and N′-hydroxypivalimidamide. LCMS m/z=496.2 [M+H]⁺; ¹H NMR (400 MHz, CDCl₃) δ 1.24 (s, 9H), 1.39-1.48 (m, 2H), 1.56-1.65 (m, 4H), 1.78-1.84 (m, 2H), 1.90-1.97 (m, 2H), 2.01-2.08 (m, 2H), 2.97 (s, 3H), 3.32-3.39 (m, 2H), 3.47-3.53 (m, 1H), 3.56-3.62 (m, 1H), 3.73-3.79 (m, 2H), 4.38-4.44 (m, 1H), 7.00-7.04 (t, J=8.5 Hz, 1H), 7.56-7.61 (m, 2H).

### Example 1.17

### Preparation of 3-(2-Fluoropropan-2-yl)-5-(4-((1r,4r)-4-(4-(Methylsulfonyl)phenoxy)cyclohexyloxy)piperidin-1-yl)-1,2,4-oxadiazole (Compound 27)

The title compound was prepared in a similar manner as described in Example 1.13 using 4-((1r,4r)-4-(4-(methylsulfonyl)phenoxy)cyclohexyloxy)piperidine-1-carbonitrile and 2-fluoro-N′-hydroxy-2-methylpropanimidamide. LCMS m/z=482.2 [M+H]⁺; ¹H NMR (400 MHz, CDCl₃) δ 1.49-1.76 (m, 6H), 1.73 (s, 3H), 1.79 (s, 3H), 1.88-1.95 (m, 2H), 1.98-2.04 (m, 2H), 2.10-2.17 (m, 2H), 3.05 (s, 3H), 3.50-3.61 (m, 3H), 3.69-3.74 (m, 1H), 3.84-3.91 (m, 2H), 4.42-4.48 (m, 1H), 7.01-7.03 (d, J=8.8 Hz, 2H), 7.86-7.88 (d, J=8.8 Hz, 2H).

### Example 1.18

### Preparation of 5-Fluoro-2-(4-((1r,4r)-4-(4-(Methylsulfonyl)phenoxy)cyclohexyloxy)piperidin-1-yl)pyridine (Compound 52)

A solution of 2,5-difluoropyridine (10.62 mg, 0.092 mmol), triethylamine (23.36 mg, 0.231 mmol) and 4-((1r,4r)-4-(4-(methylsulfonyl)phenoxy)cyclohexyloxy)piperidine hydrochloride (18 mg, 0.046 mmol) in 2-propanol (1 mL) was heated at 120° C. for 10 h. The reaction mixture was cooled, diluted with water (10 mL) and extracted with DCM. The organic layer was separated, dried over MgSO₄ and concentrated. The residue was purified by silica gel column chromatography to give the title compound (11 mg) as a white solid. LCMS m/z=449.1 [M+H]⁺; ¹H NMR (400 MHz, CDCl₃) δ 1.31-1.54 (m, 6H), 1.75-1.81 (m, 2H), 1.84-1.91 (m, 2H), 1.96-2.01 (m, 2H), 2.89 (s, 3H), 2.98-3.45 (m, 2H), 3.40-3.52 (m, 2H), 3.74-3.79 (m, 2H), 4.24-4.30 (m, 1H), 6.48-6.51 (dd, J=9.2, 3.5 Hz, 1H), 6.85-6.87 (d, J=8.8 Hz, 2H), 7.07-7.12 (m, 1H), 7.70-7.72 (d, J=8.8 Hz, 2H), 7.90 (d, J=3.2 Hz, 1H).

### Example 1.19

### Preparation of 5-Methyl-2-(4-((1r,4r)-4-(4-(Methylsulfonyl)phenoxy)cyclohexyloxy)piperidin-1-yl)pyrimidine (Compound 38)

The title compound was prepared in a similar manner as described in Example 1.18 using 4-((1r,4r)-4-(4-(methylsulfonyl)phenoxy)cyclohexyloxy)piperidine hydrochloride and 2-chloro-5-methylpyrimidine. LCMS m/z=446.2 [M+H]⁺; ¹H NMR (400 MHz, CDCl₃) δ 1.48-1.67 (m, 6H), 1.88-1.95 (m, 2H), 2.00-2.07 (m, 2H), 2.12-2.17 (m, 2H), 2.13 (s, 3H), 3.05 (s, 3H), 3.31-3.38 (m, 2H), 3.57-3.63 (m, 1H), 3.64-3.70 (m, 1H), 4.29-4.34 (m, 2H), 4.40-4.46 (m, 1H), 7.01-7.03 (d, J=8.8 Hz, 2H), 7.86-7.88 (d, J=8.8 Hz, 2H), 8.17 (s, 2H).

### Example 1.20

### Preparation of 5-Methoxy-2-(4-((1r,4r)-4-(4-(Methylsulfonyl)phenoxy)cyclohexyloxy)piperidin-1-yl)pyrimidine (Compound 70)

The title compound was prepared in a similar manner as described in Example 1.18 using 4-((1r,4r)-4-(4-(methylsulfonyl)phenoxy)cyclohexyloxy)piperidine hydrochloride and 2-chloro-5-methoxypyrimidine. LCMS m/z=462.4 [M+H]⁺; ¹H NMR (400 MHz, CDCl₃) δ 1.45-1.64 (m, 6H), 1.86-1.92 (m, 2H), 1.97-2.04 (m, 2H), 2.09-2.15 (m, 2H), 3.02 (s, 3H), 3.26-3.32 (m, 2H), 3.54-3.60 (m, 1H), 3.60-3.67 (m, 1H), 3.80 (s, 3H), 4.22-4.27 (m, 2H), 4.38-4.44 (m, 1H), 6.98-7.01 (d, J=8.8 Hz, 2H), 7.83-7.85 (d, J=8.8 Hz, 2H), 8.19 (s, 2H).

### Example 1.21

### Preparation of 3-Ethoxy-6-(4-((1r,4r)-4-(4-(Methylsulfonyl)phenoxy)cyclohexyloxy)piperidin-1-yl)pyridazine (Compound 51)

3-Chloro-6-(4-((1r,4r)-4-(4-(methylsulfonyl)phenoxy)cyclohexyloxy)piperidin-1-yl)pyridazine was prepared in a similar manner as described in Example 1.18 using 4-((1r,4r)-4-(4-(methylsulfonyl)phenoxy)cyclohexyloxy)piperidine hydrochloride and 3,6-dichloropyridazine.

A mixture of 3-chloro-6-(4-((1r,4r)-4-(4-(methylsulfonyl)phenoxy)cyclohexyloxy)piperidin-1-yl)pyridazine (5 mg, 10.73 μmol), sodium ethoxide (0.73 mg, 10.73 μmol) in EtOH (1 mL) was heated under microwave irradiation at 150° C. for 8 h. The solvent was removed under vacuum and the residue was purified by silica gel column chromatography to give the title compound (3 mg). LCMS m/z=476.3 [M+H]⁺; ¹H NMR (400 MHz, CDCl₃) δ 1.34 (t, J=7.2 Hz, 3H), 1.38-1.63 (m, 6H), 1.83-1.89 (m, 2H), 1.90-1.97 (m, 2H), 2.02-2.07 (m, 2H), 3.00 (s, 3H), 3.12-3.19 (m, 2H), 3.47-3.53 (m, 1H), 3.55-3.60 (m, 1H), 3.81-3.87 (m, 2H), 4.31-4.37 (m, 1H), 4.36-4.41 (q, J=7.2 Hz, 2H), 6.72-6.74 (d, J=9.6 Hz, 1H), 6.91-9.94 (d, J=9.0 Hz, 2H), 6.95-6.97 (d, J=9.6 Hz, 1H), 7.76-7.78 (d, J=9.0 Hz, 2H).

### Example 1.22

### Preparation of Isopropyl 4-((1r,4r)-4-(5-(Methylsulfonyl)pyridin-2-yloxy)cyclohexyloxy)piperidine-1-carboxylate (Compound 12)

A mixture of di(1H-imidazol-1-yl)methanone (32.4 mg, 0.20 mmol) and propan-2-ol (12.0 mg, 0.20 mmol) in THF (1 mL) was stirred at room temperature for 1 h. Triethylamine (50.5 mg, 0.50 mmol) and 5-(methylsulfonyl)-2-((1r,4r)-4-(piperidin-4-yloxy)cyclohexyloxy)pyridine hydrochloride (78 mg, 0.20 mmol) were then added and the mixture was heated at 150° C. for 2 h under microwave irradiation. The solvent was removed and the residue was purified by HPLC to give the title compound (35 mg) as white powder. LCMS m/z=441.4 [M+H]⁺; ¹H NMR (400 MHz, CDCl₃) δ 1.24-1.26 (d, J=6.3 Hz, 6H), 1.47-1.62 (m, 6H), 1.77-1.84 (m, 2H), 1.96-2.01 (m, 2H), 2.12-2.18 (m, 2H), 3.07 (s, 3H), 3.16-3.23 (m, 2H), 3.48-3.54 (m, 1H), 3.58-3.64 (m, 1H), 3.75-3.81 (m, 2H), 4.88-4.97 (m, 1H), 5.12-5.18 (m, 1H), 6.79-6.81 (d, J=8.7 Hz, 1H), 8.00-8.03 (dd, J=8.7, 2.6 Hz, 1H), 8.70-8.71 (d, J=2.6 Hz, 1H).

### Example 1.23

### Preparation of Isopropyl 4-((1r,4r)-4-(2-Fluoro-4-(methylsulfonyl)phenoxy)cyclohexyloxy)piperidine-1-carboxylate (Compound 13)

The title compound was prepared in a similar manner as described in Example 1.22 using 4-((1r,4r)-4-(2-fluoro-4-(methylsulfonyl)phenoxy)cyclohexyloxy)-1-methylpiperidine hydrochloride and propan-2-ol. LCMS m/z=458.2 [M+H]⁺; ¹H NMR (400 MHz, CDCl₃) δ 1.24-1.25 (d, J=6.3 Hz, 6H), 1.45-1.57 (m, 4H), 1.62-1.70 (m, 2H), 1.76-1.83 (m, 2H), 1.96-2.03 (m, 2H), 2.08-2.15 (m, 2H), 3.04 (s, 3H), 3.15-3.21 (m, 2H), 3.52-3.61 (m, 2H), 3.75-3.81 (m, 2H), 4.43-4.49 (m, 1H), 4.88-4.95 (m, 1H), 7.07-7.11 (t, J=8.8 Hz, 1H), 7.63-7.68 (m, 2H).

### Example 1.24

### Preparation of tert-Butyl 4-((1r,4r)-4-(4-(2-Cyanoethyl)phenoxy)cyclohexyloxy)piperidine-1-carboxylate (Compound 31)

The title compound was prepared in a similar manner as described in Example 1.2 (Method B) using Intermediate 1 and 3-(4-hydroxyphenyl)-propanenitrile. LCMS m/z=429.4 [M+H]⁺; ¹H NMR (400 MHz, CDCl₃) δ 1.30 (s, 9H), 1.23-1.42 (m, 6H), 1.60-1.65 (m, 2H), 1.80-1.85 (m, 2H), 1.91-1.96 (m, 2H), 2.40-2.43 (t, J=7.3 Hz, 2H), 2.71-2.74 (t, J=7.3 Hz, 2H), 2.89-2.95 (m, 2H), 3.30-3.42 (m, 2H), 3.59-3.64 (m, 2H), 4.05-4.12 (m, 1H), 6.68-6.70 (d, J=8.7 Hz, 2H), 6.96-6.98 (d, J=8.7 Hz, 2H).

### Example 1.25

### Preparation of tert-Butyl 4-((1s,4s)-4-(4-(Methylsulfonyl)phenoxy)cyclohexyloxy)piperidine-1-carboxylate (Compound 1)

The title compound was prepared in a similar manner as described in Example 1.2 (Method A) using Intermediate 1. LCMS m/z=454.2 [M+H]⁺; ¹H NMR (400 MHz, CDCl₃) δ 1.46 (s, 9H), 1.49-1.52 (m, 2H), 1.63-1.83 (m, 4H), 1.78-1.84 (m, 4H), 1.97-2.02 (m, 2H), 3.03 (s, 3H), 3.08-3.14 (m, 2H), 3.53-3.58 (m, 2H), 3.73-3.78 (m, 2H), 4.44-4.46 (m, 1H), 7.00 (d, J=12.0 Hz, 2H), 7.84 (d, J=8.0 Hz, 2H).

### Example 1.26

### Preparation of 3-Isopropyl-5-(4-((1s,4s)-4-(4-(Methylsulfonyl)phenoxy)cyclohexyloxy)piperidin-1-yl)-1,2,4-oxadiazole (Compound 3)

The title compound was prepared in a similar manner as described in Example 1.13 using 4-((1s,4s)-4-(4-(methylsulfonyl)phenoxy)cyclohexyloxy)piperidine-1-carbonitrile and N′-hydroxyisobutyrimidamide. LCMS m/z=464.3 [M+H]⁺; ¹H NMR (400 MHz, CDCl₃) δ 1.29 (d, J=4 Hz, 6H), 1.64-1.76 (m, 6H), 1.79-1.90 (m, 4H), 1.97-2.02 (m, 2H), 2.85-2.91 (m, 1H), 3.03 (s, 3H), 3.44-3.50 (m, 2H), 3.54-3.58 (m, 1H), 3.67-3.70 (m, 1H), 3.80-3.86 (m, 2H), 4.44-4.47 (m, 1H), 7.00 (d, J=12.0 Hz, 2H), 7.84 (d, J=8.0 Hz, 2H).

### Example 1.27

### Preparation of 3-Isopropyl-5-(4-((1r,4r)-4-(4-(Methylsulfonyl)phenoxy)cyclohexyloxy)piperidin-1-yl)-1,2,4-oxadiazole (Compound 4)

The title compound was prepared in a similar manner as described in Example 1.13 using 4-((1r,4r)-4-(4-(methylsulfonyl)phenoxy)cyclohexyloxy)piperidine-1-carbonitrile and N′-hydroxyisobutyrimidamide. LCMS m/z=464.3 [M+H]⁺; ¹H NMR (400 MHz, CDCl₃) δ 1.29 (d, J=8 Hz, 6H), 1.49-1.73 (m, 6H), 1.86-1.91 (m, 2H), 1.99-2.02 (m, 2H), 2.09-2.11 (m, 2H), 2.88-2.93 (m, 1H), 3.03 (s, 3H), 3.45-3.51 (m, 2H), 3.54-3.57 (m, 1H), 3.66-3.70 (m, 1H), 3.81-3.87 (m, 2H), 4.40-4.44 (m, 1H), 6.99 (d, J=8.0 Hz, 2H), 7.84 (d, J=8.0 Hz, 2H).

### Example 1.28

### Preparation of 5-Ethyl-2-(4-((1r,4r)-4-(2-Fluoro-4-(methylsulfonyl)phenoxy)cyclohexyloxy)piperidin-1-yl)pyrimidine (Compound 8)

The title compound was prepared in a similar manner as described in Example 1.2 (Method A) using (1r,4r)-4-(1-(5-ethylpyrimidin-2-yl)piperidin-4-yloxy)cyclohexanol and 1,2-difluoro-4-(methylsulfonyl)benzene. LCMS m/z=478.4 [M+H]⁺; ¹H NMR (400 MHz, CDCl₃) δ 1.19 (t, J=8 Hz, 3H), 1.50-1.68 (m, 6H), 1.87-1.91 (m, 2H), 2.01-2.07 (m, 2H), 2.10-2.14 (m, 2H), 2.47 (q, J=8 Hz, 2H), 3.04 (s, 3H), 3.35-3.43 (m, 1H), 3.58-3.62 (m, 1H), 3.62-3.69 (m, 1H), 4.24-4.28 (m, 2H), 4.45-4.49 (m, 1H), 7.09 (t, J=8 Hz, 1H), 7.65 (d, J=8 Hz, 1H), 7.67 (d, J=8 Hz, 1H), 8.19 (s, 2H).

### Example 1.29

### Preparation of 5-Ethyl-2-(4-((1r,4r)-4-(5-(Methylsulfonyl)pyridin-2-yloxy)cyclohexyloxy)piperidin-1-yl)pyrimidine (Compound 9)

The title compound was prepared in a similar manner as described in Example 1.2 (Method A) using (1r,4r)-4-(1-(5-ethylpyrimidin-2-yl)piperidin-4-yloxy)cyclohexanol and 2-bromo-5-(methylsulfonyl)pyridine. LCMS m/z=461.4 [M+H]⁺; ¹H NMR (400 MHz, CDCl₃) δ 1.19 (t, J=8 Hz, 3H), 1.48-1.62 (m, 6H), 1.87-1.93 (m, 2H), 1.99-2.02 (m, 2H), 2.14-2.17 (m, 2H), 2.47 (q, J=8 Hz, 2H), 3.06 (s, 3H), 3.33-3.40 (m, 2H), 3.52-3.57 (m, 1H), 3.65-3.69 (m, 1H), 4.27-4.31 (m, 2H), 5.15-5.19 (m, 1H), 6.79 (d, J=8 Hz, 1H), 8.00 (d, J=8 Hz, 1H), 8.70 (s, 1H).

### Example 1.30

### Preparation of 5-Ethyl-2-(4-((1s,4s)-4-(2-Fluoro-4-(methylsulfonyl)phenoxy)cyclohexyloxy)piperidin-1-yl)pyrimidine (Compound 10)

The title compound was prepared in a similar manner as described in Example 1.2 (Method A) using (1s,4s)-4-(1-(5-ethylpyrimidin-2-yl)piperidin-4-yloxy)cyclohexanol and 1,2-difluoro-4-(methylsulfonyl)benzene. LCMS m/z=478.4 [M+H]⁺; ¹H NMR (400 MHz, CDCl₃) δ 1.19 (t, J=8 Hz, 3H), 1.60-1.71 (m, 6H), 1.74-1.78 (m, 2H), 1.87-1.92 (m, 4H), 2.03-2.07 (m, 2H), 2.47 (q, J=8 Hz, 2H), 3.04 (s, 3H), 3.35-3.43 (m, 1H), 3.58-3.62 (m, 1H), 3.63-3.69 (m, 1H), 4.24-4.28 (m, 2H), 4.46-4.51 (m, 1H), 7.10 (t, J=8 Hz, 1H), 7.64 (d, J=8 Hz, 2H), 7.66 (d, J=8 Hz, 1H), 8.18 (s, 2H).

### Example 1.31

### Preparation of 5-((4-((1r,4r)-4-(2-Fluoro-4-(methylsulfonyl)phenoxy)cyclohexyloxy)piperidin-1-yl)methyl)-3-isopropyl-1,2,4-oxadiazole (Compound 14)

A mixture 4-((1r,4r)-4-(2-fluoro-4-(methylsulfonyl)phenoxy)cyclohexyloxy)piperidine hydrochloride (60 mg, 0.147 mmol), 5-(chloromethyl)-3-isopropyl-1,2,4-oxadiazole (26 mg, 0.162 mmol), and triethylamine (0.021 mL, 0.147 mmol) in 2-propanol (2.4 mL) was heated under microwave irradiation at 180° C. for 30 minutes. The mixture was concentrated in vacuo. The residue was dissolved in DCM and purified by silica gel column chromatography (EtOAc/Hexanes) to give the title compound (41 mg) as white solid. LCMS m/z=496.4 [M+H]⁺; ¹H NMR (400 MHz, CDCl₃) δ 1.35 (d, J=8 Hz, 6H), 1.46-1.51 (m, 2H), 1.60-1.70 (m, 4H), 1.84-1.87 (m, 2H), 1.96-2.01 (m, 2H), 2.09-2.11 (m, 2H), 2.35-2.39 (m, 2H), 2.81-2.84 (m, 2H), 3.04 (s, 3H), 3.08-3.13 (m, 1H), 3.42-3.44 (m, 1H), 3.49-3.52 (m, 1H), 3.82 (s, 2H), 4.43-4.46 (m, 1H), 7.08 (t, J=8 Hz, 1H), 7.64 (d, J=8.0 Hz, 1H), 7.66 (d, J=8.0 Hz, 1H).

### Example 1.32

### Preparation of 3-Isopropyl-5-((4-((1r,4r)-4-(5-(Methylsulfonyl)pyridin-2-yloxy)cyclohexyloxy)piperidin-1-yl)methyl)-1,2,4-oxadiazole (Compound 15)

The title compound was prepared in a similar manner as described in Example 1.31 using 5-(methylsulfonyl)-2-((1r,4r)-4-(piperidin-4-yloxy)cyclohexyloxy)pyridine hydrochloride. LCMS m/z=479.4 [M+H]⁺; ¹H NMR (400 MHz, CDCl₃) δ 1.35 (d, J=8 Hz, 6H), 1.47-1.53 (m, 4H), 1.64-1.70 (m, 2H), 1.85-1.88 (m, 2H), 1.95-1.98 (m, 2H), 2.11-2.14 (m, 2H), 2.34-2.39 (m, 2H), 2.81-2.86 (m, 2H), 3.06 (s, 3H), 3.08-3.13 (m, 1H), 3.41-3.48 (m, 2H), 3.82 (s, 2H), 5.13-5.17 (m, 1H), 6.78 (d, J=8 Hz, 1H), 8.00 (d, J=8.0 Hz, 1H), 8.69 (s, 1H).

### Example 1.33

### Preparation of tert-Butyl 4-((1r,4r)-4-(5-(Methylsulfonyl)pyrazin-2-yloxy)cyclohexyloxy)piperidine-1-carboxylate (Compound 17)

A mixture of tert-butyl 4-((1r,4r)-4-(5-bromopyrazin-2-yloxy)cyclohexyloxy)piperidine-1-carboxylate (70 mg, 0.153 mmol), sodium methanesulfinate (28 mg, 0.275 mmol), copper (I) trifluoromethanesulfonate benzene complex (6.95 mg, 0.014 mmol) and N₁,N₂-dimethylethane-1,2-diamine (2.70 mg, 0.031 mmol) in DMSO (0.9 mL) was heated at 110° C. for 2.5 h. The reaction mixture was diluted with water extracted with DCM (3×25 mL). The combined organic extracts were dried over Na₂SO₄, filtered and concentrated. The residue was purified by silica gel column chromatography (EtOAc/Hex) to give the title compound (45 mg) as a white solid. LCMS m/z=456.2 [M+H]⁺; ¹H NMR (400 MHz, CDCl₃) δ 1.46 (s, 9H), 1.48-1.62 (m, 6H), 1.77-1.81 (m, 2H), 1.97-2.01 (m, 2H), 2.13-2.17 (m, 2H), 3.06-3.12 (m, 2H), 3.18 (s, 3H), 3.51-3.58 (m, 2H), 3.75-3.80 (m, 2H), 5.16-5.20 (m, 1H), 8.19 (s, 1H), 8.79 (s, 1H).

### Example 1.34

### Preparation of tert-Butyl 4-((1r,4r)-4-(5-(Dimethylcarbamoyl)-6-methylpyridin-2-yloxy)cyclohexyloxy)piperidine-1-carboxylate (Compound 19)

The title compound was prepared in a similar manner as described in Example 1.2 (Method A) using Intermediate 2 and 6-fluoro-N,N,2-trimethylnicotinamide. LCMS m/z=462.4 [M+H]⁺; ¹H NMR (400 MHz, CDCl₃) δ 1.46 (s, 9H), 1.49-1.57 (m, 6H), 1.77-1.80 (m, 2H), 1.96-1.99 (m, 2H), 2.11-2.14 (m, 2H), 2.39 (s, 3H), 2.88 (s, 3H), 3.12 (s, 3H), 3.04-3.10 (m, 2H), 3.47-3.50 (m, 1H), 3.54-3.57 (m, 1H), 3.77-3.80 (m, 2H), 5.01-5.06 (m, 1H), 6.51 (d, J=8 Hz, 1H), 7.35 (d, J=8 Hz, 1H).

### Example 1.35

### Preparation of tert-Butyl 4-((1r,4r)-4-(5-(1H-1,2,4-Triazol-1-yl)pyrazin-2-yloxy)cyclohexyloxy)piperidine-1-carboxylate (Compound 29)

tert-Butyl 4-((1r,4r)-4-(5-bromopyrazin-2-yloxy)cyclohexyloxy)piperidine-1-carboxylate (125 mg, 0.274 mmol), 1H-1,2,4-triazole (38 mg, 0.548 mmol), copper(I) iodide (13 mg, 0.068 mmol), potassium phosphate (128 mg, 0.603 mmol) and N¹,N²-dimethylethane-1,2-diamine (0.016 mL, 0.151 mmol) were combined in DMF (2.0 mL). N₂ was bubbled through the mixture for 5 minutes and then the reaction mixture was stirred under N₂ at 125° C. for 16 h. The reaction mixture was added DCM and washed with water. The DCM layer was separated, dried over Na₂SO₄, and concentrated. The residue was purified by silica gel column chromatography (EtOAc/Hexane) to give the title compound (85 mg) as white solid. LCMS m/z=445.4 [M+H]⁺; ¹H NMR (400 MHz, CDCl₃) δ 1.46 (s, 9H), 1.49-1.58 (m, 6H), 1.77-1.82 (m, 2H), 1.99-2.02 (m, 2H), 2.14-2.18 (m, 2H), 3.05-3.12 (m, 2H), 3.49-3.59 (m, 2H), 3.75-3.81 (m, 2H), 5.06-5.10 (m, 1H), 7.99 (s, 1H), 8.11 (s, 1H), 8.70 (s, 1H), 8.98 (s, 1H).

### Example 1.36

### Preparation of Isopropyl 4-((1r,4r)-4-(5-(Methylsulfonyl)pyrazin-2-yloxy)cyclohexyloxy)piperidine-1-carboxylate (Compound 28)

### Step A: Preparation of tert-Butyl 4-((1r,4r)-4-(5-Bromopyrazin-2-yloxy)cyclohexyloxy)piperidine-1-carboxylate

To a solution of 5-bromopyrazin-2-ol (2.38 g, 13.63 mmol), Intermediate 1 (3.4 g, 11.36 mmol) and triphenylphosphine (3.57 g, 13.63 mmol) in THF (68 mL) at 0° C. was added dropwise DIAD (2.65 mL, 13.63 mmol). The reaction was stirred at 0° C. for 30 minutes and then stirred at room temperature for 18 h. The solvent was removed in vacuo. The residue was added DCM and washed with water. The organic layer was separated, dried over Na₂SO₄, filtered and concentrated. The residue was purified by silica gel flash silica gel column chromatography (EtOAc/Hex) to give the title compound (1.95 g) as white solid. LCMS m/z=458.2 [M+H]⁺; ¹H NMR (400 MHz, CDCl₃) δ 1.46 (s, 9H), 1.49-1.57 (m, 6H), 1.77-1.79 (m, 2H), 1.96-1.99 (m, 2H), 2.09-2.12 (m, 2H), 3.05-3.11 (m, 2H), 3.48-3.56 (m, 2H), 3.76-3.79 (m, 2H), 4.95-4.99 (m, 1H), 7.94 (s, 1H), 8.15 (s, 1H).

### Step B: Preparation of tert-Butyl 4-((1r,4r)-4-(5-(Methylsulfonyl)pyrazin-2-yloxy)cyclohexyloxy)piperidine-1-carboxylate

The title compound was prepared in a similar manner as described in Example 1.33.

### Step C: Preparation of Isopropyl 4-((1r,4r)-4-(5-(Methylsulfonyl)pyrazin-2-yloxy)cyclohexyloxy)piperidine-1-carboxylate

To a solution of tert-butyl 4-((1r,4r)-4-(5-(methylsulfonyl)pyrazin-2-yloxy)cyclohexyloxy)piperidine-1-carboxylate (260 mg, 0.571 mmol) in DCM (2.2 mL) was added hydrogen chloride (4 M in dioxane, 0.35 mL, 1.427 mmol) at room temperature. The mixture was stirred at room temperature for 5 h. The solvent was removed in vacuo. The residue was washed with ether to give 2-(methylsulfonyl)-5-((1r,4r)-4-(piperidin-4-yloxy)cyclohexyloxy)pyrazine hydrochloride (220 mg).

To a mixture of 2-(methylsulfonyl)-5-((1r,4r)-4-(piperidin-4-yloxy)cyclohexyloxy)pyrazine hydrochloride (125 mg, 0.319 mmol) in anhydrous DMF (2.5 mL) cooled to 0° C. was added triethylamine (0.265 mL, 1.914 mmol). The mixture was stirred at 0° C. for 10 minutes. Isopropyl chloroformate (1 M in toluene) was added dropwise over 5 min at 0° C. The reaction was stirred at 0° C. for 20 minutes and then at room temperature for 2 h. The mixture was diluted with DCM and washed with water. The aqueous layer was extracted with DCM (2×50 mL). The combined organic extracts were washed with water and brine, dried over Na₂SO₄, and concentrated. The residue was purified by silica gel flash silica gel column chromatography (EtOAc/Hexane) to give the title compound (65 mg) as white solid. LCMS m/z=442.2 [M+H]⁺; ¹H NMR (400 MHz, CDCl₃) δ 1.24 (d, J=8 Hz, 6H), 1.48-1.62 (m, 6H), 1.78-1.81 (m, 2H), 1.97-2.01 (m, 2H), 2.12-2.17 (m, 2H), 3.11-3.17 (m, 2H), 3.18 (s, 3H), 3.52-3.59 (m, 2H), 3.79-3.82 (m, 2H), 4.88-4.94 (m, 1H), 5.16-5.20 (m, 1H), 8.19 (s, 1H), 8.78 (s, 1H).

### Example 1.37

### Preparation of 3-(2-Fluoropropan-2-yl)-5-(4-((1r,4r)-4-(5-(Methylsulfonyl)pyrazin-2-yloxy)cyclohexyloxy)piperidin-1-yl)-1,2,4-oxadiazole (Compound 30)

The title compound was prepared in a similar manner as described in Example 1.13 using 4-((1r,4r)-4-(5-(methylsulfonyl)pyrazin-2-yloxy)cyclohexyloxy)piperidine-1-carbonitrile and 2-fluoro-N′-hydroxy-2-methylpropanimidamide. LCMS m/z=484.3 [M+H]⁺; ¹H NMR (400 MHz, CDCl₃) δ 1.71 (s, 3H), 1.76 (s, 3H), 1.58-1.74 (m, 6H), 1.86-1.92 (m, 2H), 1.98-2.00 (m, 2H), 2.13-2.15 (m, 2H), 3.18 (s, 3H), 3.47-3.55 (m, 3H), 3.69-3.72 (m, 1H), 3.82-3.88 (m, 2H), 5.18-5.22 (m, 1H), 8.19 (s, 1H), 8.79 (s, 1H).

### Example 1.38

### Preparation of 5-(4-((1r,4r)-4-(5-(Methylsulfonyl)pyrazin-2-yloxy)cyclohexyloxy)piperidin-1-yl)-3-(prop-1-en-2-yl)-1,2,4-oxadiazole (Compound 69)

The title compound was isolated as a side product from Example 1.37. LCMS m/z=464.2 [M+H]⁺.

### Example 1.39

### Preparation of 1-Methylcyclopropyl 4-((1r,4r)-4-(4-(Methylsulfonyl)phenoxy)cyclohexyloxy)piperidine-1-carboxylate (Compound 46)

The title compound was prepared in a similar manner as described in Example 1.22 using 4-((1r,4r)-4-(4-(methylsulfonyl)phenoxy)cyclohexyloxy)piperidine hydrochloride and 1-methylcyclopropanol.

LCMS m/z=452.2 [M+H]⁺; ¹H NMR (400 MHz, CDCl₃) δ 0.62 (t, J=8 Hz, 2H), 0.86 (t, J=8 Hz, 2H), 1.46-1.65 (m, 6H), 1.54 (s, 3H), 1.44-1.64 (m, 6H), 1.75-1.79 (m, 2H), 1.95-2.00 (m, 2H), 2.08-2.13 (m, 2H), 3.02 (s, 3H), 3.09-3.14 (m, 2H), 3.50-3.58 (m, 2H), 3.72-3.76 (m, 2H), 4.38-4.43 (m, 1H), 6.98 (d, J=8 Hz, 1H), 7.00 (d, J=8 Hz, 1H), 7.83 (d, J=8 Hz, 1H), 7.85 (d, J=8 Hz, 1H).

### Example 1.40

### Preparation of 1-Methylcyclopropyl 4-((1r,4r)-4-(5-(Methylsulfonyl)pyrazin-2-yloxy)cyclohexyloxy)piperidine-1-carboxylate (Compound 64)

The title compound was prepared in a similar manner as described in Example 1.22 using 2-(methylsulfonyl)-5-((1r,4r)-4-(piperidin-4-yloxy)cyclohexyloxy)pyrazine hydrochloride and 1-methylcyclopropanol. LCMS m/z=454.5 [M+H]⁺; ¹H NMR (400 MHz, CDCl₃) δ 0.62 (t, J=8 Hz, 2H), 0.86 (t, J=8 Hz, 2H), 1.46-1.65 (m, 6H), 1.53 (s, 3H), 1.76-1.80 (m, 2H), 1.97-2.00 (m, 2H), 2.12-2.16 (m, 2H), 3.09-3.15 (m, 2H), 3.18 (s, 3H), 3.50-3.59 (m, 2H), 3.73-3.76 (m, 2H), 5.16-5.20 (m, 1H), 8.19 (s, 1H), 8.78 (s, 1H).

### Example 1.41

### Preparation of Isopropyl 4-((1r,4r)-4-(5-(1H-1,2,4-Triazol-1-yl)pyrazin-2-yloxy)cyclohexyloxy)piperidine-1-carboxylate (Compound 65)

The title compound was prepared in a similar manner as described in Example 1.36 (Step C) using tert-butyl 4-((1r,4r)-4-(5-(1H-1,2,4-triazol-1-yl)pyrazin-2-yloxy)cyclohexyloxy)piperidine-1-carboxylate. LCMS m/z=431.3 [M+H]⁺; ¹H NMR (400 MHz, CDCl₃) δ 1.24 (d, J=8 Hz, 6H), 1.48-1.64 (m, 6H), 1.79-1.84 (m, 2H), 1.98-2.03 (m, 2H), 2.14-2.18 (m, 2H), 3.11-3.17 (m, 2H), 3.50-3.55 (m, 1H), 3.56-3.60 (m, 1H), 3.79-3.84 (m, 2H), 4.88-4.99 (m, 1H), 5.06-5.11 (m, 1H), 7.99 (s, 1H), 8.11 (s, 1H), 8.70 (s, 1H), 8.98 (s, 1H).

### Example 1.42

### Preparation of tert-Butyl 4-((1r,4r)-4-(4-Cyanophenoxy)cyclohexyloxy)piperidine-1-carboxylate (Compound 23)

To a solution of Intermediate 2 (0.12 g, 0.4 mmol) in DMA (2.0 mL) was added NaH (40 mg, 60%) at room temperature. After stirring for 15 min, a solution of 4-fluorobenzonitrile (50 mg, 0.4 mmol) in DMA (0.5 mL) was added. The reaction was stirred for 5 h at room temperature under N₂ atmosphere. The reaction was quenched with water to form precipitates. The solid was filtered, washed with 0.5 N HCl and H₂O, and dried to give the title compound (110 mg). LCMS m/z=401.5 [M+H]⁺; ¹H NMR (400 MHz, CDCl₃) δ 1.45 (s, 9H), 1.42-1.62 (m, 6H), 1.74-1.82 (m, 2H), 1.93-2.02 (m, 2H), 2.05-2.14 (m, 2H), 3.04-3.13 (m, 2H), 3.48-3.58 (m, 2H), 3.73-3.81 (m, 2H), 4.43-4.40 (m, 1H), 6.91 (d, J=8.8 Hz, 2H), 7.56 (d, J=8.8 Hz, 2H).

### Example 1.43

### Preparation of 5-Chloro-2-(4-((1r,4r)-4-(4-(Methylsulfonyl)phenoxy)cyclohexyloxy)piperidin-1-yl)pyrimidine (Compound 34)

A solution of 4-((1r,4r)-4-(4-(methylsulfonyl)phenoxy)cyclohexyloxy)piperidine hydrochloride (30 mg, 0.077 mmol), 5-chloro-2-iodopyrimidine (28 mg, 0.12 mmol), and Et₃N (20 mg, 0.20 mmol) in IPA (1 mL) was stirred for 3 h at 85° C. The reaction was cooled to room temperature to form a precipitate. The solid was filtered, washed with H₂O, 1 N HCl, IPA, and dried to give the title compound (30 mg). LCMS m/z=466.2 [M+H]⁺; ¹H NMR (400 MHz, CDCl₃) δ 1.45-1.65 (m, 6H), 1.84-1.92 (m, 2H), 1.97-2.04 (m, 2H), 2.08-2.16 (m, 2H), 3.02 (s, 3H), 3.38-3.46 (m, 2H), 3.54-3.60 (m, 1H), 3.63-3.70 (m, 1H), 4.18-4.26 (m, 2H), 4.37-4.45 (m, 1H), 6.99 (d, J=8.8 Hz, 2H), 7.84 (d, J=8.8 Hz, 2H), 8.20 (s, 2H).

### Example 1.44

### Preparation of 2-(4-((1r,4r)-4-(4-(Methylsulfonyl)phenoxy)cyclohexyloxy)piperidin-1-yl)-5-(trifluoromethyl)pyridine (Compound 39)

The title compound was prepared in a similar manner as described in Example 1.43. LCMS m/z=499.6 [M+H]⁺; ¹H NMR (400 MHz, CDCl₃) δ 1.45-1.65 (m, 6H), 1.86-1.94 (m, 2H), 1.96-2.04 (m, 2H), 2.08-2.16 (m, 2H), 3.02 (s, 3H), 3.32-3.40 (m, 2H), 3.54-3.60 (m, 1H), 3.65-3.72 (m, 1H), 3.98-4.06 (m, 2H), 4.38-4.45 (m, 1H), 6.65 (d, J=8.8 Hz, 1H), 6.99 (d, J=8.8 Hz, 2H), 7.60 (dd, J=8.8 and 2.5 Hz, 1H), 7.84 (d, J=8.8 Hz, 2H), 8.38 (s, 1H).

### Example 1.45

### Preparation of (3-Methyloxetan-3-yl)methyl 4-((1r,4r)-4-(4-(Methylsulfonyl)phenoxy)cyclohexyloxy)piperidine-1-carboxylate (Compound 43)

A solution of (3-methyloxetan-3-yl)methanol and CDI in THF was stirred for 1.5 h at room temperature. Then, 4-((1r,4r)-4-(4-(methylsulfonyl)phenoxy)cyclohexyloxy)piperidine hydrochloride and triethylamine were added to the reaction. The heterogeneous mixture was stirred overnight at 80° C. The reaction was diluted with H₂O and extracted with DCM. The combined organic phase was washed with H₂O and 1 N HCl, dried, and concentrated. The residue was purified by silica gel column chromatography (hexane/EtOAc=100:0 to 70:30) to give the title compound (28 mg). LCMS m/z=482.4 [M+H]⁺; ¹H NMR (400 MHz, CDCl₃) δ 1.33 (s, 3H), 1.44-1.64 (m, 6H), 1.76-1.85 (m, 2H), 1.94-2.02 (m, 2H), 2.07-2.15 (m, 2H), 3.02 (s, 3H), 3.19-3.27 (m, 2H), 3.49-3.56 (m, 1H), 3.56-3.64 (m, 1H), 3.76-3.84 (m, 2H), 4.13 (s, 2H), 4.37 (d, J=5.8 Hz, 2H), 4.38-4.44 (m, 1H), 4.56 (d, J=5.8 Hz, 2H), 6.99 (d, J=8.8 Hz, 2H), 7.84 (d, J=8.8 Hz, 2H).

### Example 1.46

### Preparation of 1-(Cyclopropylsulfonyl)-4-((1r,4r)-4-(4-(Methylsulfonyl)phenoxy)cyclohexyloxy)piperidine (Compound 50)

A solution of 4-((1r,4r)-4-(4-(methylsulfonyl)phenoxy)cyclohexyloxy)piperidine hydrochloride (25 mg, 0.064 mmol), cyclopropanesulfonyl chloride (12 mg, 0.083 mmol), and triethylamine (16 mg, 0.16 mmol) in DCM (0.5 mL) was stirred for 3 h at room temperature. After removal of the volatile solvent, the residue was purified by silica gel column chromatography (hexane/EtOAc=100:0 to 70:30) to give the title compound (19 mg). LCMS m/z=458.3 [M+H]⁺; ¹H NMR (400 MHz, CDCl₃) δ 0.94-1.0 (m, 2H), 1.15-1.19 (m, 2H), 1.44-1.65 (m, 4H), 1.67-1.75 (m, 2H), 1.84-1.93 (m, 2H), 1.95-2.03 (m, 2H), 2.07-2.15 (m, 2H), 2.22-2.30 (m, 1H), 3.02 (s, 3H), 3.16-3.24 (m, 2H), 3.47-3.55 (m, 3H), 3.57-3.63 (m, 1H), 4.38-4.44 (m, 1H), 6.99 (d, J=8.8 Hz, 2H), 7.84 (d, J=8.8 Hz, 2H).

### Example 1.47

### Preparation of 1-(Isopropylsulfonyl)-4-((1r,4r)-4-(4-(Methylsulfonyl)phenoxy)cyclohexyloxy)piperidine (Compound 55)

The title compound was prepared by the procedure described in Example 1.46. LCMS m/z=460.5 [M+H]⁺.

### Example 1.48

### Preparation of (S)-Tetrahydrofuran-3-yl 4-((1r,4r)-4-(5-(Methylsulfonyl)pyrazin-2-yloxy)cyclohexyloxy)piperidine-1-carboxylate (Compound 56)

The title compound was prepared by the procedure described in Example 1.45 using 2-(methylsulfonyl)-5-((1r,4r)-4-(piperidin-4-yloxy)cyclohexyloxy)pyrazine hydrochloride and (S)-tetrahydrofuran-3-ol. LCMS m/z=470.5 [M+H]⁺; ¹H NMR (400 MHz, CDCl₃) δ 1.47-1.66 (m, 6H), 1.74-1.84 (m, 2H), 1.94-2.04 (m, 2H), 2.10-2.19 (m, 3H), 3.18 (s, 3H), 3.15-3.23 (m, 2H), 3.49-3.56 (m, 1H), 3.56-3.63 (m, 1H), 3.71-3.81 (m, 2H), 3.80-3.94 (m, 5H), 5.14-5.22 (m, 1H), 5.23-5.28 (m, 1H), 8.19 (d, J=1.2 Hz, 1H), 8.79 (d, J=1.2 Hz, 1H).

### Example 1.49

### Preparation of Isobutyl 4-((1r,4r)-4-(4-(Methylsulfonyl)phenoxy)cyclohexyloxy)piperidine-1-carboxylate (Compound 33)

To a suspension of 4-((1r,4r)-4-(4-(methylsulfonyl)phenoxy)cyclohexyloxy)piperidine hydrochloride (24.7 mg, 0.063 mmol) and triethylamine (50 μL, 0.359 mmol) in CH₂Cl₂ (1 mL), was added isobutyl chloroformate (18.4 μL, 0.140 mmol). After stirred at room temperature for 10 min, the reaction mixture was diluted with additional CH₂Cl₂ and extracted with water. The organic phase was dried over MgSO₄, filtered, and concentrated. The residue was purified by silica gel column chromatography (hexane/EtOAc) to give the title compound (25.8 mg) as a white solid. LCMS m/z=454.2 [M+H]⁺; ¹H NMR (400 MHz, CDCl₃) δ 0.98 (d, J=6.8 Hz, 6H), 1.44-1.64 (m, 6H), 1.78-1.83 (m, 2H), 1.90-2.01 (m, 3H), 2.09-2.13 (m, 2H), 3.02 (s, 3H), 3.15-3.22 (m, 2H), 3.51-3.60 (m, 2H), 3.79-3.85 (m, 2H), 3.86 (d, J=6.7 Hz, 2H), 4.39-4.43 (m, 1H), 6.97-7.01 (m, 2H), 7.83-7.86 (m, 2H).

### Example 1.50

### Preparation of Cyclopentyl 4-((1r,4r)-4-(4-(Methylsulfonyl)phenoxy)cyclohexyloxy)piperidine-1-carboxylate (Compound 36)

A solution of cyclopentanol (21 μL, 0.231 mmol) and CDI (35.7 mg, 0.220 mmol) in THF (0.5 mL) was stirred at room temperature. After 40 min, triethylamine (40 μL, 0.287 mmol) and 4-((1r,4r)-4-(4-(methylsulfonyl)phenoxy)cyclohexyloxy)piperidine hydrochloride (30.0 mg, 0.077 mmol) was added. The suspension was stirred at 80° C. (oil bath) over night. Purification by silica gel column chromatography (hexane/EtOAc) gave the title compound (29.1 mg) as a white solid. LCMS m/z=466.4 [M+H]⁺; ¹H NMR (400 MHz, CDCl₃) δ 1.47-1.86 (m, 16H), 1.96-2.01 (m, 2H), 2.08-2.13 (m, 2H), 3.02 (s, 3H), 3.10-3.17 (m, 2H), 3.51-3.59 (m, 2H), 3.38-3.43 (m, 2H), 4.38-4.43 (m, 1H), 5.08-5.11 (m, 1H), 6.97-7.01 (m, 2H), 7.82-7.86 (m, 2H).

### Example 1.51

### Preparation of 1,1,1,3,3,3-Hexafluoropropan-2-yl 4-((1r,4r)-4-(4-(Methylsulfonyl)phenoxy)cyclohexyloxy)piperidine-1-carboxylate (Compound 42)

A solution of 1,1,1,3,3,3-hexafluoropropan-2-ol (24 μL, 0.231 mmol) and di(1H-imidazol-1-yl)methanone (CDI) (30 mg, 0.185 mmol) in THF (0.5 mL) was stirred at room temperature. After 40 min, triethylamine (43 μL, 0.309 mmol) and 4-((1r,4r)-4-(4-(methylsulfonyl)phenoxy)cyclohexyloxy)piperidine hydrochloride (23.7 mg, 0.061 mmol) was added. The suspension was stirred at 80° C. (oil bath) over night. Purification by silica gel column chromatography (hexane/EtOAc) gave the title compound (25.4 mg) as a white solid. LCMS m/z=548.4 [M+H]⁺; ¹H NMR (400 MHz, CDCl₃) δ 1.45-1.65 (m, 6H), 1.76-86 (m, 2H), 1.93-2.00 (m, 2H), 2.07-2.13 (m, 2H), 3.02 (s, 3H), 3.37-3.43 (m, 2H), 3.51-3.56 (m, 1H), 3.64-3.69 (m, 1H), 3.72-3.78 (m, 2H), 4.40-4.45 (m, 1H), 5.72-5.78 (m, 1H), 6.97-7.01 (m, 2H), 7.83-7.86 (m, 2H).

### Example 1.52

### Preparation of 3-Methyl-6-(4-((1r,4r)-4-(4-(Methylsulfonyl)phenoxy)cyclohexyloxy)piperidin-1-yl)pyridazine (Compound 41)

A mixture of 4-((1r,4r)-4-(4-(methylsulfonyl)phenoxy)cyclohexyloxy)piperidine hydrochloride (29.3 mg, 0.075 mmol), 3-chloro-6-methylpyridazine (31.3 mg, 0.243 mmol), and triethylamine (50 μL, 0.359 mmol) in iPrOH (1 mL) was heated under microwave irradiation at 190° C. for 3 h. The mixture was concentrated and the residue was purified by silica gel column chromatography (hexane/EtOAc) to give the title compound (28.1 mg) as a white solid. LCMS m/z=446.6 [M+H]⁺; ¹H NMR (400 MHz, CDCl₃) δ 1.49-1.69 (m, 4H), 1.91-2.04 (m, 6H), 2.10-2.15 (m, 2H), 2.54 (s, 3H), 3.02 (s, 3H), 3.29-3.35 (m, 2H), 3.55-3.60 (m, 1H), 3.65-3.69 (m, 1H), 3.98-4.04 (m, 2H), 4.39-4.43 (m, 1H), 6.90 (d, J=9.3 Hz, 1H), 6.98-7.01 (m, 2H), 7.08 (d, J=9.3 Hz, 1H), 7.83-7.86 (m, 2H).

### Example 1.53

### Preparation of 2-Methyl-5-(4-((1r,4r)-4-(4-(Methylsulfonyl)phenoxy)cyclohexyloxy)piperidin-1-yl)pyrazine (Compound 44)

A mixture of 4-((1r,4r)-4-(4-(methylsulfonyl)phenoxy)cyclohexyloxy)piperidine hydrochloride (24.2 mg, 0.062 mmol), 2-chloro-5-methylpyrazine (90 mg, 0.700 mmol), and triethylamine (40 μL, 0.287 mmol) in iPrOH (1 mL) was heated under microwave irradiation at 200° C. for 5 h. The mixture was concentrated and the residue was purified by silica gel column chromatography (hexane/EtOAc) to give the title compound (8.7 mg) as a white solid. LCMS m/z=446.2 [M+H]⁺; ¹H NMR (400 MHz, CDCl₃) δ 1.46-1.68 (m, 6H), 1.89-1.96 (m, 2H), 1.99-2.04 (m, 2H), 2.10-2.15 (m, 2H), 2.39 (s, 3H), 3.03 (s, 3H), 3.21-3.27 (m, 2H), 3.55-3.59 (m, 1H), 3.63-3.67 (m, 1H), 3.91-3.97 (m, 2H), 4.39-4.44 (m, 1H), 6.98-7.01 (m, 2H), 7.83-7.86 (m, 2H), 7.94 (s, 1H), 8.07 (s, 1H).

### Example 1.54

### Preparation of 2,2-Difluoro-1-(4-((1r,4r)-4-(4-(Methylsulfonyl)phenoxy)cyclohexyloxy)piperidin-1-yl)butan-1-one (Compound 48)

A mixture of 2,2-difluorobutanoic acid (19 μL, 0.153 mmol), triethylamine (64 μL, 0.460 mmol) and HATU (80 mg, 0.210 mmol) in DMF (0.5 mL) was stirred at room temperature. After 10 min, 4-((1r,4r)-4-(4-(methylsulfonyl)phenoxy)cyclohexyloxy)piperidine hydrochloride (21.1 mg, 0.054 mmol) was added. The mixture was stirred at room temperature for 4 h and then was purified by semi-prep HPLC to give the title compound (22.6 mg) as a white solid. LCMS m/z=460.2 [M+H]⁺; ¹H NMR (400 MHz, CDCl₃) δ 1.07 (t, J=7.5 Hz, 3H), 1.61-1.66 (m, 6H), 1.81-1.87 (m, 2H), 1.98-2.01 (m, 2H), 2.09-2.22 (m, 4H), 3.03 (s, 3H), 3.46-3.56 (m, 3H), 3.67-3.71 (m, 1H), 3.83-3.91 (m, 2H), 4.40-4.44 (m, 1H), 6.97-7.01 (d, 2H), 7.83-7.86 (m, 2H).

### Example 1.55

### Preparation of sec-Butyl 4-((1r,4r)-4-(4-(Methylsulfonyl)phenoxy)cyclohexyloxy)piperidine-1-carboxylate (Compound 53)

A solution of (R)-butan-2-ol (19 μL, 0.256 mmol) and CDI (37.3 mg, 0.230 mmol) in THF (0.5 mL) was stirred at room temperature. After 40 min, triethylamine (40 μL, 0.287 mmol) and 4-((1r,4r)-4-(4-(methylsulfonyl)phenoxy)cyclohexyloxy)piperidine hydrochloride (22.3 mg, 0.057 mmol) were added. The suspension was stirred at 80° C. (oil bath) over night. Purification by silica gel column chromatography (hexane/EtOAc) gave the title compound (17.9 mg) as a white solid. LCMS m/z=454.4 [M+H]⁺; ¹H NMR (400 MHz, CDCl₃) δ 0.90 (t, J=7.4 Hz, 3H), 1.21 (d, J=6.2 Hz, 3H), 1.45-1.64 (m, 8H), 1.78-1.82 (m, 2H), 1.97-2.01 (m, 2H), 2.08-2.12 (m, 2H), 3.01 (s, 3H), 3.12-3.18 (m, 2H), 3.51-3.59 (m, 2H), 3.80-3.84 (m, 2H), 4.39-4.43 (m, 1H), 4.72-4.77 (m, 1H), 6.97-7.01 (d, 2H), 7.82-7.86 (m, 2H).

### Example 1.56

### Preparation of sec-Butyl 4-((1r,4r)-4-(4-(Methylsulfonyl)phenoxy)cyclohexyloxy)piperidine-1-carboxylate (Compound 54)

The title compound was prepared in a similar manner as described in Example 1.55 using (S)-butan-2-ol and 4-((1r,4r)-4-(4-(methylsulfonyl)phenoxy)cyclohexyloxy)piperidine hydrochloride. LCMS m/z=454.5 [M+H]⁺; ¹H NMR (400 MHz, CDCl₃) δ 0.90 (t, J=7.4 Hz, 3H), 1.21 (d, J=6.2 Hz, 3H), 1.45-1.64 (m, 8H), 1.78-1.82 (m, 2H), 1.97-2.01 (m, 2H), 2.08-2.12 (m, 2H), 3.01 (s, 3H), 3.12-3.18 (m, 2H), 3.51-3.59 (m, 2H), 3.80-3.84 (m, 2H), 4.39-4.43 (m, 1H), 4.72-4.77 (m, 1H), 6.97-7.01 (d, 2H), 7.82-7.86 (m, 2H).

### Example 1.57

### Preparation of 1,1,1-Trifluoropropan-2-yl 4-((1r,4r)-4-(4-(Methylsulfonyl)phenoxy)cyclohexyloxy)piperidine-1-carboxylate (Compound 63)

A solution of 1,1,1-trifluoropropan-2-ol (20 μl, 0.175 mmol) and CDI (25.8 mg, 0.159 mmol) in THF (0.5 mL) was stirred at room temperature. After 40 min, triethylamine (40 μL, 0.287 mmol) and 4-((1r,4r)-4-(4-(methylsulfonyl)phenoxy)cyclohexyloxy)piperidine hydrochloride (20.4 mg, 0.052 mmol) were added. The suspension was stirred at 80° C. (oil bath) overnight. Purification by silica gel column chromatography (hexane/EtOAc) gave the title compound (22.4 mg) as a white solid. LCMS m/z=494.4 [M+H]⁺; ¹H NMR (400 MHz, CDCl₃) δ 1.39-1.41 (d, J=6.6 Hz, 3H), 1.45-1.65 (m, 6H), 1.77-1.82 (m, 2H), 1.96-2.01 (m, 2H), 2.09-2.13 (m, 2H), 3.02 (s, 3H), 3.25-3.30 (m, 2H), 3.51-3.55 (m, 1H), 3.60-3.63 (m, 1H), 3.73-3.79 (m, 2H), 4.39-4.43 (m, 1H), 5.21-5.27 (m, 1H), 6.97-7.01 (m, 2H), 7.83-7.86 (m, 2H).

### Example 1.58

### Preparation of 5-(2-Fluoropropan-2-yl)-3-(4-((1r,4r)-4-(5-(Methylsulfonyl)pyrazin-2-yloxy)cyclohexyloxy)piperidin-1-yl)-1,2,4-oxadiazole (Compound 62)

### Step A: Preparation of 4-((1r,4r)-4-(5-(Methylsulfonyl)pyrazin-2-yloxy)cyclohexyloxy)piperidine-1-carbonitrile

To an ice-cooled suspension of 2-(methylsulfonyl)-5-((1r,4r)-4-(piperidin-4-yloxy)cyclohexyloxy)pyrazine hydrochloride (100 mg, 0.255 mmol) and triethylamine (180 μL, 1.293 mmol) in CH₂Cl₂ (8 mL), was added a solution of cyanic bromide (32.4 mg, 0.306 mmol) in CH₂Cl₂ (2 mL). After stirring under ice-cooling for 1 h, the mixture was diluted with additional CH₂Cl₂ and extracted with water. The organic phase was dried over MgSO₄, filtered, and concentrated to give the title compound (108 mg). LCMS m/z=381.4 [M+H]⁺; ¹H NMR (400 MHz, CDCl₃) δ 1.30-2.01 (m, 8H), 2.11-2.17 (m, 2H), 3.04-3.13 (m, 3H), 3.18 (s, 3H), 3.41-3.53 (m, 4H), 3.59-3.64 (m, 1H), 5.16-5.22 (m, 1H), 8.19 (d, J=1.3 Hz, 1H), 8.79 (d, J=1.3 Hz, 1H).

### Step B: Preparation of N′-Hydroxy-4-((1r,4r)-4-(5-(methylsulfonyl)pyrazin-2-yloxy)cyclohexyloxy)piperidine-1-carboximidamide

To a solution of 4-((1r,4r)-4-(5-(methylsulfonyl)pyrazin-2-yloxy)cyclohexyloxy)piperidine-1-carbonitrile (90% pure, 105 mg, 0.248 mmol) in EtOH (1 mL), hydroxylamine (50% in water, 1 mL, 15 mmol) was added. The mixture was stirred at 60° C. for 1 h and was concentrated to give the title compound (117 mg) as a solid. LCMS m/z=414.4 [M+H]⁺.

### Step C: Preparation of 5-(2-Fluoropropan-2-yl)-3-(4-((1r,4r)-4-(5-(methylsulfonyl)pyrazin-2-yloxy)cyclohexyloxy)piperidin-1-yl)-1,2,4-oxadiazole

To a solution of 2-fluoro-2-methylpropanoic acid (18.5 mg, 0.174 mmol) in DMA (1 mL) was added CDI (33.4 mg, 0.206 mmol). After stirring at room temperature for 10 min, N′-hydroxy-4-((1r,4r)-4-(5-(methylsulfonyl)pyrazin-2-yloxy)cyclohexyloxy)piperidine-1-carboximidamide (58.5 mg, 0.141 mmol) was added. The reaction was stirred at 110° C. (oil bath) for 30 min. The resulting mixture was purified by semi-prep HPLC. Fractions containing desired product were partly concentrated and the residue was partitioned between 1 M NaOH and CH₂Cl₂. The organic phase was dried over MgSO₄, filtered, and concentrated to give the title compound (23.2 mg) as a white solid. LCMS m/z=484.4 [M+H]⁺; ¹H NMR (400 MHz, CDCl₃) δ 1.50-1.70 (m, 5H), 1.76 (s, 3H), 1.80 (s, 3H), 1.87-1.93 (m, 2H), 1.98-2.02 (m, 2H), 2.13-2.18 (m, 2H), 3.18 (s, 3H), 3.18-3.26 (m, 3H), 3.53-3.66 (m, 2H), 3.76-3.82 (m, 2H), 5.17-5.22 (m, 1H), 8.19 (d, J=1.3 Hz, 1H), 8.78 (d, J=1.3 Hz, 1H).

### Example 1.59

### Preparation of 3,3-Dimethyl-1-(4-((1r,4r)-4-(4-(Methylsulfonyl)phenoxy)cyclohexyloxy)piperidin-1-yl)butan-1-one (Compound 32)

To a solution of 4-((1r,4r)-4-(4-(methylsulfonyl)phenoxy)cyclohexyloxy)piperidine hydrochloride (0.02 g, 0.057 mmol) in DCM (1.0 mL) were added triethylamine (0.032 mL, 0.226 mmol) and 3,3-dimethylbutanoyl chloride (0.015 g, 0.113 mmol). The reaction was stirred at room temperature for 3 h. Water was added and the reaction mixture was extracted with DCM. The combined extracts were dried (sodium sulfate), filtered, and concentrated. The residue was taken up in MTBE and hexanes were added until a white solid had formed. The mother liquor was decanted off to give the title compound (17 mg). LCMS m/z=452.4 [M+H]⁺; ¹H NMR (400 MHz, CDCl₃) δ 1.06 (s, 9H), 1.44-1.86 (m, 8H), 1.94-2.04 (m, 2H), 2.06-2.15 (m, 2H), 2.29 (s, 2H), 3.02 (s, 3H), 3.26-3.34 (m, 2H), 3.50-3.57 (m, 1H), 3.60-3.67 (m, 1H), 3.81-3.94 (m, 2H), 4.38-4.45 (m, 1H), 6.99 (d, J=8.8 Hz, 2H), 7.84 (d, J=8.8 Hz, 2H).

### Example 1.60

### Preparation of tert-Butyl 2-(4-((1r,4r)-4-(4-(Methylsulfonyl)phenoxy)cyclohexyloxy)piperidin-1-yl)acetate (Compound 35)

To a solution of 4-((1r,4r)-4-(4-(methylsulfonyl)phenoxy)cyclohexyloxy)piperidine hydrochloride (0.02 g, 0.051 mmol) in 2-propanol (0.5 mL) were added triethylamine (0.021 mL, 0.154 mmol) and tert-butyl 2-bromoacetate (10.00 mg, 0.051 mmol). The reaction was stirred at room temperature for 24 h and concentrated. The residue was purified by silica gel column chromatography (0-10% MeOH in DCM) to give the title compound (16 mg). LCMS m/z=468.5 [M+H]⁺; ¹H NMR (400 MHz, CDCl₃) δ 1.47 (s, 9H), 1.49-1.72 (m, 6H), 1.80-1.91 (m, 2H), 1.94-2.03 (m, 2H), 2.06-2.15 (m, 2H), 2.29-2.43 (m, 2H), 2.76-2.88 (m, 2H), 3.02 (s, 3H), 3.09-3.14 (m, 2H), 3.38-3.53 (m, 2H), 4.35-4.43 (m, 1H), 6.99 (d, J=9.0 Hz, 2H), 7.84 (d, J=8.8 Hz, 2H).

### Example 1.61

### Preparation of (2-Methylcyclopropyl)(4-((1r,4r)-4-(4-(Methylsulfonyl)phenoxy)cyclohexyloxy)piperidin-1-yl)methanone (Compound 45)

To a solution of 4-((1r,4r)-4-(4-(Methylsulfonyl)phenoxy)cyclohexyloxy)piperidine hydrochloride (0.02 g, 0.051 mmol) in DCM (0.5 mL) were added triethylamine (0.036 mL, 0.256 mmol) and 2-methylcyclopropanecarbonyl chloride (0.012 g, 0.103 mmol). The reaction was stirred at room temperature for 24 h. The reaction mixture was washed with water, and the remaining DCM layer was dried (sodium sulfate), filtered, and concentrated. The residue was purified by silica gel column chromatography (0-10% MeOH/DCM) to give the title compound (9.1 mg). LCMS m/z=436.3 [M+H]⁺; ¹H NMR (400 MHz, CDCl₃) δ 0.53-0.61 (m, 1H), 0.81-0.92 (m, 1H), 1.09-1.19 (m, 4H), 1.24-1.76 (m, 7H), 1.78-1.90 (m, 2H), 1.94-2.05 (m, 2H), 2.06-2.17 (m, 2H), 3.03 (s, 3H), 3.29-3.40 (m, 2H), 3.51-3.59 (m, 1H), 3.61-3.70 (m, 1H), 3.90-3.99 (m, 2H), 4.38-4.46 (m, 1H), 6.99 (d, J=8.7 Hz, 2H), 7.85 (d, J=8.9 Hz, 2H).

### Example 1.62

### Preparation of 5-Chloro-2-(4-((1r,4r)-4-(4-(Methylsulfonyl)phenoxy)cyclohexyloxy)piperidin-1-yl)pyridine (Compound 49)

To a solution of 4-((1r,4r)-4-(4-(Methylsulfonyl)phenoxy)cyclohexyloxy)piperidine hydrochloride (0.02 g, 0.135 mmol) in 2-propanol (0.5 mL) were added triethylamine (0.094 mL, 0.676 mmol) and 2,5-dichloropyridine (0.105 g, 0.270 mmol). The reaction was heated to 150° C. for 10 h. The reaction mixture was filtered and the filtrate was concentrated under reduced pressure. The residue was purified by silica gel column chromatography (0-10% MeOH/DCM) to give the title compound (4.2 mg). LCMS m/z=465.3 [M+H]⁺; ¹H NMR (400 MHz, CDCl₃) δ 1.47-1.72 (m, 6H), 1.86-2.17 (m, 6H), 3.02 (s, 3H), 3.16-3.39 (m, 2H), 3.53-3.60 (m, 1H), 3.63-3.71 (m, 1H), 3.89-3.97 (m, 2H), 4.37-4.46 (m, 1H), 6.60-6.69 (m, 1H), 6.99 (d, J=8.8 Hz, 2H), 7.40-7.47 (m, 1H), 7.85 (d, J=8.8 Hz, 2H), 8.10-8.13 (m, 1H).

### Example 1.63

### Preparation of tert-Butyl 4-((1r,4r)-4-(5-Cyanopyrazin-2-yloxy)cyclohexyloxy)piperidine-1-carboxylate (Compound 68)

To a solution of tert-butyl 4-((1r,4r)-4-(5-bromopyrazin-2-yloxy)cyclohexyloxy)piperidine-1-carboxylate (0.02 g, 0.044 mmol) in DMA (0.5 mL) were added dicyanozinc (0.015 g, 0.131 mmol) and tetrakis(triphenylphosphine)palladium (5.06 mg, 4.38 mol). The reaction vessel was purged with nitrogen and then heated at 120° C. for 2 h. The reaction mixture was concentrated and the residue was partitioned between EtOAc and water. The EtOAc layer was dried (sodium sulfate), filtered, and concentrated under reduced pressure. The residue was purified by silica gel column chromatography (10-50% EtOAc/hexanes) to give the title compound (10.2 mg). LCMS m/z=403.7 [M+H]⁺; ¹H NMR (400 MHz, CDCl₃) δ 1.46 (s, 9H), 1.47-1.66 (m, 6H), 1.74-1.83 (m, 2H), 1.93-2.02 (m, 2H), 2.09-2.17 (m, 2H), 3.04-3.14 (m, 2H), 3.48-3.60 (m, 2H), 3.73-3.82 (m, 2H), 5.09-5.17 (m, 1H), 8.20 (d, J=1.3 Hz, 1H), 8.43 (d, J=1.4 Hz, 1H).

### Example 1.64

### Preparation of 4-((1r,4r)-4-(4-(Methylsulfonyl)phenoxy)cyclohexyloxy)-1-P-Tolylpiperidine (Compound 57)

To a suspension of 4-((1r,4r)-4-(4-(methylsulfonyl)phenoxy)cyclohexyloxy)piperidine hydrochloride (0.035 g, 0.090 mmol) was added p-tolylboronic acid (3.0 eq) followed by copper (II)acetate (0.049 g, 0.269 mmol) and triethylamine (0.063 mL, 0.449 mmol). The reaction was stirred at room temperature for 16 h. Purification by column silica gel chromatography (50% EtOAc/hexanes) gave the title compound (0.024 g) as a red solid. LCMS m/z=444.6 [M+H]⁺; ¹H NMR (CDCl₃, 400 MHz) δ 1.45-1.54 (m, 2H), 1.56-1.64 (m, 2H), 1.69-1.77 (m, 2H), 1.96-2.03 (m, 4H), 2.10-2.15 (m, 2H), 2.27 (s, 3H), 2.86-2.92 (m, 2H), 3.02 (s, 3H), 3.46-3.53 (m, 2H), 3.54-3.59 (m, 2H), 4.39-4.493 (m, 1H), 6.88 (d, J=8.46 Hz, 2H), 6.98-7.01 (m, 2H), 7.07 (d, J=8.21 Hz, 2H), 7.83-7.86 (m, 2H).

### Example 1.65

### Preparation of 4-((1r,4r)-4-(4-(Methylsulfonyl)phenoxy)cyclohexyloxy)-1-(4-(trifluoromethyl)phenyl)piperidine (Compound 58)

The title compound was prepared in a similar manner as described in Example 1.64. LCMS m/z=498.6 [M+H]⁺; ¹H NMR (CDCl₃, 400 MHz) δ 1.49-1.74 (m, 6H), 1.91-2.03 (m, 4H), 2.10-2.15 (m, 2H), 3.02 (s, 3H), 3.04-3.11 (m, 2H), 3.59-3.64 (m, 4H), 4.40-4.44 (m, 1H), 6.92 (d, J=8.72 Hz, 2H), 7.00 (d, J=8.97 Hz, 2H), 7.46 (d, J=8.59 Hz, 2H), 7.85 (dd, 2H).

### Example 1.66

### Preparation of 4-((1r,4r)-4-(4-(Methylsulfonyl)phenoxy)cyclohexyloxy)-1-(4-(trifluoromethoxy)phenyl)piperidine (Compound 59)

The title compound was prepared in a similar manner as described in Example 1.64 to give a brownish solid. LCMS m/z=514.6 [M+H]⁺; ¹H NMR (CDCl₃, 400 MHz) δ 1.39-1.58 (m, 4H), 1.60-1.69 (m, 2H), 1.86-1.96 (m, 4H), 2.03-2.08 (m, 2H), 2.85-2.91 (m, 2H), 2.95 (s, 3H), 3.40-3.53 (m, 4H), 4.32-4.36 (m, 1H), 6.83 (d, J=9.09 Hz, 2H), 6.92 (d, 2H), 7.03 (d, 2H), 7.77 (d, 2H).

### Example 1.67

### Preparation of 1-(4-Methoxyphenyl)-4-((1r,4r)-4-(4-(Methylsulfonyl)phenoxy)cyclohexyloxy)piperidine (Compound 60)

The title compound was prepared by the procedure described in Example 1.64 to give a tan solid. LCMS=460.5 [M+H]⁺; ¹H NMR (CDCl₃, 400 MHz) δ 1.49-1.62 (m, 4H), 1.72-1.78 (m, 2H), 1.95-2.03 (m, 4H), 2.11-2.15 (m, 2H), 2.79-2.85 (m, 2H), 3.02 (s, 3H), 3.36-3.42 (m, 2H), 3.51-3.58 (m, 2H), 3.77 (s, 3H), 4.38-4.43 (m, 1H), 6.80-6.87 (m, 2H), 6.89-6.95 (m, 2H), 6.97-7.03 (m, 2H), 7.83-7.86 (m, 2H).

### Example 1.68

### Preparation of 4-((1r,4r)-4-(4-(Methylsulfonyl)phenoxy)cyclohexyloxy)-1-(3-(trifluoromethyl)phenyl)piperidine (Compound 61)

The title compound was prepared by the procedure described in Example 1.64 to give a yellowish solid. LCMS m/z=498.5 [M+H]⁺; ¹H NMR (CDCl₃, 400 MHz) δ 1.46-1.65 (m, 4H), 1.68-1.76 (m, 2H), 1.93-2.03 (m, 4H), 2.10-2.15 (m, 2H), 2.98-3.05 (m, 2H), 3.03 (s, 3H), 3.53-3.63 (m, 4H), 4.40-4.44 (m, 1H), 6.98-7.01 (m, 2H), 7.06 (dd, J=12.19, 8.40 Hz, 2H), 7.12 (s, 1H), 7.30-7.34 (t, J=7.89 Hz, 1H), 7.83-7.86 (m, 2H).

### Example 1.69

### Preparation of 1-(4-Fluorophenyl)-4-((1r,4r)-4-(4-(Methylsulfonyl)phenoxy)cyclohexyloxy)piperidine (Compound 66)

To a solution of 4-((1r,4r)-4-(4-(methylsulfonyl)phenoxy)cyclohexyloxy)piperidine hydrochloride (30 mg, 0.077 mmol) in dichloroethane (1 mL) were added 4-fluorophenylboronic acid (43.1 mg, 0.308 mmol), diacetoxycopper (27.9 mg, 0.154 mmol) and triethylamine (0.043 mL, 0.308 mmol). The reaction was stirred at room temperature for 24 h. The reaction mixture was quenched with water and the organic layer was separated. The aqueous layer was extracted with dichloromethane (twice) and the combined organics were concentrated in vacuo. The residue was purified by silica gel column chromatography to give the title compound (12.6 mg) as a white solid. LCMS m/z=448.2 [M+H]⁺; ¹H NMR (400 MHz, CDCl₃) δ 1.46-1.54 (m, 2H), 1.56-1.65 (m, 2H), 1.69-1.77 (m, 2H), 1.94-2.03 (m, 4H), 2.11-2.15 (m, 2H), 2.84-2.90 (m, 2H), 3.03 (s, 3H), 3.39-3.44 (m, 2H), 3.52-3.59 (m, 2H), 4.38-4.44 (m, 1H), 6.87-6.97 (m, 4H), 6.98-7.02 (m, 2H), 7.83-7.86 (m, 2H).

### Example 1.70

### Preparation of 1-(4-Chloro-2-fluorophenyl)-4-((1r,4r)-4-(4-(Methylsulfonyl)phenoxy)cyclohexyloxy)piperidine (Compound 67)

To a solution of 4-((1r,4r)-4-(4-(methylsulfonyl)phenoxy)cyclohexyloxy)piperidine hydrochloride (30 mg, 0.077 mmol) in dichloroethane (1 mL) were added 4-chloro-2-fluorophenylboronic acid (53.7 mg, 0.308 mmol), diacetoxycopper (27.9 mg, 0.154 mmol), and triethylamine (0.043 mL, 0.308 mmol). The reaction was stirred at room temperature for 24 h followed by 50° C. for an additional 24 h. The reaction mixture was quenched with water and the organic layer was separated. The aqueous layer was extracted with dichloromethane (twice) and the combined organics were concentrated in vacuo. The residue was purified by silica gel column chromatography to give the title compound (1.5 mg) as a white solid. LCMS m/z=482.2 [M+H]⁺; ¹H NMR (400 MHz, CDCl₃) δ 1.46-1.55 (m, 2H), 1.56-1.65 (m, 2H), 1.72-1.81 (m, 2H), 1.94-2.03 (m, 4H), 2.11-2.15 (m, 2H), 2.81-2.87 (m, 2H), 3.03 (s, 3H), 3.27-3.32 (m, 2H), 3.52-3.60 (m, 2H), 4.38-4.44 (m, 1H), 6.87 (t, J=8.0 Hz, 1H), 6.98-7.05 (m, 4H), 7.83-7.87 (m, 2H).

### Example 1.71

### Preparation of tert-Butyl 4-((1r,4r)-4-(4-(1H-1,2,4-Triazol-1-yl)phenoxy)cyclohexyloxy)piperidine-1-carboxylate (Compound 47)

To a solution of tert-butyl 4-((1s,4s)-4-hydroxycyclohexyloxy)piperidine-1-carboxylate (0.210 g, 0.701 mmol) was added 4′-(1H-1,2,4-triazol-1-yl)phenol (0.147 g, 0.912 mmol) to give a suspension. Triphenylphosphine (0.239 g, 0.912 mmol) was added followed by dropwise addition of diisopropylazodicarboxylate (0.180 mL, 0.912 mmol) at 24° C. to give an amber solution. After stirred at 24° C. for 15 h, the reaction mixture was diluted with EtOAc (50 mL), washed with water (10 mL), brine (10 mL), dried over MgSO₄, and concentrated. The residue was purified HPLC. The HPLC fractions were collected, the pH was adjusted to pH 7 with saturated aqueous NaHCO₃ (3 mL), and extracted with EtOAc (50 mL). The organic layer was washed with water (10 mL), brine (10 mL), dried over MgSO₄, and concentrated to give the title compound (0.069 g) as a white solid. LCMS m/z=443.2 [M+H]⁺; ¹H NMR (400 MHz, CD₃CN) δ 1.38 (m, 4H), 1.42 (s, 9H), 1.77 (m, 3H), 1.50 (m, 3H), 2.08 (m, 2H), 3.04 (m, 2H), 3.56 (m, 2H), 3.66-3.79 (m, 2H), 4.39 (m, 1H), 7.05 (d, J=9.09 Hz, 2H), 7.62 (d, J=9.09 Hz, 2H), 8.01 (s, 1H), 8.63 (s, 1H).

### Resolution Via HPLC:

Column: reversed phase semi-preparative, 250×20 mm ID, 5 μm particle size.
Eluent: A: 0.1% trifluoroacetic acid in water; B: 0.1% trifluoroacetic acid in acetonitrile.
Gradient: 5-95% B, 30 min Flow: 20 mL/minute.

### Detector: 254 nm

### Retention Time: 25 min

### Example 1.72

### Preparation of tert-Butyl 4-((1r,4r)-4-(5-(Isopropylsulfonyl)pyrazin-2-yloxy)cyclohexyloxy)piperidine-1-carboxylate (Compound 72)

tert-Butyl 4-((1r,4r)-4-(5-bromopyrazin-2-yloxy)cyclohexyloxy)piperidine-1-carboxylate (0.02 g, 0.044 mmol), sodium propane-2-sulfinate (0.011 g, 0.088 mmol), copper (I) trifluoromethanesulfonate benzene complex (3.97 mg, 7.89 μmol), and N¹,N²-dimethylethane-1,2-diamine (1.545 mg, 0.018 mmol) were suspended in DMSO (0.5 mL) and heated to 110° C. for 2.5 h. The reaction mixture was diluted with water and extracted with ethyl acetate. The combined extracts were dried, filtered, and concentrated. The residue was purified by silica gel column chromatography (10-50% EtOAc/hexanes) to give the title compound (9.1 mg). LCMS m/z=484.3 [M+H]⁺; ¹H NMR (400 MHz, CDCl₃) δ 1.35 (d, J=6.9 Hz, 6H), 1.46 (s, 9H), 1.41-1.67 (m, 6H), 1.74-1.84 (m, 2H), 1.94-2.03 (m, 2H), 2.11-2.19 (m, 2H), 3.04-3.13 (m, 2H), 3.48-3.62 (m, 3H), 3.71-3.83 (m, 2H), 5.13-5.22 (m, 1H), 8.21 (d, J=1.3 Hz, 1H), 8.75 (d, J=1.3 Hz, 1H).

### Example 1.73

### Preparation of 1,3-Difluoropropan-2-yl 4-((1r,4r)-4-(5-(Methylsulfonyl)pyrazin-2-yloxy)cyclohexyloxy)piperidine-1-carboxylate (Compound 71)

The title compound was prepared in a similar manner as described in Example 1.22 using 2-(methylsulfonyl)-5-((1r,4r)-4-(piperidin-4-yloxy)cyclohexyloxy)pyrazine hydrochloride and 1,3-difluoropropan-2-ol. LCMS m/z=478.2 [M+H]⁺; ¹H NMR (400 MHz, CDCl₃) δ 1.48-1.66 (m, 6H), 1.77-1.84 (m, 2H), 1.95-2.01 (m, 2H), 2.12-2.17 (m, 2H), 3.18 (s, 3H), 3.23-3.30 (m, 2H), 3.50-3.56 (m, 1H), 3.59-3.65 (m, 1H), 3.75-3.81 (m, 2H), 4.55-4.56 (d, J=4.9 Hz, 2H), 4.66-4.67 (d, J=4.9 Hz, 2H), 5.04-5.22 (m, 2H), 8.19 (d, J=1.3 Hz, 1H), 8.79 (d, J=1.3 Hz, 1H).

### Example 1.74

### Preparation of 2,2-Difluoro-1-(4-((1r,4r)-4-(5-(Methylsulfonyl)pyrazin-2-yloxy)cyclohexyloxy)piperidin-1-yl)butan-1-one (Compound 73)

To a stirred solution of 2,2-difluorobutanoic acid (15.20 mg, 0.122 mmol) and HATU (46.6 mg, 0.122 mmol) in DMF (2 mL) was added N-ethyl-N-isopropylpropan-2-amine (39.6 mg, 0.306 mmol). The reaction mixture was stirred for 10 min and added 2-(methylsulfonyl)-5-((1r,4r)-4-(piperidin-4-yloxy)cyclohexyloxy)pyrazine hydrochloride (40 mg, 0.102 mmol). The mixture was stirred at room temperature for 1 h. The mixture was then diluted with water and extracted with EtOAc. The organic layer was dried over MgSO₄ and concentrated. The residue was purified by silica gel column chromatography to give the title compound (45 mg) as a white solid. LCMS m/z=462.2 [M+H]⁺; ¹H NMR (400 MHz, CDCl₃) δ 1.09 (t, J=7.5 Hz, 3H), 1.51-1.68 (m, 6H), 1.83-1.89 (m, 2H), 1.97-2.03 (m, 2H), 2.10-2.24 (m, 4H), 3.20 (s, 3H), 3.45-3.58 (m, 3H), 3.69-3.74 (m, 1H), 3.83-3.96 (m, 2H), 5.18-5.24 (m, 1H), 8.21 (d, J=1.3 Hz, 1H), 8.80 (d, J=1.3 Hz, 1H).

### Example 1.75

### Preparation of (S)-1-Hydroxypropan-2-yl 4-((1r,4r)-4-(5-(Methylsulfonyl)pyrazin-2-yloxy)cyclohexyloxy)piperidine-1-carboxylate (Compound 76)

### Step A: Preparation of (S)-1-(Benzyloxy)propan-2-yl 4-((1r,4r)-4-(5-(methylsulfonyl)pyrazin-2-yloxy)cyclohexyloxy)piperidine-1-carboxylate

A mixture of (S)-1-(benzyloxy)propan-2-ol (31 μL, 0.194 mmol) and CDI (22.0 mg, 0.136 mmol) in 0.5 ml THF was stirred at room temperature. After 40 min, 2-(methylsulfonyl)-5-((1r,4r)-4-(piperidin-4-yloxy)cyclohexyloxy)pyrazine hydrochloride (25.6 mg, 0.065 mmol) and triethylamine (44 μL, 0.316 mmol) were added and mixture was stirred at 80° C. After 3 days, mixture was purified by Biotage™ (hexane/AcOEt, 10 g SiO₂) to give (S)-1-(benzyloxy)propan-2-yl 4-((1r,4r)-4-(5-(methylsulfonyl)pyrazin-2-yloxy)cyclohexyloxy)piperidine-1-carboxylate (33.3 mg, 0.061 mmol, 93% yield) as a white solid. Exact mass calculated for C₂₇H₃₇N₃O₇S: 547.24. found: LCMS m/z=548.4 [M+H]⁺; ¹H NMR (400 MHz, CDCl₃) δ 1.26 (d, J=6.5 Hz, 3H), 1.48-1.66 (m, 6H), 1.75-1.80 (m, 2H), 1.96-2.01 (m, 2H), 2.13-2.18 (m 2H), 3.15-3.21 (m, 5H), 3.47-3.64 (m, 4H), 3.77-3.82 (m, 2H), 4.51-4.60 (m, 2H), 5.01-5.05 (m, 1H), 5.16-5.20 (m, 1H), 7.26-7.36 (m, 5H), 8.19 (d, J=1.3 Hz, 1H), 8.79 (d, J=1.3 Hz, 1H).

### Step B: Preparation of (S)-1-Hydroxypropan-2-yl 4-((1r,4r)-4-(5-(Methylsulfonyl)pyrazin-2-yloxy)cyclohexyloxy)piperidine-1-carboxylate (Compound 76)

To a solution of (S)-1-(benzyloxy)propan-2-yl 4-((1r,4r)-4-(5-(methylsulfonyl)pyrazin-2-yloxy)cyclohexyloxy)piperidine-1-carboxylate (33 mg, 0.060 mmol) in 2 ml THF, 10% palladium on carbon (Degussa Type, 30 mg) was added. Hydrogen was bubbled through the suspension for 1 min and it was then stirred at room temperature under an H₂-atmosphere (balloon). After stirring over night, the temperature was raised to 50° C. and the suspension was further stirred under an H₂-atmosphere for 6 h. The suspension was filtered through Celite™ and the filtrate was purified using semi-prep HPLC (5-95% CH₃CN in 30 min). Fractions containing the desired product were partly concentrated and the residue was partitioned between 1 M NaHCO₃ and CH₂Cl₂. Organic phases were dried over MgSO₄, filtered, and concentrated to give the title compound (8.7 mg, 0.019 mmol, 31.6% yield) as a white solid. Exact mass was calculated for C₂₀H₃₁N₃O₇S: 457.19. found: LCMS m/z=458.2 [M+H]⁺; ¹H NMR (400 MHz, CDCl₃) δ 1.26 (d, J=6.7 Hz, 3H), 1.48-1.66 (m, 6H), 1.78-1.83 (m, 2H), 1.96-2.01 (m, 2H), 2.13-2.18 (m, 2H), 2.62-2.65 (m, 1H), 3.18-3.26 (m, 5H), 3.51-3.80 (m, 6H), 4.89-4.93 (m, 1H), 5.16-5.30 (m, 1H), 8.19 (d, J=1.3 Hz, 1H), 8.79 (d, J=1.3 Hz, 1H).

### Example 1.76

### Preparation of Isopropyl 4-((1r,4r)-4-(5-(Pyridazin-4-yl)pyrazin-2-yloxy)cyclohexyloxy)-piperidine-1-carboxylate (Compound 77)

A mixture of isopropyl 4-((1r,4r)-4-(5-bromopyrazin-2-yloxy)cyclohexyloxy)piperidine-1-carboxylate (100 mg, 0.181 mmol), 4-(tributylstannyl)pyridazine (167 mg, 0.452 mmol) and a catalytic amount of Pd(PPh₃)₂Cl₂ (6.35 mg, 9.04 μmol) in dioxane (1 mL) was heated at 110° C. for 8 hr. The mixture was allowed to cool to room temperature, diluted with EtOAc, and washed with water, then brine. The organic phase was dried over Na₂CO₃, filtered and concentrated to give the crude product, which was purified by flash chromatography using hexane and EtOAc (20% to 100% 10 column volumes and 100% 5 column volumes) to give isopropyl 4-((1r,4r)-4-(5-(pyridazin-4-yl)pyrazin-2-yloxy)cyclohexyloxy)piperidine-1-carboxylate (50 mg, 0.113 mmol, 62.6% yield), which is further purified by recrystallization. Exact mass calculated for C₂₃H₃₁N₅O₄: 441.5. found LCMS m/z=442.4 [M+H]⁺. ¹H NMR (400 MHz, CDCl₃) δ 1.26-1.27 (d, J=6.3 Hz, 6H), 1.50-1.69 (m, 6H), 1.81-1.86 (m, 2H), 2.01-2.07 (m, 2H), 2.18-2.22 (m, 2H), 3.14-3.20 (m, 2H), 3.52-3.64 (m, 2H), 3.82-3.85 (m, 2H), 4.91-4.97 (m, 1H), 5.12-5.19 (m, 1H), 8.00-8.02 (dd, J=5.6, 2.4 Hz, 1H), 8.31-8.32 (d, J=1.5 Hz, 1H), 8.68 (d, J=1.5 Hz, 1H), 9.28-9.30 (dd, J=5.6, 1.4 Hz, 1H), 9.78 (dd, J=2.5, 1.3 Hz, 1H).

### Example 1.77

### Preparation of isopropyl 4-((1r,4r)-4-(5-(1,1-dioxo-thiomorpholin-4-yl)pyrazin-2-yloxy)cyclohexyloxy)piperidine-1-carboxylate (Compound 78)

A mixture isopropyl 4-((1r,4r)-4-(5-chloropyrazin-2-yloxy)cyclohexyloxy)piperidine-1-carboxylate (50 mg, 0.126 mmol), diacetoxypalladium (2.8 mg, 0.013 mmol), thiomorpholine-1,1-dioxide (17 mg, 0.126 mmol), sodium 2-methylpropan-2-olate (29 mg, 0.302 mmol), and 1,1′-bis(di-t-butylphosphino)ferrocene (12 mg, 0.025 mmol) in 1,4-dioxane (0.8 mL) was heated at 100° C. for 1 h under microwave irradiation. LCMS showed product had formed and no starting material remained. Water was added to the reaction mixture and it was extracted with DCM (3×15 mL). The organic layer was rinsed with brine, dried with Na₂SO₄ and concentrated to give a brown oil. The brown oil was purified using Biotage™ flash chromatography and a 25 g SNAP™ column, 20-100% EtOAc-hexanes, 15 column volumes. Fractions containing product were combined and concentrated to give the title compound (25 mg, 0.050 mmol, 40.1% yield) as a light brown solid. ¹H NMR (CDCl₃, 400 MHz) δ 1.23 (d, J=4 Hz, 6H), 1.46-1.55 (m, 6H), 1.77-1.82 (m, 2H), 1.96-2.01 (m, 2H), 2.09-2.14 (m, 2H), 3.06 (t, J=4 Hz, 4H), 3.09-3.16 (m, 2H), 3.48-3.52 (m, 1H), 3.54-3.59 (m, 1H), 3.78-3.84 (m, 2H), 4.06 (t, J=4 Hz, 4H), 4.85-4.94 (m, 2H), 7.73 (s, 1H), 7.82 (s, 1H), LCMS m/z: 497.6 [M+H]⁺.

### Example 1.78

### Preparation of isopropyl 4-((1r,4r)-4-(5-morpholinopyrazin-2-yloxy)cyclohexyloxy)piperidine-1-carboxylate (Compound 79)

A mixture isopropyl 4-((1r,4r)-4-(5-chloropyrazin-2-yloxy)cyclohexyloxy)piperidine-1-carboxylate (30 mg, 0.075 mmol), diacetoxypalladium (1.69 mg), morpholine (6.57 mg, 0.075 mmol), sodium 2-methylpropan-2-olate (17 mg, 0.181 mmol), and 1,1′-bis(di-t-butylphosphino)ferrocene (7.15 mg, 0.015 mmol) in 1,4-dioxane (0.603 ml) was heated at 100° C. for 1 h under microwave irradiation. LCMS showed product formed. Water was added to the reaction mixture and it was extracted with DCM (3×15 mL). The organic layer was washed with brine, then treated with sodium sulfate and concentrated to give a brown oil. The brown oil was purified using Biotage™ flash chromatography and a 10 g SNAP™ column, 10-100% EtOAc-hexanes, 15 column volumes. Fractions containing product were combined and concentrated to give the title compound (13 mg, 0.029 mmol, 38.4% yield) as a white solid. ¹H NMR (CDCl₃, 400 MHz) δ 1.25 (d, J=8 Hz, 6H), 1.45-1.56 (m, 6H), 1.77-1.82 (m, 2H), 1.96-2.00 (m, 2H), 2.09-2.13 (m, 2H), 3.09-3.16 (m, 2H), 3.37 (t, J=4 Hz, 4H), 3.46-3.49 (m, 1H), 3.55-3.59 (m, 1H), 3.80-3.83 (m, 2H), 3.85 (t, J=4 Hz, 4H), 4.83-4.94 (m, 2H), 7.63 (s, 1H), 7.82 (s, 1H), LCMS m/z: 449.4 [M+H]⁺.

### Example 1.79

### Preparation of 1-fluoropropan-2-yl 4-((1r,4r)-4-(5-(methylsulfonyl)pyrazin-2-yloxy)cyclohexyloxy)piperidine-1-carboxylate (Compound 81)

The title compound was prepared in a similar manner as described in Example 1.57 using 2-(methylsulfonyl)-5-((1r,4r)-4-(piperidin-4-yloxy)cyclohexyloxy)pyrazine hydrochloride and 1-fluoropropan-2-ol. ¹H NMR (CDCl₃, 400 MHz) δ 1.29 (d, J=8 Hz, 3H), 1.49-1.66 (m, 6H), 1.78-1.84 (m, 2H), 1.96-2.01 (m, 2H), 2.13-2.17 (m, 2H), 3.18 (s, 3H), 3.20-3.24 (m, 2H), 3.50-3.55 (m, 1H), 3.60-3.63 (m, 1H), 3.77-3.82 (m, 2H), 4.31-4.53 (m, 2H), 4.98-5.07 (m, 1H), 5.16-5.21 (m, 1H), 8.19 (s, 1H), 8.79 (s, 1H), LCMS m/z: 460.4 [M+H]⁺.

### Example 1.80

### Preparation of 1-fluoropropan-2-yl 4-((1r,4r)-4-(5-(methylsulfonyl)pyrazin-2-yloxy)cyclohexyloxy)piperidine-1-carboxylate (Compound 81, Enantiomer 1)

The racemic 1-fluoropropan-2-yl 4-((1r,4r)-4-(5-(methylsulfonyl)pyrazin-2-yloxy)cyclohexyloxy)piperidine-1-carboxylate (see Example 1.79) was separated using Chiral HPLC:

Column Chiralpak IA column, 250 mm L×20 mm ID

Gradient: 20% IPA/Hexanes

Detector: 254 nm

Retention Time (Compound 81, Enantiomer 1)=34.17 min

¹H NMR (CDCl₃, 400 MHz) δ 1.29 (d, J=8 Hz, 3H), 1.48-1.66 (m, 6H), 1.78-1.83 (m, 2H), 1.96-2.01 (m, 2H), 2.13-2.17 (m, 2H), 3.18 (s, 3H), 3.20-3.24 (m, 2H), 3.51-3.55 (m, 1H), 3.58-3.62 (m, 1H), 3.77-3.82 (m, 2H), 4.31-4.53 (m, 2H), 4.98-5.06 (m, 1H), 5.16-5.21 (m, 1H), 8.19 (s, 1H), 8.79 (s, 1H), LCMS m/z: 460.4 [M+H]⁺.

### Example 1.81

### Preparation of 1-fluoropropan-2-yl 4-((1r,4r)-4-(5-(methylsulfonyl)pyrazin-2-yloxy)cyclohexyloxy)piperidine-1-carboxylate (Compound 81, Enantiomer 2)

The racemic 1-fluoropropan-2-yl 4-((1r,4r)-4-(5-(methylsulfonyl)pyrazin-2-yloxy)cyclohexyloxy)piperidine-1-carboxylate (see Example 1.79) was separated using Chiral HPLC:

Column: Chiralpak IA column, 250 mm L×20 mm ID

Gradient: 20% IPA/Hexanes

Detector: 254 nm

Retention Time (Compound 81, Enantiomer 2)=49.82 min

¹H NMR (CDCl₃, 400 MHz) δ 1.29 (d, J=8 Hz, 3H), 1.49-1.66 (m, 6H), 1.78-1.84 (m, 2H), 1.96-2.01 (m, 2H), 2.13-2.17 (m, 2H), 3.18 (s, 3H), 3.20-3.24 (m, 2H), 3.50-3.55 (m, 1H), 3.60-3.63 (m, 1H), 3.77-3.82 (m, 2H), 4.31-4.53 (m, 2H), 4.98-5.07 (m, 1H), 5.16-5.21 (m, 1H), 8.19 (s, 1H), 8.79 (s, 1H), LCMS m/z: 460.4 [M+H]⁺.

### Example 1.82

### Preparation of 1,1,1-trifluoropropan-2-yl 4-((1r,4r)-4-(5-(methylsulfonyl)pyrazin-2-yloxy)cyclohexyloxy)piperidine-1-carboxylate (Compound 80)

The title compound was prepared in a similar manner as described in Example 1.57 using 2-(methylsulfonyl)-5-((1r,4r)-4-(piperidin-4-yloxy)cyclohexyloxy)pyrazine hydrochloride and 1,1,1-trifluoropropan-2-ol. ¹H NMR (CDCl₃, 400 MHz) δ 1.40 (d, J=8 Hz, 3H), 1.49-1.66 (m, 6H), 1.77-1.83 (m, 2H), 1.95-2.01 (m, 2H), 2.12-2.17 (m, 2H), 3.18 (s, 3H), 3.25-3.29 (m, 2H), 3.51-3.55 (m, 1H), 3.60-3.64 (m, 1H), 3.73-3.79 (m, 2H), 5.16-5.27 (m, 2H), 8.19 (s, 1H), 8.79 (s, 1H), LCMS m/z: 496.4 [M+H]⁺.

### Example 1.83

### Preparation of 1,1,1-trifluoropropan-2-yl 4-((1r,4r)-4-(5-(methylsulfonyl)pyrazin-2-yloxy)cyclohexyloxy)piperidine-1-carboxylate (Compound 80, Enantiomer 1)

The racemic 1,1,1-trifluoropropan-2-yl 4-((1r,4r)-4-(5-(methylsulfonyl)pyrazin-2-yloxy)cyclohexyloxy)piperidine-1-carboxylate (70 mg, see Example 1.82) was separated using Chiral HPLC:

Column: Chiralpak IA column, 250 mm L×20 mm ID

Gradient: 15% IPA/Hexanes

Detector: 254 nm

Retention Time for (Compound 80, Enantiomer 1)=29.64 min

¹H NMR (CDCl₃, 400 MHz) δ 1.40 (d, J=8 Hz, 3H), 1.50-1.66 (m, 6H), 1.78-1.83 (m, 2H), 1.95-2.01 (m, 2H), 2.12-2.17 (m, 2H), 3.18 (s, 3H), 3.25-3.29 (m, 2H), 3.51-3.55 (m, 1H), 3.60-3.64 (m, 1H), 3.73-3.78 (m, 2H), 5.16-5.26 (m, 2H), 8.19 (s, 1H), 8.79 (s, 1H), LCMS m/z: 496.4 [M+H]⁺.

### Example 1.84

### Preparation of 1,1,1-trifluoropropan-2-yl 4-((1r,4r)-4-(5-(methylsulfonyl)pyrazin-2-yloxy)cyclohexyloxy)piperidine-1-carboxylate (Compound 80, Enantiomer 2)

The racemic trans-1,1,1-trifluoropropan-2-yl 4-(4-(5-(methylsulfonyl)pyrazin-2-yloxy)cyclohexyloxy)piperidine-1-carboxylate (70 mg, see Example 1.82) was separated using Chiral HPLC:

Column: Chiralpak IA column, 250 mm L×20 mm ID; Gradient: 15% IPA/Hexanes; Detector: 254 nm; Retention Time for (Compound 80, Enantiomer 2)=36.63 min

¹H NMR (400 MHz, CDCl₃) δ 1.40 (d, J=8 Hz, 3H), 1.49-1.66 (m, 6H), 1.78-1.83 (m, 2H), 1.96-2.01 (m, 2H), 2.13-2.17 (m, 2H), 3.18 (s, 3H), 3.25-3.29 (m, 2H), 3.51-3.55 (m, 1H), 3.61-3.64 (m, 1H), 3.74-3.78 (m, 2H), 5.16-5.28 (m, 2H), 8.19 (s, 1H), 8.79 (s, 1H), LCMS m/z: 496.4 [M+H]⁺.

### Example 1.85

### Preparation of (S)-1,1,1-Trifluoropropan-2-yl 4-((1r,4r)-4-(5-(Methylsulfonyl)pyrazin-2-yloxy)cyclohexyloxy)piperidine-1-carboxylate (Compound 82)

A solution of (S)-1,1,1-trifluoropropan-2-ol (0.5 g, 4.44 mmol) and CDI (0.72 g, 4.44 mmol) in THF (14 mL) was stirred for 1.5 h at room temperature. To the resulting solution was added 2-(methylsulfonyl)-5-((1r,4r)-4-(piperidin-4-yloxy)cyclohexyloxy)pyrazine hydrochloride (see Example 1.36, Step C, 0.58 g, 1.48 mmol) and TEA (0.75 g, 7.40 mmol). The heterogeneous mixture was stirred overnight at 80° C. The resulting mixture was diluted with H₂O and extracted with DCM (3×). The combined organics were washed with H₂O and 1 N HCl, dried with anhydrous MgSO₄, and concentrated. The solid residue was triturated with IPA to give the title compound (0.61 g, 83%) as a white powder. Exact mass calculated for C₂₀H₂₈F₃N₃O₆S: 495.1. found LCMS m/z=496.2 [M+H]⁺. ¹H NMR (400 MHz, CDCl₃) δ 1.40 (d, J=6.6 Hz, 3H), 1.47-1.66 (m, 6H), 1.75-1.86 (bs, 2H), 1.94-2.02 (m, 2H), 2.10-2.19 (m, 2H), 3.18 (s, 3H), 3.21-3.33 (bs, 2H), 3.48-3.56 (m, 1H), 3.58-3.66 (m, 1H), 3.69-3.83 (m, 2H), 5.15-5.29 (m, 2H), 8.19 (d, J=1.2 Hz, 1H), 8.79 (d, J=1.2 Hz, 1H).

### Example 1.86

### Preparation of (R)-1,1,1-Trifluoropropan-2-yl 4-((1r,4r)-4-(5-(Methylsulfonyl)pyrazin-2-yloxy)cyclohexyloxy)piperidine-1-carboxylate (Compound 83)

A solution of (R)-1,1,1-trifluoropropan-2-ol (320 μL, 1.513 mmol) and di(1H-imidazol-1-yl)methanone (320 mg, 1.973 mmol) in 15 mL THF was stirred at room temperature. After 40 min, 2-(methylsulfonyl)-5-((1r,4r)-4-(piperidin-4-yloxy)cyclohexyloxy)pyrazine hydrochloride (see Example 1.36, Step C, 593 mg, 1.513 mmol) and triethylamine (640 μL, 4.60 mmol) were added. After stirring at 80° C. (oil bath) overnight, a pre-stirred solution (for 40 min) of (R)-1,1,1-trifluoropropan-2-ol (300 μL) and CDI (300 mg, 1.85 mmol) in 1 mL THF was added and stirred overnight. The resulting mixture was purified by chromatography (SiO₂, hexane/EtOAc) to give (R)-1,1,1-trifluoropropan-2-yl 4-((1r,4r)-4-(5-(methylsulfonyl)pyrazin-2-yloxy)cyclohexyloxy)piperidine-1-carboxylate (648 mg, 1.308 mmol, 86% yield) as a white solid. Exact mass calculated for C₂₀H₂₈F₃N₃O₆S: 495.17. found: LCMS m/z=496.4 [M+H]⁺; ¹H NMR (400 MHz, CDCl₃) δ 1.40 (d, J=6.3 Hz, 3H), 1.48-1.66 (m, 6H), 1.78-1.86 (m, 2H), 1.96-2.01 (m, 2H), 2.13-2.17 (m, 2H), 3.18 (s, 3H), 3.20-3.31 (m, 2H), 3.50-3.56 (m, 1H), 3.60-3.65 (m, 1H), 3.73-3.79 (m, 2H), 5.16-5.27 (m, 2H), 8.19 (d, J=1.3 Hz, 1H), 8.79 (d, J=1.3 Hz, 1H).

### Example 1.87

### Preparation of Isopropyl 4-((1r,4r)-4-(2-Methylpyridin-3-yloxy)cyclohexyloxy)piperidine-1-carboxylate (Compound 84)

### Step A: Preparation of 2-Methyl-3-((1r,4r)-4-(1-methylpiperidin-4-yloxy)cyclohexyloxy)pyridine

To a solution of (1s,4s)-4-(1-methylpiperidin-4-yloxy)cyclohexanol (690 mg, 3.23 mmol), 2-methylpyridin-3-ol (500 mg, 4.58 mmol), and triphenylphosphine (975 mg, 3.72 mmol) in THF (10 mL) cooled in an ice bath was added (E)-diisopropyl diazene-1,2-dicarboxylate (700 μL, 3.56 mmol). The mixture was allowed to warm to room temperature. After stirring for 3 h, the resulting mixture was partitioned between 1 M NaOH and CH₂Cl₂. The organic phase was concentrated and the residue was purified by gradient HPLC (5-70% CH₃CN/H₂O, 40 min). Fractions containing the desired product were partly concentrated and the residue was partitioned between 1 M NaOH and CH₂Cl₂. The combined organic phases were dried over MgSO₄, filtered, and concentrated to give 2-methyl-3-((1r,4r)-4-(1-methylpiperidin-4-yloxy)cyclohexyloxy)pyridine (40% pure, 265 mg, 11% yield) and used without further purification in Example 1.87, Step B. Exact mass calculated for C₁₈H₂₈N₂O₂: 304.22. found: LCMS m/z=305.2 [M+H]⁺.

### Step B: Preparation of Isopropyl 4-((1r,4r)-4-(2-methylpyridin-3-yloxy)cyclohexyloxy)piperidine-1-carboxylate

To a solution of 2-methyl-3-((1r,4r)-4-(1-methylpiperidin-4-yloxy)cyclohexyloxy)pyridine (crude product from Example 1.87, Step A, 40% pure, 260 mg, 0.34 mmol) in 5 mL CH₂Cl₂, was added isopropyl carbonochloridate (1.0 mL, 1.000 mmol) and DIEA (0.2 mL, 1.145 mmol). After stirring at room temperature overnight, the resulting mixture was partitioned between 1 M NaOH and CH₂Cl₂. The organic phase was concentrated and the residue was purified by gradient HPLC (5-95% CH₃CN/H₂O, 50 min). Fractions containing the desired product were partly concentrated and the residues were partitioned between 1 M NaOH and CH₂Cl₂. The combined organic phases were dried over MgSO₄, filtered, and concentrated to give isopropyl 4-((1r,4r)-4-(2-methylpyridin-3-yloxy)cyclohexyloxy)piperidine-1-carboxylate (34.2 mg, 0.091 mmol, 26.6% yield) as a sticky solid. Exact mass calculated for C₂₁H₃₂N₂O₄: 376.24. found: LCMS m/z=377.4 [M+H]⁺; ¹H NMR (400 MHz, CDCl₃) δ 1.23 (d, J=6.3 Hz, 6H), 1.43-1.64 (m, 6H), 1.77-1.81 (m, 2H), 1.94-1.99 (m, 2H), 2.06-2.11 (m, 2H), 2.46 (s, 3H), 3.11-3.18 (m, 2H), 3.53-3.59 (m, 2H), 3.78-3.82 (m, 2H), 4.27-4.31 (m, 1H), 4.88-4.94 (m, 1H), 7.04-7.09 (m, 2H), 8.05-8.07 (m, 1H).

### Example 1.88

### Preparation of Isopropyl 4-((1r,4r)-4-(6-(Cyclopropylsulfonyl)-2-methylpyridin-3-yloxy)cyclohexyloxy)piperidine-1-carboxylate (Compound 85)

### Step A: Preparation of 6-(Cyclopropylsulfonyl)-2-methylpyridin-3-ol

To a solution of 6-(cyclopropylsulfonyl)-2-methylpyridin-3-amine (500 mg, 2.35 mmol) in THF (6.6 mL) at −5° C. (ice/NaCl) was added boron trifluoride diethyl etherate (1.16 mL, 9.42 mmol). To the resulting solution at −5° C. was added tert-butyl nitrite (0.97 mL, 8.24 mmol) dropwise over 15 minutes. The mixture was stirred at −5° C. for 2 h. The mixture was diluted with MTBE and concentrated under high vacuum to obtain a dark brown crude sticky solid. To the crude product was added glacial acetic acid (8 mL, 141 mmol) and water (0.42 mL, 23.56 mmol) and the mixture was heated to reflux (115° C.). After heating at 115° C. overnight the LCMS showed the presence of the desired product (m/z=214 [M+H]⁺), a side product (m/z=216 [M+H]⁺) and no starting material. The mixture was concentrated in vacuo to remove 80% of acetic acid. The aqueous layer was extracted with DCM (2×50 mL). The aqueous layer was neutralized with saturated NaHCO₃ (aq) to pH 4-5, extracted with DCM (2×50 mL). The organic layer were combined and concentrated in vacuo to obtain crude product. The crude product was purified by Biotage™ flash chromatography using 25 g SNAP™ column, 10-100% EtOAc-Hexane, 8 column volumes. Fractions containing pure product were combined and concentrated to provide 6-(cyclopropylsulfonyl)-2-methylpyridin-3-ol (255 mg, 1.196 mmol, 50.8%) as light brown solid. ¹H NMR (CDCl₃, 400 MHz) δ 1.03-1.07 (m, 2H), 1.32-1.37 (m, 2H), 2.56 (s, 3H), 2.77-2.83 (m, 1H), 7.23 (d, J=8 Hz, 1H), 7.74 d, J=8 Hz, 1H), LCMS m/z=214.0 [M+H]⁺.

### Step B: Preparation of 6-(Cyclopropylsulfonyl)-2-methyl-3-((1r,4r)-4-(1-methylpiperidin-4-yloxy)cyclohexyloxy)pyridine

To a solution of 6-(cyclopropylsulfonyl)-2-methylpyridin-3-ol (210 mg, 0.985 mmol), (1s,4s)-4-(1-methylpiperidin-4-yloxy)cyclohexanol (175 mg, 0.821 mmol), and triphenylphosphine (258 mg, 0.985 mmol) in THF (4.9 mL) at 0° C. was added DIAD (0.19 mL, 0.985 mmol) dropwise. The mixture was stirred at 0° C. for 1 h and stirred at room temperature for 16 h. LCMS showed formation of the desired product and starting material (i.e., 6-(cyclopropylsulfonyl)-2-methylpyridin-3-ol). To the resulting mixture was added (1s,4s)-4-(1-methylpiperidin-4-yloxy)cyclohexanol (175 mg, 0.821 mmol) and triphenylphosphine (258 mg, 0.985 mmol), cooled to 0° C., followed by DIAD (0.10 mL, 0.492 mmol) dropwise and stirred at room temperature for 5 h. LCMS showed the title compound and starting material (˜5% present). The mixture was evaporated under reduced pressure. DCM was added to the residue and washed with water. The DCM layer was separated, dried over Na₂SO₄, filtered, and concentrated to obtain crude product. The crude product was purified by Biotage™ flash chromatography using 25 g SNAP™ column, 0-10% MeOH—CH₂Cl₂ (1% triethylamine), 10 column volumes. Fractions with pure product were combined and concentrated to obtain the product (oil, 135 mg) as a mixture of 6-(cyclopropylsulfonyl)-2-methyl-3-((1r,4r)-4-(1-methylpiperidin-4-yloxy)cyclohexyloxy)pyridine (30%) and 4-(cyclohex-3-enyloxy)-1-methylpiperidine and was used in Example 1.88, Step C without further purification; LCMS m/z=409.3 [M+H]⁺.

### Step C: Preparation of Isopropyl 4-((1r,4r)-4-(6-(Cyclopropylsulfonyl)-2-methylpyridin-3-yloxy)cyclohexyloxy)piperidine-1-carboxylate

A mixture of 6-(cyclopropylsulfonyl)-2-methyl-3-(cis-4-(1-methylpiperidin-4-yloxy)cyclohexyloxy)pyridine (30% pure) (130 mg, 0.318 mmol) in 2.9 mL of anhydrous DCM and N-ethyl-N-isopropylpropan-2-amine (0.14 mL, 0.795 mmol) was added isopropyl chloroformate (1.0 M in toluene) solution (0.63 mL, 0.636 mmol) dropwise at room temperature and stirred at room temperature overnight. LCMS showed product formed (25%) and starting material. To the reaction mixture, excess of isopropyl chloroformate (1.0 M in toluene) (0.4 mL) and DCE (1,2-dichloroethane solvent) was added and reaction was stirred at 50° C. for 16 h. LCMS showed complete formation of product and no starting material. The reaction mixture was diluted with DCM and washed with water. The DCM layer was separated, dried with Na₂SO₄, concentrated, and purified by Biotage™ flash chromatography using 10 g SNAP column, 0-100% EtOAc-hexane, 20 column volumes. The fractions containing pure product were combined and concentrated to obtain isopropyl 4-((1r,4r)-4-(6-(cyclopropylsulfonyl)-2-methylpyridin-3-yloxy)cyclohexyloxy)piperidine-1-carboxylate as a white solid (38 mg, 0.079 mmol, 83%). ¹H NMR (400 MHz, CDCl₃) δ 0.99-1.05 (m, 2H), 1.24 (d, J=8 Hz, 6H), 1.32-1.36 (m, 2H), 1.50-1.58 (m, 6H), 1.61-1.69 (m, 2H), 1.77-1.84 (m, 2H), 1.93-1.99 (m, 2H), 2.08-2.13 (m, 2H), 2.51 (s, 3H), 2.76-2.83 (m, 1H), 3.12-3.19 (m, 2H), 3.54-3.60 (m, 2H), 3.77-3.82 (m, 2H), 4.41-4.46 (m, 1H), 4.88-4.94 (m, 1H), 7.14 (d, J=8 Hz, 1H), 7.80 (d, J=8 Hz, 1H), LCMS m/z: 481.4 [M+1]⁺.

### Example 1.89

### Preparation of Phenyl 4-((1r,4r)-4-(5-(Methylsulfonyl)pyrazin-2-yloxy)cyclohexyloxy)piperidine-1-carboxylate (Compound 86)

To a solution of 2-((1r,4r)-4-(1-methylpiperidin-4-yloxy)cyclohexyloxy)-5-(methylsulfonyl)pyrazine (8.4 g, 22.7 mmol), N-ethyl-N-isopropylpropan-2-amine (15 mL, 86 mmol), and CH₂Cl₂ (200 mL) cooled in an ice bath was added phenyl carbonochloridate (8.6 mL, 68.3 mmol). The mixture was allowed to warm to room temperature. After 5 h, the mixture was diluted with additional CH₂Cl₂ and extracted with water. The aqueous phase was removed. The organic phase was dried over MgSO₄, filtered, and concentrated. The residue was purified by Biotage™ flash chromatography (SiO₂, hexane/EtOAc) to give phenyl 4-((1r,4r)-4-(5-(methylsulfonyl)pyrazin-2-yloxy)cyclohexyloxy)piperidine-1-carboxylate (5.1 g, 10.7 mmol, 47.2% yield) as a white solid. Exact mass calculated for C₂₃H₂₉N₃O₆S: 475.18. found: LCMS m/z=476.4 [M+H]⁺; ¹H NMR (400 MHz, CDCl₃) δ 1.51-1.69 (m, 4H), 1.86-1.92 (m, 2H), 1.99-2.03 (m, 2H), 2.14-2.19 (m, 2H), 3.19 (s, 3H), 3.23-3.48 (m, 2H), 3.54-3.58 (m, 2H), 3.65-3.69 (m, 2H), 3.85-3.98 (m, 2H), 5.18-5.22 (m, 1H), 7.09-7.11 (m, 2H), 7.17-7.21 (m, 1H), 7.33-7.37 (m, 2H), 8.19 (d, J=1.3 Hz, 1H), 8.79 (d, J=1.3 Hz, 1H).

### Example 1.90

### Preparation of 5-Isopropyl-3-(4-((1r,4r)-4-(5-(methylsulfonyl)pyrazin-2-yloxy)cyclohexyloxy)piperidin-1-yl)-1,2,4-oxadiazole (Compound 87)

### Step A: Preparation of N′-Hydroxy-4-((1r,4r)-4-(5-(Methylsulfonyl)pyrazin-2-yloxy)cyclohexyloxy)piperidine-1-carboximidamide

A mixture of 4-((1r,4r)-4-(5-(methylsulfonyl)pyrazin-2-yloxy)cyclohexyloxy)piperidine-1-carbonitrile (see Example 1.58, Step A, 349 mg, 0.917 mmol), hydroxylamine (50% in water, 4 mL) and EtOH (4 mL) was stirred at 60° C. (oil bath) for 30 min. The mixture was concentrated to give N′-hydroxy-4-((1r,4r)-4-(5-(methylsulfonyl)pyrazin-2-yloxy)cyclohexyloxy)piperidine-1-carboximidamide (377 mg, 0.638 mmol, 69.6% yield) as a white solid and used without further purification in Example 1.90, Step B. Exact mass calculated for C₁₇H₂₇N₅O₅S: 413.17. found: LCMS m/z=414.4 [M+H]⁺.

### Step B: Preparation of 5-Isopropyl-3-(4-((1r,4r)-4-(5-(methylsulfonyl)pyrazin-2-yloxy)cyclohexyloxy)piperidin-1-yl)-1,2,4-oxadiazole

A mixture of isobutyric acid (101 μl, 1.089 mmol) and di(1H-imidazol-1-yl)methanone (225 mg, 1.388 mmol) in 5 mL DMA was stirred at room temperature. After 15 min, N′-hydroxy-4-((1r,4r)-4-(5-(methylsulfonyl)pyrazin-2-yloxy)cyclohexyloxy)piperidine-1-carboximidamide from Example 1.90, Step A (70% pure, 340 mg, 0.58 mmol) was added and stirred at 110° C. (oil bath) for 3 h. The mixture was purified by prep HPLC (5-95% CH₃CN in 60 min). Fractions containing the desired product were partly concentrated and residue was partitioned between 1 M NaOH and CH₂Cl₂. The organic phases were dried over MgSO₄, filtered, and concentrated to give 5-isopropyl-3-(4-((1r,4r)-4-(5-(methylsulfonyl)pyrazin-2-yloxy)cyclohexyloxy)piperidin-1-yl)-1,2,4-oxadiazole (100.7 mg, 0.216 mmol, 26.3% yield) as a white solid. Exact mass calculated for C₂₁H₃₁N₅O₅S: 465.20. found: LCMS m/z=466.4 [M+H]⁺; ¹H NMR (400 MHz, CDCl₃) δ 1.35 (d, J=7.1 Hz, 6H), 1.52-1.69 (m, 6H), 1.87-1.93 (m, 2H), 1.98-2.02 (m, 2H), 2.13-2.19 (m, 2H), 3.03-3.10 (m, 1H), 3.08-3.21 (m, 5H), 3.52-3.64 (m, 2H), 3.75-3.81 (m, 2H), 5.16-5.22 (m, 1H), 8.19 (d, J=1.3 Hz, 1H), 8.79 (d, J=1.3 Hz, 1H).

### Example 1.91

### Preparation of 3-Isopropyl-5-(4-((1r,4r)-4-(5-(methylsulfonyl)pyrazin-2-yloxy)cyclohexyloxy)piperidin-1-yl)-1,2,4-oxadiazole (Compound 88)

### Step A: Preparation of 4-((1r,4r)-4-(5-(Methylsulfonyl)pyrazin-2-yloxy)cyclohexyloxy)piperidine-1-carbonitrile

To a solution of 2-((1r,4r)-4-(1-methylpiperidin-4-yloxy)cyclohexyloxy)-5-(methylsulfonyl)pyrazine (1.15 g, 3.11 mmol) and DIEA (0.81 g, 6.22 mmol) in DCM (12 mL) was added a solution of cyanic bromide (0.346 g, 3.27 mmol) in DCM (1 mL) at room temperature. The resulting mixture was stirred for 30 min at the same temperature and quenched with H₂O. The mixture was diluted with DCM, washed with H₂O and 1 N HCl, dried, and concentrated. The residue was purified by column chromatography to give the title compound (0.74 g, 63%). Exact mass calculated for C₁₇H₂₄N₄O₄S: 380.1. found LCMS m/z=381.2 [M+H]⁺.

### Step B: Preparation of 3-isopropyl-5-(4-((1r,4r)-4-(5-(methylsulfonyl)pyrazin-2-yloxy)cyclohexyloxy)piperidin-1-yl)-1,2,4-oxadiazole

To a solution of 4-((1r,4r)-4-(5-(methylsulfonyl)pyrazin-2-yloxy)cyclohexyloxy)piperidine-1-carbonitrile (0.46 g, 1.21 mmol) and (Z)—N′-hydroxyisobutyrimidamide (0.21 g, 2.06 mmol) in THF (8 mL) was added 0.5 M ZnCl₂ (4.11 mL, 2.06 mmol) in THF. The mixture was stirred for 3 h at room temperature. The conversion of the starting material to the corresponding intermediate (mass=483) was monitored by LCMS. To the above reaction was added 4 M HCl (2.11 mL, 8.46 mmol) in dioxane. The mixture was stirred overnight at 65° C. LCMS confirmed completion of the reaction. The reaction mixture was diluted with EtOAc, washed with H₂O and 1 N HCl, dried with MgSO₄, and concentrated to give a solid residue. The solid residue was triturated with IPA to give the title compound (0.43 g, 76%) as a white powder. Exact mass calculated for C₂₁H₃₁N₅O₅S: 465.2. found LCMS m/z=466.4 [M+H]⁺. ¹H NMR (400 MHz, CDCl₃) δ 1.28 (d, J=6.9 Hz, 6H), 1.48-1.73 (m, 6H), 1.84-1.92 (m, 2H), 1.95-2.04 (m, 2H), 2.11-2.19 (m, 2H), 2.88 (septet, J=6.9 Hz, 1H), 3.18 (s, 3H), 3.40-3.48 (m, 2H), 3.51-3.58 (m, 1H), 3.64-3.71 (m, 1H), 3.80-3.87 (m, 2H), 5.15-5.23 (m, 1H), 8.19 (d, J=1.2 Hz, 1H), 8.79 (d, J=1.2 Hz, 1H).

### Example 1.92

### Preparation of 3-(4-((1r,4r)-4-(5-(1H-1,2,4-Triazol-1-yl)pyrazin-2-yloxy)cyclohexyloxy)piperidin-1-yl)-5-(2-fluoropropan-2-yl)-1,2,4-oxadiazole (Compound 89)

### Step A: Preparation of 4-((1r,4r)-4-(5-(1H-1,2,4-triazol-1-yl)pyrazin-2-yloxy)cyclohexyloxy)-N-hydroxypiperidine-1-carboximidamide

4-((1r,4r)-4-(5-(1H-1,2,4-triazol-1-yl)pyrazin-2-yloxy)cyclohexyloxy)piperidine-1-carbonitrile (0.347 g, 0.939 mmol) was suspended in ethanol (4.0 mL) and 50% aqueous hydroxylamine (4.0 mL, 60.6 mmol) was added. The heterogeneous suspension was warmed to 60° C. and stirred for 15 minutes. Solid 4-((1r,4r)-4-(5-(1H-1,2,4-triazol-1-yl)pyrazin-2-yloxy)cyclohexyloxy)-N-hydroxypiperidine-1-carboximidamide (214 mg) was collected upon filtration of the crude reaction mixture. The filtrate was concentrated to a solid and then washed with EtOH/DCM/hexanes [1:1:1] to give an additional 84 mg of 4-((1r,4r)-4-(5-(1H-1,2,4-triazol-1-yl)pyrazin-2-yloxy)cyclohexyloxy)-N-hydroxypiperidine-1-carboximidamide. LCMS m/z=403.2 [M+H]⁺.

### Step B: Preparation of 3-(4-((1r,4r)-4-(5-(1H-1,2,4-Triazol-1-yl)pyrazin-2-yloxy)cyclohexyloxy)piperidin-1-yl)-5-(2-fluoropropan-2-yl)-1,2,4-oxadiazole

A solution of 2-fluoro-2-methylpropanoic acid (0.094 g, 0.889 mmol) and CDI (0.144 g, 0.889 mmol) in DMA (5.0 mL) at room temperature was stirred for 10 minutes and 4-((1r,4r)-4-(5-(1H-1,2,4-triazol-1-yl)pyrazin-2-yloxy)cyclohexyloxy)-N-hydroxypiperidine-1-carboximidamide (0.298 g, 0.740 mmol) was added. The reaction was warmed to 60° C. and stirred for 15 minutes and allowed to cool to room temperature. An additional amount of 2-fluoro-2-methylpropanoic acid (0.094 g, 0.889 mmol) and CDI (0.144 g, 0.889 mmol) were added and the reaction was heated to 60° C. and stirred for 15 min The resulting mixture was concentrated and partitioned between DCM and water and 1.0M HCl was added. The DCM was removed and the aqueous mixture was extracted twice with 10% IPA in chloroform. The extracts were dried (sodium sulfate), filtered, and concentrated. The crude product was purified by reverse phase HPLC to give 60 mg of the title compound. LCMS m/z=473.4 [M+H]⁺. ¹H NMR (400 MHz, CDCl₃) δ 1.48-1.71 (m, 6H), 1.79 (d, J=21.4 Hz, 6H), 1.86-1.95 (m, 2H), 1.97-2.06 (m, 2H), 2.13-2.22 (m, 2H), 3.19-3.27 (m, 2H), 3.51-3.59 (m, 1H), 3.60-3.68 (m, 1H), 3.76-3.84 (m, 2H), 5.06-5.14 (m, 1H), 8.00 (d, J=1.2 Hz, 1H), 8.18 (s, 1H), 8.71 (d, J=1.3 Hz, 1H), 9.10 (s, 1H).

### Example 1.93

### Preparation of 2,2,3,3-Tetrafluorocyclobutyl 4-((1r,4r)-4-(5-(methylsulfonyl)pyrazin-2-yloxy)cyclohexyloxy)piperidine-1-carboxylate (Compound 90)

A solution of 2,2,3,3-tetrafluorocyclobutanol (88 mg, 0.612 mmol) and CDI (99 mg, 0.612 mmol) in THF (4.0 mL) at room temperature was stirred for 1.5 h. To the solution was added 2-(methylsulfonyl)-5-((1r,4r)-4-(piperidin-4-yloxy)cyclohexyloxy)pyrazine hydrochloride (80 mg, 0.204 mmol) and TEA (103 mg, 1.02 mmol) and the resulting heterogeneous mixture was stirred overnight at 80° C. The reaction was diluted with H₂O and extracted with DCM (2×). The combined organic was washed with H₂O and 1 N HCl, dried with MgSO₄, and concentrated to give a solid residue. The solid residue was triturated with IPA to give the title compound (75 mg, 70%). Exact mass calculated for C₂₁H₂₇F₄N₃O₆S: 525.1. found LCMS m/z=526.2 [M+H]⁺. ¹H NMR (400 MHz, CDCl₃) δ 1.48-1.67 (m, 6H), 1.75-1.86 (bs, 2H), 1.94-2.02 (m, 2H), 2.11-2.19 (m, 2H), 2.45-2.61 (m, 1H), 2.88-3.02 (m, 1H), 3.18 (s, 3H), 3.23-3.38 (bs, 2H), 3.49-3.56 (m, 1H), 3.60-3.68 (bs, 1H), 3.67-3.81 (bs, 2H), 5.13-5.24 (m, 2H), 8.19 (d, J=1.2 Hz, 1H), 8.79 (d, J=1.2 Hz, 1H).

### Example 1.94

### Preparation of 1,1,1,3,3,3-Hexafluoropropan-2-yl 4-((1r,4r)-4-(5-(methylsulfonyl)pyrazin-2-yloxy)cyclohexyloxy)piperidine-1-carboxylate (Compound 91)

A solution of 1,1,1,3,3,3-hexafluoropropan-2-ol (0.060 mL, 0.536 mmol) and di(1H-imidazol-1-yl)methanone (50 mg, 0.308 mmol) in 1 mL THF at room temperature was stirred for 40 min. To the solution was added 2-(methylsulfonyl)-5-((1r,4r)-4-(piperidin-4-yloxy)cyclohexyloxy)pyrazine hydrochloride (70 mg, 0.179 mmol) and triethylamine (0.124 mL, 0.893 mmol) and the mixture was stirred under microwave irradiation at 150° C. for 1 h. The resulting mixture was purified by Biotage™ flash chromatography (SiO₂, hexane/EtOAc) to give 1,1,1,3,3,3-hexafluoropropan-2-yl 4-((1r,4r)-4-(5-(methylsulfonyl)pyrazin-2-yloxy)cyclohexyloxy)piperidine-1-carboxylate (78.2 mg, 0.142 mmol, 80% yield) as a white solid. Exact mass calculated for C₂₀H₂₅F₆N₃O₆S: 549.14. found: LCMS m/z=550.2 [M+H]⁺; ¹H NMR (400 MHz, CDCl₃) δ 1.48-1.67 (m, 6H), 1.78-1.86 (m, 2H), 1.96-2.01 (m, 2H), 2.13-2.18 (m, 2H), 3.18 (s, 3H), 3.37-3.44 (m, 2H), 3.51-3.55 (m, 1H), 3.65-3.69 (m, 1H), 3.72-3.78 (m, 2H), 5.17-5.21 (m, 1H), 5.72-5.78 (m, 1H), 8.19 (d, J=1.3 Hz, 1H), 8.79 (d, J=1.3 Hz, 1H).

### Example 1.95

### Preparation of Isopropyl 4-((1r,4r)-4-(6-bromopyridin-3-yloxy)cyclohexyloxy)piperidine-1-carboxylate (Compound 92)

### Step A: Preparation of 2-Bromo-5-((1r,4r)-4-(1-methylpiperidin-4-yloxy)cyclohexyloxy)pyridine

To a solution of (1s,4s)-4-(1-methylpiperidin-4-yloxy)cyclohexanol (426 mg, 1.997 mmol), 6-bromopyridin-3-ol (510 mg, 2.93 mmol), and triphenylphosphine (850 mg, 3.24 mmol) in 10 mL THF cooled in an ice bath was added (E)-diisopropyl diazene-1,2-dicarboxylate (656 μL, 3.32 mmol). The mixture was allowed to warm to room temperature, stirred for 2 h, and purified by Biotage™ flash chromatography (SiO₂, hexane/EtOAc gradient first, followed by CH₂Cl₂/MeOH/NEt₃ 100:10:1). Fractions containing the desired product were combined and concentrated. The resulting residue was re-purified by HPLC (5-95% CH₃CN in 40 min). Fractions containing the desired product were partly concentrated and residue was partitioned between 1 M NaOH and CH₂Cl₂. The organic phases were dried over MgSO₄, filtered, and concentrated to give 2-bromo-5-((1r,4r)-4-(1-methylpiperidin-4-yloxy)cyclohexyloxy)pyridine (155 mg, 0.420 mmol, 21.0% yield). Exact mass calculated for C₁₇H₂₅BrN₂O₂: 368.18. found: LCMS m/z=369.2 [M+H]⁺; ¹H NMR (400 MHz, CDCl₃) δ 1.47-1.66 (m, 6H), 1.83-1.87 (m, 2H), 1.95-1.99 (m, 2H), 2.06-2.12 (m, 4H), 2.27 (s, 3H), 2.70-2.73 (m, 2H), 3.38-3.51 (m, 2H), 4.25-4.29 (m, 1H), 7.06-7.09 (m, 1H), 7.34-7.36 (m, 1H), 8.03-8.04 (m, 1H).

### Step B: Preparation of Isopropyl 4-((1r,4r)-4-(6-bromopyridin-3-yloxy)cyclohexyloxy)piperidine-1-carboxylate

To a solution of 2-bromo-5-((1r,4r)-4-(1-methylpiperidin-4-yloxy)cyclohexyloxy)pyridine (153 mg, 0.414 mmol) in 1 mL CH₂Cl₂ was added isopropyl carbonochloridate (1 mL, 1.000 mmol) and DIEA (0.217 mL, 1.243 mmol). After stirring at room temperature for 3 h, the resulting mixture was purified by Biotage™ flash chromatography (SiO₂, hexane/EtOAc) to provide isopropyl 4-((1r,4r)-4-(6-bromopyridin-3-yloxy)cyclohexyloxy)piperidine-1-carboxylate (149 mg, 0.338 mmol, 81% yield) as a white solid. Exact mass calculated for C₂₀H₂₉BrN₂O₄: 440.13. found: LCMS m/z=441.4 [M+H]⁺; ¹H NMR (400 MHz, CDCl₃) δ 1.23 (d, J=6.3 Hz, 6H), 1.44-1.61 (m, 6H), 1.77-1.81 (m, 2H), 1.94-1.99 (m, 2H), 2.06-2.11 (m, 2H), 3.11-3.17 (m, 2H), 3.50-3.58 (m, 2H), 3.78-3.82 (m, 2H), 4.27-4.31 (m, 1H), 4.88-4.94 (m, 1H), 7.06-7.09 (m, 1H), 7.34-7.36 (m, 1H), 8.03-8.04 (m, 1H).

### Example 1.96

### Preparation of Isopropyl 4-((1r,4r)-4-(6-(methylsulfonyl)pyridin-3-yloxy)cyclohexyloxy)piperidine-1-carboxylate (Compound 93)

A mixture of isopropyl 4-((1r,4r)-4-(6-bromopyridin-3-yloxy)cyclohexyloxy)piperidine-1-carboxylate (see Example 1.95, 148 mg, 0.335 mmol), sodium methanesulfinate (144 mg, 1.411 mmol), copper (I) trifluoromethanesulfonate benzene complex (20 mg, 0.040 mmol), and N¹,N²-dimethylethane-1,2-diamine (10 μL, 0.091 mmol) in 2 mL DMSO was stirred at 120° C. (oil bath). The resulting mixture was purified by Biotage™ flash chromatography (SiO₂, hexane/EtOAc) to give isopropyl 4-((1r,4r)-4-(6-(methylsulfonyl)pyridin-3-yloxy)cyclohexyloxy)piperidine-1-carboxylate (81.4 mg, 0.185 mmol, 55.1% yield). Exact mass calculated for C₂₁H₃₂N₂O₆S: 440.20. found: LCMS m/z=441.4 [M+H]⁺; ¹H NMR (400 MHz, CDCl₃) δ 1.23 (d, J=6.3 Hz, 6H), 1.45-1.55 (m, 4H), 1.59-1.67 (m, 2H), 1.77-1.82 (m, 2H), 1.96-2.01 (m, 2H), 2.06-2.11 (m, 2H), 3.11-3.17 (m, 5H), 3.50-3.58 (m, 2H), 3.78-3.82 (m, 2H), 4.27-4.31 (m, 1H), 4.88-4.94 (m, 1H), 7.06-7.09 (m, 1H), 7.34-7.36 (m, 1H), 8.03-8.04 (m, 1H).

### Example 1.97

### Preparation of Isopropyl 4-((1S,4r)-4-(4-((S)-2-amino-3-(3,3-difluoroazetidin-1-yl)-3-oxopropyl)phenoxy)cyclohexyloxy)piperidine-1-carboxylate (Compound 94) as the Hydrochloride salt

### Step A: Preparation of (S)-Methyl 2-(tert-Butoxycarbonylamino)-3-(4-hydroxyphenyl)propanoate

A mixture of (S)-methyl 2-amino-3-(4-hydroxyphenyl)propanoate (2.17 g, 11.12 mmol), sodium bicarbonate (0.934 g, 11.12 mmol), and BOC-anhydride (2.447 mL, 10.54 mmol) in 50 mL EtOH was stirred at room temperature overnight. The mixture was partitioned between EtOAc and water. The aqueous phase was removed. The organic phase was dried over MgSO₄, filtered, and concentrated. The residue was purified by Biotage™ flash chromatography (SiO₂, hexane/EtOAc) to give (S)-methyl 2-(tert-butoxycarbonylamino)-3-(4-hydroxyphenyl)propanoate (1.63 g, 5.52 mmol, 49.7% yield) as a white solid. Exact mass calculated for C₁₅H₂₁NO₅: 295.33. found: LCMS m/z=296.2 [M+H]⁺; ¹H NMR (400 MHz, CDCl₃) δ 1.24 (s, 9H), 2.94-3.06 (m, 2H), 3.71 (s, 3H), 4.52-4.56 (m, 1H), 4.97-4.99 (m, 1H), 5.37 (s, 1H), 6.73 (d, J=8.2 Hz, 2H), 6.95-6.98 (m, 2H).

### Step B: Preparation of (S)-Methyl 2-(tert-Butoxycarbonylamino)-3-(4-((1r,4S)-4-(1-methylpiperidin-4-yloxy)cyclohexyloxy)phenyl)propanoate

To a solution of (1s,4s)-4-(1-methylpiperidin-4-yloxy)cyclohexanol (414 mg, 1.941 mmol), (S)-methyl 2-(tert-butoxycarbonylamino)-3-(4-hydroxyphenyl)propanoate (860 mg, 2.91 mmol), and triphenylphosphine (840 mg, 3.20 mmol) in 10 mL THF was added DIAD (620 μL, 3.19 mmol). After stirring at room temperature overnight, the mixture was partitioned between water and CH₂Cl₂. the aqueous phase was removed. The organic phases were dried over MgSO₄, filtered, and concentrated. The residue was purified by Biotage™ flash chromatography (SiO₂, hexane/EtOAc gradient first and then CH₂Cl₂/MeOH/NEt₃ 100:10:1) to give the title compound (40% pure, 460 mg, 0.375 mmol, 19.32% yield) and was used without further purification in Example 1.97, Step C. Exact mass calculated for C₂₇H₄₂N₂O₆: 490.30. found: LCMS m/z=491.4 (M+H).

### Step C: Preparation of Isopropyl 4-((1S,4r)-4-(4-((S)-2-(tert-Butoxycarbonylamino)-3-methoxy-3-oxopropyl)phenoxy)cyclohexyloxy)piperidine-1-carboxylate

To a solution of (S)-methyl 2-(tert-butoxycarbonylamino)-3-(4-((1r,4S)-4-(1-methylpiperidin-4-yloxy)cyclohexyloxy)phenyl)propanoate (see Example 1.97, Step B, ca. 40% pure, 460 mg, ca. 0.37 mmol) and DIEA (340 μL, 1.947 mmol) in 10 mL CH₂Cl₂ cooled in an ice bath was added isopropyl carbonochloridate (1500 μL, 1.500 mmol). The mixture was allowed to warm to room temperature. After stirring overnight, the resulting mixture was partitioned between water and CH₂Cl₂. The aqueous phase was removed. The organic phase was dried over MgSO₄, filtered, and concentrated. The residue was purified by HPLC (5-95% CH₃CN in 30 min+10 min 95% CH₃CN). The fractions containing the desired product were combined, partially concentrated, and residue was partitioned between 1 M NaOH and CH₂Cl₂. The organic phase was dried over MgSO₄, filtered, and concentrated to give isopropyl 4-((1S,4r)-4-(4-((S)-2-(tert-butoxycarbonylamino)-3-methoxy-3-oxopropyl)phenoxy)cyclohexyloxy)piperidine-1-carboxylate (94.2 mg, 0.167 mmol, 45.2% yield) as a white solid. Exact mass calculated for C₃₀H₄₆N₂O₈: 562.33. found: LCMS m/z=563.6 [M+H]⁺; ¹H NMR (400 MHz, CDCl₃) δ 1.23 (d, J=6.3 Hz, 6H), 1.41-1.57 (m, 15H), 1.78-1.81 (m, 2H), 1.95-2.00 (m, 2H), 2.04-2.10 (m, 2H), 2.98-3.16 (m, 4H), 3.49-3.57 (m, 2H), 3.71 (s, 3H), 3.79-3.83 (m, 2H), 4.20-4.24 (m, 1H), 4.52-4.56 (m, 1H), 4.88-4.99 (m, 2H), 6.80-6.82 (m, 2H), 7.00-7.02 (m, 2H).

### Step D: Preparation of Isopropyl 4-((1S,4r)-4-(4-((S)-2-(tert-Butoxycarbonylamino)-3-(3,3-difluoroazetidin-1-yl)-3-oxopropyl)phenoxy)cyclohexyloxy)piperidine-1-carboxylate

To a solution of isopropyl 4-((1S,4r)-4-(4-((S)-2-(tert-butoxycarbonylamino)-3-methoxy-3-oxopropyl)phenoxy)cyclohexyloxy)piperidine-1-carboxylate (91.8 mg, 0.163 mmol) in 2 mL THF/MeOH/H₂O (3:1:1), lithium hydroxide hydrate (22.3 mg, 0.531 mmol) was added. After stirring at room temperature for 1.5 h, the mixture was concentrated and dried under high vacuum. The resulting crude (S)-2-(tert-butoxycarbonylamino)-3-(4-((1r,4S)-4-(1-(isopropoxycarbonyl)piperidin-4-yloxy)cyclohexyloxy)phenyl)propanoic acid (≦0.163 mmol) was dissolved in 2 mL DMF, 3,3-difluoroazetidine hydrochloride (36 mg, 0.278 mmol), triethylamine (116 μL, 0.833 mmol), and HATU (130 mg, 0.342 mmol) were added. After stirring at room temperature for 30 min, the mixture was purified by HPLC (5-95% CH₃CN in 30 min+20 min 95% CH₃CN). Fractions containing the product were concentrated, extracted, and further purified by Biotage™ flash chromatography (SiO₂, hexane/EtOAc) to give isopropyl 4-((1S,4r)-4-(4-((S)-2-(tert-butoxycarbonylamino)-3-(3,3-difluoroazetidin-1-yl)-3-oxopropyl)phenoxy)cyclohexyloxy)piperidine-1-carboxylate (58.1 mg, 0.093 mmol, 57.1% yield) as a white solid. Exact mass calculated for C₃₂H₄₇N₃O₇: 623.73. found: LCMS m/z=624.6 [M+H]⁺; ¹H NMR (400 MHz, CDCl₃) δ 1.23 (d, J=6.3 Hz, 6H), 1.41-1.57 (m, 15H), 1.78-1.81 (m, 2H), 1.95-2.00 (m, 2H), 2.04-2.10 (m, 2H), 2.80-2.86 (m, 1H), 2.95-3.00 (m, 1H), 3.13-3.19 (m, 2H), 3.25-3.35 (m, 1H), 3.46-3.61 (m, 2H), 3.79-3.83 (m, 2H), 4.10-4.33 (m, 5H), 4.88-4.94 (m, 1H), 5.31-5.34 (m, 1H), 6.84 (d, J=8.6 Hz, 2H), 7.10 (d, J=8.6 Hz, 2H).

### Step E: Preparation of Isopropyl 4-((1S,4r)-4-(4-((S)-2-Amino-3-(3,3-difluoroazetidin-1-yl)-3-oxopropyl)phenoxy)cyclohexyloxy)piperidine-1-carboxylate Hydrochloride

To a solution of isopropyl 4-((1S,4r)-4-(4-((S)-2-(tert-butoxycarbonylamino)-3-(3,3-difluoroazetidin-1-yl)-3-oxopropyl)phenoxy)cyclohexyloxy)piperidine-1-carboxylate (55.1 mg, 0.088 mmol) in 1 mL CH₂Cl₂, was added hydrogen chloride (4 M dioxane, 1 mL, 4.00 mmol). After stirring at room temperature overnight, the mixture was concentrated and purified by HPLC (5-95% CH₃CN in 30 min). Fractions containing the product were combined and concentrated. The residue was treated with 4 M HCl (1 mL), concentrated, and dried under high vacuum to give isopropyl 4-((1S,4r)-4-(4-((S)-2-amino-3-(3,3-difluoroazetidin-1-yl)-3-oxopropyl)phenoxy)cyclohexyloxy)piperidine-1-carboxylate hydrochloride (29.1 mg, 0.052 mmol, 58.8% yield) as a white solid. Exact mass calculated for C₂₇H₃₉F₂N₃O₅: 523.29. found: LCMS m/z=524.6 [M+H]⁺; ¹H NMR (400 MHz, CD₃OD) δ 1.23 (d, J=6.3 Hz, 6H), 1.41-1.55 (m, 6H), 1.80-1.85 (m, 2H), 1.99-2.08 (m, 4H), 2.95-2.98 (m, 1H), 2.95-3.00 (m, 1H), 3.06-3.20 (m, 3H), 3.56-3.69 (m, 2H), 3.75-3.80 (m, 2H), 4.05-4.09 (m, 1H), 4.20-4.41 (m, 4H), 4.81-4.88 (m, 1H), 6.94 (d, J=8.6 Hz, 2H), 7.18 (d, J=8.6 Hz, 2H).

### Example 1.98

### Preparation of Isopropyl 4-((1r,4r)-4-(6-(1H-1,2,4-triazol-1-yl)pyridin-3-yloxy)cyclohexyloxy)piperidine-1-carboxylate (Compound 95)

A mixture of isopropyl 4-((1r,4r)-4-(6-bromopyridin-3-yloxy)cyclohexyloxy)piperidine-1-carboxylate (60 mg, 0.136 mmol), 1H-1,2,4-triazole (50 mg, 0.724 mmol), N¹,N²-dimethylethane-1,2-diamine (10 μl, 0.091 mmol), copper(I) iodide (5 mg, 0.026 mmol), and potassium phosphate (70 mg, 0.330 mmol) in 1 mL DMA was heated under microwave irradiation at 220° C. for 1 h. The mixture was purified by HPLC (5-95% CH₃CN in 30 min). Fractions containing the desired product were combined, partially concentrated and the residue was partitioned between 1 M NaHCO₃ and CH₂Cl₂. The organic phase was dried over MgSO₄, filtered, and concentrated to give isopropyl 4-((1r,4r)-4-(6-(1H-1,2,4-triazol-1-yl)pyridin-3-yloxy)cyclohexyloxy)piperidine-1-carboxylate (22.3 mg, 0.052 mmol, 38.2% yield) as a white solid. Exact mass calculated for C₂₂H₃₁N₅O₄: 429.24. found: LCMS m/z=430.4 [M+H]⁺; ¹H NMR (400 MHz, CDCl₃) δ 1.23 (d, J=6.3 Hz, 6H), 1.44-1.65 (m, 6H), 1.77-1.82 (m, 2H), 1.98-2.02 (m, 2H), 2.10-2.15 (m, 2H), 3.11-3.18 (m, 2H), 3.51-3.61 (m, 2H), 3.79-3.83 (m, 2H), 4.33-4.39 (m, 1H), 4.88-4.94 (m, 1H), 7.36-7.39 (m, 1H), 7.80-7.82 (d, J=8.9 Hz, 1H), 8.06-8.10 (m, 2H). 9.01 (s, 1H).

### Example 1.99

### Preparation of 5-(4-((1r,4r)-4-(5-(1H-1,2,4-Triazol-1-yl)pyrazin-2-yloxy)cyclohexyloxy)piperidin-1-yl)-3-isopropyl-1,2,4-oxadiazole (Compound 96)

### Step A: Preparation of 2-((1r,4r)-4-(1-Methylpiperidin-4-yloxy)cyclohexyloxy)-5-(1H-1,2,4-triazol-1-yl)pyrazine

To a suspension of 2-bromo-5-((1r,4r)-4-(1-methylpiperidin-4-yloxy)cyclohexyloxy)pyrazine (0.5 g, 1.35 mmol), 1H-1,2,4-triazole (0.121 g, 1.76 mmol), potassium phosphate (0.631 g, 2.97 mmol), copper iodide (0.064 g, 0.338 mmol) and N¹,N²-dimethylethane-1,2-diamine (0.080 mL, 0.743 mmol) in DMF (10.0 mL) was bubbled nitrogen gas for 5 minutes. The reaction was heated to 220° C. by microwave irradiation for 30 minutes. The reaction was diluted with water and filtered to give 338 mg of the title compound. LCMS m/z=359.4 [M+H]⁺.

### Step B: Preparation of 4-((1r,4r)-4-(5-(1H-1,2,4-triazol-1-yl)pyrazin-2-yloxy)cyclohexyloxy)piperidine-1-carbonitrile

To a solution of 2-((1r,4r)-4-(1-methylpiperidin-4-yloxy)cyclohexyloxy)-5-(1H-1,2,4-triazol-1-yl)pyrazine (0.425 g, 1.19 mmol) in DCM (4.0 mL) was added DIEA (1.035 mL, 5.93 mmol) and cyanic bromide (0.138 g, 1.30 mmol). The resulting reaction was stirred at room temperature for 1 h and then diluted with DCM and washed two times with 1.0M HCl. The DCM layer was dried over sodium sulfate, filtered, and concentrated to give 347 mg of the title compound. LCMS m/z=370.2 [M+H]⁺.

### Step C: Preparation of 5-(4-((1r,4r)-4-(5-(1H-1,2,4-triazol-1-yl)pyrazin-2-yloxy)cyclohexyloxy)piperidin-1-yl)-3-isopropyl-1,2,4-oxadiazole

To a solution of 4-((1r,4r)-4-(5-(1H-1,2,4-triazol-1-yl)pyrazin-2-yloxy)cyclohexyloxy)piperidine-1-carbonitrile (0.100 g, 0.271 mmol) and (E)-N′-hydroxyisobutyrimidamide (0.055 g, 0.542 mmol) in DCM (3.0 mL) was added 0.5 M zinc(II) chloride THF solution (1.084 mL, 0.542 mmol). The mixture was stirred for 3 h at room temperature. 4.0 M HCl in dioxane (0.3 mL) was added and the reaction was warmed to 40° C. The reaction was stirred for 2 d at this temperature, warmed to 60° C., and stirred for 4 h. The reaction mixture was diluted with water and filtered to give 30 mg of the title compound. LCMS m/z=455.2 [M+H]⁺; ¹H NMR (400 MHz, CDCl₃) δ 1.29 (d, J=6.9 Hz, 6H), 1.48-1.75 (m, 6H), 1.85-1.93 (m, 2H), 1.97-2.05 (m, 2H), 2.12-2.21 (m, 2H), 2.90 (septet, J=6.9 Hz, 1H), 3.42-3.58 (m, 3H), 3.65-3.73 (m, 1H), 3.80-3.89 (m, 2H), 5.05-5.14 (m, 1H), 7.99 (d, J=1.4 Hz, 1H), 8.11 (s, 1H), 8.70 (d, J=1.3 Hz, 1H), 8.98 (s, 1H).

### Example 1.100

### Preparation of isopropyl 4-((1R,4r)-4-(4-((R)-2-Amino-3-(3,3-difluoroazetidin-1-yl)-3-oxopropyl)phenoxy)cyclohexyloxy)piperidine-1-carboxylate (Compound 97)

### Step A: Preparation of (R)-methyl 2-(tert-butoxycarbonylamino)-3-(4-hydroxyphenyl)propanoate

A mixture of (R)-methyl 2-amino-3-(4-hydroxyphenyl)propanoate (2.28 g, 11.68 mmol), sodium bicarbonate (2.8 g, 33.3 mmol), and di-tert-butyl dicarbonate (2.8 g, 12.83 mmol) in 50 mL EtOH was stirred at room temperature overnight. The resulting mixture was partitioned between EtOAc and water. The aqueous phase was removed. The organic phase was dried over MgSO₄, filtered, and concentrated. The residue was purified by Biotage™ flash chromatography (SiO₂, hexane/EtOAc) to give (R)-methyl 2-(tert-butoxycarbonylamino)-3-(4-hydroxyphenyl)propanoate (2.0 g, 6.77 mmol, 58.0% yield) as a white solid. Exact mass calculated for C₁₅H₂₁NO₅: 295.33. found: LCMS m/z=296.2 [M+H]⁺; ¹H NMR (400 MHz, CDCl₃) δ 1.24 (s, 9H), 2.94-3.06 (m, 2H), 3.71 (s, 3H), 4.52-4.56 (m, 1H), 4.97-4.99 (m, 1H), 5.37 (s, 1H), 6.73 (d, J=8.2 Hz, 2H), 6.95-6.98 (m, 2H).

### Step B: Preparation of (R)-Methyl 2-(tert-butoxycarbonylamino)-3-(4-((1r,4R)-4-(1-methylpiperidin-4-yloxy)cyclohexyloxy)phenyl)propanoate

To a solution of (1s,4s)-4-(1-methylpiperidin-4-yloxy)cyclohexanol (439 mg, 2.06 mmol), (R)-methyl 2-(tert-butoxycarbonylamino)-3-(4-hydroxyphenyl)propanoate (883 mg, 2.99 mmol), and triphenylphosphine (741 mg, 2.83 mmol) in 10 mL THF cooled in an ice bath was added DIAD (500 μL, 2.40 mmol). After stirring at room temperature overnight, the resulting mixture was partitioned between water and CH₂Cl₂. The aqueous phase was removed. The organic phase was dried over MgSO₄, filtered, and concentrated. The residue was purified by Biotage™ flash chromatography (SiO₂, hexane/EtOAc gradient first and then CH₂Cl₂/MeOH/NEt₃ 100:10:1). Fractions containing the desired product were concentrated and the residue was further purified by HPLC (5-95% CH₃CN in 20 min+20 min 95% CH₃CN) to give the title compound (236 mg, 0.481 mmol, 23% yield). Exact mass calculated for C₂₇H₄₂N₂O₆: 490.30. found: LCMS m/z=491.6 (M+H). ¹H NMR (400 MHz, CDCl₃) δ 1.41-1.57 (m, 15H), 1.82-1.87 (m, 2H), 1.96-2.00 (m, 2H), 2.07-2.11 (m, 4H), 2.26 (s, 3H), 2.70-2.74 (m, 2H), 2.98-3.03 (m, 2H), 3.37-3.48 (m, 2H), 3.71 (s, 3H), 4.19-4.23 (m, 1H), 4.52-4.55 (m, 1H), 4.92-4.95 (m, 1H), 6.79-6.82 (m, 2H), 7.00-7.02 (m, 2H).

### Step C: Preparation of Isopropyl 4-((1R,4r)-4-(4-((R)-2-(tert-Butoxycarbonylamino)-3-methoxy-3-oxopropyl)phenoxy)cyclohexyloxy)piperidine-1-carboxylate

To a solution of (R)-methyl 2-(tert-butoxycarbonylamino)-3-(4-((1r,4R)-4-(1-methylpiperidin-4-yloxy)cyclohexyloxy)phenyl)propanoate (220 mg, 0.448 mmol) in 3 mL CH₂Cl₂ cooled in an ice bath was added isopropyl carbonochloridate (1 mL, 1.000 mmol) and N-ethyl-N-isopropylpropan-2-amine (58.0 mg, 0.448 mmol). After stirring at room temperature overnight, the mixture was purified by Biotage™ flash chromatography (SiO₂, hexane/EtOAc) to give the title compound (253 mg, 0.450 mmol, 100% yield) as a white solid. Exact mass calculated for C₃₀H₄₆N₂O₈: 562.33. found: LCMS m/z=563.6 [M+H]⁺; ¹H NMR (400 MHz, CDCl₃) δ 1.23 (d, J=6.3 Hz, 6H), 1.41-1.57 (m, 15H), 1.78-1.81 (m, 2H), 1.95-2.00 (m, 2H), 2.04-2.10 (m, 2H), 2.98-3.16 (m, 4H), 3.49-3.57 (m, 2H), 3.71 (s, 3H), 3.79-3.83 (m, 2H), 4.20-4.24 (m, 1H), 4.52-4.56 (m, 1H), 4.88-4.99 (m, 2H), 6.80-6.82 (m, 2H), 7.00-7.02 (m, 2H).

### Step D: Preparation of Isopropyl 4-((1R,4r)-4-(4-((R)-2-(tert-Butoxycarbonylamino)-3-(3,3-difluoroazetidin-1-yl)-3-oxopropyl)phenoxy)cyclohexyloxy)piperidine-1-carboxylate

To a solution of isopropyl 4-((1R,4r)-4-(4-((R)-2-(tert-butoxycarbonylamino)-3-methoxy-3-oxopropyl)phenoxy)cyclohexyloxy)piperidine-1-carboxylate (249 mg, 0.443 mmol) in 5 mL of THF/MeOH/H₂O (3:1:1) was added lithium hydroxide hydrate (64.8 mg, 1.544 mmol). After stirring at room temperature for 2 h, the mixture was concentrated and dried under high vacuum. The crude (R)-2-(tert-butoxycarbonylamino)-3-(4-((1r,4R)-4-(1-(isopropoxycarbonyl)piperidin-4-yloxy)cyclohexyloxy)phenyl)propanoic acid (241 mg, 0.44 mmol) was dissolved in 5 mL DMF and 3,3-difluoroazetidine hydrochloride (103 mg, 0.795 mmol), triethylamine (184 μL, 1.322 mmol), and 2-(3H-[1,2,3]triazolo[4,5-b]pyridin-3-yl)-1,1,3,3-tetramethylisouronium hexafluorophosphate(V) (340 mg, 0.894 mmol) were added. After stirring at room temperature for 15 min, the mixture was purified by HPLC (5-95% CH₃CN in 30 min+10 min 95% CH₃CN). The fractions containing the desired product were partially concentrated and the residue was partitioned between 1 M NaOH and CH₂Cl₂. The aqueous phase was removed. The organic phase was dried over MgSO₄, filtered, and concentrated to give the title compound (222 mg, 0.356 mmol, 81% yield). Exact mass calculated for C₃₂H₄₇N₃O₇: 623.73. found: LCMS m/z=624.6 [M+H]⁺; ¹H NMR (400 MHz, CDCl₃) δ 1.23 (d, J=6.3 Hz, 6H), 1.41-1.57 (m, 15H), 1.78-1.81 (m, 2H), 1.95-2.00 (m, 2H), 2.04-2.10 (m, 2H), 2.80-2.86 (m, 1H), 2.95-3.00 (m, 1H), 3.13-3.19 (m, 2H), 3.25-3.35 (m, 1H), 3.46-3.61 (m, 2H), 3.79-3.83 (m, 2H), 4.10-4.33 (m, 5H), 4.88-4.94 (m, 1H), 5.31-5.34 (m, 1H), 6.84 (d, J=8.6 Hz, 2H), 7.10 (d, J=8.6 Hz, 2H).

### Step E: Preparation of Isopropyl 4-((1R,4r)-4-(4-((R)-2-Amino-3-(3,3-difluoroazetidin-1-yl)-3-oxopropyl)phenoxy)cyclohexyloxy)piperidine-1-carboxylate Hydrochloride

To a solution of isopropyl 4-((1R,4r)-4-(4-((R)-2-(tert-butoxycarbonylamino)-3-(3,3-difluoroazetidin-1-yl)-3-oxopropyl)phenoxy)cyclohexyloxy)piperidine-1-carboxylate (220 mg, 0.353 mmol) in 2 mL CH₂Cl₂ was added hydrogen chloride (4 M in dioxane, 3 mL, 12.00 mmol). After stirring at room temperature for 4 h, the mixture was concentrated and dried under high vacuum to give the title compound (178 mg, 0.318 mmol, 90% yield) as a white solid. Exact mass calculated for C₂₇H₃₉F₂N₃O₅: 523.29. found: LCMS m/z=524.6 [M+H]⁺; ¹H NMR (400 MHz, CD₃OD) δ 1.23 (d, J=6.3 Hz, 6H), 1.41-1.55 (m, 6H), 1.80-1.85 (m, 2H), 1.99-2.08 (m, 4H), 2.95-2.98 (m, 1H), 2.95-3.00 (m, 1H), 3.06-3.20 (m, 3H), 3.56-3.69 (m, 2H), 3.75-3.80 (m, 2H), 4.05-4.09 (m, 1H), 4.20-4.41 (m, 4H), 4.81-4.88 (m, 1H), 6.94 (d, J=8.6 Hz, 2H), 7.18 (d, J=8.6 Hz, 2H).

### Example 1.101

### Preparation of 5-((1r,4r)-4-(1-(3-Isopropyl-1,2,4-oxadiazol-5-yl)piperidin-4-yloxy)cyclohexyloxy)-N,N-dimethylpyrazine-2-carboxamide (Compound 98)

To a stirred solution of (1r,4r)-4-(1-(3-isopropyl-1,2,4-oxadiazol-5-yl)piperidin-4-yloxy)cyclohexanol (265 mg, 0.857 mmol) in THF (3 mL) cooled in an ice-water bath under nitrogen was added 1 M potassium tert-butoxide in THF (943 μL, 0.943 mmol). The reaction mixture was stirred for 30 min and 5-chloro-N,N-dimethylpyrazine-2-carboxamide (175 mg, 0.943 mmol) (prepared as the same manner as described in Example 1.106, Step C) in THF (2 mL) was added. The mixture was stirred for 3 h at room temperature and water (1 mL) was added. The resulting mixture was partially evaporated, diluted with water, and extracted with ethyl acetate. The combined organics were dried over anhydrous Na₂SO₄, filtered, and concentrated. The residue was purified by column chromatography with 75% ethyl acetate/hexanes, and further purified by semi preparative HPLC (25-80% CH₃CN/H₂O with 0.1% TFA) to give the title compound (213 mg, 0.465 mmol, 54% yield) as a white solid. Exact mass calculated for C₂₃H₃₄N₆O₄: 458.3. found: LCMS m/z=459.4 [M+H]⁺; ¹H NMR (400 MHz, CDCl₃) δ 1.28 (d, J=7.0 Hz, 6H), 1.48-1.73 (m, 6H), 1.85-1.92 (m, 2H), 1.96-2.04 (m, 2H), 2.12-2.18 (m, 2H), 2.84-2.92 (m, 1H), 3.13 (s, 3H), 3.17 (s, 3H), 3.40-3.48 (m, 2H), 3.50-3.56 (m, 1H), 3.65-3.72 (m, 1H), 3.80-3.88 (m, 2H), 5.07-5.14 (m, 1H), 8.07 (d, J=1.3 Hz, 1H), 8.51 (d, J=1.3 Hz, 1H).

### Example 1.102

### Preparation of (R)-1,1,1-Trifluoropropan-2-yl 4-((1r,4r)-4-(5-(ethylsulfonyl)pyrazin-2-yloxy)cyclohexyloxy)piperidine-1-carboxylate (Compound 99)

### Step A: Preparation of tert-Butyl 4-((1r,4r)-4-(5-(ethylsulfonyl)pyrazin-2-yloxy)cyclohexyloxy)piperidine-1-carboxylate

A mixture of tert-butyl 4-((1r,4r)-4-(5-bromopyrazin-2-yloxy)cyclohexyloxy)piperidine-1-carboxylate (0.5 g, 1.1 mmol), sodium ethanesulfinate (0.254 g, 2.2 mmol), (CuOTf)₂PhH (50 mg, 0.1 mmol), N¹,N²-dimethylethane-1,2-diamine (19 mg, 0.22 mmol) in DMSO (8.0 mL) was microwaved for 1.5 h at 130° C. The reaction was diluted with H₂O, extracted with DCM/IPA (9/1). The combined organic layer was washed with 1 N HCl and brine, dried with MgSO₄, and concentrated. The residue was purified by column chromatography (silica gel, hexane/EtOAc/MeOH=100/0/0 to 70/25/5) to give the title compound (0.4 g, 78%).

Exact mass calculated for C₂₂H₃₅N₃O₆S: 469.2. found LCMS m/z=470.6 [M+H]⁺. ¹H NMR (400 MHz, CDCl₃) δ 1.32 (t, J=7.6 Hz, 3H), 1.46 (s, 9H), 1.46-1.67 (m, 6H), 1.74-1.83 (m, 2H), 1.94-2.02 (m, 2H), 2.11-2.19 (m, 2H), 3.05-3.13 (m, 2H), 3.33 (q, J=7.6 Hz, 2H), 3.49-3.60 (m, 2H), 3.73-3.82 (m, 2H), 5.14-5.21 (m, 1H), 8.19 (d, J=1.2 Hz, 1H), 8.77 (d, J=1.2 Hz, 1H).

### Step B: Preparation of 2-(Ethylsulfonyl)-5-((1r,4r)-4-(piperidin-4-yloxy)cyclohexyloxy)pyrazine Hydrochloride

To a solution of tert-butyl 4-((1r,4r)-4-(5-(ethylsulfonyl)pyrazin-2-yloxy)cyclohexyloxy)piperidine-1-carboxylate (0.39 g, 0.83 mmol) in dioxane (5 mL) was added 4N-HCl (3.11 mL, 12.5 mmol) in dioxane. The reaction was stirred for 3 h at room temperature. In the course of the reaction, the desired product was precipitated as an HCl salt. The product was filtered and washed with ether, and dried to give the desired product (0.30 g, 89%).

Exact mass calculated for C₁₇H₂₇N₃O₄S: 369.1. found LCMS m/z=370.1 [M+H]⁺.

### Step C: Preparation of (R)-1,1,1-Trifluoropropan-2-yl 4-((1r,4r)-4-(5-(ethylsulfonyl)pyrazin-2-yloxy)cyclohexyloxy)piperidine-1-carboxylate

The title compound was prepared using (R)-1,1,1-trifluoropropan-2-ol and 2-(ethylsulfonyl)-5-((1r,4r)-4-(piperidin-4-yloxy)cyclohexyloxy)pyrazine hydrochloride and a similar procedure as described in Example 1.93, using. Exact mass calculated for C₂₁H₃₀F₃N₃O₆S: 509.1. found LCMS m/z=510.4 [M+H]⁺. ¹H NMR (400 MHz, CDCl₃) δ 1.32 (t, J=7.4 Hz, 3H), 1.40 (d, J=6.6 Hz, 3H), 1.48-1.66 (m, 6H), 1.74-1.86 (bs, 2H), 1.94-2.03 (m, 2H), 2.11-2.19 (m, 2H), 3.20-3.35 (bs, 2H), 3.33 (q, J=7.4 Hz, 2H), 3.49-3.56 (m, 1H), 3.59-3.66 (m, 1H), 3.69-3.84 (m, 2H), 5.15-5.29 (m, 2H), 8.20 (d, J=1.2 Hz, 1H), 8.77 (d, J=1.2 Hz, 1H).

### Example 1.103

### Preparation of isopropyl 4-((1r,4r)-4-(6-Bromo-2-methylpyridin-3-yloxy)cyclohexyloxy)piperidine-1-carboxylate (Compound 100)

### Step A: Preparation of (R)-Methyl 2-(tert-Butoxycarbonylamino)-3-(4-hydroxyphenyl)propanoate

To a solution of (1s,4s)-4-(1-methylpiperidin-4-yloxy)cyclohexanol (6.05 g, 28.4 mmol) in MeOH was added 6-bromo-2-methylpyridin-3-ol (5.33 g, 28.4 mmol), triphenylphosphine (8.18 g, 31.2 mmol) and followed by (E)-diisopropyl diazene-1,2-dicarboxylate (6.31 g, 31.2 mmol) under Ar at room temperature. The reaction was stirred for 4 h at room temperature. The resulting solution was evaporated and purified by Biotage™ flash chromatography using SNAP 340G (EtOAc/Hex 0-100% in 40 min and then 0-10% MeOH in DCM with 1% TEA added in 40 min) to give 6-bromo-2-methyl-3-((1r,4r)-4-(1-methylpiperidin-4-yloxy)cyclohexyloxy)pyridine (40% pure, 6.9 g, 12.60 mmol, 25% yield). Used as crude in step B. Exact mass calculated for C₁₈H₂₇BrN₂O₂: 382.13. found: LCMS m/z=383.4 [M+H]⁺.

### Step B: Preparation of Isopropyl 4-((1r,4r)-4-(6-Bromo-2-methylpyridin-3-yloxy)cyclohexyloxy)piperidine-1-carboxylate

To a solution of 6-bromo-2-methyl-3-((1r,4r)-4-(1-methylpiperidin-4-yloxy)cyclohexyloxy)pyridine (40% pure from step A, 2.3 g, 2.4 mmol) in 60 mL CH₂Cl₂, isopropyl carbonochloridate (6 mL, 6.00 mmol) and N-ethyl-N-isopropylpropan-2-amine (1.3 mL, 7.46 mmol) were added. After stirring overnight, the solution was extracted with water. The aqueous phase was removed. The organic phase was dried over MgSO₄, filtered, and concentrated. The residue was purified by Biotage™ flash chromatography (SiO₂, hexane/EtOAc) to give isopropyl 4-((1r,4r)-4-(6-bromo-2-methylpyridin-3-yloxy)cyclohexyloxy)piperidine-1-carboxylate (0.926 g, 2.033 mmol, 85% yield) as a colorless oil. Exact mass calculated for C₂₁H₃₁BrN₂O₄: 454.15. found: LCMS m/z=455.2 [M+H]⁺; ¹H NMR (400 MHz, CDCl₃) δ 1.23 (d, J=6.3 Hz, 6H), 1.44-1.63 (m, 6H), 1.77-1.81 (m, 2H), 1.92-1.97 (m, 2H), 2.04-2.09 (m, 2H), 2.42 (s, 3H), 3.11-3.18 (m, 2H), 3.52-3.59 (m, 2H), 3.78-3.82 (m, 2H), 4.22-4.28 (m, 1H), 4.88-4.94 (m, 1H), 6.97 (d, J=8.6 Hz, 1H), 7.20-7.23 (dd, J=8.6, 0.5 Hz, 1H).

### Example 1.104

### Preparation of isopropyl 4-((1r,4r)-4-(2-Methyl-6-(methylsulfonyl)pyridin-3-yloxy)cyclohexyloxy)piperidine-1-carboxylate (Compound 101)

A mixture of isopropyl 4-((1r,4r)-4-(6-bromo-2-methylpyridin-3-yloxy)cyclohexyloxy)piperidine-1-carboxylate (see Example 1.103, 370 mg, 0.812 mmol), sodium methanesulfinate (305 mg, 2.99 mmol), copper (I) trifluoromethanesulfonate benzene complex (42 mg, 0.083 mmol), and N¹,N²-dimethylethane-1,2-diamine (20 μL, 0.186 mmol) in 4 mL DMSO was heated under microwave irradiation at 120° C. for 2 h. The mixture was partitioned between EtOAc and water. The aqueous phase was removed. The organic phase was dried over MgSO₄, filtered, and concentrated. Residue was purified by Biotage™ flash chromatography (SiO₂, hexane/EtOAc) to give isopropyl 4-((1r,4r)-4-(2-methyl-6-(methylsulfonyl)pyridin-3-yloxy)cyclohexyloxy)piperidine-1-carboxylate (341 mg, 0.750 mmol, 92% yield) as a white solid. Exact mass calculated for C₂₂H₃₄N₂O₆S: 454.21. found: LCMS m/z=455.2 [M+H]⁺; ¹H NMR (400 MHz, CDCl₃) δ 1.23 (d, J=6.3 Hz, 6H), 1.44-1.69 (m, 6H), 1.77-1.81 (m, 2H), 1.92-1.97 (m, 2H), 2.08-2.14 (m, 2H), 2.51 (s, 3H), 3.12-3.2 (m, 5H), 3.54-3.61 (m, 2H), 3.78-3.82 (m, 2H), 4.42-4.48 (m, 1H), 4.88-4.94 (m, 1H), 7.17 (d, J=8.6 Hz, 1H), 7.89 (d, J=8.6 Hz, 1H).

### Example 1.105

### Preparation of isopropyl 4-((1r,4r)-4-(2-Methyl-6-(pyrimidin-5-yl)pyridin-3-yloxy)cyclohexyloxy)piperidine-1-carboxylate (Compound 102)

A mixture of isopropyl 4-((1r,4r)-4-(6-bromo-2-methylpyridin-3-yloxy)cyclohexyloxy)piperidine-1-carboxylate (see Example 1.103, 125 mg, 0.274 mmol), pyrimidin-5-ylboronic acid (60.2 mg, 0.486 mmol), cesium carbonate (178 mg, 0.546 mmol), and bis(tri-t-butylphosphine)palladium(0) (5 mg, 9.78 μmol) in 4.5 mL dioxane/water (10:1) was heated under microwave irradiation at 120° C. for 1 h. The mixture was partitioned between water and EtOAc. The aqueous phase was removed. The organic phase was dried over MgSO₄, filtered, and concentrated. Residue was purified by Biotage™ flash chromatography (SiO₂, hexane/EtOAc) to give isopropyl 4-((1r,4r)-4-(2-methyl-6-(pyrimidin-5-yl)pyridin-3-yloxy)cyclohexyloxy)piperidine-1-carboxylate (69.2 mg, 0.152 mmol, 55.5% yield) as a white solid. Exact mass calculated for C₂₅H₃₄N₄O₄: 454.26. found: LCMS m/z=455.2 [M+H]⁺; ¹H NMR (400 MHz, CDCl₃) δ 1.23 (d, J=6.3 Hz, 6H), 1.48-1.67 (m, 6H), 1.77-1.81 (m, 2H), 1.96-2.01 (m, 2H), 2.10-2.15 (m, 2H), 2.54 (s, 3H), 3.12-3.19 (m, 2H), 3.56-3.60 (m, 2H), 3.79-3.82 (m, 2H), 4.37-4.39 (m, 1H), 4.90-4.94 (m, 1H), 7.18 (d, J=8.6 Hz, 1H), 7.53 (d, J=8.6 Hz, 1H), 9.18 (s, 1H), 9.26 (s, 2H).

### Example 1.106

### Preparation of (5-((1r,4r)-4-(1-(3-Isopropyl-1,2,4-oxadiazol-5-yl)piperidin-4-yloxy)cyclohexyloxy)pyrazin-2-yl)(3-methoxyazetidin-1-yl)methanone (Compound 103)

### Step A: Preparation of 4-((1r,4r)-4-hydroxycyclohexyloxy)piperidine-1-carbonitrile

To a stirred solution of (1r,4r)-4-(1-methylpiperidin-4-yloxy)cyclohexanol (6 g, 28.1 mmol) in DCM (50 mL) was added DIEA (17.15 mL, 98 mmol), followed by cyanic bromide in DCM (14.06 mL, 42.2 mmol). The reaction mixture was stirred at room temperature for 1 h under nitrogen. Water was added, and the mixture was extracted with DCM. The combined organics were dried over anhydrous Na₂SO₄, filtered then concentrated. The residue was purified by column chromatography with 2% methanol/ethyl acetate to give the title compound (4.476 g, 19.96 mmol, 71% yield) as off-white solid. Exact mass calculated for C₁₂H₂₀N₂O₂: 224.2. found: LCMS m/z=225.2 [M+H]⁺; ¹H NMR (400 MHz, CDCl₃) δ 1.28-1.41 (m, 5H), 1.63-1.72 (m, 2H), 1.81-2.00 (m, 6H), 3.05-3.11 (m, 2H), 3.32-3.45 (m, 3H), 3.55-3.60 (m, 1H), 3.65-3.72 (m, 1H).

### Step B: Preparation of (1r,4r)-4-(1-(3-isopropyl-1,2,4-oxadiazol-5-yl)piperidin-4-yloxy)cyclohexanol

To a solution of 4-((1r,4r)-4-hydroxycyclohexyloxy)piperidine-1-carbonitrile (4.476 g, 19.96 mmol) in anhydrous DCM (100 mL) was added (Z)—N′-hydroxyisobutyrimidamide (3.67 g, 35.9 mmol), followed by a solution of 0.5 M ZnCl₂ in THF (120 mL, 59.9 mmol). The reaction mixture was stirred at room temperature for 3 h under nitrogen. The resulting mixture was concentrated to give the intermediate. To a suspension of the residue in anhydrous THF (100 mL), was slowly added 4 M HCl in dioxane (29.9 mL, 120 mmol). The solution was heated at 60° C. for 12 h. The resulting mixture was partially evaporated, water was added, neutralized with saturated NaHCO₃ aqueous solution, and extracted with DCM. The combined organics were dried over anhydrous Na₂SO₄, filtered then concentrated. The residue was purified by column chromatography with 100% ethyl acetate to give the title compound (5.77 g, 18.65 mmol, 93% yield). Exact mass calculated for C₁₆H₂₇N₃O₃: 309.2. found: LCMS m/z=310.4 [M+H]⁺; ¹H NMR (400 MHz, CDCl₃) δ 1.26 (d, J=7.0 Hz, 6H), 1.26-1.41 (m, 5H), 1.60-1.70 (m, 2H), 1.81-2.00 (m, 6H), 2.84-2.91 (m, 1H), 3.36-3.45 (m, 3H), 3.62-3.73 (m, 2H), 3.78-3.88 (m, 2H).

### Step C: Preparation of (5-Chloropyrazin-2-yl)(3-methoxyazetidin-1-yl)methanone

To a suspension of 5-chloropyrazine-2-carboxylic acid (365 mg, 2.302 mmol) in anhydrous DCM (6 mL) was added oxalyl dichloride (1.381 mL, 2.76 mmol), followed by a few drops of DMF. The reaction mixture was stirred at room temperature overnight. Solvent was evaporated, the residue was dissolved in anhydrous DCM (6 mL), 3-methoxyazetidine hydrochloride (341 mg, 2.76 mmol) was added, followed by DIEA (1.404 mL, 8.06 mmol). The reaction mixture was stirred at room temperature for 5 h. Solvent was evaporated, the residue was dissolved in ethyl acetate, washed with 5% citric acid solution, saturated NaHCO₃ aqueous solution, brine, and dried over anhydrous Na₂SO₄. The mixture was filtered and concentrated. The residue was purified by column chromatography with 60% ethyl acetate/hexanes to give the title compound (269 mg, 1.18 mmol, 51% yield) as an off-white solid. Exact mass calculated for C₉H₁₀ClN₃O₂: 227.1. found: LCMS m/z=228.0 [M+H]⁺; ¹H NMR (400 MHz, CDCl₃) δ 3.33 (s, 3H), 4.09-4.13 (m, 1H), 4.25-4.30 (m, 1H), 4.37-4.42 (m, 1H), 4.48-4.54 (m, 1H), 4.79-4.84 (m, 1H), 8.53 (d, J=1.3 Hz, 1H), 9.10 (d, J=1.3 Hz, 1H).

### Step D: Preparation of (5-((1r,4r)-4-(1-(3-Isopropyl-1,2,4-oxadiazol-5-yl)piperidin-4-yloxy)cyclohexyloxy)pyrazin-2-yl)(3-methoxyazetidin-1-yl)methanone

To a stirred solution of (1r,4r)-4-(1-(3-isopropyl-1,2,4-oxadiazol-5-yl)piperidin-4-yloxy)cyclohexanol (150 mg, 0.485 mmol) in THF (2 mL) cooled in an ice-water bath under an atmosphere of nitrogen was added 1 M potassium tert-butoxide in THF (0.533 mL, 0.533 mmol). The reaction mixture was stirred for 30 min and (5-chloropyrazin-2-yl)(3-methoxyazetidin-1-yl)methanone (121 mg, 0.533 mmol) in THF (1 mL) was added. The mixture was stirred for 5 h at room temperature, water (1 mL) was added, and partially evaporated. The concentrate was diluted with water and extracted with ethyl acetate. The combined organics were dried over anhydrous Na₂SO₄, filtered, and concentrated. The residue was purified by semi preparative HPLC (25-80% CH₃CN/H₂O with 0.1% TFA) to give the title compound (138 mg, 0.276 mmol, 57% yield) as white solid. Exact mass calculated for C₂₅H₃₆N₆O₅: 500.3. found: LCMS m/z=501.6 [M+H]⁺; ¹H NMR (400 MHz, CDCl₃) δ 1.28 (d, J=7.0 Hz, 6H), 1.48-1.73 (m, 6H), 1.85-1.92 (m, 2H), 1.96-2.04 (m, 2H), 2.12-2.18 (m, 2H), 2.84-2.92 (m, 1H), 3.33 (s, 3H), 3.40-3.48 (m, 2H), 3.50-3.56 (m, 1H), 3.65-3.70 (m, 1H), 3.80-3.88 (m, 2H), 4.05-4.10 (m, 1H), 4.22-4.28 (m, 1H), 4.33-4.39 (m, 1H), 4.45-4.50 (m, 1H), 4.46-4.82 (m, 1H), 5.07-5.14 (m, 1H), 8.03 (d, J=1.3 Hz, 1H), 8.82 (d, J=1.3 Hz, 1H).

### Example 1.107

### Preparation of Isopropyl 4-((1r,4r)-4-(6-(ethylsulfonyl)-2-methylpyridin-3-yloxy)cyclohexyloxy)piperidine-1-carboxylate (Compound 104)

A mixture of isopropyl 4-((1r,4r)-4-(6-bromo-2-methylpyridin-3-yloxy)cyclohexyloxy)piperidine-1-carboxylate (139 mg, 0.305 mmol), sodium ethanesulfinate (138 mg, 1.188 mmol), copper (I) trifluoromethanesulfonate benzene complex (20 mg, 0.040 mmol), and N¹,N²-dimethylethane-1,2-diamine (10 μL, 0.093 mmol) in 3 mL DMSO was heated under microwave irradiation at 120° C. for 2 h. The resulting mixture was partitioned between EtOAc and water. The aqueous phase was removed. The organic phase was dried over MgSO₄, filtered, and concentrated. The residue was purified by Biotage™ flash chromatography (hexane/EtOAc) to give isopropyl 4-((1r,4r)-4-(6-(ethylsulfonyl)-2-methylpyridin-3-yloxy)cyclohexyloxy)piperidine-1-carboxylate (129 mg, 0.275 mmol, 90% yield). Exact mass calculated for C₂₃H₃₆N₂O₆S: 468.61. found: LCMS m/z=469.4 [M+H]⁺; ¹H NMR (400 MHz, CDCl₃) δ 1.23 (d, J=6.3 Hz, 6H), 1.28 (d, J=7.6 Hz, 3H), 1.49-1.57 (m, 3H), 1.61-1.69 (m, 3H), 1.77-1.81 (m, 2H), 1.93-1.98 (m, 2H), 2.08-2.13 (m, 2H), 2.50 (s, 3H), 3.12-3.19 (m, 2H), 3.35 (t, J=7.6 Hz, 2H), 3.55-3.60 (m, 2H), 3.78-3.81 (m, 2H), 4.42-4.46 (m, 1H), 4.88-4.94 (m, 1H), 7.17 (d, J=8.6 Hz, 1H), 7.89 (d, J=8.6 Hz, 1H).

### Example 1.108

### Preparation of 3-Isopropyl-5-(4-((1r,4r)-4-(2-methyl-6-(methylsulfonyl)pyridin-3-yloxy)cyclohexyloxy)piperidin-1-yl)-1,2,4-oxadiazole (Compound 105)

### Step A: Preparation of 2-Methyl-3-((1r,4r)-4-(1-methylpiperidin-4-yloxy)cyclohexyloxy)-6-(methylsulfonyl)pyridine

The title compound was prepared using 6-bromo-2-methyl-3-((1r,4r)-4-(1-methylpiperidin-4-yloxy)cyclohexyloxy)pyridine and sodium methanesulfinate and a similar procedure as described in Example 102, Step A. Exact mass calculated for C₁₉H₃₀N₂O₄S: 382.1. found LCMS m/z=383.4 [M+H]⁺.

### Step B: Preparation of 2-methyl-6-(methylsulfonyl)-3-((1r,4r)-4-(piperidin-4-yloxy)cyclohexyloxy)pyridine

To a solution of 2-methyl-3-((1r,4r)-4-(1-methylpiperidin-4-yloxy)cyclohexyloxy)-6-(methylsulfonyl)pyridine (0.65 g, 1.7 mmol) in DCM (8 mL) was added 1-chloroethyl carbonochloridate (0.73 g, 5.1 mmol) followed by DIEA (0.66 g, 5.1 mmol). The reaction was stirred for 1 h at 40° C. and then cooled to room temperature. The resulting mixture was washed with saturated NaHCO₃ (2×), and brine. The organics were concentrated and the residue was dissolved in MeOH (8 mL), and then refluxed for 1 h to give 2-methyl-6-(methylsulfonyl)-3-((1r,4r)-4-(piperidin-4-yloxy)cyclohexyloxy)pyridine. After removal of the volatile solvents, the resulting solid was used in Example 1.108, Step C without further purification.

### Step C: Preparation of 4-((1r,4r)-4-(2-methyl-6-(methylsulfonyl)pyridin-3-yloxy)cyclohexyloxy)piperidine-1-carbonitrile

To a solution of 2-methyl-6-(methylsulfonyl)-3-((1r,4r)-4-(piperidin-4-yloxy)cyclohexyloxy)pyridine (0.4 g, 1.09 mmol) and DIEA (0.28 g, 2.18 mmol) in DCM (7.0 mL) was added CNBr (0.15 g, 1.41 mmol) in DCM (1.0 mL). The reaction was stirred for 30 min at room temperature. The reaction was washed with H₂O, 1 N HCl, and brine. The organic was dried with MgSO₄ and concentrated. The residue was purified by column chromatography to give the title compound (0.28 g, 66%). Exact mass calculated for C₁₉H₂₇N₃O₄S: 393.1. found LCMS m/z=394.2 [M+H]⁺.

### Step D: Preparation of 3-isopropyl-5-(4-((1r,4r)-4-(2-methyl-6-(methylsulfonyl)pyridin-3-yloxy)cyclohexyloxy)piperidin-1-yl)-1,2,4-oxadiazole

The title compound was prepared using 4-((1r,4r)-4-(2-methyl-6-(methylsulfonyl)pyridin-3-yloxy)cyclohexyloxy)piperidine-1-carbonitrile and a similar procedure as described in Example 1.91, Step B. Exact mass calculated for C₂₃H₃₄N₄O₅S: 478.2. found LCMS m/z=479.4 [M+H]⁺; ¹H NMR (400 MHz, CDCl₃) δ 1.28 (d, J=7.0 Hz, 6H), 1.50-1.60 (m, 2H), 1.62-1.73 (m, 4H), 1.84-2.01 (m, 4H), 2.07-2.15 (m, 2H), 2.51 (s, 3H), 2.88 (septet, J=6.9 Hz, 1H), 3.18 (s, 3H), 3.41-3.49 (m, 2H), 3.57-3.71 (m, 2H), 3.79-3.87 (m, 2H), 4.43-4.50 (m, 1H), 7.17 (d, J=8.6 Hz, 1H), 7.89 (d, J=8.5 Hz, 1H).

### Example 1.109

### Preparation of Isopropyl 4-((1r,4r)-4-(6-cyano-2-methylpyridin-3-yloxy)cyclohexyloxy)piperidine-1-carboxylate (Compound 106)

A mixture of isopropyl 4-((1r,4r)-4-(6-bromo-2-methylpyridin-3-yloxy)cyclohexyloxy)piperidine-1-carboxylate (see Example 1.103, 160 mg, 0.351 mmol), dicyanozinc (207 mg, 1.763 mmol), and bis(tri-t-butylphosphine)palladium(0) (10 mg, 0.020 mmol) in 4 mL DMA was heated under microwave irradiation at 220° C. for 1 h. The mixture was partitioned between water and EtOAc. The aqueous phase was removed. The organic phase was dried over MgSO₄, filtered, and concentrated. The residue was purified by Biotage™ flash chromatography (SiO₂, hexane/EtOAc) to give isopropyl 4-((1r,4r)-4-(6-cyano-2-methylpyridin-3-yloxy)cyclohexyloxy)piperidine-1-carboxylate (91.7 mg, 0.228 mmol, 65.0% yield) as a white solid. Exact mass calculated for C₂₂H₃₁N₃O₄: 401.50. found: LCMS m/z=402.6 [M+H]⁺; ¹H NMR (400 MHz, CDCl₃) δ 1.23 (d, J=6.3 Hz, 6H), 1.49-1.58 (m, 3H), 1.60-1.68 (m, 3H), 1.77-1.81 (m, 2H), 1.93-1.98 (m, 2H), 2.07-2.13 (m, 2H), 2.47 (s, 3H), 3.12-3.19 (m, 2H), 3.54-3.61 (m, 2H), 3.78-3.81 (m, 2H), 4.39-4.46 (m, 1H), 4.88-4.94 (m, 1H), 7.07 (d, J=8.6 Hz, 1H), 7.50 (d, J=8.6 Hz, 1H).

### Example 1.110

### Preparation of (S)-1-(Benzyloxy)propan-2-yl 4-((1r,4r)-4-(2-Methyl-6-(methylsulfonyl)pyridin-3-yloxy)cyclohexyloxy)piperidine-1-carboxylate (Compound 107)

A solution of (S)-1-(benzyloxy)propan-2-ol (100 μL, 0.628 mmol) and di(1H-imidazol-1-yl)methanone (81.2 mg, 0.501 mmol) in 1 mL THF was stirred at room temperature. After 40 min, 2-methyl-6-(methylsulfonyl)-3-((1r,4r)-4-(piperidin-4-yloxy)cyclohexyloxy)pyridine (49.2 mg, 0.134 mmol) and triethylamine (100 μL, 0.717 mmol) were added. The mixture was heated under microwave irradiation at 120° C. for 3 h and purified by HPLC (5-95% CH₃CN in 30 min+10 min 95% CH₃CN). Fractions containing the desired product were partially concentrated and the residue was partitioned between 1 M NaOH and CH₂Cl₂. The combined organic phases were dried over MgSO₄, filtered, and concentrated to give (S)-1-(benzyloxy)propan-2-yl 4-((1r,4r)-4-(2-methyl-6-(methylsulfonyl)pyridin-3-yloxy)cyclohexyloxy)piperidine-1-carboxylate (36.9 mg, 0.066 mmol, 49.3% yield). Exact mass calculated for C₂₉H₄₀N₂O₇S: 560.26. found: LCMS m/z=561.6 [M+H]⁺; ¹H NMR (400 MHz, CDCl₃) δ 1.26 (d, J=6.4 Hz, 3H), 1.46-1.69 (m, 6H), 1.77-1.81 (m, 2H), 1.93-1.98 (m, 2H), 2.07-2.13 (m, 2H), 2.51 (s, 3H), 3.12-3.2 (m, 5H), 3.47-3.60 (m, 4H), 3.77-3.81 (m, 2H), 4.43-4.47 (m, 1H), 4.51-4.60 (m, 2H), 5.01-5.05 (m, 1H), 7.17 (d, J=8.6 Hz, 1H), 7.25-7.36 (m, 5H), 7.89 (d, J=8.6 Hz, 1H).

### Example 1.111

### Preparation of (S)-1-Hydroxypropan-2-yl 4-((1r,4r)-4-(2-Methyl-6-(methylsulfonyl)pyridin-3-yloxy)cyclohexyloxy)piperidine-1-carboxylate (Compound 108)

To a solution of (S)-1-(benzyloxy)propan-2-yl 4-((1r,4r)-4-(2-methyl-6-(methylsulfonyl)pyridin-3-yloxy)cyclohexyloxy)piperidine-1-carboxylate (see Example 1.110, 33.3 mg, 0.059 mmol) in 2 mL THF, was added palladium on carbon (Degussa type, 10%, 50% water, 84 mg, 0.039 mmol). To the resulting suspension was bubbled hydrogen gas for 1 min and stirred under a hydrogen atmosphere (balloon) at 50° C. (oil bath). After 2 h, the mixture was filtered through Celite® and washed with additional THF. The filtrate was concentrated and dried under high vacuum to give (S)-1-hydroxypropan-2-yl 4-((1r,4r)-4-(2-methyl-6-(methylsulfonyl)pyridin-3-yloxy)cyclohexyloxy)piperidine-1-carboxylate (26.4 mg, 0.056 mmol, 94% yield) as a white solid. Exact mass calculated for C₂₂H₃₄N₂O₇S: 470.21. found: LCMS m/z=471.4 [M+H]⁺; ¹H NMR (400 MHz, CDCl₃) δ 1.25 (d, J=6.5 Hz, 3H), 1.49-1.69 (m, 7H), 1.77-1.81 (m, 2H), 1.93-1.97 (m, 2H), 2.07-2.13 (m, 2H), 2.51 (s, 3H), 3.20-3.26 (m, 5H), 3.57-3.80 (m, 6H), 4.43-4.47 (m, 1H), 4.89-4.93 (m, 1H), 7.17 (d, J=8.6 Hz, 1H), 7.89 (d, J=8.6 Hz, 1H).

### Example 1.112

### Preparation of (R)-1,1,1-Trifluoropropan-2-yl 4-((1r,4r)-4-(2-Methyl-6-(methylsulfonyl)pyridin-3-yloxy)cyclohexyloxy)piperidine-1-carboxylate (Compound 109)

To a solution of (R)-1,1,1-trifluoropropan-2-ol (55% chemical purity containing the corresponding ether as an impurity) (0.231 g, 1.113 mmol) and 1,1′-carbonyldiimidazole (0.176 g, 1.086 mmol) in anhydrous THF (3 mL) stirred at room temperature (1 h) was added 2-methyl-6-(methylsulfonyl)-3-((1r,4r)-4-(piperidin-4-yloxy)cyclohexyloxy)pyridine (0.1 g, 0.271 mmol), followed by triethylamine (0.132 mL, 0.950 mmol). The heterogeneous mixture was stirred at 70° C. for 20 h, cooled to room temperature, diluted with water, and extracted with DCM. The combined organics were concentrated. The residue was purified by column chromatography with 45% ethyl acetate/hexanes to give the title compound (112 mg, 0.22 mmol, 81% yield) as a white solid. Exact mass calculated for C₂₂H₃₁F₃N₂O₆S: 508.2. found: LCMS m/z=509.4 [M+H]⁺; ¹H NMR (400 MHz, CDCl₃) δ 1.38 (d, J=6.7 Hz, 3H), 1.48-1.68 (m, 6H), 1.72-1.85 (m, 2H), 1.91-1.98 (m, 2H), 2.05-2.15 (m, 2H), 2.49 (s, 3H), 3.16 (s, 3H), 3.20-3.33 (m, 2H), 3.55-3.65 (m, 2H), 3.68-3.80 (m, 2H), 4.42-4.48 (m, 1H), 5.17-5.28 (m, 1H), 7.17 (d, J=8.6 Hz, 1H), 7.87 (d, J=8.5 Hz, 1H).

### Example 1.113

### Preparation of (S)-1,1,1-Trifluoropropan-2-yl 4-((1r,4r)-4-(2-Methyl-6-(methylsulfonyl)pyridin-3-yloxy)cyclohexyloxy)piperidine-1-carboxylate (Compound 110)

To a solution of (S)-1,1,1-trifluoropropan-2-ol (60% chemical purity containing the corresponding ether as an impurity) (0.212 g, 1.113 mmol) and 1,1′-carbonyldiimidazole (0.176 g, 1.086 mmol) in anhydrous THF (3 mL) stirred at room temperature (1 h) was added 2-methyl-6-(methylsulfonyl)-3-((1r,4r)-4-(piperidin-4-yloxy)cyclohexyloxy)pyridine (0.1 g, 0.271 mmol), followed by triethylamine (0.132 mL, 0.950 mmol). The heterogeneous mixture was stirred at 70° C. for 20 h, cooled to room temperature, diluted with water, and extracted with DCM. The combined organics were concentrated. The residue was purified by column chromatography with 45% ethyl acetate/hexanes to give the title compound (116 mg, 0.228 mmol, 84% yield) as a white solid. Exact mass calculated for C₂₂H₃₁F₃N₂O₆S: 508.2. found: LCMS m/z=509.4 [M+H]⁺; ¹H NMR (400 MHz, CDCl₃) δ 1.38 (d, J=6.7 Hz, 3H), 1.48-1.68 (m, 6H), 1.72-1.85 (m, 2H), 1.91-1.98 (m, 2H), 2.05-2.15 (m, 2H), 2.49 (s, 3H), 3.16 (s, 3H), 3.20-3.33 (m, 2H), 3.55-3.65 (m, 2H), 3.68-3.80 (m, 2H), 4.42-4.48 (m, 1H), 5.17-5.28 (m, 1H), 7.17 (d, J=8.6 Hz, 1H), 7.87 (d, J=8.5 Hz, 1H).

### Example 1.114

### Preparation of (1s,4s)-4-(1-Methylpiperidin-4-yloxy)cyclohexanol and (1r,4r)-4-(1-Methylpiperidin-4-yloxy)cyclohexanol

### Step A: Preparation of 4-(1,4-Dioxaspiro[4.5]decan-8-yloxy)pyridine

To a suspension of sodium 2-methylpropan-2-olate (238 g, 2478 mmol) in DMSO (700 mL) was added a solution of 1,4-dioxaspiro[4.5]decan-8-ol (196 g, 1239 mmol) in DMSO (300 mL) followed by addition of 4-chloropyridine hydrochloride (186 g, 1239 mmol) portion wise while maintaining the temperature below 40° C. (water bath). The mixture was then heated at 80° C. for 4 h until the complete disappearance of the starting material. The mixture was then quenched with 100 mL of water and partially concentrated to remove most of the DMSO (water bath at 80° C.). The resulting viscous material was then diluted with water and extracted with EtOAc three times. The organic layers were combined and dried over MgSO₄ and concentrated to give 4-(1,4-dioxaspiro[4.5]decan-8-yloxy)pyridine as a beige solid. The solid was transferred to a vacuum filter cup, washed with a minimum amount of MTBE twice at room temperature, dried under reduced pressure to give 4-(1,4-dioxaspiro[4.5]decan-8-yloxy)pyridine (254.5 g, 87% yield) as a light-brown solid. The mother liquor was concentrated to give a wet solid which was triturated with MTBE, the solid was filtered, washed with minimum amount of MTBE to give 4-(1,4-dioxaspiro[4.5]decan-8-yloxy)pyridine (14 g, 4.8% yield) as a light-brown solid. Exact mass calculated for C₁₃H₁₇NO₃: 235.3. found LCMS m/z=236.0 [M+H]⁺.

### Step B: Preparation of 4-(Pyridin-4-yloxy)cyclohexanone

To a solution of 4-(1,4-dioxaspiro[4.5]decan-8-yloxy)pyridine (169.5 g, 720 mmol) in THF (1500 mL) was added water (1500 mL), followed by 6 M HCl solution in water (240 mL, 1441 mmol). The reaction mixture was stirred at 50° C. overnight. LCMS showed 95% conversion to the product and 5% starting material. The reaction mixture was cooled to 24° C., most of the organic solvent was removed under reduced pressure at 30° C. to give the aqueous solution (1150 mL) to which water (650 mL) was added. The solution was adjusted to pH 6 by addition of solid potassium phosphate tribasic (194 g, 914 mmol) at room temperature portion-wise while stirring over 10 min to give a turbid mixture. The mixture was then adjusted to pH 7-8 by addition of solid dibasic potassium phosphate (159 g, 914 mmol) at room temperature portion-wise while stirring over about 10 min. The resulting mixture was stirred for 20 min at RT then extracted with 12% IPA-DCM (3×900 mL, 1×450 mL), the organic extracts were combined, dried over MgSO₄, filtered, and concentrated to give 4-(pyridin-4-yloxy)cyclohexanone (140.03 g, 100%) as a beige solid (contains 5 mol % SM). Exact mass calculated for C₁₁H₁₃NO₂: 191.2. found LCMS m/z=192.0 [M+H]⁺.

### Step C: Preparation of (1s,4s)-4-(Pyridin-4-yloxy)cyclohexanol and (1r,4r)-4-(Pyridin-4-yloxy)cyclohexanol

To a 3-neck round bottom flask, equipped with a mechanic stirrer, thermometer, and nitrogen gas inlet, with one neck used for chemical addition and gas out, was added 4-(pyridin-4-yloxy)cyclohexanone (162 g, 847 mmol) and MeOH (2000 mL) and cooled to 11° C. To the cooled solution was slowly added 1/4 of the total amount of sodium borohydride (38.46 g, 1017 mmol, 1.2 equiv), during the addition the temperature of the solution rose to 36° C. After allowing the reaction mixture to cool down to 15° C. another 1/4 of NaBH₄ was added (the temperature during the addition did not change significantly). The remaining amount of NaBH₄ was added into two portions. The cooling bath was removed and the reaction mixture was stirred at 18° C. for 1 h (crude LCMS showed ratio of cis/trans=57:42 and small amount of product and borane complex). The reaction was diluted with water, acidified to pH 2-3 with 6 M HCl, and stirred for 30 min. The mixture was concentrated to remove a substantial amount of the MeOH and solid KOH was added to adjust to pH to 8 to form a white precipitate. The white precipitate was collected to give the cis enriched product (80-85% pure). The filtrate was extracted with IPA/DCM (1:3), dried over anhydrous Na₂SO₄, filtered and concentrated to give the mixture of cis/trans. The major cis product was triturated with acetone. After several triturations, the solid was filtered to give pure cis (˜87 g). The mixture containing the cis/trans products was also triturated with acetone to provide a precipitate. The precipitate was collected to give trans enriched product (70-80% pure), which was then triturated with acetone to give the pure trans product (˜25 g). After isolating the cis and trans products, the remaining cis/trans mixture weighed approximately 35 g. Total weight of cis, trans, and cis/trans mixture of the products was about 147 g, yield 89.6%. Exact mass calculated for C₁₁H₁₅NO₂: 193.1. found: LCMS m/z=194.0 [M+H]⁺; ¹H NMR for cis (400 MHz, CDCl₃) δ ppm 1.62-1.82 (m, 7H), 1.99-2.08 (m, 2H), 3.78-3.86 (m, 1H), 4.45-4.51 (m, 1H), 6.78 (dd, J=4.8 and 1.5 Hz, 2H), 8.40 (dd, J=4.8 and 1.5 Hz, 2H); ¹H NMR for trans (400 MHz, CDCl₃) δ ppm 1.42-1.54 (m, 2H), 1.55-1.65 (m, 3H), 2.00-2.08 (m, 2H), 2.08-2.18 (m, 2H), 3.78-3.87 (m, 1H), 4.35-4.42 (m, 1H), 6.77 (dd, J=4.8 and 1.5 Hz, 2H), 8.40 (dd, J=4.8 and 1.5 Hz, 2H).

### Step D: Preparation of 4-((1s,4s)-4-Hydroxycyclohexyloxy)-1-methylpyridinium Iodide

To a suspension of (1s,4s)-4-(pyridin-4-yloxy)cyclohexanol (30.0 g, 155 mmol) in DCM/DMA (1:1) (392 mL) and THF (80 mL) was slowly added iodomethane (12.6 mL, 202 mmol) at room temperature. After stirring over night at room temperature the suspension became a clear solution. The LCMS showed the reaction was complete. The solvents were removed under reduced pressure and dried under high vacuum to give the crude product of 4-((1s,4s)-4-hydroxycyclohexyloxy)-1-methylpyridinium iodide (72.2 g, containing DMA solvent) and which was used in the next step without further purification. LCMS m/z=208.3 [M]⁺.

### Step E: Preparation of (1s,4s)-4-(1-Methyl-1,2,3,6-tetrahydropyridin-4-yloxy)cyclohexanol

To a solution of 4-((1s,4s)-4-hydroxycyclohexyloxy)-1-methylpyridinium iodide (crude, has DMA solvent) (72 g, 215 mmol) in methanol (540 mL) cooled down in an ice-water bath was slowly added sodium borohydride (40.6 g, 1074 mmol). Vigorous effervescence was observed while addition. After the addition, the reaction mixture was stirred at room temperature overnight. LCMS showed complete formation of product and no starting material. The reaction mixture was diluted with water and resulting mixture was concentrated under reduced pressure to remove a portion of the MeOH. The mixture was extracted with IPA/DCM (1:4). The combined organics were washed with sat. NaHCO₃, dried over anhydrous Na₂SO₄, filtered, and concentrated to give the crude oil that solidified to a white solid upon standing. The solid was triturated with ethyl acetate and filtered to give (1s,4s)-4-(1-methyl-1,2,3,6-tetrahydropyridin-4-yloxy)cyclohexanol as white solid (25.0 g). The filtrate was concentrated to give the oil, which was purified by biotage column chromatography, 50 g SNAP™, (0-10% methanol/DCM 5 column volumes with 1% triethylamine and then at 10% methanol/DCM 5 column volumes) to give the additional product (2.1 g). ¹H NMR (CDCl₃, 400 MHz) δ ppm 1.50-1.58 (m, 2H), 1.64-1.69 (m, 4H), 1.90-1.97 (m, 2H), 2.21-2.25 (m, 2H), 2.36 (s, 3H), 2.58 (t, J=4 Hz, 2H), 2.96-2.98 (m, 2H), 3.69-3.74 (m, 1H), 4.05-4.09 (m, 1H), 4.56-4.58 (m, 1H), LCMS m/z=212.0 [M+H]⁺.

### Step F: Preparation of (1s,4s)-4-(1-Methylpiperidin-4-yloxy)cyclohexanol

To solution of (1s,4s)-4-(1-methyl-1,2,3,6-tetrahydropyridin-4-yloxy)cyclohexanol (27.1 g, 128 mmol) in 100 mL MeOH was added Pd/C (10% in 50% water, Degussa type) (18 g, 169 mmol). The suspension was shaken under an atmosphere of hydrogen at 50 psi for 22 h in a Parr shaker. The suspension was filtered through Celite® and washed thoroughly with MeOH. The filtrate was concentrated and dried under high vacuum to give (1s,4s)-4-(1-methylpiperidin-4-yloxy)cyclohexanol (25.5 g, 120 mmol, 93% yield) as a white solid. ¹H NMR (CDCl₃, 400 MHz) δ ppm 1.49-1.56 (m, 2H), 1.58-1.87 (m, 10H), 2.10-2.17 (m, 2H), 2.27 (s, 3H), 2.68-2.73 (m, 2H), 3.36-3.40 (m, 1H), 3.45-3.50 (m, 1H), 3.72-3.77 (m, 1H) LCMS m/z=214.0 [M+H]⁺.

### Step G: Preparation of (1r,4r)-4-(1-Methylpiperidin-4-yloxy)cyclohexanol

(1r,4r)-4-(1-Methylpiperidin-4-yloxy)cyclohexanol was prepared in a similar manner as described in Example 1.114, Step D to Step F starting with (1r,4r)-4-(pyridin-4-yloxy)cyclohexanol.

### Example 1.115

### Preparation of Isopropyl 4-((1r,4r)-4-(3-Methyl-5-(methylsulfonyl)pyrazin-2-yloxy)cyclohexyloxy)piperidine-1-carboxylate (Compound III)

### Step A: Preparation of 5-Bromo-3-methylpyrazin-2-ol

To a solution of 3-methylpyrazin-2-ol (1.5 g, 13.62 mmol) in anhydrous DMF (20 mL) was added N-bromosuccinimide (2.67 g, 14.98 mmol) at 0° C. The reaction mixture was slowly warmed to room temperature and stirred overnight. The resulting mixture was poured into water, and extracted with IPA/DCM (1/5). The combined organics were dried over anhydrous Na₂SO₄, filtered then concentrated. The residue was purified by column chromatography with 80% ethyl acetate/hexanes to give the title compound as white solid (1.97 g, 10.42 mmol, 77% yield). Exact mass calculated for C₅H₅BrN₂O: 188.0. found: LCMS m/z=189.0 [M+H]⁺; ¹H NMR (400 MHz, DMSO-d₆) δ ppm 2.27 (d, J=0.5 Hz, 3H), 7.73 (s, 1H), 12.3 (s, 1H).

### Step B: Preparation of 5-Bromo-3-methyl-2-((1r,4r)-4-(1-methylpiperidin-4-yloxy)cyclohexyloxy)pyrazine

(1s,4s)-4-(1-Methylpiperidin-4-yloxy)cyclohexanol (1.2 g, 5.63 mmol) was dissolved in THF (10 mL), 5-bromo-3-methylpyrazin-2-ol (1.170 g, 6.19 mmol) and triphenylphosphine (1.771 g, 6.75 mmol) were added, then cooled down in an ice-water bath. DIAD (1.329 ml, 6.75 mmol) was added dropwise under N₂. The reaction mixture was slowly warmed to room temperature, stirred overnight, and concentrated. The residue was purified by preparative HPLC (15-80% CH₃CN/H₂O with 0.1% TFA over 30 min) and column chromatography (5% methanol/ethyl acetate containing 1% Et₃N) to give the title compound (525 mg, 1.366 mmol, 24.3% yield). Exact mass calculated for C₁₇H₂₆BrN₃O₂: 383.1. found: LCMS m/z=384.2 [M+H]⁺; ¹H NMR (400 MHz, CDCl₃) δ ppm 1.45-1.62 (m, 4H), 1.65-1.75 (m, 2H), 1.91-2.05 (m, 4H), 2.05-2.15 (m, 2H), 2.39 (s, 3H), 2.36-2.42 (m, 2H), 2.42 (d, J=0.7 Hz, 3H), 2.78-2.85 (m, 2H), 3.46-3.54 (m, 2H), 4.95-5.02 (m, 1H), 7.98 (d, J=0.6 Hz, 1H).

### Step C: Preparation of 3-Methyl-2-((1r,4r)-4-(1-methylpiperidin-4-yloxy)cyclohexyloxy)-5-(methylsulfonyl)pyrazine

Sodium methanesulfinate (349 mg, 3.42 mmol) and copper(I) trifluoromethanesulfonate benzene complex (103 mg, 0.205 mmol) in a reaction vial was degassed, 5-bromo-3-methyl-2-((1r,4r)-4-(1-methylpiperidin-4-yloxy)cyclohexyloxy)pyrazine (525 mg, 1.366 mmol) in DMSO (5 mL) was added, followed by N1,N2-dimethylethane-1,2-diamine (0.044 ml, 0.410 mmol). The reaction mixture was heated at 120° C. overnight. The mixture was added water, saturated NH₄Cl solution, and saturated NaHCO₃ solution to adjust pH to 8. The mixture was extracted with IPA/DCM (1/6). The combined organics were dried over anhydrous Na₂SO₄, filtered then concentrated. The residue was purified by column chromatography (10% MeOH/EtOAc with 1% Et₃N) to give the title compound (476 mg, 1.241 mmol, 91% yield) as an off-white solid. Exact mass calculated for C₁₈H₂₉N₃O₄S: 383.2. found: LCMS m/z=384.4 [M+H]⁺; ¹H NMR (400 MHz, CDCl₃) δ ppm 1.50-1.68 (m, 5H), 1.75-1.85 (m, 2H), 1.93-2.00 (m, 3H), 2.10-2.20 (m, 4H), 2.51 (s, 6H), 2.88-2.98 (m, 2H), 3.17 (s, 3H), 3.48-3.54 (m, 1H), 3.55-3.65 (m, 1H), 5.17-5.20 (m, 1H), 8.62 (s, 1H).

### Step D: Preparation of Isopropyl 4-((1r,4r)-4-(3-Methyl-5-(methylsulfonyl)pyrazin-2-yloxy)cyclohexyloxy)piperidine-1-carboxylate

To a stirred solution of 3-methyl-2-((1r,4r)-4-(1-methylpiperidin-4-yloxy)cyclohexyloxy)-5-(methylsulfonyl)pyrazine (350 mg, 0.913 mmol) in anhydrous DCM (5 mL) was added a 1 M solution of isopropyl chloroformate in toluene (1825 μL, 1.825 mmol) at room temperature, followed by DIEA (397 μL, 2.282 mmol). The reaction mixture was stirred at room temperature overnight, diluted with DCM, washed with 1 N HCl aqueous solution and water, dried over anhydrous Na₂SO₄, filtered then concentrated. The residue was purified by column chromatography with 40% ethyl acetate/hexanes to give the title compound (345 mg, 0.757 mmol, 83% yield) as a white solid. Exact mass calculated for C₂₁H₃₃N₃O₆S: 455.2. found: LCMS m/z=456.4 [M+H]⁺; ¹H NMR (400 MHz, CDCl₃) δ ppm 1.21 (d, J=6.2 Hz, 6H), 1.45-1.65 (m, 6H), 1.73-1.80 (m, 2H), 1.90-1.98 (m, 2H), 2.08-2.16 (m, 2H), 2.48 (d, J=0.5 Hz, 3H), 3.08-3.15 (m, 2H), 3.14 (s, 3H), 3.50-3.58 (m, 2H), 3.72-3.80 (m, 2H), 4.84-4.91 (m, 1H), 5.14-5.22 (m, 1H), 8.59 (d, J=0.6 Hz, 1H).

### Example 1.116

### Preparation of 2-Methyl-6-(methylsulfonyl)-3-((1r,4r)-4-(1-(((R)-1,1,1-trifluoropropan-2-yloxy)carbonyl)piperidin-4-yloxy)cyclohexyloxy)pyridine 1-oxide (Compound 119)

### Step A: Preparation of (R)-1,1,1-Trifluoropropan-2-yl 4-((1r,4r)-4-(6-bromo-2-methylpyridin-3-yloxy)cyclohexyloxy)piperidine-1-carboxylate

A solution of (R)-1,1,1-trifluoropropan-2-ol (42% by weight in Et₂O) (2.111 g, 7.77 mmol) and 1,1′-carbonyldiimidazole (1.229 g, 7.58 mmol) in anhydrous THF (5 mL) was stirred at room temperature for 1 h, 6-bromo-2-methyl-3-((1r,4r)-4-(piperidin-4-yloxy)cyclohexyloxy)pyridine (0.7 g, 1.895 mmol) was added, followed by triethylamine (0.923 ml, 6.63 mmol). The reaction mixture was stirred at 65° C. for overnight, cooled, diluted with water, and extracted with ethyl acetate. The combined organics were concentrated. The residue was purified by column chromatography (20% ethyl acetate/hexanes) to give the title compound as colorless oil (0.9 g, 1.767 mmol, 93% yield). Exact mass calculated for C₂₁H₂₈BrF₃N₂O₄: 508.1. found: LCMS m/z=509.2 [M+H]⁺; ¹H NMR (400 MHz, CDCl₃) δ ppm 1.40 (d, J=6.6 Hz, 3H), 1.45-1.65 (m, 6H), 1.74-1.85 (m, 2H), 1.91-1.98 (m, 2H), 2.02-2.10 (m, 2H), 2.43 (s, 3H), 3.22-3.35 (m, 2H), 3.52-3.65 (m, 2H), 3.70-3.80 (m, 2H), 4.23-4.29 (m 1H), 5.20-5.28 (m, 1H), 6.98 (d, J=8.4 Hz, 1H), 7.22 (d, J=8.4 Hz, 1H).

### Step B: Preparation of 6-Bromo-2-methyl-3-((1r,4r)-4-(1-(((R)-1,1,1-trifluoropropan-2-yloxy)carbonyl)piperidin-4-yloxy)cyclohexyloxy)pyridine 1-Oxide

(R)-1,1,1-trifluoropropan-2-yl 4-((1r,4r)-4-(6-bromo-2-methylpyridin-3-yloxy)cyclohexyloxy)piperidine-1-carboxylate (400 mg, 0.785 mmol) was dissolved in DCM, mCPBA (352 mg, 1.571 mmol) was added. The reaction mixture was stirred at room temperature overnight. Saturated aqueous NaHCO₃ was added. The mixture was extracted with DCM. The combined organics were dried over anhydrous Na₂SO₄, filtered then concentrated. The residue was purified by column chromatography (65% ethyl acetate/hexanes) to give the title compound as a white solid (280 mg, 0.533 mmol, 67.9% yield). Exact mass calculated for C₂₁H₂₈BrF₃N₂O₅: 524.1. found: LCMS m/z=525.6 [M+H]⁺; ¹H NMR (400 MHz, CDCl₃) δ ppm 1.40 (d, J=6.6 Hz, 3H), 1.45-1.65 (m, 6H), 1.74-1.85 (m, 2H), 1.91-1.98 (m, 2H), 2.04-2.12 (m, 2H), 2.51 (s, 3H), 3.22-3.35 (m, 2H), 3.52-3.65 (m, 2H), 3.70-3.80 (m, 2H), 4.28-4.35 (m 1H), 5.20-5.28 (m, 1H), 6.72 (d, J=9.1 Hz, 1H), 7.43 (d, J=9.1 Hz, 1H).

### Step C: Preparation of 2-Methyl-6-(methylsulfonyl)-3-((1r,4r)-4-(1-(((R)-1,1,1-trifluoropropan-2-yloxy)carbonyl)piperidin-4-yloxy)cyclohexyloxy)pyridine 1-Oxide

Sodium methanesulfinate (121 mg, 1.190 mmol) and copper(I) trifluoromethanesulfonate benzene complex (23.95 mg, 0.048 mmol) in a microwave vial was degassed, 6-bromo-2-methyl-3-((1r,4r)-4-(1-(((R)-1,1,1-trifluoropropan-2-yloxy)carbonyl)piperidin-4-yloxy)cyclohexyloxy)pyridine 1-oxide (250 mg, 0.476 mmol) in DMSO (3 mL) was added, followed by N1,N2-dimethylethane-1,2-diamine (10.24 μL, 0.095 mmol). The reaction was heated at 120° C. for 1 h under microwave irradiation. The mixture was purified by preparative HPLC (45-90% CH₃CN/H₂O with 0.1% TFA). The combined fractions were neutralized with saturated aqueous NaHCO₃, extracted with DCM. The combined organics were dried over anhydrous Na₂SO₄, filtered then concentrated to give the title compound as white solid (75 mg, 0.143 mmol, 30% yield). Exact mass calculated for C₂₂H₃₁F₃N₂O₇S: 524.2. found: LCMS m/z=525.3 [M+H]⁺; ¹H NMR (400 MHz, CDCl₃) δ ppm 1.40 (d, J=6.6 Hz, 3H), 1.50-1.72 (m, 6H), 1.74-1.85 (m, 2H), 1.91-1.98 (m, 2H), 2.07-2.15 (m, 2H), 2.46 (s, 3H), 3.22-3.35 (m, 2H), 3.47 (s, 3H), 3.56-3.67 (m, 2H), 3.70-3.80 (m, 2H), 4.46-4.54 (m 1H), 5.20-5.28 (m, 1H), 6.90 (d, J=9.1 Hz, 1H), 7.92 (d, J=9.1 Hz, 1H).

### Example 1.117

### Preparation of (R)-1,1,1-Trifluoropropan-2-yl 4-((1r,4r)-4-(3-Methyl-5-(methylsulfonyl)pyrazin-2-yloxy)cyclohexyloxy)piperidine-1-carboxylate (Compound 112)

A solution of (R)-1,1,1-trifluoropropan-2-ol (93 mg, 0.812 mmol) and CDI (0.132 mg, 0.812 mmol) in THF (3 mL) was stirred for 1.5 h at room temperature. Then 3-methyl-5-(methylsulfonyl)-2-((1r,4r)-4-(piperidin-4-yloxy)cyclohexyloxy)pyrazine (100 mg, 0.271 mmol) in THF (2 mL) was added, followed by Et₃N (0.12 mL, 1.083 mmol). The heterogeneous mixture was stirred overnight at 75° C. and cooled to room temperature. After removal of the solvent, the residue was dissolved in DCM. The organic was washed with water and 1 N HCl aqueous solution, dried, and concentrated. The residue was purified by column chromatography to give the title compound (95 mg, 0.186 mmol, 68.9% yield). Exact mass calculated for C₂₁H₃₀F₃N₃O₆S: 509.2. found: LCMS m/z=510.4 [M+H]⁺; ¹H NMR (400 MHz, CDCl₃) δ ppm 1.40 (d, J=6.6 Hz, 3H), 1.50-1.72 (m, 6H), 1.74-1.85 (m, 2H), 1.91-1.98 (m, 2H), 2.10-2.18 (m, 2H), 2.51 (s, 3H), 3.17 (s, 3H), 3.20-3.32 (m, 2H), 3.50-3.65 (m, 2H), 3.70-3.80 (m, 2H), 5.20-5.28 (m, 2H), 8.63 (s, 1H).

### Example 1.118

### Preparation of Isopropyl 4-((1r,4r)-4-(4-(3,3-Difluoroazetidin-1-ylsulfonyl)phenoxy)cyclohexyloxy)piperidine-1-carboxylate (Compound 113)

### Step A: Preparation of 3,3-difluoro-1-(4-fluorophenylsulfonyl)azetidine

To a solution of 4-fluorobenzene-1-sulfonyl chloride (250 mg, 1.285 mmol) in DCM (4 mL) was added 3,3-difluoroazetidine hydrochloride (183 mg, 1.413 mmol), and followed by Et₃N (0.428 mL, 3.21 mmol). The reaction was stirred for 1 h at room temperature, diluted with DCM, washed with water and 1 N HCl aqueous solution, dried, and concentrated to give the title compound (290 mg, 1.154 mmol, 90% yield), which was used without further purification. Exact mass calculated for C₉H₈F₃NO₂S: 250.0. found: LCMS m/z=251.0 [M+H]⁺; ¹H NMR (400 MHz, CDCl₃) δ ppm 4.17 (t, J=12.0 Hz, 4H), 7.26-7.30 (m, 2H), 7.88-7.92 (m, 2H).

### Step B: Preparation of 4-((1r,4r)-4-(4-(3,3-Difluoroazetidin-1-ylsulfonyl)phenoxy)cyclohexyloxy)-1-methylpiperidine

To a solution of (1r,4r)-4-(1-methylpiperidin-4-yloxy)cyclohexanol (220 mg, 1.031 mmol) in DMF (2 mL) was added NaH (60% in oil) (37 mg, 1.547 mmol). The reaction was stirred for 20 min at room temperature under N₂, and then 3,3-difluoro-1-(4-fluorophenylsulfonyl)azetidine (285 mg, 1.134 mmol) in DMF (1 mL) was added. The reaction was stirred overnight, and quenched with water, diluted with DCM, washed with saturated NaHCO₃ solution, dried, and concentrated. The residue was purified by column chromatography to give the title compound (290 mg, 0.652 mmol, 63.3% yield). Exact mass calculated for C₂₁H₃₀F₂N₂O₄S: 444.4. found: LCMS m/z=445.6 [M+H]⁺; ¹H NMR (400 MHz, CDCl₃) δ ppm 1.45-1.70 (m, 7H), 1.80-1.90 (m, 2H), 1.94-2.02 (m, 2H), 2.10-2.20 (m, 3H), 2.29 (s, 3H), 2.65-2.75 (m, 2H), 3.36-3.54 (m, 2H), 4.13 (t, J=12.0 Hz, 4H), 4.35-4.42 (m, 1H), 6.99-7.03 (m, 2H), 7.75-7.80 (m, 2H).

### Step C: Preparation of 4-((1r,4r)-4-(4-(3,3-Difluoroazetidin-1-ylsulfonyl)phenoxy)cyclohexyloxy)piperidine

To a solution of 4-((1r,4r)-4-(4-(3,3-difluoroazetidin-1-ylsulfonyl)phenoxy)cyclohexyloxy)-1-methylpiperidine (280 mg, 0.63 mmol) in DCM (3 mL) was added 1-chloroethyl carbonochloridate (270 mg, 1.89 mmol) under N₂ atmosphere. And then, DIEA (219 μL, 1.26 mmol) was added into the reaction. The reaction was refluxed for 2 h, and cooled to room temperature. The reaction was washed with saturated NaHCO₃ aqueous solution twice and brine, and concentrated. The residue was dissolved in anhydrous MeOH (5 mL), and then refluxed for 1 h. After removal of the volatile solvent, the residue was diluted with DCM, washed with saturated NaHCO₃ aqueous solution, dried, and concentrated to give the title compound (240 mg, 0.557 mmol, 89% yield), which was used without further purification.

### Step D: Preparation of Isopropyl 4-((1r,4r)-4-(4-(3,3-Difluoroazetidin-1-ylsulfonyl)phenoxy)cyclohexyloxy)piperidine-1-carboxylate

To a solution of 4-((1r,4r)-4-(4-(3,3-difluoroazetidin-1-ylsulfonyl)phenoxy)cyclohexyloxy)piperidine (70 mg, 0.163 mmol) in DCM was added a 1 M solution of isopropyl carbonochloridate in toluene (0.26 mL, 0.26 mmol), and followed by Et₃N (54 μL, 0.406 mmol). The reaction was stirred for 1 h at room temperature. After removal of the solvent, the residue was purified by preparative TLC plate to give the title compound (50 mg, 0.097 mmol, 59.5% yield). Exact mass calculated for C₂₄H₃₄F₂N₂O₆S: 516.2. found: LCMS m/z=517.4 [M+H]⁺; ¹H NMR (400 MHz, CDCl₃) δ ppm 1.24 (d, J=6.3 Hz, 6H), 1.44-1.64 (m, 6H), 1.75-1.83 (m, 2H), 1.95-2.05 (m, 2H), 2.08-2.15 (m, 2H), 3.10-3.20 (m, 2H), 3.50-3.60 (m, 2H), 3.75-3.83 (m, 2H), 4.13 (t, J=12.0 Hz, 4H), 4.38-4.50 (m, 1H), 4.90-4.95 (m, 1H), 6.99-7.03 (m, 2H), 7.75-7.80 (m, 2H).

### Example 1.119

### Preparation of Isopropyl 4-((1r,4r)-4-(4-Methyl-6-(methylsulfonyl)pyridin-3-yloxy)cyclohexyloxy)piperidine-1-carboxylate (Compound 120)

To a solution of 4-methyl-5-((1r,4r)-4-(1-methylpiperidin-4-yloxy)cyclohexyloxy)-2-(methylsulfonyl)pyridine (260 mg, 0.680 mmol) in CH₂Cl₂ (5 ml), isopropyl carbonochloridate (1.0 mL, 1.000 mmol) and DIEA (0.2 mL, 1.145 mmol) were added. After stirring at room temperature overnight, the reaction mixture was extracted with 1 M NaOH and CH₂Cl₂. The organic phases were combined and concentrated. The residue was purified by HPLC (5-95% CH₃CN in 50 min). Fractions containing the title compound were partly concentrated and the residue was extracted with 1 M NaOH and CH₂Cl₂. The organic phases were dried over MgSO₄, filtered, and concentrated to give the title compound (200 mg, 0.440 mmol, 64.7% yield) as a sticky solid. Exact mass calculated for C₂₂H₃₄N₂O₆S: 454.2. found LCMS m/z=455.2 [M+H]⁺. ¹H NMR (400 MHz, CDCl₃) δ ppm 1.28-1.29 (d, J=6.3 Hz, 6H), 1.53-1.61 (m, 4H), 1.67-1.74 (m, 2H), 1.81-1.86 (m, 2H), 1.98-2.04 (m, 2H), 2.16-2.22 (m, 2H), 2.34 (s, 3H), 3.18-3.23 (m, 2H), 3.22 (s, 3H), 3.60-3.64 (m, 2H), 3.81-3.86 (m, 2H), 4.58-4.63 (m, 1H), 4.93-4.98 (m, 1H), 7.92 (s, 1H), 8.26 (s, 1H).

### Example 1.120

### Preparation of Isopropyl 4-((1r,4r)-4-(5-Methyl-6-(methylsulfonyl)pyridin-3-yloxy)cyclohexyloxy)piperidine-1-carboxylat (Compound 121)

To a solution of 3-methyl-5-((1r,4r)-4-(1-methylpiperidin-4-yloxy)cyclohexyloxy)-2-(methylsulfonyl)pyridine (700 mg, 1.830 mmol) in CH₂Cl₂ (5 ml), isopropyl carbonochloridate (1.0 mL, 1.000 mmol) and DIEA (0.2 mL, 1.145 mmol) were added. After stirring at room temperature overnight, the mixture was extracted with 1 M NaOH and CH₂Cl₂. Organic phases were concentrated and the residue was purified by HPLC (5-95% CH₃CN in 50 min). Fractions containing the title compound were partly concentrated and the residue was extracted with 1 M NaOH and CH₂Cl₂. The organic phases were dried over MgSO₄, filtered, and concentrated to give the title compound (400 mg, 0.880 mmol, 48.1% yield) as a sticky solid. Exact mass calculated for C₂₂H₃₄N₂O₆S: 454.2. found LCMS m/z=455.2 [M+H]⁺; ¹H NMR (400 MHz, CDCl₃) δ ppm 1.28-1.29 (d, J=6.3 Hz, 6H), 1.50-1.57 (m, 4H), 1.61-1.68 (m, 4H), 1.81-1.85 (m, 2H), 1.99-2.04 (m, 2H), 2.13-2.16 (m, 2H), 2.73 (s, 3H), 3.16-3.22 (m, 2H), 3.35 (s, 3H), 3.56-3.63 (m, 2H), 4.44-4.49 (m, 1H), 4.92-4.98 (m, 1H), 7.12 (s, 1H), 8.07 (s, 1H).

### Example 1.121

### Preparation of 5-Ethyl-2-(4-((1r,4r)-4-(2-methyl-6-(methylsulfonyl)pyridin-3-yloxy)cyclohexyloxy)piperidin-1-yl)pyrimidine (Compound 114)

To 2-methyl-6-(methylsulfonyl)-3-((1r,4r)-4-(piperidin-4-yloxy)cyclohexyloxy)pyridine (130 mg, 0.353 mmol), 2-chloro-5-ethylpyrimidine (60 mg, 0.423 mmol) was added 2-propanol (2.98 mL) in a microwave vial. N-ethyl-N-isopropylpropan-2-amine (0.185 mL, 1.058 mmol) was added to the reaction mixture. The mixture was heated at 160° C. for 45 minutes under microwave irradiation. The mixture was concentrated under vacuum to obtain the residue which was diluted with water and extracted with DCM (2×25 mL). DCM layers were combined, dried with Na₂SO₄ and concentrated. The residue was purified by flash column chromatography (10-80% EtOAc-Hexanes) to give the title compound (95 mg, 0.200 mmol, 56.7% yield) as light brown gum. Exact mass calculated for C₂₄H₃₄N₄O₄S: 474.2. found LCMS m/z=475.4 [M+H]⁺; ¹H NMR (400 MHz, CDCl₃) δ ppm 1.19 (t, J=8 Hz, 3H), 1.49-1.69 (m, 6H), 1.88-1.92 (m, 2H), 1.96-2.04 (m, 2H), 2.10-2.15 (m, 2H), 2.46 (q, J=8 Hz, 2H), 2.51 (s, 3H), 3.18 (s, 3H), 3.35-3.39 (m, 2H), 3.61-3.69 (m, 2H), 4.26-4.31 (m, 2H), 4.43-4.48 (m, 1H), 7.17 (d, J=8 Hz, 1H), 7.89 (d, J=8 Hz, 1H), 8.18 (s, 2H).

### Example 1.122

### Preparation of 2-Methyl-3-((1r,4r)-4-(1-(5-methylpyridin-2-yl)piperidin-4-yloxy)cyclohexyloxy)-6-(methylsulfonyl)pyridin (Compound 117)

A mixture 2-methyl-6-(methylsulfonyl)-3-((1r,4r)-4-(piperidin-4-yloxy)cyclohexyloxy)pyridine (100 mg, 0.271 mmol), diacetoxypalladium (6.0 mg), 2-bromo-5-methylpyridine (47 mg, 0.271 mmol), sodium 2-methylpropan-2-olate (63 mg, 0.651 mmol), and 1,1′-Bis(di-t-butylphosphino)ferrocene (26 mg, 0.054 mmol) in 1,4-dioxane (3.0 mL) was heated at 105° C. for 3 h. Water was added to the reaction mixture and extracted with DCM (2×25 mL). The organic layer was rinsed with brine, then treated with Na₂SO₄ and concentrated. The residue was purified by flash column chromatography (10-80% EtOAc-Hexanes) to give the title compound (23 mg, 0.050 mmol, 18.4% yield) as light brown gum. Exact mass calculated for C₂₄H₃₃N₃O₄S: 459.2. found LCMS m/z=460.2 [M+H]⁺; ¹H NMR (400 MHz, CDCl₃) δ ppm 1.23-1.70 (m, 6H), 1.91-2.04 (m, 4H), 2.10-2.19 (m, 2H), 2.34 (s, 3H), 2.51 (s, 3H), 3.13-3.18 (m, 2H), 3.23 (s, 3H), 3.60-3.62 (m, 2H), 3.92-3.97 (m, 2H), 4.43-4.48 (m, 1H), 6.62 (d, J=8 Hz, 1H), 7.17 (d, J=8 Hz, 1H), 7.30 (d, J=8 Hz, 1H), 7.89 (d, J=8 Hz, 1H), 8.01 (s, 1H).

### Example 1.123

### Preparation of 5-Methyl-2-(4-((1r,4r)-4-(2-methyl-6-(methylsulfonyl)pyridin-3-yloxy)cyclohexyloxy)piperidin-1-yl)pyrimidine (Compound 115)

To 2-methyl-6-(methylsulfonyl)-3-((1r,4r)-4-(piperidin-4-yloxy)cyclohexyloxy)pyridine (130 mg, 0.353 mmol), 2-chloro-5-methylpyrimidine (54 mg, 0.423 mmol) was added 2-propanol (3.0 mL) in a microwave vial. N-ethyl-N-isopropylpropan-2-amine (0.185 mL, 1.058 mmol) was added to the mixture. The reaction mixture was heated at 180° C. for 60 minutes under microwave irradiation. Water was added to the reaction mixture and extracted with DCM (3×25 mL). The organic layer were combined, rinsed with brine, treated with Na₂SO₄ and concentrated. The residue was purified by flash column chromatography (10-80% EtOAc-Hexanes) to give the title compound (65 mg, 0.141 mmol, 40.0% yield). Exact mass calculated for C₂₃H₃₂N₄O₄S: 460.2. found LCMS m/z=461.2 [M+H]⁺; ¹H NMR (400 MHz, CDCl₃) δ ppm 1.50-1.70 (m, 6H), 1.86-1.92 (m, 2H), 1.95-2.00 (m, 2H), 2.10-2.15 (m, 2H), 2.14 (s, 3H), 2.51 (s, 3H), 3.18 (s, 3H), 3.34-3.37 (m, 2H), 3.60-3.68 (m, 2H), 4.25-4.30 (m, 2H), 4.42-4.48 (m, 1H), 7.17 (d, J=8 Hz, 1H), 7.89 (d, J=8 Hz, 1H), 8.15 (s, 2H).

### Example 1.124

### Preparation of S-Isopropyl 4-((1r,4r)-4-(2-Methyl-6-(methylsulfonyl)pyridin-3-yloxy)cyclohexyloxy)piperidine-1-carbothioat (Compound 116)

To a suspension of 2-methyl-6-(methylsulfonyl)-3-((1r,4r)-4-(piperidin-4-yloxy)cyclohexyloxy)pyridine (300 mg, 0.814 mmol) in DCM (7.0 mL) was added S-isopropyl chloroformate (0.11 mL, 0.855 mmol) and triethylamine (0.4 mL, 2.85 mmol) at 0° C. The mixture was stirred at room temperature for 1 h. The mixture was quenched with water and the organic layer was separated. The aqueous layer was extracted with DCM (2×25 mL) and the combined organics were concentrated under vacuum. The residue was purified by flash column chromatography (10-80% EtOAc-Hexanes) to give the title compound (245 mg, 0.521 mmol, 63.9% yield). Exact mass calculated for C₂₂H₃₄N₂O₅S₂: 470.2. found LCMS m/z=471.6 [M+H]⁺; ¹H NMR (400 MHz, CDCl₃) δ ppm 1.34 (d, J=8 Hz, 6H), 1.50-1.69 (m, 6H), 1.77-1.84 (m, 2H), 1.93-1.97 (m, 2H), 2.07-2.13 (m, 2H), 2.50 (s, 3H), 3.18 (s, 3H), 3.30-3.37 (m, 2H), 3.60-3.67 (m, 1H), 3.56-3.60 (m, 2H), 3.76-3.80 (m, 2H), 4.43-4.48 (m, 1H), 7.17 (d, J=8 Hz, 1H), 7.89 (d, J=8 Hz, 1H).

### Example 1.125

### Preparation of 3-((1r,4r)-4-(1-(5-Ethylpyridin-2-yl)piperidin-4-yloxy)cyclohexyloxy)-2-methyl-6-(methylsulfonyl)pyridin (Compound 118)

### Step A: Preparation of 1-(6-(4-((1r,4r)-4-(2-Methyl-6-(methylsulfonyl)pyridin-3-yloxy)cyclohexyloxy)piperidin-1-yl)pyridin-3-yl)ethanone

To 2-methyl-6-(methylsulfonyl)-3-((1r,4r)-4-(piperidin-4-yloxy)cyclohexyloxy)pyridine (200 mg, 0.543 mmol), 1-(6-chloropyridin-3-yl)ethanone (101 mg, 0.651 mmol) was added 2-propanol (4.0 mL) in a microwave vial. To the mixture was added N-ethyl-N-isopropylpropan-2-amine (0.28 mL, 1.628 mmol). The reaction was heated at 180° C. for 60 minutes under microwave irradiation. The mixture was concentrated under vacuum and the resulting residue was purified by flash column chromatography (10-90% EtOAc-Hexanes) to give the title compound (125 mg, 0.256 mmol, 47.2% yield) as an off white solid. Exact mass calculated for C₂₅H₃₃N₃O₅S: 487.2. found LCMS m/z=488.4 [M+H]⁺; ¹H NMR (400 MHz, CDCl₃) δ ppm 1.49-1.50 (m, 2H), 1.52-1.68 (m, 4H), 1.88-1.99 (m, 4H), 2.10-2.15 (m, 2H), 2.51 (s, 3H), 2.50 (s, 3H), 3.18 (s, 3H), 3.46-3.50 (m, 2H), 3.62-3.66 (m, 1H), 3.69-3.74 (m, 1H), 4.03-4.08 (m, 2H), 4.45-4.48 (m, 1H), 6.65 (d, J=8 Hz, 1H), 7.18 (d, J=8 Hz, 1H), 7.89 (d, J=8 Hz, 1H), 8.02 (d, J=4 Hz, 1H), 8.75 (s, 1H).

### Step B: Preparation of 3-((1r,4r)-4-(1-(5-Ethylpyridin-2-yl)piperidin-4-yloxy)cyclohexyloxy)-2-methyl-6-(methylsulfonyl)pyridine

To a mixture of 1-(6-(4-((1r,4r)-4-(2-methyl-6-(methylsulfonyl)pyridin-3-yloxy)cyclohexyloxy)piperidin-1-yl)pyridin-3-yl)ethanone (95 mg, 0.195 mmol) and trifluoroacetic acid (1.5 mL) was added triethylsilane (0.12 mL, 0.779 mmol). The reaction mixture was stirred at 55° C. for 16 h. To the reaction mixture was added excess triethylsilane (0.12 mL, 0.779 mmol) and stirred at 55° C. for 16 h. The mixture was poured in ice and pH was adjusted to 8-9 with saturated NaHCO₃ aqueous solution. The aqueous layer was extracted with DCM (2×25 mL), dried with Na₂SO₄ and the solvent was removed under reduced pressure. The residue was purified by flash column chromatography (10-90% EtOAc-Hexanes) to give the title compound (70 mg, 0.148 mmol, 76% yield). Exact mass calculated for C₂₅H₃₅N₃O₄S: 473.2. found LCMS m/z=474.4 [M+H]⁺; ¹H NMR (400 MHz, CDCl₃) δ ppm 1.19 (t, J=8 Hz, 3H), 1.50-1.57 (m, 2H), 1.58-1.70 (m, 4H), 1.90-2.00 (m, 4H), 2.09-2.14 (m, 2H), 2.51 (s, 3H), 2.52 (q, J=8 Hz, 2H), 3.13-3.15 (m, 2H), 3.18 (s, 3H), 3.60-3.64 (m, 2H), 3.93-3.99 (m, 2H), 4.43-4.47 (m, 1H), 6.65 (d, J=8 Hz, 1H), 7.17 (d, J=8 Hz, 1H), 7.33 (d, J=8 Hz, 1H), 7.89 (d, J=8 Hz, 1H), 8.03 (s, 1H).

### Example 1.126

### Preparation of 1,1,1-Trifluoro-2-methylpropan-2-yl 4-((1r,4r)-4-(2-Methyl-6-(methylsulfonyl)pyridin-3-yloxy)cyclohexyloxy)piperidine-1-carboxylate (Compound 122)

A solution of 1,1,1-trifluoro-2-methylpropan-2-ol (235 mg, 1.832 mmol) and 1,1′-carbonyldiimidazole (264 mg, 1.628 mmol) in anhydrous THF (2.5 mL) was stirred at room temperature for 40 h. Triethylamine (0.34 mL, 2.442 mmol), solid 2-methyl-6-(methylsulfonyl)-3-((1r,4r)-4-(piperidin-4-yloxy)cyclohexyloxy)pyridine (150 mg, 0.407 mmol) and anhydrous THF (3.1 mL) were added. The reaction was stirred at 90° C. for 20 h and cooled down. The was removed under vacuum. The residue was diluted with water, extracted with DCM (3×50 mL). The combined organics were dried with Na₂SO₄, and concentrated. The residue was purified by flash column chromatography (10-80% EtOAc/Hexanes) to give the title compound (64 mg, 0.122 mmol, 30.1% yield) as white solid. Exact mass calculated for C₂₃H₃₃F₃N₂O₆S: 522.2. found LCMS m/z=523.4 [M+H]⁺; ¹H NMR (400 MHz, CDCl₃) δ ppm 1.49-1.57 (m, 4H), 1.61-1.66 (m, 2H), 1.68 (s, 6H), 1.76-1.80 (m, 2H), 1.93-1.98 (m, 2H), 2.08-2.13 (m, 2H), 2.51 (s, 3H), 3.18 (s, 3H), 3.19-3.23 (m, 2H), 3.56-3.61 (m, 2H), 3.74-3.70 (m, 2H), 4.43-4.47 (m, 1H), 7.17 (d, J=8 Hz, 1H), 7.89 (d, J=8 Hz, 1H).

### Example 1.127

### Preparation of 1-Methylcyclopropyl 4-((1r,4r)-4-(2-Methyl-6-(methylsulfonyl)pyridin-3-yloxy)cyclohexyloxy)piperidine-1-carboxylat (Compound 123)

To a solution of 2-methyl-6-(methylsulfonyl)-3-((1r,4r)-4-(piperidin-4-yloxy)cyclohexyloxy)pyridine (150 mg, 0.407 mmol) and 2,5-dioxopyrrolidin-1-yl 1-methylcyclopropyl carbonate in DCM (3.0 mL) was added triethylamine (0.22 mL, 1.628 mmol). The reaction was stirred at room temperature for 2 h. The mixture was cooled down, diluted with water, and extracted with DCM (2×25 mL). The combined organics were dried with Na₂SO₄, and concentrated under vacuum. The residue was purified by flash column chromatography (10-80% EtOAc/Hexanes) to give the title compound (75 mg, 0.161 mmol, 39.5% yield) as a white solid. Exact mass calculated for C₂₃H₃₄N₂O₆S: 466.21. found LCMS m/z=467.5 [M+H]⁺; ¹H NMR (400 MHz, CDCl₃) δ ppm 0.60-0.64 (m, 2H), 0.84-0.89 (m, 2H), 1.47-1.54 (m, 4H), 1.52 (s, 3H), 1.60-1.68 (m, 2H), 1.75-1.79 (m, 2H), 1.92-1.97 (m, 2H), 2.07-2.12 (m, 2H), 2.50 (s, 3H), 3.11-3.13 (m, 2H), 3.17 (s, 3H), 3.53-3.60 (m, 2H), 3.73-3.77 (m, 2H), 4.42-4.47 (m, 1H), 7.17 (d, J=8 Hz, 1H), 7.89 (d, J=8 Hz, 1H).

### Example 1.128

### Preparation of S-Cyclopropyl 4-((1r,4r)-4-(2-Methyl-6-(methylsulfonyl)pyridin-3-yloxy)cyclohexyloxy)piperidine-1-carbothioate (Compound 124)

To a solution of S-cyclopropyl 1H-imidazole-1-carbothioate (103 mg, 0.611 mmol) and 2-methyl-6-(methylsulfonyl)-3-((1r,4r)-4-(piperidin-4-yloxy)cyclohexyloxy)pyridine (150 mg, 0.407 mmol) in THF (4 mL) was added triethylamine (0.28 mL, 2.035 mmol). The mixture was stirred at 90° C. for 16 h. The mixture was cooled down, concentrated under vacuum, diluted with water, and extracted with DCM (3×25 mL). The combined organics were dried with Na₂SO₄, and concentrated under vacuum. The residue was purified by column chromatography (0-80% EtOAc/hexanes) to give the title compound (48 mg, 0.102 mmol, 25.2% yield) as a white solid. Exact mass calculated for C₂₂H₃₂N₂O₅S₂: 468.18. found LCMS m/z=469.4 [M+H]⁺; ¹H NMR (400 MHz, CDCl₃) δ ppm 0.58-0.62 (m, 2H), 1.01-1.06 (m, 2H), 1.49-1.69 (m, 6H), 1.77-1.84 (m, 2H), 1.92-1.98 (m, 2H), 2.07-2.17 (m, 3H), 2.50 (s, 3H), 3.18 (s, 3H), 3.22-3.45 (m, 2H), 3.55-3.61 (m, 1H), 3.62-3.67 (m, 1H), 3.72-3.81 (m, 2H), 4.44-4.48 (m, 1H), 7.17 (d, J=8 Hz, 1H), 7.89 (d, J=8 Hz, 1H).

### Example 1.129

### Preparation of 2-(Methylsulfonyl)-5-((1r,4r)-4-(1-((1-(trifluoromethyl)cyclopropyl)methyl)piperidin-4-yloxy)cyclohexyloxy)pyrazine (Compound 125)

### Step A. Preparation of N-methoxy-N-methyl-1-(trifluoromethyl)cyclopropane carboxamide

A mixture of 1-(trifluoromethyl)cyclopropanecarboxylic acid (2.2 g, 14.3 mmol), HATU (5.7 g, 15 mmol), and Et₃N (1.45 g, 14.3 mmol) in ACN (10 mL) was stirred for 10 min at room temperature. N,O-dimethylhydroxylamine hydrochloride (1.53 g, 15.7 mmol) was added and followed by Et₃N (1.74 g, 16.8 mmol). The reaction was stirred for 3 h at room temperature, diluted with EtOAc, washed with 1N HCl (twice) and brine, dried, and concentrated. The residue was purified by column chromatography to give the title compound (2.2 g, 78%). Exact mass calculated for C₇H₁₀F₃NO₂: 197.1. found LCMS m/z=198.2 [M+H]⁺.

### Step B. Preparation of 1-(trifluoromethyl)cyclopropanecarbaldehyde

Powdered LiAlH₄ (385 mg, 10.1 mmol) was added to anhydrous Et₂O (10 mL) and cooled to 0° C. under inert atmosphere. N-methoxy-N-methyl-1-(trifluoromethyl)cyclopropanecarboxamide (2.0 g, 10.1 mmol) in Et₂O (4 mL) was added dropwise to the cloudy LAH solution over 3 min with vigorous stirring. The reaction was stirred for 1 h at the same temperature, quenched carefully with H₂O (0.45 mL), added NaOH (15 wt % in water, 0.45 mL) dropwise, and followed by H₂O again (0.45 mL). The reaction slurry was filtered through a pad of Celite, and washed with Et₂O (2×10 mL). About 2/3 of the volatile solvent was carefully removed under ˜0.5 atm without using a heating bath. 1-(Trifluoromethyl)cyclopropanecarbaldehyde in Et₂O was used in the next step without further purification due to its volatility.

### Step C. Preparation of 2-(Methylsulfonyl)-5-((1r,4r)-4-(1-((1-(trifluoromethyl)cyclopropyl)methyl)piperidin-4-yloxy)cyclohexyloxy)pyrazine (Compound 125)

To a suspension of 2-(methylsulfonyl)-5-((1r,4r)-4-(piperidin-4-yloxy)cyclohexyloxy)pyrazine (120 mg, 0.34 mmol) in DCM (2 mL) was added Et₃N (26 μL, 0.195 mmol), followed by 1-(trifluoromethyl)cyclopropanecarbaldehyde (60 mg, 0.43 mmol) and AcOH (41 mg, 0.68 mmol). The mixture was stirred for 10 min at room temperature, and then NaBH(OAc)₃ (180 mg, 0.85 mmol) was added into the reaction. The reaction was stirred overnight at 30° C., and quenched with saturated NaHCO₃ (0.6 mL). The reaction was diluted with H₂O, extracted with DCM (twice). The combined organics were washed with saturated NaHCO₃ and brine, dried, and concentrated. The residue was purified by preparative TLC to give the title compound (35 mg, 22%). Exact mass calculated for C₂₁H₃₀F₃N₃O₄S: 477.2. found LCMS m/z=478.4 [M+H]⁺; ¹H NMR (400 MHz, CDCl₃) δ ppm 0.61-0.66 (m, 2H), 0.94-0.99 (m, 2H), 1.45-1.65 (m, 6H), 1.77-1.85 (m, 2H), 1.93-2.01 (m, 2H), 2.10-2.19 (m, 4H), 2.52 (s, 2H), 2.71-2.79 (m, 2H), 3.18 (s, 3H), 3.35-3.43 (m, 1H), 3.46-3.53 (m, 1H), 5.12-5.20 (m, 1H), 8.18 (d, J=1.2 Hz, 1H), 8.78 (d, J=1.2 Hz, 1H).

### Example 1.130

### Preparation of 2-(Methylsulfonyl)-5-((1r,4r)-4-(1-(2,2,3,3-tetrafluoropropyl)piperidin-4-yloxy)cyclohexyloxy)pyrazine (Compound 126)

A mixture of 2-(methylsulfonyl)-5-((1r,4r)-4-(piperidin-4-yloxy)cyclohexyloxy)pyrazine (0.11 g, 0.31 mmol), 2,2,3,3-tetrafluoropropyl trifluoromethanesulfonate (0.10 g, 0.38 mmol) and Et₃N (62 μL, 0.47 mmol) in IPA (2 mL) was heated under microwave irradiation for 1 h at 140° C. While cooling the reaction to room temperature, the product was precipitated. The solid was filtered, washed with IPA and hexane, and dried to give the title compound (0.1 g, 68%).

Exact mass calculated for C₁₉H₂₇F₄N₃O₄S: 469.2. found LCMS m/z=470.4 [M+H]⁺; ¹H NMR (400 MHz, CDCl₃) δ ppm 1.44-1.65 (m, 6H), 1.77-1.85 (m, 2H), 1.93-2.01 (m, 2H), 2.10-2.18 (m, 2H), 2.41 (dd, J=9.6 and 3.0 Hz, 2H), 2.81-2.92 (m, 4H), 3.18 (s, 3H), 3.48-3.45 (m, 1H), 3.46-3.53 (m, 1H), 5.13-5.20 (m, 1H), 6.00 (tt, J=53.6 and 5.3 Hz, 1H), 8.18 (d, J=1.2 Hz, 1H), 8.78 (d, J=1.2 Hz, 1H).

### Example 1.131

### Preparation of 2-(Methylsulfonyl)-5-((1r,4r)-4-(1-((1-(trifluoromethyl)cyclobutyl)methyl)piperidin-4-yloxy)cyclohexyloxy)pyrazine (Compound 127)

### Step A: Preparation of (1-(trifluoromethyl)cyclobutyl)methanol

To an ice-cooled solution of 1-(trifluoromethyl)cyclobutanecarboxylic acid (1.1 g, 6.54 mmol) in Et₂O (15 mL), 2 M Lithium aluminum hydride in THF (5 mL, 10.00 mmol) was added slowly. After stirring for 1 h under ice-cooling, the mixture was allowed to warm to room temperature. After 18 h, the mixture was quenched with wet Et₂O and then, 20 mL of 2 M HCl was added. The mixture was transferred into a separatory funnel and extracted four times with ca. 100 mL Et₂O. Combined organic phases were dried over MgSO₄, filtered, and concentrated to give the title compound (ca. 37% pure, 1.96 g, 4.71 mmol, 71.9% yield) as a colorless liquid (which still contained ca. 27% THF and ca. 36% Et₂O). ¹H NMR (400 MHz, CDCl₃) δ ppm 1.67 (m, 1H), 1.96-2.05 (m, 4H), 2.24-2.32 (m, 2H), 3.81-3.82 (d, J=6.1 Hz, 2H).

### Step B: Preparation of (1-(Trifluoromethyl)cyclobutyl)methanol

To an ice-cooled solution of (1-(trifluoromethyl)cyclobutyl)methanol (1.96 g, 4.71 mmol) and DIEA (1.61 mL, 9.22 mmol) in 10 mL CH₂Cl₂, Ms-Cl (0.540 mL, 6.93 mmol) was added. The mixture was stirred under ice-cooling for 1 h and then at room temperature for 2 h. The mixture was transferred into a separatory funnel and extracted with 1 M NaOH and CH₂Cl₂. Organic phases were dried over MgSO₄, filtered, and concentrated to give the title compound (ca. 20% pure, 4.8 g, 4.13 mmol, 88% yield) as a liquid (contained ca. 61% CH₂Cl₂, ca. 18% DIEA and ca. 1% THF). ¹H NMR (400 MHz, CDCl₃) δ ppm 2.01-2.11 (m, 4H), 2.34-2.42 (m, 2H), 3.06 (s, 3H), 4.36 (s, 2H).

### Step C: Preparation of 2-(Methylsulfonyl)-5-((1r,4r)-4-(1-((1-(trifluoromethyl)cyclobutyl)methyl)piperidin-4-yloxy)cyclohexyloxy)pyrazine (Compound 127)

A mixture of 2-(methylsulfonyl)-5-((1s,4s)-4-(piperidin-4-yloxy)cyclohexyloxy)pyrazine (114 mg, 0.321 mmol), (1-(trifluoromethyl)cyclobutyl)methyl methanesulfonate (400 μL, 0.344 mmol), and Cs₂CO₃ (107 mg, 0.328 mmol) in CH₃CN (5 mL) was heated under microwave irradiation at 120° C. for 38 h. The mixture was purified by HPLC (5-60% CH₃CN+0.5% TFA). Fractions containing desired product were partly concentrated and residue was extracted with 1 M NaOH and CH₂Cl₂. Organic phases were dried over MgSO₄, filtered, and concentrated. The residue was purified by column chromatography (SiO₂, CH₂Cl₂/7M NH₃ in MeOH gradient) to give the title compound (1.4 mg, 2.85 μmol, 0.888% yield) as a white solid. Exact mass calculated for C₂₂H₃₂F₃N₃O₄S: 491.21. found: LCMS m/z=492.6 [M+H]⁺; ¹H NMR (400 MHz, CDCl₃) δ ppm 1.50-1.67 (m, 6H), 1.81-2.33 (m, 14H), 2.50 (s, 2H), 2.75-2.79 (m, 2H), 3.18 (s, 3H), 3.39-3.51 (m, 2H), 5.15-5.20 (m, 1H), 8.18 (d, J=1.2 Hz, 1H), 8.78 (d, J=1.2 Hz, 1H).

### Example 1.132

### Preparation of 2-(Methylsulfonyl)-5-((1r,4r)-4-(1-(2,2,2-trifluoroethyl)piperidin-4-yloxy)cyclohexyloxy)pyrazine (Compound 128)

A mixture of 2-(methylsulfonyl)-5-((1s,4s)-4-(piperidin-4-yloxy)cyclohexyloxy)pyrazine (36.7 mg, 0.103 mmol), 1,1,1-trifluoro-2-iodoethane (20 μL, 0.205 mmol), and DIEA (46 μL, 0.263 mmol) in 3 mL DMF was heated under microwave irradiation at 120° C. for 1 h. The mixture was purified by HPLC (5-70% CH₃CN+0.5% TFA). Fractions containing desired product were partly concentrated and residue was extracted with 1 M NaOH and CH₂Cl₂. Organic phases were dried over MgSO₄, filtered, and concentrated to the title compound (6.1 mg, 0.014 mmol, 14% yield) as a white solid. Exact mass calculated for C₁₈H₂₆F₃N₃O₄S: 437.16. found: LCMS m/z=438.4 [M+H]⁺; ¹H NMR (400 MHz, CDCl₃) δ ppm 1.50-1.67 (m, 6H), 1.81-1.86 (m, 2H), 1.96-2.00 (m, 2H), 2.12-2.17 (m, 2H), 2.47-2.53 (m, 2H), 2.85-2.90 (m, 2H), 2.97 (q, J=9.7 Hz, 2H), 3.18 (s, 3H), 3.42-3.51 (m, 2H), 5.15-5.19 (m, 1H), 8.18 (d, J=1.2 Hz, 1H), 8.79 (d, J=1.2 Hz, 1H).

### Example 2

### In Vivo Effects of Representative Compounds of the Present Invention on Glucose Homeostasis in Male Diabetic ZDF Rats (Oral Glucose Tolerance Test (oGTT))

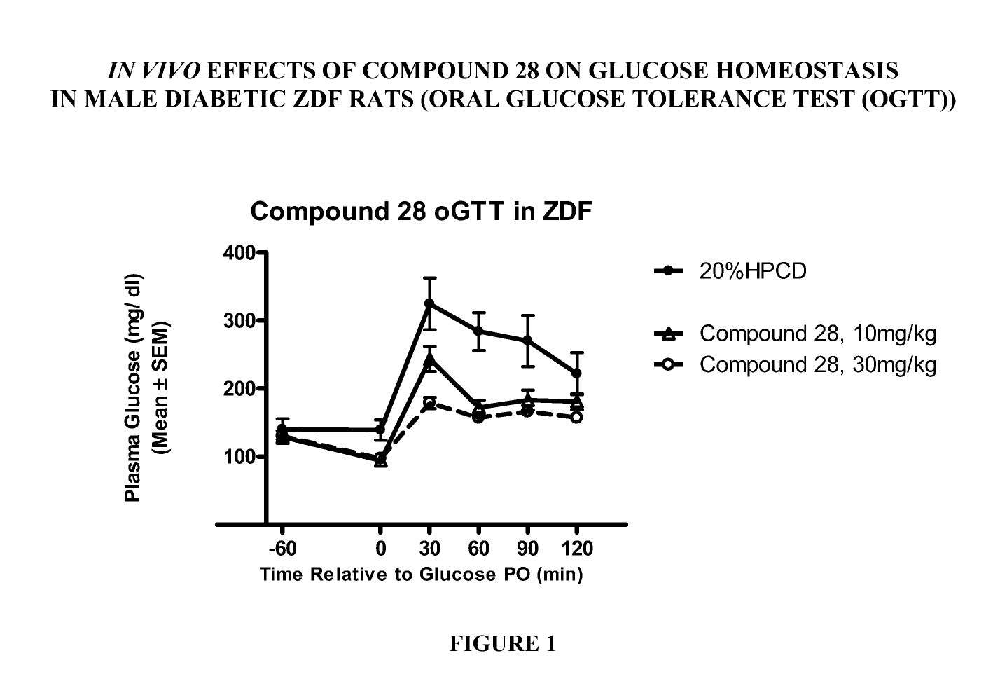

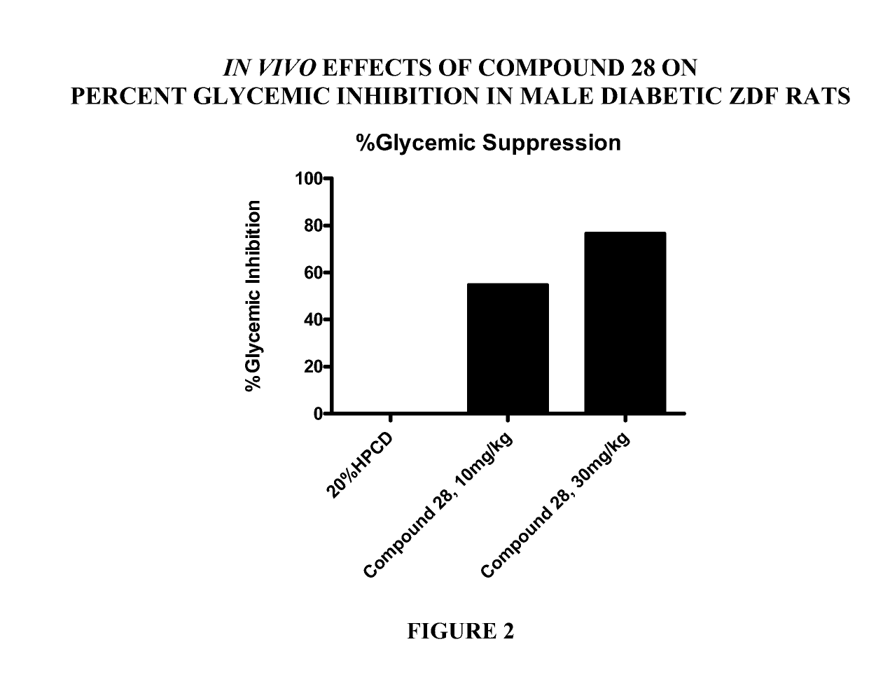

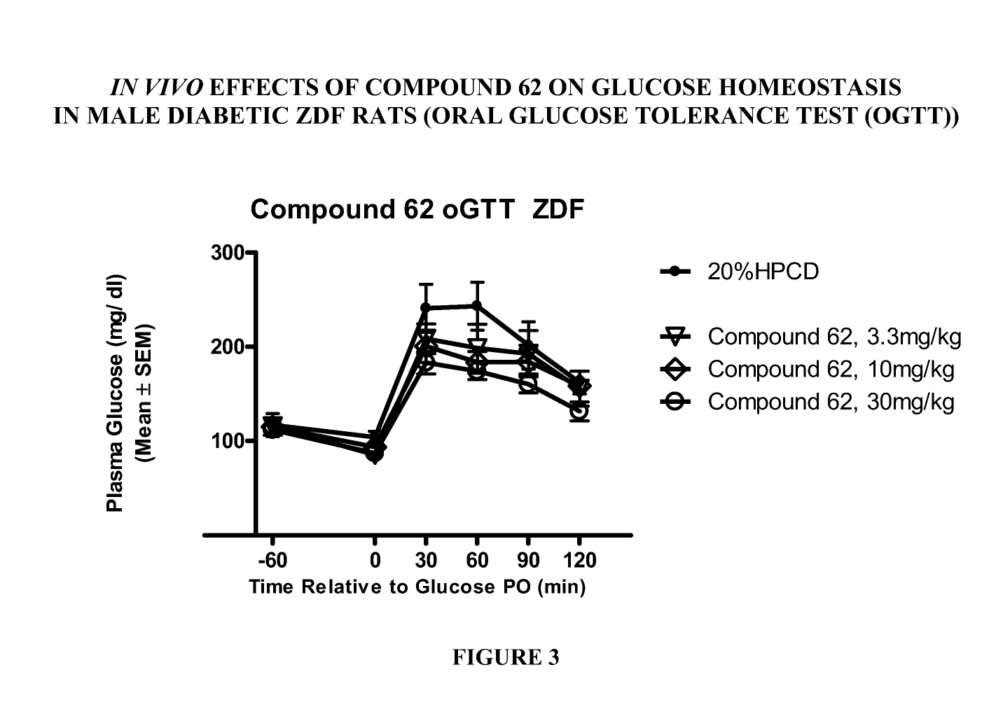

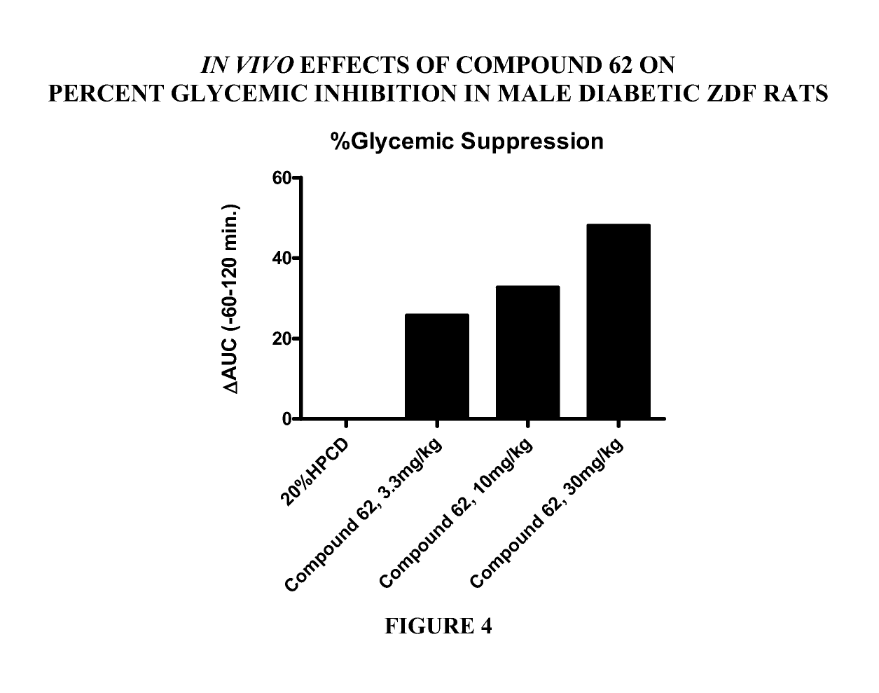

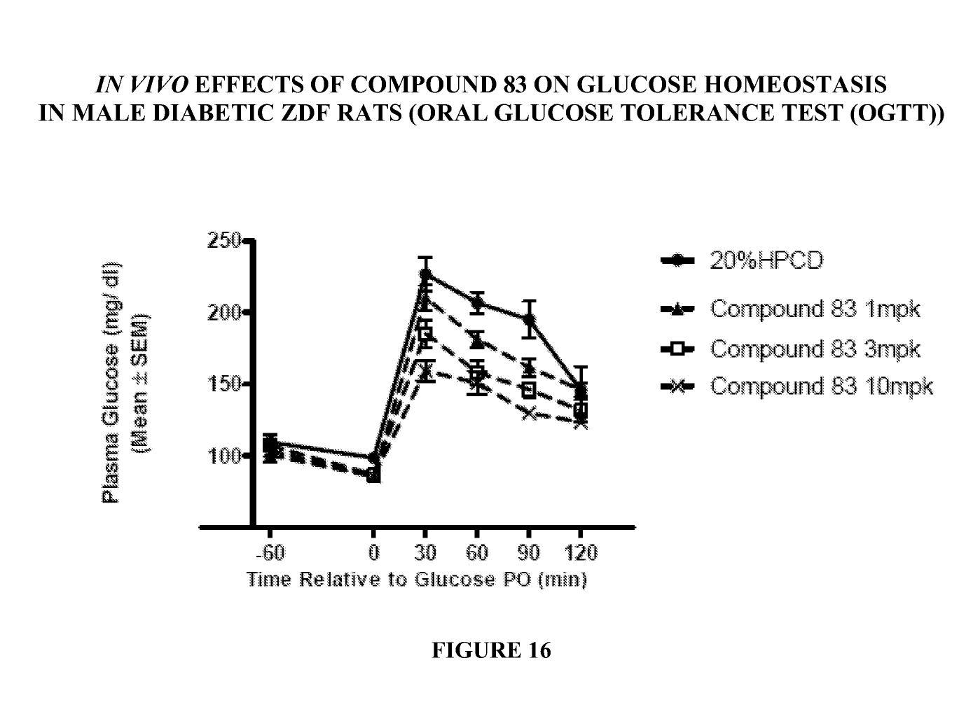

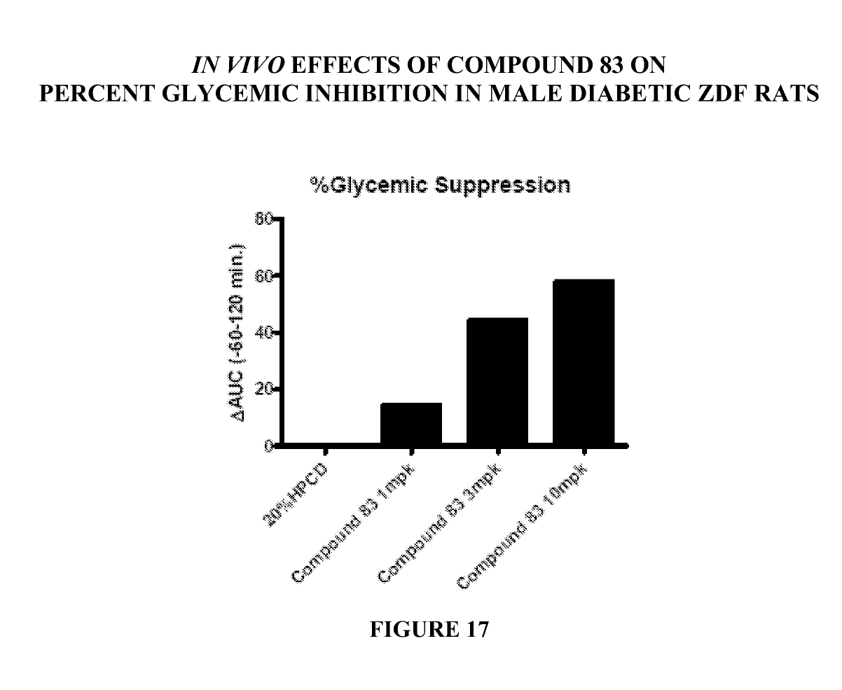

FIGS. 1 to 4, 16 and 17 show results of three independent experiments with three representative GPR119 agonists of the present invention, namely, Compound 28, Compound 62, and Compound 83. In each experiment, male ZDF rats (approximately 10-week old) were fasted for 18 hours and randomly grouped (n=6) to receive a GPR119 agonist (Compound 28, Compound 62, or Compound 83) at indicated doses (mg/kg body weight). Compounds were delivered orally via a gavage needle (p.o., volume 4 mL/kg) 1 h prior to glucose bolus (3 g/kg) (time=−60 min in FIGS. 1, 3, and 16), with a separate group receiving vehicle (20% hydroxypropyl-beta-cyclodextrin) as control in each experiment. At time 0 min the glucose bolus was administered. Levels of blood glucose were assessed using a glucometer (One-Touch Ultra™, LifeScan) at time −60 min (prior to compound administration), at 0 min (time when glucose bolus was given), and at 30, 60, 90, 120 min post glucose bolus. The plasma glucose excursion curves are given in FIGS. 1, 3, and 16, and glucose excursion AUC (area under the curve) reductions in compound treated animals relative to vehicle control are given in FIGS. 2, 4, and 17, and in Table 1. These results demonstrated that the GPR119 agonists, Compound 28, Compound 62, and Compound 83 lowered blood glucose after challenge with glucose in diabetic ZDF rats.

TABLE 1

| | % Inhibition of Glucose Excursion |
|---|---|
| Compound # | (dose: mg/kg) |
| 28 | 54.7% (10) |
| 28 | 76.5% (30) |
| 62 | 25.7% (3.3) |
| 62 | 32.7% (10) |
| 62 | 48.0% (30) |
| 83 | 14.4% (1.0) |
| 83 | 44.3% (3.0) |
| 83 | 57.9% (10) |

### Example 3

### In Vivo Effects of Representative Compounds of the Present Invention on Glucose Homeostasis (Oral Glucose Tolerance Test (oGTT) in Male 129SVE Mice

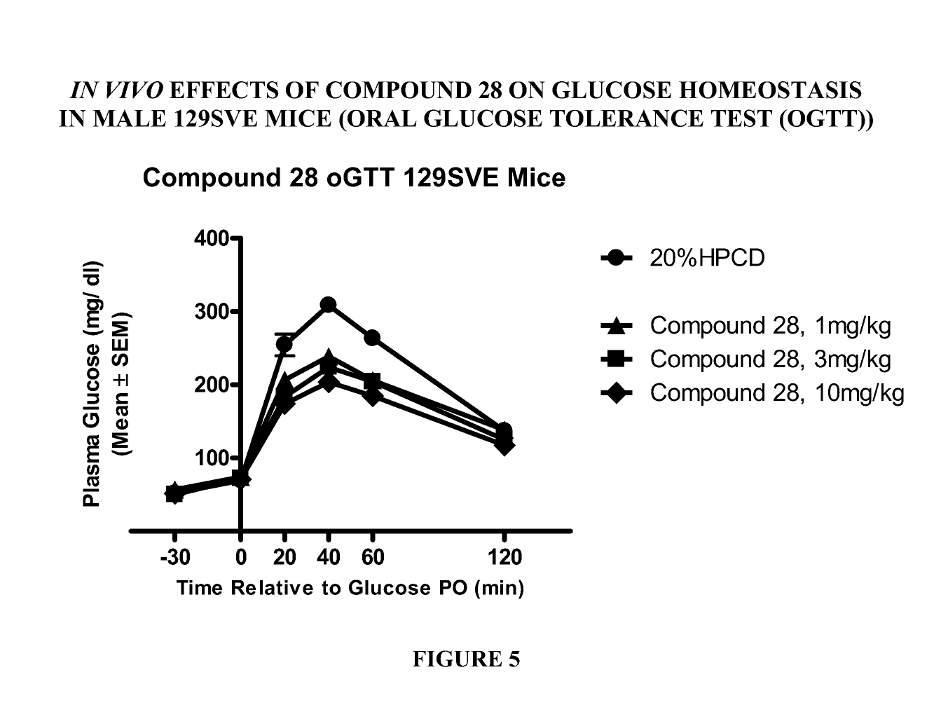

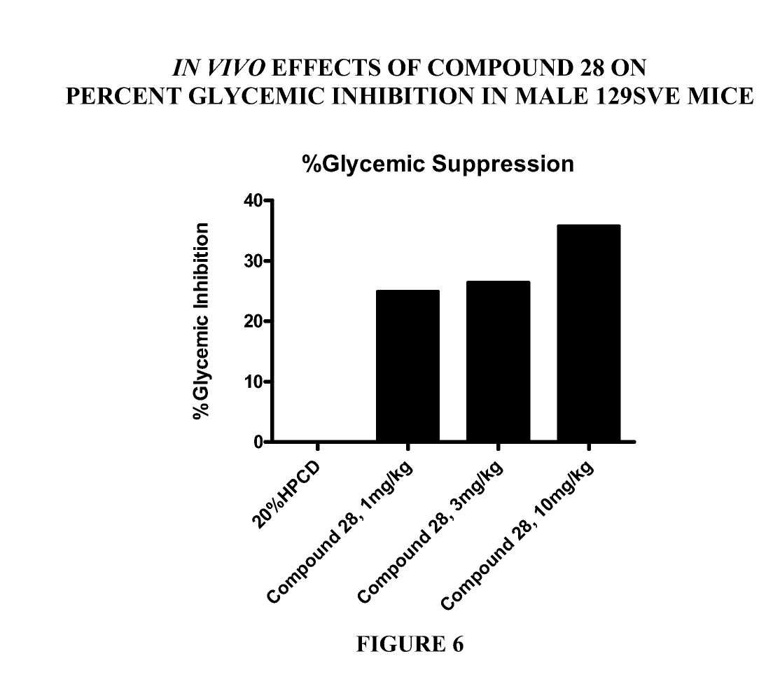

Male 129SVE mice (approximately 8-week old) were fasted for 18 h and randomly grouped (n=6) to receive a GPR119 agonist (Compound 28) at 1, 3, or 10 mg/kg (mg/kg body weight). The compound was delivered orally via a gavage needle (p.o., volume 4 mL/kg) 30 minutes prior to glucose bolus (3 g/kg) (time=−30 min in FIG. 5), with a separate group receiving vehicle (20% hydroxypropyl-beta-cyclodextrin) as control. At time 0 min the glucose bolus was administered. Levels of blood glucose were assessed using a glucometer (One-Touch Ultra™, LifeScan) at time −30 minute (prior to compound administration), at 0 min (at time when glucose bolus was given), and at 20, 40, 60, 120 minutes post glucose bolus. The plasma glucose excursion curve is given in FIG. 5, and glucose excursion AUC (area under the curve) reduction in compound treated animals relative to vehicle control is given in FIG. 6, and in Table 2. These results demonstrated that the GPR119 agonist, Compound 28, lowered blood glucose after challenge with glucose in 129SVE mice.

TABLE 2

| Compound Dose | % Inhibition of Glucose Excursion |
|---|---|
| 1 mg/kg | 24.9% |
| 3 mg/kg | 26.4% |
| 10 mg/kg | 35.7% |

### Example 4

### In Vivo Effects of Representative Compounds of the Present Invention on Incretin Hormone GIP Release

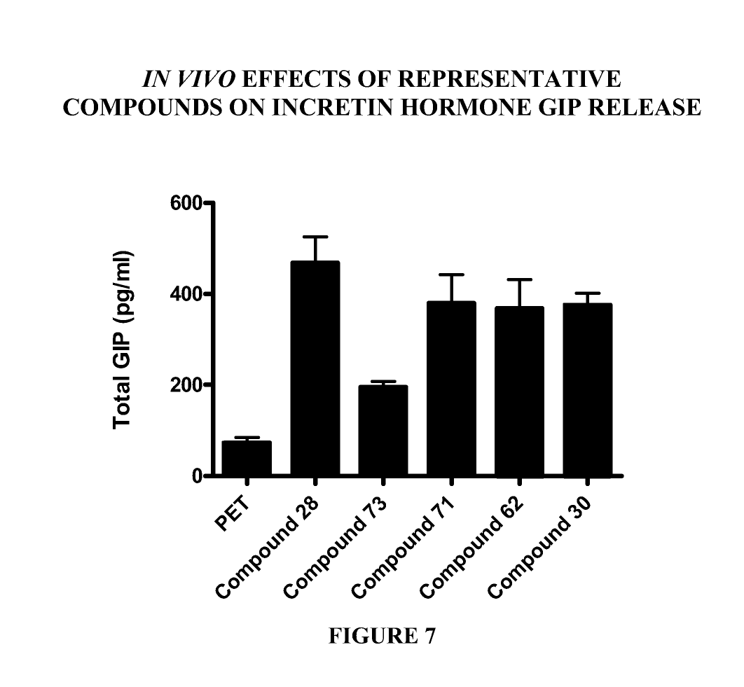

Male 129SVE mice (approximately 8-week old) were fasted for 18 h and randomly grouped (n=6) to receive a GPR119 agonist (Compound 28, Compound 73, Compound 71, Compound 62, and Compound 30) at 10 mpk dose (mg/kg body weight). Compounds were delivered orally via a gavage needle (p.o., volume 4 mL/kg), and after 45 min a blood sample was collected to determine plasma total GIP levels. A separate group received vehicle (PET: 80% PEG:10% Ethanol:10% Tween80™) as control. Plasma GIP levels were determined using a Total GIP ELISA kit from Millipore. The results are given in FIG. 7 and Table 3.

TABLE 3
Total Plasma GIP in Mice

| Compound # | Total GIP, pg/mL (dose, mg/kg) |
|---|---|
| PET Vehicle | 72.5 |
| 28 | 468.1 |
| 73 | 195.2 |
| 71 | 379.8 |
| 62 | 368.5 |
| 30 | 375.7 |

### Example 5

### Homogeneous Time-Resolved Fluorescence (HTRF®) Assay for Direct cAMP Measurement

GPR119 agonists were evaluated in an HTRF® cAMP detection assay according to the manufacturer's instructions (Cisbio, cAMP Dynamic 2 Assay Kit; #62AM4PEJ) using CHO-K1 cells stably expressing the GPR119 receptor. Briefly, CHO-K1 cells were transduced with a lentiviral vector encoding the nucleotide sequence of GPR119 (NCBI mRNA and protein reference sequences: NM_{—}178471.2 & NP_{—}848566, (GPR119 has also been referred to as Glucose-Dependent Insulinotropic Receptor (GDIR)). The N-terminus of the GPR119 nucleotide sequence was modified to replace the first, methionine-coding codon with a nucleotide sequence coding for a standard, nine amino acid, hemagglutinin tag. Following transduction, cells expressing the GPR119 receptor were isolated and a single clone was isolated following standard dilution-cloning procedures. On the day of the assay, cultured CHO-GPR119 cells were harvested, suspended in assay buffer and plated into 384-well assay plates (PerkinElmer Proxiplate #6008280) at a density of 2,000 cells per well. A cAMP standard curve was added to each plate. Test compounds were solubilized in DMSO, serially diluted in DMSO and then diluted in assay buffer before adding to the cells. Test compounds were evaluated in triplicate, using 10-point, 5-fold serial dilutions starting at 10 μM. The final DMSO concentration in the assay was 0.5%. Compounds and cells were incubated for 1 h at room temperature and then detection reagents were added to each well (cAMP-D2 in cell lysis buffer, followed by europium cryptate-labeled anti-cAMP antibody). Plates were then incubated at room temperature for 1 h prior to reading. Time-resolved fluorescence measurements were collected on PerkinElmer Envision™ or BMG Pherastar™ microplate readers. The compound N-(2-fluoro-4-(methylsulfonyl)phenyl)-6-(4-(3-isopropyl-1,2,4-oxadiazol-5-yl)piperidin-1-yl)-5-nitropyrimidin-4-amine was used as a positive control in each runset while assay buffer containing 0.5% DMSO was used as the negative control. The HTRF® assay was used to determine EC₅₀ values for GPR119 agonists.

Certain representative compounds of the present invention and their corresponding EC₅₀ values are shown in Table B.

TABLE B

| Compound No. | EC₅₀ hGPR119 (nM) |
|---|---|
| 1 | 266 |
| 5 | 8 |
| 13 | 19 |
| 35 | 426 |
| 48 | 11 |
| 69 | 31 |
| 77 | 281 |
| 83 | 3 |
| 98 | 152 |
| 116 | 5 |

Each of the compounds described in Table A were observed to have an hGPR119 EC₅₀ value ranging from about 1 nM to about 25 μM.

### Example 6

### Powder X-Ray Diffraction

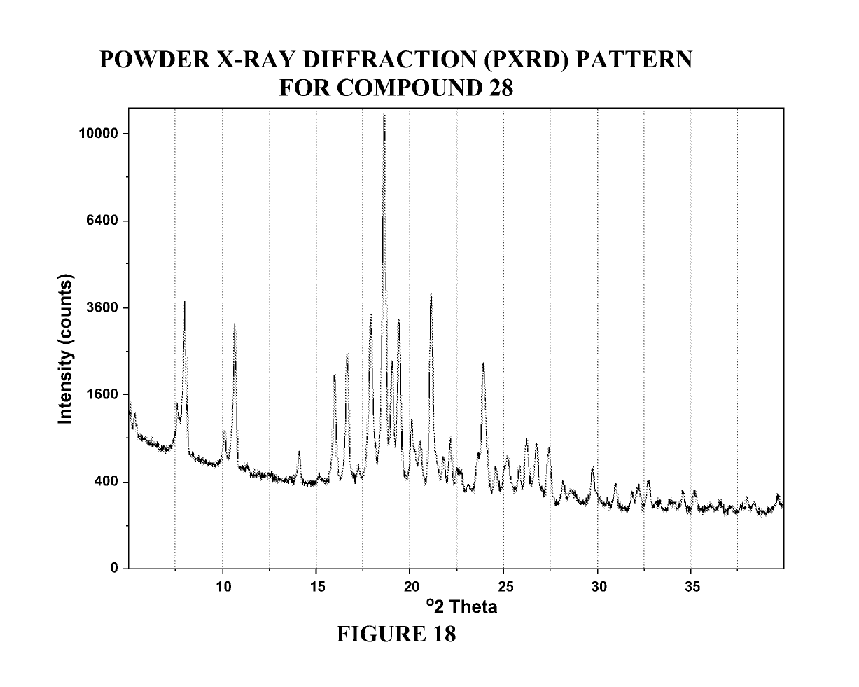

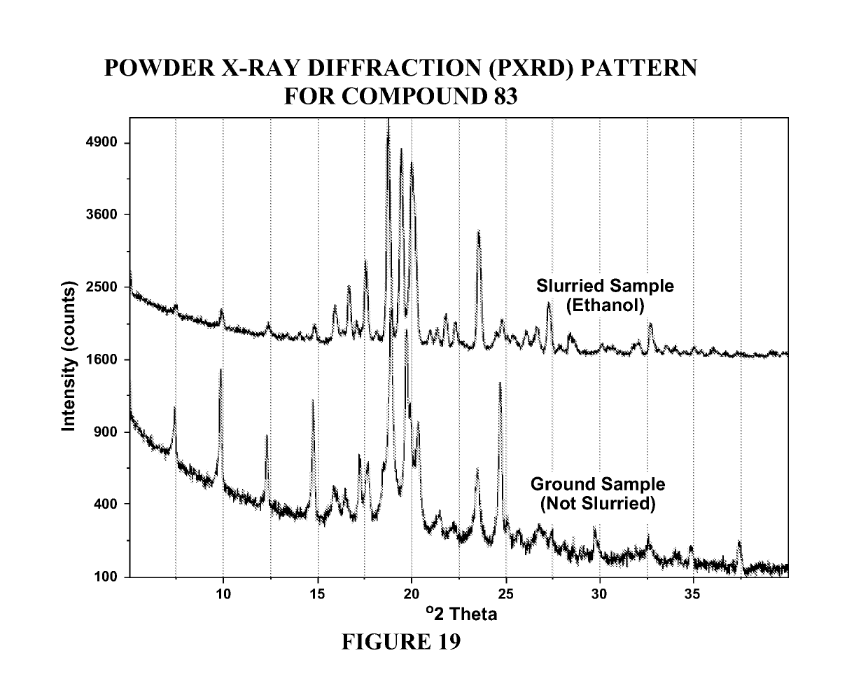

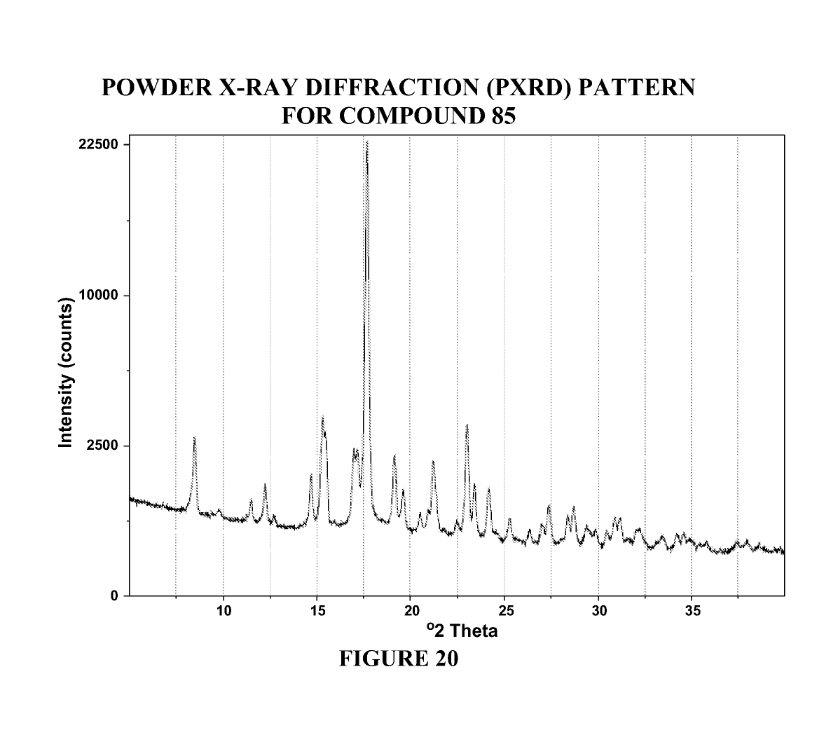

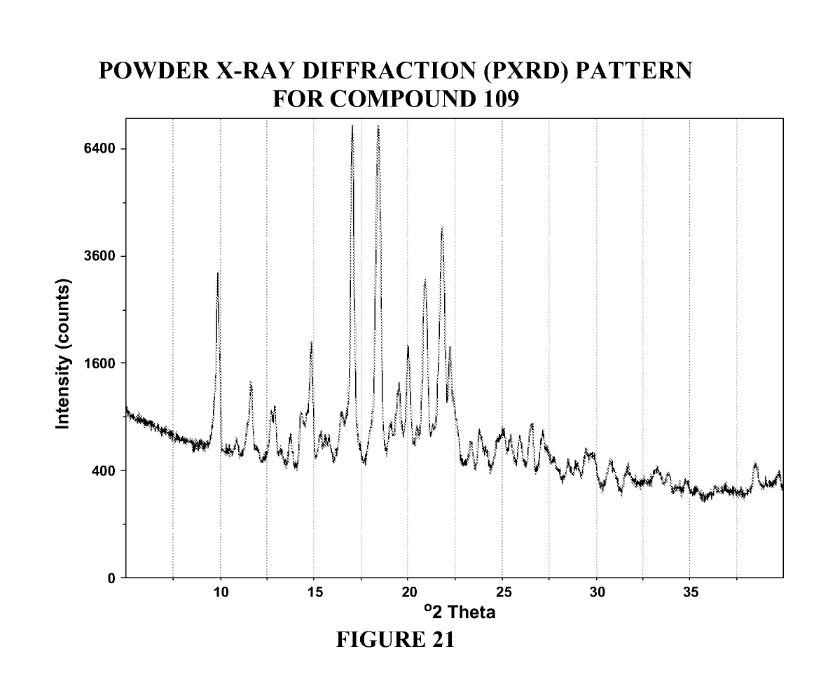

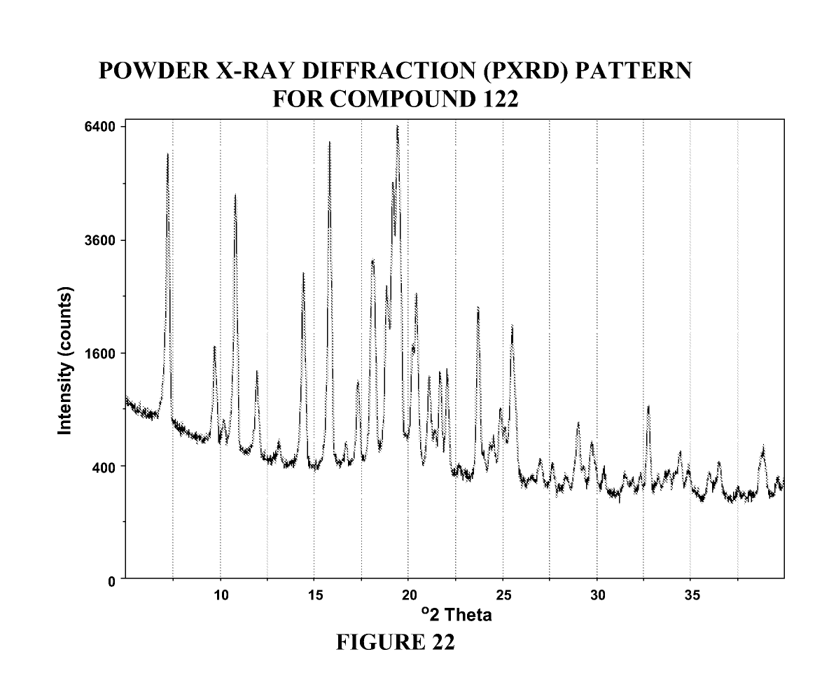

Powder X-ray Diffraction (PXRD) data were collected on an X'Pert PRO MPD powder diffractometer (PANalytical, Inc.) with a Cu source set at 45 kV and 40 mA, Cu(Kα) radiation and an X'Celerator detector. Samples were added to the sample holder and smoothed flat with a spatula and weigh paper. With the samples spinning, X-ray diffractograms were obtained by a 12-min scan over the 2-theta range 5-40° 2θ. Diffraction data were viewed and analyzed with the X'Pert Data Viewer Software, version 1.0a and X'Pert HighScore Software, version 1.0b. FIG. 18 shows a powder X-ray diffraction (PXRD) pattern for Compound 28. FIG. 19 shows a powder X-ray diffraction (PXRD) pattern (a sample prepared after a slurry in ethanol and another that was ground) for Compound 83. FIG. 20 shows a powder X-ray diffraction (PXRD) pattern for Compound 85. FIG. 21 shows a powder X-ray diffraction (PXRD) pattern for Compound 109. FIG. 22 shows a powder X-ray diffraction (PXRD) pattern for Compound 122.

### Example 7

### Differential Scanning calorimetry and Thermal Gravimetric Analysis

A. Differential Scanning Calorimetry

Differential scanning calorimetry (DSC) studies were conducted using a TA Instruments, Q2000 at heating rate 10° C./min The instruments were calibrated for temperature and energy using the melting point and enthalpy of fusion of an indium standard. Thermal events (desolvation, melting, etc.) were evaluated using Universal Analysis 2000 software, version 4.1D, Build 4.1.0.16.

B. Thermal Gravimetric Analysis.

Thermogravimetric analyses (TGA) were conducted using a TA Instruments TGA Q500 or Q5000 at heating rate 10° C./min The instruments were calibrated using a standard weight for the balance, and Alumel and Nickel standards for the furnace (Curie point measurements). Thermal events such as weight-loss are calculated using the Universal Analysis 2000 software, version 4.1D, Build 4.1.0.16.

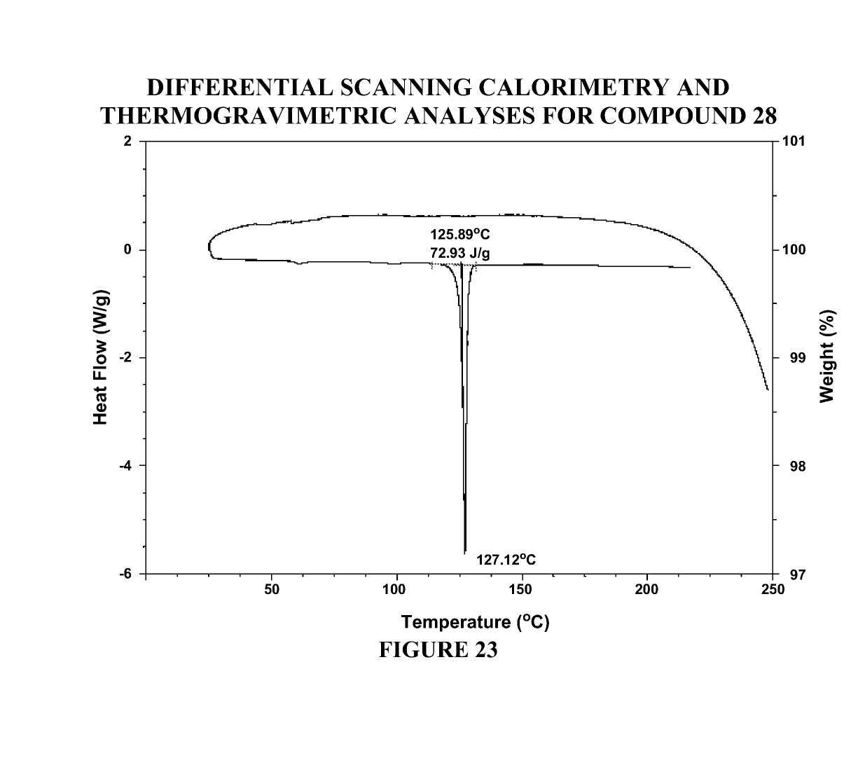

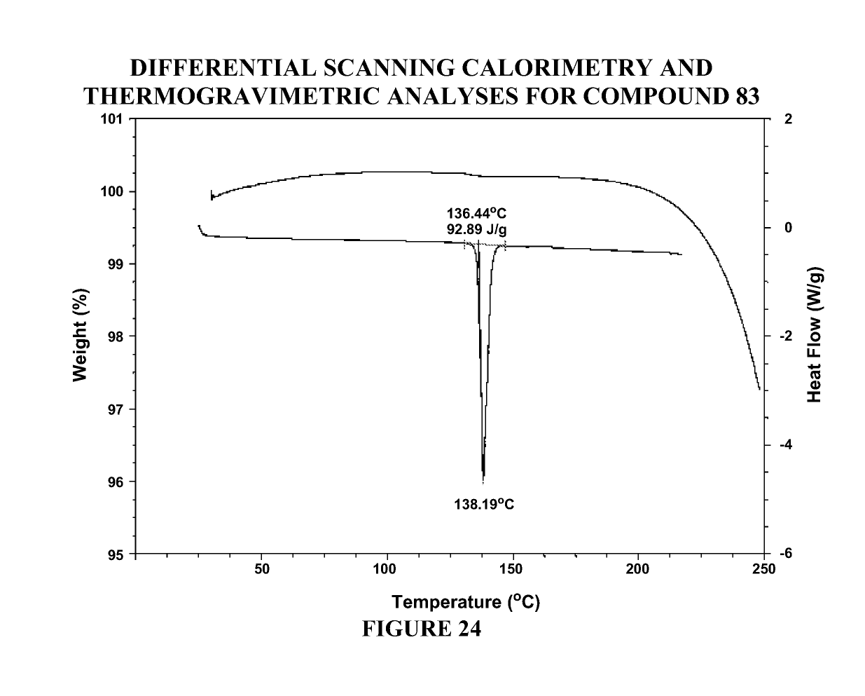

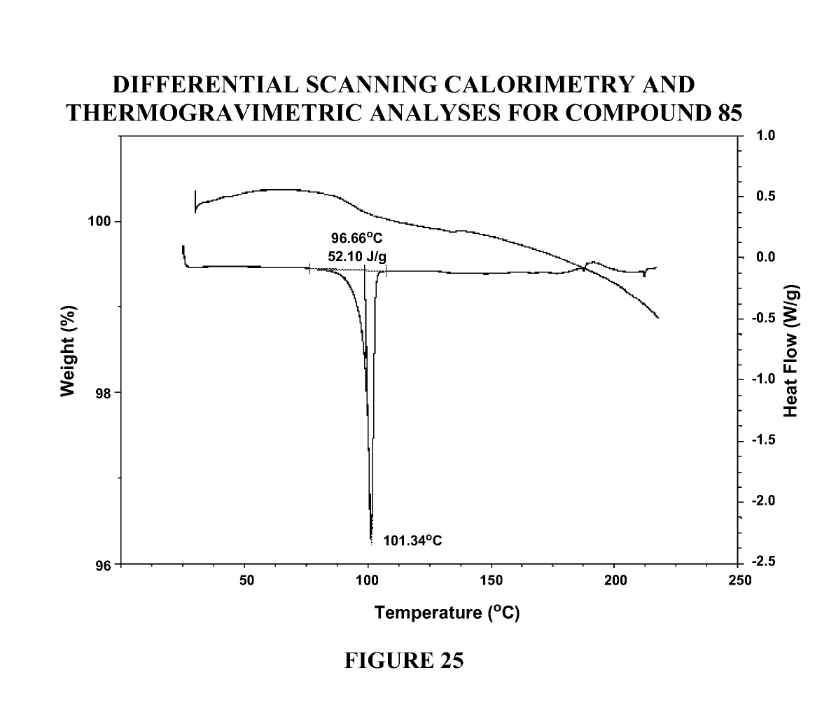

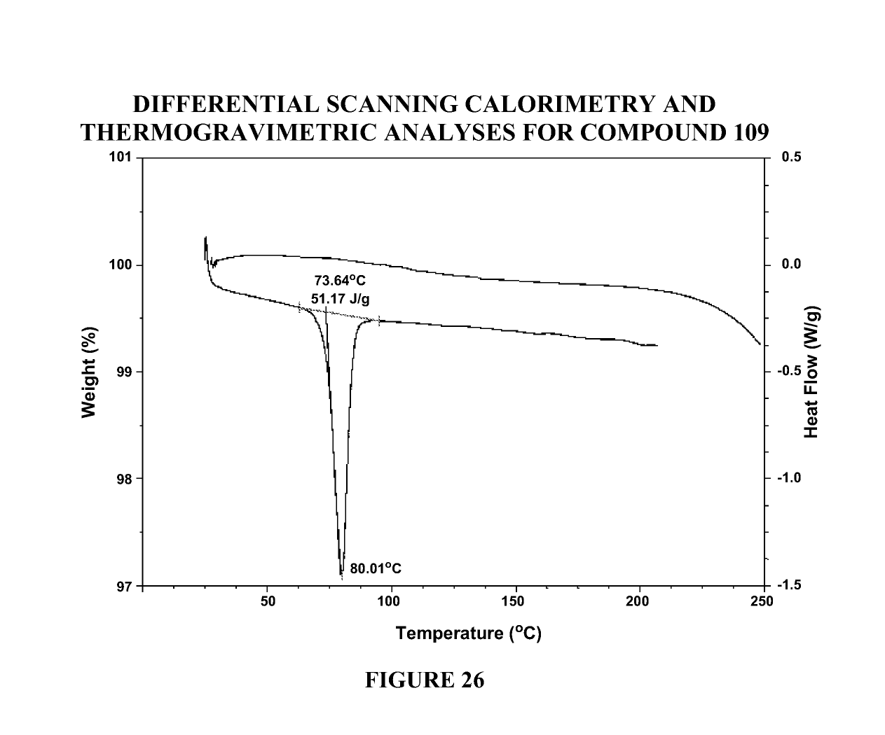

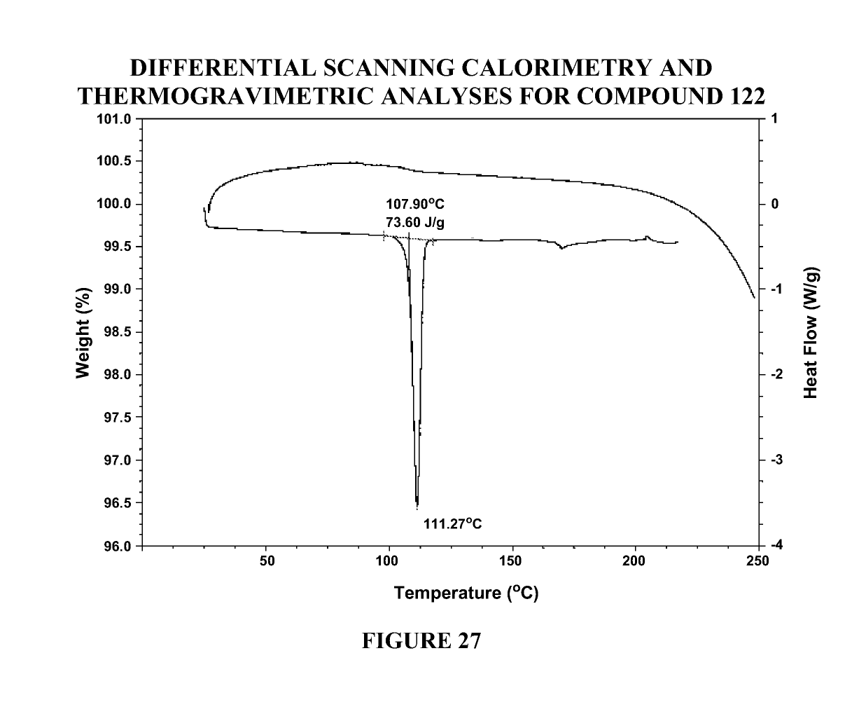

FIG. 23 shows the TGA and DSC for Compound 28. FIG. 24 shows the TGA and DSC for Compound 83. FIG. 25 shows the TGA and DSC for Compound 85. FIG. 26 shows the TGA and DSC for Compound 109. FIG. 27 shows the TGA and DSC for Compound 122.

Those skilled in the art will recognize that various modifications, additions, and substitutions to the illustrative examples set forth herein can be made without departing from the spirit of the invention and are, therefore, considered within the scope of the invention.

Citation of any reference throughout this application is not to be construed as an admission that such reference is prior art to the present application.

1. A compound selected from compounds of Formula (Ic) and pharmaceutically acceptable salts, solvates, and hydrates thereof: 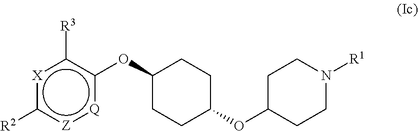
wherein:
Q is N or CR⁴;
Z is N or CR⁵;
X is N, N(O), or CR⁶;
R¹ is selected from the group consisting of H, S(O)₂R⁷, C(O)R⁷, CH₂R⁸, C(O)OR⁹, and C(O)SR⁹; or R¹ is selected from the group consisting of heteroaryl and phenyl, each optionally substituted with one or more substituents selected independently from the group consisting of C₂-C₆ alkenyl, C₁-C₆ alkoxy, C₁-C₆ alkyl, halogen, C₁-C₆ haloalkoxy, and C₁-C₆ haloalkyl;
R² is selected from the group consisting of H, C₁-C₆ alkyl, cyano, C₃-C₆ cycloalkyl, halogen, C₁-C₆ haloalkyl, heteroaryl, heterocyclyl, S(O)ₙR¹¹, and C(O)NR¹²R¹³; wherein said C₁-C₆ alkyl is optionally substituted with one or more substituents selected independently from the group consisting of amino, C₁-C₆ alkylsulfonyl, cyano, and C(O)NR¹²R¹³; said C₃-C₆ cycloalkyl is optionally substituted with C(O)NR¹²R¹³; said heteroaryl is optionally substituted with C₁-C₆ alkyl; and said heterocyclyl is optionally substituted with one or more substituents selected independently from the group consisting of amino, C₁-C₆ alkylsulfonyl, hydroxyl, and halogen;
R³, R⁴, R⁵, and R⁶ are each independently selected from the group consisting of H, C₁-C₆ alkyl, C₁-C₆ alkylsulfonyl, and halogen;
R⁷ is selected from the group consisting of C₁-C₆ alkyl, C₃-C₆ cycloalkyl, and C₁-C₆ haloalkyl; wherein said C₃-C₆ cycloalkyl is optionally substituted with one or more C₁-C₆ alkyl;
R⁸ is selected from the group consisting of C₃-C₆ cycloalkyl, C₁-C₆ haloalkyl, heteroaryl, phenyl, and C(O)OR⁹; wherein said C₃-C₆ cycloalkyl and said heteroaryl are each optionally substituted with one or more substituents selected independently from the group consisting of C₁-C₆ haloalkyl and C₁-C₆ alkyl;
R⁹ is selected from the group consisting of C₁-C₆ alkyl, C₃-C₆ cycloalkyl, C₁-C₆ haloalkyl, heterocyclyl, and phenyl; said C₁-C₆ alkyl and said C₃-C₆ cycloalkyl are each optionally substituted with one or more substituents selected independently from the group consisting of C₁-C₆ alkyl, halogen, hydroxyl, C₁-C₆ alkoxy, and R¹⁰, wherein said C₁-C₆ alkoxy is optionally substituted with phenyl;
R¹⁰ is heterocyclyl optionally substituted with C₁-C₆ alkyl;
R¹¹ is selected from the group consisting of C₁-C₆ alkyl, C₃-C₆ cycloalkyl, C₁-C₆ haloalkyl, and heterocyclyl; wherein said C₁-C₆ alkyl and heterocyclyl are each optionally substituted with one or more substituents selected independently from the group consisting of halogen, hydroxyl, and NR¹²R¹³; and said heterocyclyl is optionally substituted with one or more substituents selected independently from the group consisting of amino, C₁-C₆ alkyl, and hydroxyl;
R¹² and R¹³ are each independently selected from the group consisting of H, C₁-C₆ alkyl, and C₃-C₆ cycloalkyl; wherein said C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, hydroxyl, and C₁-C₆ alkoxy; and
n is 0, 1, or 2.

2-3. (canceled)

4. The compound according to claim 1, wherein:
X is N or CR⁶;
R¹ is selected from the group consisting of H, S(O)₂R⁷, C(O)R⁷, CH₂R⁸, and C(O)OR⁹; or R¹ is selected from the group consisting of heteroaryl and phenyl, each optionally substituted with one or more substituents selected independently from the group consisting of C₂-C₆ alkenyl, C₁-C₆ alkoxy, C₁-C₆ alkyl, halogen, C₁-C₆ haloalkoxy, and C₁-C₆ haloalkyl;
R² is selected from the group consisting of H, C₁-C₆ alkyl, cyano, C₃-C₆ cycloalkyl, halogen, C₁-C₆ haloalkyl, heteroaryl, heterocyclyl, S(O)₂R¹¹, and C(O)NR¹²R¹³; wherein said C₁-C₆ alkyl is optionally substituted with one or more substituents selected independently from the group consisting of amino, C₁-C₆ alkylsulfonyl, cyano, and C(O)NR¹²R¹³; said C₃-C₆ cycloalkyl is optionally substituted with C(O)NR¹²R¹³; said heteroaryl is optionally substituted with C₁-C₆ alkyl; and said heterocyclyl is optionally substituted with one or more substituents selected independently from the group consisting of amino, C₁-C₆ alkylsulfonyl, hydroxyl, and halogen;
R⁸ is selected from the group consisting of heteroaryl, phenyl, and C(O)OR⁹; wherein said heteroaryl is optionally substituted with C₁-C₆ alkyl;
R⁹ is selected from the group consisting of C₁-C₆ alkyl, C₃-C₆ cycloalkyl, C₁-C₆ haloalkyl, heterocyclyl, and phenyl; wherein said C₁-C₆ alkyl is optionally substituted with one or more substituents selected independently from the group consisting of hydroxyl, C₁-C₆ alkoxy, and R¹⁰, wherein said C₁-C₆ alkoxy is optionally substituted with phenyl; and said C₃-C₆ cycloalkyl is optionally substituted with one or more substituents selected independently from the group consisting of C₁-C₆ alkyl and halogen; and
R¹¹ is selected from the group consisting of C₁-C₆ alkyl, C₃-C₆ cycloalkyl, C₁-C₆ haloalkyl, and heterocyclyl; wherein said C₁-C₆ alkyl is optionally substituted with one or more substituents selected independently from the group consisting of hydroxyl and NR¹²R¹³; and said heterocyclyl is optionally substituted with one or more substituents selected independently from the group consisting of amino, C₁-C₆ alkyl, and hydroxyl.

5. The compound according to claim 1, wherein:
X is N or CR⁶;
R¹ is selected from the group consisting of H, S(O)₂R⁷, C(O)R⁷, CH₂R⁸, and C(O)OR⁹; or R¹ is heteroaryl or phenyl, each optionally substituted with one or more substituents selected independently from the group consisting of C₂-C₆ alkenyl, C₁-C₆ alkoxy, C₁-C₆ alkyl, halogen, C₁-C₆ haloalkoxy, and C₁-C₆ haloalkyl;
R² is selected from the group consisting of H, C₁-C₆ alkyl, cyano, C₃-C₆ cycloalkyl, halogen, C₁-C₆ haloalkyl, heteroaryl, heterocyclyl, S(O)₂R¹¹, and C(O)NR¹²R¹³; wherein said C₁-C₆ alkyl is optionally substituted with one or more substituents selected independently from the group consisting of amino, C₁-C₆ alkylsulfonyl, cyano, and C(O)NR¹²R¹³; said C₃-C₆ cycloalkyl is optionally substituted with C(O)NR¹²R¹³; said heteroaryl is optionally substituted with C₁-C₆ alkyl; and said heterocyclyl is optionally substituted with one or more substituents selected independently from the group consisting of amino, C₁-C₆ alkylsulfonyl, hydroxyl, and halogen;
R⁸ is selected from the group consisting of heteroaryl, phenyl, and C(O)OR⁹; wherein said heteroaryl is optionally substituted with C₁-C₆ alkyl;
R⁹ is selected from the group consisting of C₁-C₆ alkyl, C₃-C₆ cycloalkyl, C₁-C₆ haloalkyl, and heterocyclyl; wherein said C₁-C₆ alkyl is optionally substituted with one or more substituents selected independently from the group consisting of hydroxyl and R¹⁰; and said C₃-C₆ cycloalkyl is optionally substituted with one C₁-C₆ alkyl substituent;
R¹¹ is selected from the group consisting of C₁-C₆ alkyl, C₃-C₆ cycloalkyl, C₁-C₆ haloalkyl, and heterocyclyl; wherein said C₁-C₆ alkyl is optionally substituted with one or more substituents selected independently from the group consisting of hydroxyl and NR¹²R¹³, and said heterocyclyl is optionally substituted with one or more substituents selected independently from the group consisting of amino, C₁-C₆ alkyl, and hydroxyl; and
R¹² and R¹³ are each independently selected from the group consisting of H, C₁-C₆ alkyl, and C₃-C₆ cycloalkyl; wherein said C₁-C₆ alkyl is optionally substituted with hydroxyl; or R¹² and R¹³ together with the nitrogen to which they are both bonded form a heterocyclyl optionally substituted with one or more substituents selected independently from the group consisting of cyano, halogen, and hydroxyl.

6. The compound according to claim 1, wherein R¹ is selected from the group consisting of H, S(O)₂R⁷, C(O)R⁷, CH₂R⁸, C(O)OR⁹, and C(O)SR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of prop-1-en-2-yl, ethoxy, methoxy, tert-butyl, ethyl, isopropyl, methyl, chloro, fluoro, trifluoromethoxy, 2-fluoropropan-2-yl, and trifluoromethyl;
R⁷ is selected from the group consisting of 2,2-dimethylpropyl, isopropyl, 2-methylcyclopropyl, 1,1-difluoropropyl, and cyclopropyl;
R⁸ is selected from the group consisting of 1,2,4-oxadiazolyl, cyclopropyl, 1,1,2,2-tetrafluoroethyl, cyclobutyl, trifluoromethyl, and C(O)OR⁹; wherein said 1,2,4-oxadiazole, cyclopropyl, and cyclobutyl are each optionally substituted with one group selected from the group consisting of isopropyl and trifluoromethyl; and
R⁹ is selected from the group consisting of isobutyl, isopropyl, sec-butyl, tert-butyl, cyclopentyl, (3-methyloxetan-3-yl)methyl, 1-methylcyclopropyl, 2-methylcyclopropyl, 1,3-difluoropropan-2-yl, 1-fluoropropan-2-yl, 1,1,1,3,3,3-hexafluoropropan-2-yl, 1,1,1-trifluoropropan-2-yl, tetrahydrofuran-3-yl, 1-hydroxypropan-2-yl, phenyl, 2,2,3,3-tetrafluorocyclobutyl, 1-(benzyloxy)propan-2-yl, 1,1,1-trifluoro-2-methylpropan-2-yl, and cyclopropyl.

7. The compound according to claim 1, wherein R¹ is selected from the group consisting of H, cyclopropylsulfonyl, isopropylsulfonyl, 3-isobutyryl, 3,3-dimethylbutanoyl, 2-methylcyclopropanecarbonyl, 2,2-difluorobutanoyl, (3-isopropyl-1,2,4-oxadiazol-5-yl)methyl, 2-tert-butoxy-2-oxoethyl, tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (tetrahydrofuran-3-yloxy)carbonyl, (1,1,1-trifluoropropan-2-yloxy)carbonyl, (1,3-difluoropropan-2-yloxy)carbonyl, (1-fluoropropan-2-yloxy)carbonyl, 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, 5-methoxy-pyrimidin-2-yl, (1-hydroxypropan-2-yloxy)carbonyl, phenoxycarbonyl, 5-(2-fluoropropan-2-yl)-1,2,4-oxadiazol-3-yl, (2,2,3,3-tetrafluorocyclobutoxy)carbonyl, (1-(benzyloxy)propan-2-yloxy)carbonyl, isopropylthiocarbonyl, 5-methylpyridin-2-yl, 5-ethylpyridin-2-yl, (1,1,1-trifluoro-2-methylpropan-2-yloxy)carbonyl, cyclopropylthiocarbonyl, (1-(trifluoromethyl)cyclopropyl)methyl, 2,2,3,3-tetrafluoropropyl, (1-(trifluoromethyl)cyclobutyl)methyl, and 2,2,2-trifluoroethyl.

8. The compound according to claim 1, wherein:
R¹ is C(O)OR⁹; and
R⁹ is selected from the group consisting of isobutyl, isopropyl, sec-butyl, tert-butyl, cyclopentyl, (3-methyloxetan-3-yl)methyl, 1-methylcyclopropyl, 1,3-difluoropropan-2-yl, 1-fluoropropan-2-yl, 1,1,1,3,3,3-hexafluoropropan-2-yl, 1,1,1-trifluoropropan-2-yl, tetrahydrofuran-3-yl, and 1-hydroxypropan-2-yl.

9. The compound according to claim 1, wherein:
R¹ is selected from the group consisting of tert-butoxycarbonyl, isopropoxycarbonyl, isobutoxycarbonyl, cyclopentyloxycarbonyl, (1,1,1,3,3,3-hexafluoropropan-2-yloxy)carbonyl, ((3-methyloxetan-3-yl)methoxy)carbonyl, (1-methylcyclopropoxy)carbonyl, sec-butoxycarbonyl, (tetrahydrofuran-3-yloxy)carbonyl, (1,1,1-trifluoropropan-2-yloxy)carbonyl, (1,3-difluoropropan-2-yloxy)carbonyl, (1-fluoropropan-2-yloxy)carbonyl, (1-hydroxypropan-2-yloxy)carbonyl, phenoxycarbonyl, (2,2,3,3-tetrafluorocyclobutoxy)carbonyl, and (1-(benzyloxy)propan-2-yloxy)carbonyl.

10. The compound according to claim 1, wherein:
R¹ is C(O)OR⁹;
R⁹ is C₁-C₆ alkyl, C₃-C₆ cycloalkyl, C₁-C₆ haloalkyl, or heterocyclyl; wherein said C₁-C₆ alkyl is optionally substituted with hydroxyl or R¹⁰; and said C₃-C₆ cycloalkyl is optionally substituted with one C₁-C₆ alkyl substituent; and
R¹⁰ is heterocyclyl optionally substituted with one C₁-C₆ alkyl substituent.

11. The compound according to claim 1, wherein:
R¹ is 1,2,4-oxadiazolyl, phenyl, pyrimidinyl, pyridinyl, pyridazinyl, or pyrazinyl, each optionally substituted with one or two substituents selected independently from the group consisting of C₂-C₆ alkenyl, C₁-C₄ alkoxy, C₁-C₆ alkyl, halogen, C₁-C₄ haloalkoxy, and C₁-C₆ haloalkyl.

12. The compound according to claim 1, wherein:
R¹ is 1,2,4-oxadiazolyl, phenyl, pyrimidinyl, pyridinyl, pyridazinyl, or pyrazinyl, each optionally substituted with one or two substituents selected independently from the group consisting of prop-1-en-2-yl, ethoxy, methoxy, tert-butyl, ethyl, isopropyl, methyl, chloro, fluoro, trifluoromethoxy, 2-fluoropropan-2-yl, and trifluoromethyl.

13. The compound according to claim 1, wherein:
R¹ is selected from the group consisting of 3-isopropyl-1,2,4-oxadiazol-5-yl, 5-isopropyl-1,2,4-oxadiazol-3-yl, 3-(2-fluoropropan-2-yl)-1,2,4-oxadiazol-5-yl, 3-tert-butyl-1,2,4-oxadiazol-5-yl, 3-(prop-1-en-2-yl)-1,2,4-oxadiazol-5-yl, p-tolyl, 4-(trifluoromethyl)phenyl, 4-(trifluoromethoxy)phenyl, 4-methoxyphenyl, 3-(trifluoromethyl)phenyl, 4-fluorophenyl, 4-chloro-2-fluorophenyl, 5-ethyl-pyrimidin-2-yl, 5-chloro-pyrimidin-2-yl, 5-methyl-pyrimidin-2-yl, 5-(trifluoromethyl)pyridin-2-yl, 3-methyl-pyridazin-6-yl, 2-methyl-pyrazin-5-yl, 5-chloro-pyridin-2-yl, 3-ethoxy-pyridazin-6-yl, 5-fluoro-pyridin-2-yl, and 5-methoxy-pyrimidin-2-yl.

14. The compound according to claim 1, wherein:
R² is selected from the group consisting of H, C₁-C₆ alkyl, cyano, halogen, heteroaryl, heterocyclyl, S(O)ₙR¹¹, and C(O)NR¹²R¹³; wherein said C₁-C₆ alkyl is optionally substituted with one or more substituents selected independently from the group consisting of amino, cyano, and C(O)NR¹²R¹³;
R¹¹ is selected from the group consisting of C₁-C₆ alkyl, C₃-C₆ cycloalkyl, and heterocyclyl; wherein said heterocyclyl is optionally substituted with one or two halogens; and
R¹² and R¹³ are each independently C₁-C₆ alkyl; or R¹² and R¹³ together with the nitrogen to which they are both bonded form a heterocyclyl optionally substituted with one or more substituents selected independently from the group consisting of halogen and C₁-C₆ alkoxy.

15. The compound according to claim 1, wherein:
R² is selected from the group consisting of H, methyl, isopropylsulfonyl, methylsulfonyl, cyano, dimethylcarbamoyl, bromo, fluoro, pyridazin-4-yl, 1H-1,2,4-triazol-1-yl, 1,1-dioxo-thiomorpholin-4-yl, morpholin-4-yl, 2-cyanoethyl, cyclopropylsulfonyl, 2-amino-3-(3,3-difluoroazetidin-1-yl)-3-oxopropyl, ethylsulfonyl, pyrimidin-5-yl, 3-methoxyazetidine-1-carbonyl, and 3,3-difluoroazetidin-1-ylsulfonyl.

16. The compound according to claim 1, wherein:
R² is selected from the group consisting of H, C₁₋₆ alkyl, cyano, halogen, heteroaryl, heterocyclyl, S(O)₂R¹¹, and C(O)NR¹²R¹³; wherein C₁-C₆ alkyl is optionally substituted with cyano;
R¹¹ is C₁-C₆ alkyl; and
R¹² and R¹³ are each independently C₁-C₆ alkyl.

17. The compound according to claim 1, wherein:
R² is selected from the group consisting of H, methyl, isopropylsulfonyl, methylsulfonyl, cyano, dimethylcarbamoyl, bromo, fluoro, pyridazin-4-yl, 1H-1,2,4-triazol-1-yl, 1,1-dioxo-thiomorpholin-4-yl, morpholin-4-yl, and 2-cyanoethyl.

18. The compound according to claim 1, wherein R³, R⁴, R⁵, and R⁶ are each independently selected from the group consisting of H, methyl, methylsulfonyl, and fluoro.

19. The compound according to claim 1, wherein:
R² is selected from the group consisting of H, C₁-C₆ alkyl, cyano, halogen, heteroaryl, heterocyclyl, S(O)₂R¹¹, and C(O)NR¹²R¹³; wherein C₁₋₆ alkyl is optionally substituted with cyano;
R³ is H or C₁₋₄ alkylsulfonyl;
R⁴ is H or halogen;
R⁵ is H, halogen, or C₁₋₆ alkyl;
R⁶ is H or halogen;
R¹¹ is C₁₋₆ alkyl; and
R¹² and R¹³ are each independently C₁₋₆ alkyl.

20. The compound according to claim 1, wherein:
R² is selected from the group consisting of H, methyl, isopropylsulfonyl, methylsulfonyl, cyano, dimethylcarbamoyl, bromo, fluoro, pyridazin-4-yl, 1H-1,2,4-triazol-1-yl, 1,1-dioxo-thiomorpholin-4-yl, morpholin-4-yl, and 2-cyanoethyl;
R³ is H or methylsulfonyl;
R⁴ is H or fluoro;
R⁵ is H, fluoro, or methyl; and
R⁶ is H or fluoro.

21. The compound according to claim 1, selected from compounds of Formula (Ii) and pharmaceutically acceptable salts, solvates, and hydrates thereof: 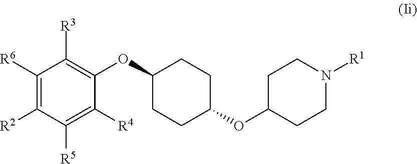
wherein:
R¹ is selected from the group consisting of S(O)₂R⁷, C(O)R⁷, CH₂R⁸, and C(O)OR⁹; or R¹ is selected from the group consisting of 1,2,4-oxadiazolyl, phenyl, pyrimidinyl, pyridinyl, pyridazinyl, and pyrazinyl, each optionally substituted with one or two substituents selected independently from the group consisting of C₁-C₄ alkoxy, C₁-C₆ alkyl, halogen, C₁-C₄ haloalkoxy, and C₁-C₆ haloalkyl;
R² is selected from the group consisting of H, C₁-C₆ alkyl, cyano, heteroaryl, and S(O)₂R¹¹; wherein said C₁-C₆ alkyl is optionally substituted with one or more substituents selected independently from the group consisting of amino, cyano, and C(O)NR¹²R¹³;
R³, R⁴, R⁵, and R⁶ are each independently selected from the group consisting of H, C₁-C₆ alkylsulfonyl, and halogen;
R⁷ is selected from the group consisting of C₁-C₆ alkyl, C₃-C₆ cycloalkyl, and C₁-C₆ haloalkyl; wherein said C₃-C₆ cycloalkyl is optionally substituted with one C₁-C₆ alkyl substituent;
R⁸ is selected from the group consisting of a five-membered heteroaryl and C(O)OR⁹; wherein said five-membered heteroaryl is optionally substituted with one C₁-C₆ alkyl substituent;
R⁹ is selected from the group consisting of C₁-C₆ alkyl, C₃-C₆ cycloalkyl, and C₁-C₆ haloalkyl; wherein said C₁-C₆ alkyl is optionally substituted with one R¹⁰ substituent; and said C₃-C₆ cycloalkyl is optionally substituted with one C₁-C₆ alkyl substituent;
R¹⁰ is heterocyclyl optionally substituted with one C₁-C₆ alkyl substituent;
R¹¹ is C₁-C₆ alkyl; and
R¹² and R¹³ together with the nitrogen to which they are both bonded form a heterocyclyl optionally substituted with one or two halogens.

22. The compound according to claim 1, selected from compounds of Formula (Ii) and pharmaceutically acceptable salts, solvates, and hydrates thereof: 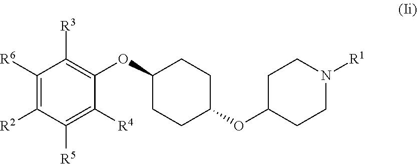
wherein:
R¹ is S(O)₂R⁷, C(O)R⁷, CH₂R⁸, or C(O)OR⁹; or R¹ is 1,2,4-oxadiazolyl, phenyl, pyrimidinyl, pyridinyl, pyridazinyl, or pyrazinyl, wherein each are optionally substituted with one or two substituents selected independently from the group consisting of C₁₋₄ alkoxy, C₁₋₆ alkyl, halogen, C₁₋₄ haloalkoxy, and C₁₋₆ haloalkyl;
R² is selected from the group consisting of H, cyano, heteroaryl, and S(O)₂R¹¹; wherein C₁₋₆ alkyl is optionally substituted with one cyano substituent;
R³, R⁴, R⁵, and R⁶ are each independently selected from the group consisting of H, C₁₋₆ alkylsulfonyl, and halogen;
R⁷ is C₁₋₆ alkyl, or C₃₋₆ cycloalkyl, or C₁₋₆ haloalkyl; wherein said C₃₋₆ cycloalkyl is optionally substituted with one C₁₋₆ alkyl substituent;
R⁸ is a five-membered heteroaryl, or C(O)OR⁹; wherein said five-membered heteroaryl is optionally substituted with one C₁₋₆ alkyl substituent;
R⁹ is C₁₋₆ alkyl, C₃₋₆ cycloalkyl, or C₁₋₆ haloalkyl; wherein said C₁₋₆ alkyl is optionally substituted with one R¹⁰ substituent; and said C₃₋₆ cycloalkyl is optionally substituted with C₁₋₆ alkyl;
R¹⁰ is heterocyclyl optionally substituted with one C₁₋₆ alkyl substituent; and
R¹¹ is C₁₋₆ alkyl.

23. The compound according to claim 1, selected from compounds of Formula (Ic) and pharmaceutically acceptable salts, solvates, and hydrates thereof: 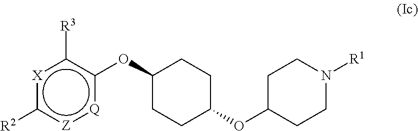
wherein:
Q is N; Z is CR⁵; and X is CR⁶; or
Q is N; Z is CR⁵; and X is N; or
Q is N; Z is N; and X is CR⁶; or
Q is CR⁴; Z is CR⁵; and X is N or N(O);
R¹ is selected from the group consisting of C(O)R⁷, CH₂R⁸, C(O)OR⁹, and C(O)SR⁹; or R¹ is selected from the group consisting of 1,2,4-oxadiazolyl, pyrimidinyl, and pyridinyl each optionally substituted with one substituent selected from the group consisting of C₂-C₆ alkenyl, C₁-C₆ alkyl, and C₁-C₆ haloalkyl;
R² is selected from the group consisting of H, cyano, halogen, heteroaryl, heterocyclyl, S(O)₂R¹¹, and C(O)NR¹²R¹³;
R³, R⁴, R⁵, and R⁶ are each independently selected from the group consisting of H and C₁-C₆ alkyl;
R⁷ is C₁-C₆ haloalkyl;
R⁸ is selected from the group consisting of C₃-C₆ cycloalkyl, C₁-C₆ haloalkyl, and a five-membered heteroaryl, wherein said C₃-C₆ cycloalkyl and said five-membered heteroaryl are each optionally substituted with one substituent selected from C₁-C₆ haloalkyl and C₁-C₆ alkyl;
R⁹ is selected from the group consisting of C₁-C₆ alkyl, C₃-C₆ cycloalkyl, C₁-C₆ haloalkyl, heterocyclyl, and phenyl; wherein said C₁-C₆ alkyl and said C₃-C₆ cycloalkyl are each optionally substituted with one or more substituents selected independently from the group consisting of C₁-C₆ alkyl, halogen, hydroxyl, C₁-C₆ alkoxy, and R¹⁰, wherein C₁-C₆ alkoxy is optionally substituted with phenyl;
R¹¹ is selected from the group consisting of C₁-C₆ alkyl and C₃-C₆ cycloalkyl; and
R¹² and R¹³ are each independently selected from the group consisting of C₁-C₆ alkyl; or R¹² and R¹³ together with the nitrogen to which they are both bonded form a heterocyclyl optionally substituted with one C₁-C₆ alkoxy substituent.

24. The compound according to claim 1, selected from compounds of Formula (Ic) and pharmaceutically acceptable salts, solvates, and hydrates thereof: 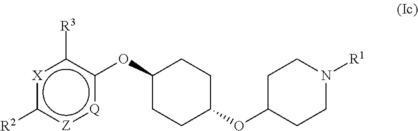
wherein:
Q is N; Z is CR⁵; and X is CR⁶; or
Q is N; Z is CR⁵; and X is N; or
Q is N; Z is N; and X is CR⁶;
R¹ is C(O)R⁷, CH₂R⁸, or C(O)OR⁹; or R¹ is 1,2,4-oxadiazolyl or pyrimidinyl, wherein each are optionally substituted with one substituent selected from the group consisting of C₂₋₆ alkenyl, C₁₋₆ alkyl, and C₁₋₆ haloalkyl;
R² is selected from the group consisting of cyano, halogen, heteroaryl, heterocyclyl, S(O)₂R¹¹, and C(O)NR¹²R¹³;
R³, R⁴, R⁵, and R⁶ are each independently selected from the group consisting of H and C₁₋₆ alkyl;
R⁷ is C₁₋₆ haloalkyl;
R⁸ is a five-membered heteroaryl optionally substituted with one C₁₋₆ alkyl substituent;
R⁹ is C₁₋₆ alkyl, C₃₋₆ cycloalkyl, C₁₋₆ haloalkyl, or heterocyclyl; wherein said C₁₋₆ alkyl is optionally substituted with hydroxyl; and said C₃₋₆ cycloalkyl is optionally substituted with one C₁₋₆ alkyl substituent;
R¹¹ is C₁₋₆ alkyl; and
R¹² and R¹³ are each independently C₁₋₆ alkyl.

25. The compound according to claim 1, selected from compounds of Formula (IIe) and pharmaceutically acceptable salts, solvates, and hydrates thereof: 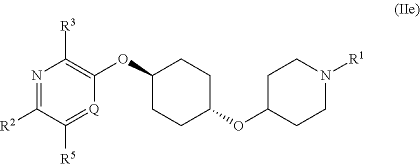
wherein:
Q is N or CR⁴;
R¹ is selected from the group consisting of C(O)R⁷ and C(O)OR⁹; or R¹ is 1,2,4-oxadiazolyl optionally substituted with one substituent selected from the group consisting of C₂-C₆ alkenyl, C₁-C₆ alkyl, and C₁-C₆ haloalkyl;
R² is selected from the group consisting of H, cyano, heteroaryl, heterocyclyl, S(O)₂R¹¹, and C(O)NR¹²R¹³;
R³, R⁴, and R⁵ are each independently selected from the group consisting of H and C₁-C₆ alkyl;
R⁷ is C₁-C₆ haloalkyl;
R⁹ is selected from the group consisting of C₁-C₆ alkyl, C₃-C₆ cycloalkyl, C₁-C₆ haloalkyl, heterocyclyl, and phenyl; wherein said C₁-C₆ alkyl is optionally substituted with one or more substituents selected independently from the group consisting of hydroxyl and C₁-C₆ alkoxy, wherein C₁-C₆ alkoxy is optionally substituted with phenyl; and said C₃-C₆ cycloalkyl is optionally substituted with one or more substituents selected independently from the group consisting of C₁-C₆ alkyl and halogen;
R¹¹ is selected from the group consisting of C₁-C₆ alkyl and C₃-C₆ cycloalkyl; and
R¹² and R¹³ are each independently C₁-C₆ alkyl; or R¹² and R¹³ together with the nitrogen to which they are both bonded form a heterocyclyl group optionally substituted with one C₁-C₆ alkoxy substituent.

26. The compound according to claim 1, selected from compounds of Formula (IIe) and pharmaceutically acceptable salts, solvates, and hydrates thereof: 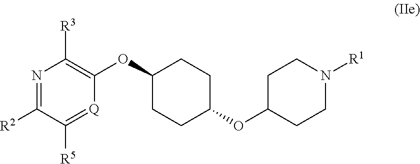
wherein:
Q is N or CR⁴;
R¹ is selected from the group consisting of C(O)R⁷ and C(O)OR⁹; or R¹ is 1,2,4-oxadiazolyl optionally substituted with one substituent selected from the group consisting of prop-1-en-2-yl, 2-fluoropropan-2-yl, and isopropyl;
R² is selected from the group consisting of H, isopropylsulfonyl, methylsulfonyl, cyano, pyridazin-4-yl, 1H-1,2,4-triazol-1-yl, 1,1-dioxo-thiomorpholin-4-yl, morpholin-4-yl, cyclopropylsulfonyl, bromo, dimethylcarbamoyl, ethylsulfonyl, pyrimidin-5-yl, and 3-methoxyazetidine-1-carbonyl;
R³, R⁴, and R⁵ are each independently selected from the group consisting of H and methyl;
R⁷ is 1,1-difluoropropyl; and
R⁹ is selected from the group consisting of isopropyl, tert-butyl, 1-methylcyclopropyl, 1,3-difluoropropan-2-yl, 1-fluoropropan-2-yl, tetrahydrofuran-3-yl, 1-hydroxypropan-2-yl, phenyl, 2,2,3,3-tetrafluorocyclobutyl, 1,1,1,3,3,3-hexafluoropropan-2-yl, 1,1,1-trifluoropropan-2-yl, 1-(benzyloxy)propan-2-yl, and 1-hydroxypropan-2-yl.

27. The compound according to claim 1, selected from compounds of Formula (Is) and pharmaceutically acceptable salts, solvates, and hydrates thereof: 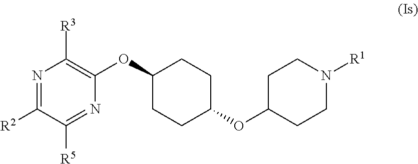
wherein:
R¹ is C(O)R⁷ or C(O)OR⁹; or R¹ is 1,2,4-oxadiazolyl optionally substituted with one substituent selected from the group consisting of C₂₋₆ alkenyl, and C₁₋₆ haloalkyl;
R² is selected from the group consisting of cyano, heteroaryl, heterocyclyl, and S(O)₂R¹¹;
R³ and R⁵ are each H;
R⁷ is C₁₋₆ haloalkyl;
R⁹ is C₁₋₆ alkyl, C₃₋₆ cycloalkyl, C₁₋₆ haloalkyl, or heterocyclyl; wherein said C₁₋₆ alkyl is optionally substituted with hydroxyl; and said C₃₋₆ cycloalkyl is optionally substituted with one C₁₋₆ alkyl substituent; and
R¹¹ is C₁₋₆ alkyl.

28. A compound according to claim 1 selected from the following compounds and pharmaceutically acceptable salts, solvates, and hydrates thereof:
tert-butyl 4-((1s,4s)-4-(4-(methylsulfonyl)phenoxy)cyclohexyloxy)piperidine-1-carboxylate;
tert-butyl 4-((1r,4r)-4-(4-(methylsulfonyl)phenoxy)cyclohexyloxy)piperidine-1-carboxylate;
3-isopropyl-5-(4-((1s,4s)-4-(4-(methylsulfonyl)phenoxy)cyclohexyloxy)piperidin-1-yl)-1,2,4-oxadiazole;
3-isopropyl-5-(4-((1r,4r)-4-(4-(methylsulfonyl)phenoxy)cyclohexyloxy)piperidin-1-yl)-1,2,4-oxadiazole;
tert-butyl 4-((1r,4r)-4-(2-fluoro-4-(methylsulfonyl)phenoxy)cyclohexyloxy)piperidine-1-carboxylate;
tert-butyl 4-((1s,4s)-4-(2-fluoro-4-(methylsulfonyl)phenoxy)cyclohexyloxy)piperidine-1-carboxylate;
tert-butyl 4-((1r,4r)-4-(5-(methylsulfonyl)pyridin-2-yloxy)cyclohexyloxy)piperidine-1-carboxylate;
5-ethyl-2-(4-((1r,4r)-4-(2-fluoro-4-(methylsulfonyl)phenoxy)cyclohexyloxy)piperidin-1-yl)pyrimidine;
5-ethyl-2-(4-((1r,4r)-4-(5-(methylsulfonyl)pyridin-2-yloxy)cyclohexyloxy)piperidin-1-yl)pyrimidine;
5-ethyl-2-(4-((1s,4s)-4-(2-fluoro-4-(methylsulfonyl)phenoxy)cyclohexyloxy)piperidin-1-yl)pyrimidine;
tert-butyl 4-((1s,4s)-4-(5-(methylsulfonyl)pyridin-2-yloxy)cyclohexyloxy)piperidine-1-carboxylate;
isopropyl 4-((1r,4r)-4-(5-(methylsulfonyl)pyridin-2-yloxy)cyclohexyloxy)piperidine-1-carboxylate;
isopropyl 4-((1r,4r)-4-(2-fluoro-4-(methylsulfonyl)phenoxy)cyclohexyloxy)piperidine-1-carboxylate;
5-((4-((1r,4r)-4-(2-fluoro-4-(methylsulfonyl)phenoxy)cyclohexyloxy)piperidin-1-yl)methyl)-3-isopropyl-1,2,4-oxadiazole;
3-isopropyl-5-((4-((1r,4r)-4-(5-(methylsulfonyl)pyridin-2-yloxy)cyclohexyloxy)piperidin-1-yl)methyl)-1,2,4-oxadiazole;
5-(4-((1r,4r)-4-(2-fluoro-4-(methylsulfonyl)phenoxy)cyclohexyloxy)piperidin-1-yl)-3-(2-fluoropropan-2-yl)-1,2,4-oxadiazole;
tert-butyl 4-((1r,4r)-4-(5-(methylsulfonyl)pyrazin-2-yloxy)cyclohexyloxy)piperidine-1-carboxylate;
3-(2-fluoropropan-2-yl)-5-(4-((1r,4r)-4-(5-(methylsulfonyl)pyridin-2-yloxy)cyclohexyloxy)piperidin-1-yl)-1,2,4-oxadiazole;
tert-butyl 4-((1r,4r)-4-(5-(dimethylcarbamoyl)-6-methylpyridin-2-yloxy)cyclohexyloxy)piperidine-1-carboxylate;
tert-butyl 4-((1r,4r)-4-(6-bromopyridazin-3-yloxy)cyclohexyloxy)piperidine-1-carboxylate;
tert-butyl 4-((1r,4r)-4-(3-fluoro-4-(methylsulfonyl)phenoxy)cyclohexyloxy)piperidine-1-carboxylate;
tert-butyl 4-((1r,4r)-4-(5-fluoro-2-(methylsulfonyl)phenoxy)cyclohexyloxy)piperidine-1-carboxylate;
tert-butyl 4-((1r,4r)-4-(4-cyanophenoxy)cyclohexyloxy)piperidine-1-carboxylate;
tert-butyl 4-((1r,4r)-4-(6-(methylsulfonyl)pyridazin-3-yloxy)cyclohexyloxy)piperidine-1-carboxylate;
3-tert-butyl-5-(4-((1r,4r)-4-(2-fluoro-4-(methylsulfonyl)phenoxy)cyclohexyloxy)piperidin-1-yl)-1,2,4-oxadiazole;
3-tert-butyl-5-(4-((1r,4r)-4-(4-(methylsulfonyl)phenoxy)cyclohexyloxy)piperidin-1-yl)-1,2,4-oxadiazole;
3-(2-fluoropropan-2-yl)-5-(4-((1r,4r)-4-(4-(methylsulfonyl)phenoxy)cyclohexyloxy)piperidin-1-yl)-1,2,4-oxadiazole;
isopropyl 4-((1r,4r)-4-(5-(methylsulfonyl)pyrazin-2-yloxy)cyclohexyloxy)piperidine-1-carboxylate;
tert-butyl 4-((1r,4r)-4-(5-(1H-1,2,4-triazol-1-yl)pyrazin-2-yloxy)cyclohexyloxy)piperidine-1-carboxylate;
3-(2-fluoropropan-2-yl)-5-(4-((1r,4r)-4-(5-(methylsulfonyl)pyrazin-2-yloxy)cyclohexyloxy)piperidin-1-yl)-1,2,4-oxadiazole;
tert-butyl 4-((1r,4r)-4-(4-(2-cyanoethyl)phenoxy)cyclohexyloxy)piperidine-1-carboxylate;
3,3-dimethyl-1-(4-((1r,4r)-4-(4-(methylsulfonyl)phenoxy)cyclohexyloxy)piperidin-1-yl)butan-1-one;
isobutyl 4-((1r,4r)-4-(4-(methylsulfonyl)phenoxy)cyclohexyloxy)piperidine-1-carboxylate;
5-chloro-2-(4-((1r,4r)-4-(4-(methylsulfonyl)phenoxy)cyclohexyloxy)piperidin-1-yl)pyrimidine;
tert-butyl 2-(4-((1r,4r)-4-(4-(methylsulfonyl)phenoxy)cyclohexyloxy)piperidin-1-yl)acetate;
cyclopentyl 4-((1r,4r)-4-(4-(methylsulfonyl)phenoxy)cyclohexyloxy)piperidine-1-carboxylate;
5-isopropyl-3-(4-((1r,4r)-4-(4-(methylsulfonyl)phenoxy)cyclohexyloxy)piperidin-1-yl)-1,2,4-oxadiazole;
5-methyl-2-(4-((1r,4r)-4-(4-(methylsulfonyl)phenoxy)cyclohexyloxy)piperidin-1-yl)pyrimidine;
2-(4-((1r,4r)-4-(4-(methylsulfonyl)phenoxy)cyclohexyloxy)piperidin-1-yl)-5-(trifluoromethyl)pyridine;
2-methyl-1-(4-((1r,4r)-4-(4-(methylsulfonyl)phenoxy)cyclohexyloxy)piperidin-1-yl)propan-1-one;
3-methyl-6-(4-((1r,4r)-4-(4-(methylsulfonyl)phenoxy)cyclohexyloxy)piperidin-1-yl)pyridazine;
1,1,1,3,3,3-hexafluoropropan-2-yl 4-((1r,4r)-4-(4-(methylsulfonyl)phenoxy)cyclohexyloxy)piperidine-1-carboxylate;
(3-methyloxetan-3-yl)methyl-4-((1r,4r)-4-(4-(methylsulfonyl)phenoxy)cyclohexyloxy)piperidine-1-carboxylate;
2-methyl-5-(4-((1r,4r)-4-(4-(methylsulfonyl)phenoxy)cyclohexyloxy)piperidin-1-yl)pyrazine;
(2-methylcyclopropyl)(4-((1r,4r)-4-(4-(methylsulfonyl)phenoxy)cyclohexyloxy)piperidin-1-yl)methanone;
1-methylcyclopropyl-4-((1r,4r)-4-(4-(methylsulfonyl)phenoxy)cyclohexyloxy)piperidine-1-carboxylate;
tert-butyl 4-((1r,4r)-4-(4-(1H-1,2,4-triazol-1-yl)phenoxy)cyclohexyloxy)piperidine-1-carboxylate;
2,2-difluoro-1-(4-((1r,4r)-4-(4-(methylsulfonyl)phenoxy)cyclohexyloxy)piperidin-1-yl)butan-1-one;
5-chloro-2-(4-((1r,4r)-4-(4-(methylsulfonyl)phenoxy)cyclohexyloxy)piperidin-1-yl)pyridine;
1-(cyclopropylsulfonyl)-4-(1r,4r)-4-(4-(methylsulfonyl)phenoxy)cyclohexyloxy)piperidine;
3-ethoxy-6-(4-((1r,4r)-4-(4-(methylsulfonyl)phenoxy)cyclohexyloxy)piperidin-1-yl)pyridazine;
5-fluoro-2-(4-((1r,4r)-4-(4-(methylsulfonyl)phenoxy)cyclohexyloxy)piperidin-1-yl)pyridine;
(R)-sec-butyl 4-((1r,4r)-4-(4-(methylsulfonyl)phenoxy)cyclohexyloxy)piperidine-1-carboxylate;
(S)-sec-butyl 4-((1r,4r)-4-(4-(methylsulfonyl)phenoxy)cyclohexyloxy)piperidine-1-carboxylate;
1-(isopropylsulfonyl)-4-(1r,4r)-4-(4-(methylsulfonyl)phenoxy)cyclohexyloxy)piperidine;
(S)-tetrahydrofuran-3-yl-4-((1r,4r)-4-(5-(methylsulfonyl)pyrazin-2-yloxy)cyclohexyloxy)piperidine-1-carboxylate;
4-((1r,4r)-4-(4-(methylsulfonyl)phenoxy)cyclohexyloxy)-1-p-tolylpiperidine;
4-((1r,4r)-4-(4-(methylsulfonyl)phenoxy)cyclohexyloxy)-1-(4-(trifluoromethyl)phenyl)piperidine;
4-((1r,4r)-4-(4-(methylsulfonyl)phenoxy)cyclohexyloxy)-1-(4-(trifluoromethoxy)phenyl)piperidine;
1-(4-methoxyphenyl)-4-((1r,4r)-4-(4-(methylsulfonyl)phenoxy)cyclohexyloxy)piperidine;
4-((1r,4r)-4-(4-(methylsulfonyl)phenoxy)cyclohexyloxy)-1-(3-(trifluoromethyl)phenyl)piperidine;
5-(2-fluoropropan-2-yl)-3-(4-((1r,4r)-4-(5-(methylsulfonyl)pyrazin-2-yloxy)cyclohexyloxy)piperidin-1-yl)-1,2,4-oxadiazole;
1,1,1-trifluoropropan-2-yl 4-((1r,4r)-4-(4-(methylsulfonyl)phenoxy)cyclohexyloxy)piperidine-1-carboxylate;
1-methylcyclopropyl 4-((1r,4r)-4-(5-(methylsulfonyl)pyrazin-2-yloxy)cyclohexyloxy)piperidine-1-carboxylate;
isopropyl 4-((1r,4r)-4-(5-(1H-1,2,4-triazol-1-yl)pyrazin-2-yloxy)cyclohexyloxy)piperidine-1-carboxylate;
1-(4-fluorophenyl)-4-((1r,4r)-4-(4-(methylsulfonyl)phenoxy)cyclohexyloxy)piperidine;
1-(4-chloro-2-fluorophenyl)-4-((1r,4r)-4-(4-(methylsulfonyl)phenoxy)cyclohexyloxy)piperidine;
tert-butyl 4-((1r,4r)-4-(5-cyanopyrazin-2-yloxy)cyclohexyloxy)piperidine-1-carboxylate;
5-(4-((1r,4r)-4-(5-(methylsulfonyl)pyrazin-2-yloxy)cyclohexyloxy)piperidin-1-yl)-3-(prop-1-en-2-yl)-1,2,4-oxadiazole;
5-methoxy-2-(4-((1r,4r)-4-(4-(methylsulfonyl)phenoxy)cyclohexyloxy)piperidin-1-yl)pyrimidine;
1,3-difluoropropan-2-yl 4-((1r,4r)-4-(5-(methylsulfonyl)pyrazin-2-yloxy)cyclohexyloxy)piperidine-1-carboxylate;
tert-butyl 4-((1r,4r)-4-(5-(isopropylsulfonyl)pyrazin-2-yloxy)cyclohexyloxy)piperidine-1-carboxylate;
2,2-difluoro-1-(4-((1r,4r)-4-(5-(methylsulfonyl)pyrazin-2-yloxy)cyclohexyloxy)piperidin-1-yl)butan-1-one;
(S)-1-fluoropropan-2-yl 4-((1r,4r)-4-(5-(methylsulfonyl)pyrazin-2-yloxy)cyclohexyloxy)piperidine-1-carboxylate;
(R)-1-fluoropropan-2-yl 4-((1r,4r)-4-(5-(methylsulfonyl)pyrazin-2-yloxy)cyclohexyloxy)piperidine-1-carboxylate;
(S)-1-hydroxypropan-2-yl 4-((1r,4r)-4-(5-(methylsulfonyl)pyrazin-2-yloxy)cyclohexyloxy)piperidine-1-carboxylate;

29. A compound according to claim 1 selected from the following compounds and pharmaceutically acceptable salts, solvates, and hydrates thereof:
isopropyl 4-((1r,4r)-4-(5-(pyridazin-4-yl)pyrazin-2-yloxy)cyclohexyloxy)-piperidine-1-carboxylate;
isopropyl 4-((1r,4r)-4-(5-(1,1-dioxo-thiomorpholin-4-yl)pyrazin-2-yloxy)cyclohexyloxy)piperidine-1-carboxylate;
isopropyl 4-((1r,4r)-4-(5-morpholinopyrazin-2-yloxy)cyclohexyloxy)piperidine-1-carboxylate;
1,1,1-trifluoropropan-2-yl 4-((1r,4r)-4-(5-(methylsulfonyl)pyrazin-2-yloxy)cyclohexyloxy)piperidine-1-carboxylate;
1-fluoropropan-2-yl 4-((1r,4r)-4-(5-(methylsulfonyl)pyrazin-2-yloxy)cyclohexyloxy)piperidine-1-carboxylate;
(S)-1,1,1-trifluoropropan-2-yl 4-((1r,4r)-4-(5-(methylsulfonyl)pyrazin-2-yloxy)cyclohexyloxy)piperidine-1-carboxylate;
(R)-1,1,1-trifluoropropan-2-yl 4-((1r,4r)-4-(5-(methylsulfonyl)pyrazin-2-yloxy)cyclohexyloxy)piperidine-1-carboxylate;
isopropyl 4-((1r,4r)-4-(2-methylpyridin-3-yloxy)cyclohexyloxy)piperidine-1-carboxylate;
isopropyl 4-((1r,4r)-4-(6-(cyclopropylsulfonyl)-2-methylpyridin-3-yloxy)cyclohexyloxy)piperidine-1-carboxylate;
phenyl 4-((1r,4r)-4-(5-(methylsulfonyl)pyrazin-2-yloxy)cyclohexyloxy)piperidine-1-carboxylate;
5-isopropyl-3-(4-((1r,4r)-4-(5-(methylsulfonyl)pyrazin-2-yloxy)cyclohexyloxy)piperidin-1-yl)-1,2,4-oxadiazole;
3-isopropyl-5-(4-((1r,4r)-4-(5-(methylsulfonyl)pyrazin-2-yloxy)cyclohexyloxy)piperidin-1-yl)-1,2,4-oxadiazole;
3-(4-((1r,4r)-4-(5-(1H-1,2,4-triazol-1-yl)pyrazin-2-yloxy)cyclohexyloxy)piperidin-1-yl)-5-(2-fluoropropan-2-yl)-1,2,4-oxadiazole;
2,2,3,3-tetrafluorocyclobutyl 4-((1r,4r)-4-(5-(methylsulfonyl)pyrazin-2-yloxy)cyclohexyloxy)piperidine-1-carboxylate;
1,1,1,3,3,3-hexafluoropropan-2-yl 4-((1r,4r)-4-(5-(methylsulfonyl)pyrazin-2-yloxy)cyclohexyloxy)piperidine-1-carboxylate;
isopropyl 4-((1r,4r)-4-(6-bromopyridin-3-yloxy)cyclohexyloxy)piperidine-1-carboxylate;
isopropyl 4-((1r,4r)-4-(6-(methylsulfonyl)pyridin-3-yloxy)cyclohexyloxy)piperidine-1-carboxylate;
isopropyl 4-((1S,4r)-4-(4-((S)-2-amino-3-(3,3-difluoroazetidin-1-yl)-3-oxopropyl)phenoxy)cyclohexyloxy)piperidine-1-carboxylate;
isopropyl 4-((1r,4r)-4-(6-(1H-1,2,4-triazol-1-yl)pyridin-3-yloxy)cyclohexyloxy)piperidine-1-carboxylate;
5-(4-((1r,4r)-4-(5-(1H-1,2,4-triazol-1-yl)pyrazin-2-yloxy)cyclohexyloxy)piperidin-1-yl)-3-isopropyl-1,2,4-oxadiazole;
isopropyl 4-((1R,4r)-4-(4-((R)-2-amino-3-(3,3-difluoroazetidin-1-yl)-3-oxopropyl)phenoxy)cyclohexyloxy)piperidine-1-carboxylate;
5-((1r,4r)-4-(1-(3-isopropyl-1,2,4-oxadiazol-5-yl)piperidin-4-yloxy)cyclohexyloxy)-N,N-dimethylpyrazine-2-carboxamide;
(R)-1,1,1-trifluoropropan-2-yl 4-((1r,4r)-4-(5-(ethylsulfonyl)pyrazin-2-yloxy)cyclohexyloxy)piperidine-1-carboxylate;
isopropyl 4-((1r,4r)-4-(6-bromo-2-methylpyridin-3-yloxy)cyclohexyloxy)piperidine-1-carboxylate;
isopropyl 4-((1r,4r)-4-(2-methyl-6-(methylsulfonyl)pyridin-3-yloxy)cyclohexyloxy)piperidine-1-carboxylate;
isopropyl 4-((1r,4r)-4-(2-methyl-6-(pyrimidin-5-yl)pyridin-3-yloxy)cyclohexyloxy)piperidine-1-carboxylate;
(5-((1r,4r)-4-(1-(3-isopropyl-1,2,4-oxadiazol-5-yl)piperidin-4-yloxy)cyclohexyloxy)pyrazin-2-yl)(3-methoxyazetidin-1-yl)methanone;
isopropyl 4-((1r,4r)-4-(6-(ethylsulfonyl)-2-methylpyridin-3-yloxy)cyclohexyloxy)piperidine-1-carboxylate;
3-isopropyl-5-(4-((1r,4r)-4-(2-methyl-6-(methylsulfonyl)pyridin-3-yloxy)cyclohexyloxy)piperidin-1-yl)-1,2,4-oxadiazole;
isopropyl 4-((1r,4r)-4-(6-cyano-2-methylpyridin-3-yloxy)cyclohexyloxy)piperidine-1-carboxylate;
(S)-1-(benzyloxy)propan-2-yl 4-((1r,4r)-4-(2-methyl-6-(methylsulfonyl)pyridin-3-yloxy)cyclohexyloxy)piperidine-1-carboxylate;
(S)-1-hydroxypropan-2-yl 4-((1r,4r)-4-(2-methyl-6-(methylsulfonyl)pyridin-3-yloxy)cyclohexyloxy)piperidine-1-carboxylate;
(R)-1,1,1-trifluoropropan-2-yl 4-((1r,4r)-4-(2-methyl-6-(methylsulfonyl)pyridin-3-yloxy)cyclohexyloxy)piperidine-1-carboxylate; and
(S)-1,1,1-trifluoropropan-2-yl 4-((1r,4r)-4-(2-methyl-6-(methylsulfonyl)pyridin-3-yloxy)cyclohexyloxy)piperidine-1-carboxylate.

30. A compound according to claim 1 selected from the following compounds and pharmaceutically acceptable salts, solvates, and hydrates thereof:
isopropyl 4-((1r,4r)-4-(3-methyl-5-(methylsulfonyl)pyrazin-2-yloxy)cyclohexyloxy)piperidine-1-carboxylate;
(R)-1,1,1-trifluoropropan-2-yl 4-((1r,4r)-4-(3-methyl-5-(methylsulfonyl)pyrazin-2-yloxy)cyclohexyloxy)piperidine-1-carboxylate;
isopropyl 4-((1r,4r)-4-(4-(3,3-difluoroazetidin-1-ylsulfonyl)phenoxy)cyclohexyloxy)piperidine-1-carboxylate;
5-ethyl-2-(4-((1r,4r)-4-(2-methyl-6-(methylsulfonyl)pyridin-3-yloxy)cyclohexyloxy)piperidin-1-yl)pyrimidine;
5-methyl-2-(4-((1r,4r)-4-(2-methyl-6-(methylsulfonyl)pyridin-3-yloxy)cyclohexyloxy)piperidin-1-yl)pyrimidine;
S-isopropyl 4-((1r,4r)-4-(2-methyl-6-(methylsulfonyl)pyridin-3-yloxy)cyclohexyloxy)piperidine-1-carbothioate;
2-methyl-3-((1r,4r)-4-(1-(5-methylpyridin-2-yl)piperidin-4-yloxy)cyclohexyloxy)-6-(methylsulfonyl)pyridine;
3-((1r,4r)-4-(1-(5-ethylpyridin-2-yl)piperidin-4-yloxy)cyclohexyloxy)-2-methyl-6-(methylsulfonyl)pyridine;
2-methyl-6-(methylsulfonyl)-3-((1r,4r)-4-(1-(((R)-1,1,1-trifluoropropan-2-yloxy)carbonyl)piperidin-4-yloxy)cyclohexyloxy)pyridine 1-oxide;
isopropyl 4-((1r,4r)-4-(4-methyl-6-(methylsulfonyl)pyridin-3-yloxy)cyclohexyloxy)piperidine-1-carboxylate;
isopropyl 4-((1r,4r)-4-(5-methyl-6-(methylsulfonyl)pyridin-3-yloxy)cyclohexyloxy)piperidine-1-carboxylate;
1,1,1-trifluoro-2-methylpropan-2-yl-4-((1r,4r)-4-(2-methyl-6-(methylsulfonyl)pyridin-3-yloxy)cyclohexyloxy)piperidine-1-carboxylate;
1-methylcyclopropyl 4-((1r,4r)-4-(2-methyl-6-(methylsulfonyl)pyridin-3-yloxy)cyclohexyloxy)piperidine-1-carboxylate;
S-cyclopropyl 4-((1r,4r)-4-(2-methyl-6-(methylsulfonyl)pyridin-3-yloxy)cyclohexyloxy)piperidine-1-carbothioate;
2-(methylsulfonyl)-5-((1r,4r)-4-(1-((1-(trifluoromethyl)cyclopropyl)methyl)piperidin-4-yloxy)cyclohexyloxy)pyrazine;
2-(methylsulfonyl)-5-((1r,4r)-4-(1-(2,2,3,3-tetrafluoropropyl)piperidin-4-yloxy)cyclohexyloxy)pyrazine;
2-(methylsulfonyl)-5-((1r,4r)-4-(1-((1-(trifluoromethyl)cyclobutyl)methyl)piperidin-4-yloxy)cyclohexyloxy)pyrazine; and
2-(methylsulfonyl)-5-((1r,4r)-4-(1-(2,2,2-trifluoroethyl)piperidin-4-yloxy)cyclohexyloxy)pyrazine.

31. A composition comprising a compound according to claim 1.

32. A composition comprising a compound according to claim 1 and a pharmaceutically acceptable carrier.

33. A method for preparing a composition comprising the step of admixing a compound according to claim 1 and a pharmaceutically acceptable carrier.

34. A method for preparing a composition comprising the step of admixing a compound according to claim 1, a second pharmaceutical agent, and a pharmaceutically acceptable carrier, wherein said second pharmaceutical agent is selected from the group consisting of: an inhibitor of DPP-IV, a biguanide, an alpha-glucosidase inhibitor, a sulfonylurea, a SGLT2 inhibitor, and a meglitinide.

35. A pharmaceutical product selected from the group consisting of: a pharmaceutical composition, a formulation, a dosage form, a combined preparation, a twin pack, and a kit; comprising a compound according to claim 1 and a second pharmaceutical agent selected from the group consisting of: an inhibitor of DPP-IV, a biguanide, an alpha-glucosidase inhibitor, a sulfonylurea, a SGLT2 inhibitor, and a meglitinide.

36. A method for the treatment of a disorder selected from the group consisting of: a GPR119-receptor-related disorder; a condition ameliorated by increasing a blood incretin level; a condition characterized by low bone mass; a neurological disorder; a metabolic-related disorder; type 2 diabetes; and obesity; in an individual; comprising administering to said individual in need thereof, a therapeutically effective amount of: a compound according to claim 1.

37-40. (canceled)

41. A method for the treatment of a disorder selected from the group consisting of: a GPR119-receptor-related disorder; a condition ameliorated by increasing a blood incretin level; a condition characterized by low bone mass; a neurological disorder; a metabolic-related disorder; type 2 diabetes; and obesity; in an individual; comprising administering to said individual in need thereof, a therapeutically effective amount of a compound according to claim 1 in combination with a therapeutically effective amount of a second pharmaceutical agent; wherein said second pharmaceutical agent is selected from: an inhibitor of DPP-IV, a biguanide, an alpha-glucosidase inhibitor, a sulfonylurea, a SGLT2 inhibitor, and a meglitinide.

42-49. (canceled)

50. The method according to claim 34; wherein said second pharmaceutical agent is selected from the group consisting of:
A. an inhibitor of DPP-IV selected from the following inhibitors of DPP-IV and pharmaceutically acceptable salts, solvates, and hydrates thereof:
3 (R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3 (R)-aminopiperidin-1-yl]-3-methyl-2,4-dioxo-1,2,3,4-tetrahydropyrimidin-1-ylmethyl]benzonitrile;
8-[3 (R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-yl boronic acid;
4(S)-fluoro-1-[2-[(1R,3S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5difluoropiperidin-2-one;
(R)-2-((6-(3-aminopiperidin-1-yl)-3-methyl-2,4-dioxo-3,4-dihydropyrimidin-1 (2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine;
B. a biguanide selected from the following biguanides, and pharmaceutically acceptable salts, solvates, and hydrates thereof:
(phenylethyl)biguanide;
dimethylbiguanide;
butylbiguanide; and
1-(p-chlorophenyl)-5-isopropylbiguanide;
C. an alpha-glucosidase inhibitor selected from the following alpha-glucosidase inhibitors, and pharmaceutically acceptable salts, solvates, and hydrates thereof:
(2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;
3R,4R,5S)-1-(2-hydroxyethyl)-2-(hydroxymethyl)piperidine-3,4,5-triol; and
2S,3R,4S,5S)-5-(1,3-dihydroxypropan-2-ylamino)-1-(hydroxymethyl)cyclohexane-1,2,3,4-tetraol;
D. a sulfonylurea selected from the following sulfonylureas, and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide;
E. a SGLT2 inhibitor selected from the following SGLT2 inhibitors, and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl((2R,3S,4S,5R,6S)-3,4,5-trihydroxy-6-(2-(4-methoxybenzyl)phenoxy)tetrahydro-2H-pyran-2-yl)methyl carbonate; and
F. a meglitinide selected from the following meglitinides, and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-((3aR,7aS)-1H-isoindol-2(3H,3aH,4H,5H,6H,7H,7aH)-yl)-4-oxobutanoic acid.
